SEQUENCE LISTING (156 sequences)

SEQ ID NO: 1 - DNA, 1407 bp, Unknown
  Obtained from environmental sample
atgactgtctatcaactcttgtttacggccgctttggctggtacagcacttgctgcccctcttgtcgaggaacgccaggcttgcgccagccagtgggcccagtgtggtggcttcagctggaatggtgctacttgctgccagtctggtagttactgtagcaagatcaatgactattactctcagtgtattcctggagagggtcccgccacttccaagacaagcacgcttcctgcttctaccaccaccagcaagccgacttccacttccactgctggtacttcttccactacgaagcctccacctgctggaagcggcactgccacttatagcggaaacccctactctggtgttaacctttgggccaacagctactatcgctcagaggttaccaacttggccatccccaagttgagcggtgccatggccacggctgctgccaaggtcgctgatgttccctcttatcagtggatggactctttcgagcacatctccctgatggaggatactcttgttgacattcgaaaggccaaccaggctggtggtaactacgccggccagtttgtcgtctatgatctccctgatcgtgactgcgctgctgccgcttccaacggagagtattcccttgacaaggatggtgccaacaagtacaagaactacatcaacactatcaagaagatcatccagagctactctgatatccgaatcctccttgttattgagcctgactccctggctaacctggtcaccaacatggatgttgccaagtgcgccaaggcccatgatgcgtacatcagcctgacgaactacgctgtcacggaactgaacctacccaacgtcgccatgtatcttgatgcaggccacgctggctggctcggctggcccaacaaccaaggccctgctgcgaagctctttgctagcatctacaaggatgccggcaagccagctgcgctccgtggactcgccaccaacgttgctaactacaacgcctggagcctcagcagtgctcccccttatacccaaggcgcctccatctacgacgagaagagtttcattcacgcaatgggtcctctcctggagcagaatggctggcctggcgctcacttcattaccgaccagggccgttctggcaagcagcccaccggccagatccagtggggtgactggtgcaactccaaaggcactggctttggtatccgtccctctgccaacactggtgacagcctcctcgatgcttttgtctgggtcaagcctggtggtgagtctgatggtacctcggacacgagtgctacccgttacgactaccactgcggtgcttctgccgctcttcagccggcacctgaggcaggaacctggttccaggcctacttcgagcagcttcttaccaatgccaacccttcgttcctgtaa

SEQ ID NO: 2 - protein, 468 aa, Unknown
  Obtained from environmental sample
MTVYQLLFTAALAGTALAAPLVEERQACASQWAQCGGFSWNGATCCQSGSYCSKINDYYSQCIPGEGPATSKTSTLPASTTTSKPTSTSTAGTSSTTKPPPAGSGTATYSGNPYSGVNLWANSYYRSEVTNLAIPKLSGAMATAAAKVADVPSYQWMDSFEHISLMEDTLVDIRKANQAGGNYAGQFVVYDLPDRDCAAAASNGEYSLDKDGANKYKNYINTIKKIIQSYSDIRILLVIEPDSLANLVTNMDVAKCAKAHDAYISLTNYAVTELNLPNVAMYLDAGHAGWLGWPNNQGPAAKLFASIYKDAGKPAALRGLATNVANYNAWSLSSAPPYTQGASIYDEKSFIHAMGPLLEQNGWPGAHFITDQGRSGKQPTGQIQWGDWCNSKGTGFGIRPSANTGDSLLDAFVWVKPGGESDGTSDTSATRYDYHCGASAALQPAPEAGTWFQAYFEQLLTNANPSFL

SEQ ID NO: 3 - protein, 470 aa, Unknown
  Obtained from environmental sample.
MRYTWSVAAALLPCAIQAQQTLYGQCGGQGYSGLTSCVAGATCSTVNEYYAQCTPAAGSATSTTLKTTTTTAGATTTTTSKTSASQTSTTKTSTSTASTTTATTTASASGNPFSGYQLYVNPYYSSEVASLAIPSLTGTLSSLQAAATAAAKVPSFVWLDVAAKVPTMATYLADIKAQNAAGANPPVAGQFVVYDLPDRDCAALASNGEYSIANNGVANYKAYIDSIRKVLVQYSDVHTILVIEPDSLANLVTNLNVAKCANAQSAYLECTNYALEQLNLPNVAMYLDAGHAGWLGWPANQQPAANLYASVYKNASSPAAVRGLATNVANYNAFTIASCPSYTQGNSVCDEQQYINAIAPLLSAQGFNAHFIVDTGRNGKQPTGQQAWGDWCNVINTGFGVRPTTNTGDALVDAFVWVKPGGESDGTSDSSATRYDAHCGYSDALQPAPEAGTWFQAYFVQLLSNANPAF

SEQ ID NO: 4 - protein, 472 aa, Unknown
  Obtained from environmental sample
MVVGILATLATLATLAASVPLEERQSCSSVWGQCGGQNWAGPFCCASGSTCVYSNDYYSQCLPGTASSSSSTRASSTTSRVSSATSTRSSSSTPPPASSTTPAPPVGSGTATYSGNPFAGVTPWANSFYASEVSTLAIPSLTGAMATAAAAVAKVPSFMWLDTLDKTPLMSSTLSDIRAANKAGGNYAGQFVVYDLPDRDCAAAASNGEYSIADGGVAKYKNYIDTIRGIVTTFSDVRILLVIEPDSLANLVTNLATPKCSNAQSAYLECINYAITQLNLPNVAMYLDAGHAGWLGWPANQDPAAQLFANVYKNASSPRAVRGLATNVANYNAWNITTPPSYTQGNAVYNEKLYIHALGPLLANHGWSNAFFITDQGRSGKQPTGQLEWGNWCNAVGTGFGIRPSANTGDSLLDSFVWIKPGGECDGTSNSSAPRFDYHCASADALQPAPQAGSWFQAYFVQLLTNANPSFL

SEQ ID NO: 5 - protein, 470 aa, Unknown; identical to SEQ ID NO: 3
  Obtained from environmental sample

SEQ ID NO: 6 - protein, 469 aa, Aspergillus aculeatus
MRYTLSLAAALLPCAIQAQQTLYGQCGGQGYSGLTSCVAGATCSTVNEYYAQCTPAAGATSTTLKTTTTTAGATTTTTTKSSASQTSTTKTSTGTVSTTTATTTASASGNPFSGYQLYVNPYYSSEVASLAIPSLTGTLSSLQAAATAAAKVPSFVWLDVAAKVPTMATYLADIKAQNAAGANPPIAGQFVVYDLPDRDCAALASNGEYSIANNGVANYKAYIDSIRKVLVQYSDVHTILVIEPDSLANLVTNLNVAKCANAQSAYLECTNYALEQLNLPNVAMYLDAGHAGWLGWPANQQPAANLYASVYKNASSPAAVRGLATNVANYNAFTISSCPSYTQGNSVCDEQQYINAIAPLLSAQGFDAHFIVDTGRNGKQPTGQQAWGDWCNVINTGFGVRPTTSTGDALVDAFVWVKPGGESDGTSDSSATRYDAHCGYSDALQPAPEAGTWFQAYFVQLLTNANPAF

SEQ ID NO: 7 - protein, 455 aa, Artificial Sequence
  Obtained from environmental sample
MVPLEERQSCSSVWGQCGGQNWAGPFCCASGSTCVYSNDYYSQCLPGTASSSSSTRASSTTSRVSSATSTRSSSSTPPPASSTTPAPPVGSGTATYSGNPFAGVTPWANSFYASEVSTLAIPSLTGAMATAAAAVAKVPSFMWLDTLDKTPLMSSTLSDIRAANKAGGNYAGQFVVYDLPDRDCAAAASNGEYSIADGGVAKYKNYIDTIRGIVTTFSDVRILLVIEPDSLANLVTNLATPKCSNAQSAYLECINYAITQLNLPNVAMYLDAGHAGWLGWPANQDPAAQLFANVYKNASSPRAVRGLATNVANYNAWNITTPPSYTQGNAVYNEKLYIHALGPLLANHGWSNAFFITDQGRSGKQPTGQLEWGNWCNAVGTGFGIRPSANTGDSLLDSFVWIKPGGECDGTSNSSAPRFDYHCASADALQPAPQAGSWFQAYFVQLLTNANPSFL

SEQ ID NO: 8 - protein, 454 aa, Trichoderma reesei
MVPLEERQACSSVWGQCGGQNWSGPTCCASGSTCVYSNDYYSQCLPGAASSSSSTRAASTTSRVSPTTSRSSSATPPPGSTTTRVPPVGSGTATYSGNPFVGVTPWANAYYASEVSSLAIPSLTGAMATAAAAVAKVPSFMWLDTLDKTPLMEQTLADIRTANKNGGNYAGQFVVYDLPDRDCAALASNGEYSIADGGVAKYKNYIDTIRQIVVEYSDIRTLLVIEPDSLANLVTNLGTPKCANAQSAYLECINYAVTQLNLPNVAMYLDAGHAGWLGWPANQDPAAQLFANVYKNASSPRALRGLATNVANYNGWNITSPPSYTQGNAVYNEKLYIHAIGPLLANHGWSNAFFITDQGRSGKQPTGQQQWGDWCNVIGTGFGIRPSANTGDSLLDSFVWVKPGGECDGTSDSSAPRFDSHCALPDALQPAPQAGAWFQAYFVQLLTNANPSFL

SEQ ID NO: 9 - protein, 492 aa, Trichoderma reesei
MVSFTSLLAGVAAISGVLAAPAAEVEPVAVEKREAEAEAVPLEERQACSSVWGQCGGQNWSGPTCCASGSTCVYSNDYYSQCLPGAASSSSSTRAASTTSRVSPTTSRSSSATPPPGSTTTRVPPVGSGTATYSGNPFVGVTPWANAYYASEVSSLAIPSLTGAMATAAAAVAKVPSFMWLDTLDKTPLMEQTLADIRTANKNGGNYAGQFVVYDLPDRDCAALASNGEYSIADGGVAKYKNYIDTIRQIVVEYSDIRTLLVIEPDSLANLVTNLGTPKCANAQSAYLECINYAVTQLNLPNVAMYLDAGHAGWLGWPANQDPAAQLFANVYKNASSPRALRGLATNVANYNGWNITSPPSYTQGNAVYNEKLYIHAIGPLLANHGWSNAFFITDQGRSGKQPTGQQQWGDWCNVIGTGFGIRPSANTGDSLLDSFVWVKPGGECDGTSDSSAPRFDSHCALPDALQPAPQAGAWFQAYFVQLLTNANPSFL

SEQ ID NO: 10 - protein, 472 aa, Artificial Sequence
  Obtained from environmental sample
MKTNLFLFLIFSLLLSLSSAEQACSSVWGQCGGQNWSGPTCCASGSTCVYSNDYYSQCLPGAASSSSSTRAASTTSRVSPTTSRSSSATPPPGSTTTRVPPVGSGTATYSGNPFVGVTPWANAYYASEVSSLAIPSLTGAMATAAAAVAKVPSFMWLDTLDKTPLMEQTLADIRTANKNGGNYAGQFVVYDLPDRDCAALASNGEYSIADGGVAKYKNYIDTIRQIVVEYSDIRTLLVIEPDSLANLVTNLGTPKCANAQSAYLECINYAVTQLNLPNVAMYLDAGHAGWLGWPANQDPAAQLFANVYKNASSPRALRGLATNVANYNGWNITSPPSYTQGNAVYNEKLYIHAIGPLLANHGWSNAFFITDQGRSGKQPTGQQQWGDWCNVIGTGFGIRPSANTGDSLLDSFVWVKPGGECDGTSDSSAPRFDSHCALPDALQPAPQAGAWFQAYFVQLLTNANPSFLKDEL

SEQ ID NO: 11 - protein, 468 aa, Unknown; identical to SEQ ID NO: 2
  Obtained from environmental sample

SEQ ID NO: 12 - protein, 472 aa, Unknown; identical to SEQ ID NO: 4
  Obtained from environmental sample

SEQ ID NO: 13 - protein, 474 aa, Unknown
  Obtained from environmental sample
MGVHKLFLATALFGLAVAAPIVEERESCATQGQCGGINWNGVTCCESGSYCSKINDYYFQCLSGSNPTTSKTSSLPISTTTSLITKSSSTASSTKTSAGSSTTASPTVGAGTATYSGNPYSGVNLWANGYYRSEVSTLAIPSLSGAMATAAAKVAEVPSFQWMDSYAHISLMEDTLADIRKANQAGGNYAGQFVVYDLPERDCAAAASNGEYSLDNDGANKYKNYINRVKTIIQSYSDIRIILVIEPDSLANLVTNMNVAKCSKAHDAYLSLTNYAVTALNLPNVAMYLDAGHAGWLGWPANQSPAAQLFAGVYKDAGKPSSLRGLVTNVANYNGWSLSTAPSYTQGNSIYDEKSFIHAMGPLLEQNGWAGAQFITDQGRSGKQPTGQAQWGDWCNAKGTGFGIRPSANTGDSLLDAFVWVKPGGESDGTSDTTAARYDYHCGYSDALQPAPEAGTWFQAYFVQLLTNANPSFL

SEQ ID NO: 14 - protein, 471 aa, Unknown
  Obtained from environmental sample
MIVGILTTLATLATLAASVPLEERQACSSVWGQCGGQNWSGPTCCASGSTCVYSNDYYSQCLPGAASSSSSTRAASTTSRVSPTTSRSSSATPPPGSTTTRVPPVGSGTATYSGNPFVGVTPWANAYYASEVSSLAIPSLTGAMATAAAAVAKVPSFMWLDTLDKTPLMEQTLADIRTANKNGGNYAGQFVVYDLPDRDCAALASNGEYSIADGGVAKYKNYIDTIRQIVVEYSDIRTLLVIEPDSLANLVTNLGTPKCANAQSAYLECINYAVTQLNLPNVAMYLDAGHAGWLGWPANQDPAAQLFANVYKNASSPRALRGLATNVANYNGWNITSPPSYTQGNAVYNEKLYIHAIGPLLANHGWSNAFFITDQGRSGKQPTGQQQWGDWCNVIGTGFGIRPSANTGDSLLDSFVWVKPGGECDGTSDSSAPRFDSHCALPDALQPAPQAGAWFQAYFVQLLTNANPSFL

SEQ ID NO: 15 - protein, 470 aa, Trichoderma reesei
MIVGILTTLATLATLAASVPLEERQACSSVWGQCGGQNWSGPTCCASGSTCVYSNDYYSQCLPGAASSSSSTRAASTTRVSPTTSRSSSATPPPGSTTTRVPPVGSGTATYSGNPFVGVTPWANAYYASEVSSLAIPSLTGAMATAAAAVAKVPSFMWLDTLDKTPLMEQTLADIRTANKNGGNYAGQFVVYDLPDRDCAALASNGEYSIADGGVAKYKNYIDTIRQIVVEYSDIRTLLVIEPDSLANLVTNLGTPKCANAQSAYLECINYAVTQLNLPNVAMYLDAGHAGWLGWPANQDPAAQLFANVYKNASSPRALRGLATNVANYNGWNITSPPSYTQGNAVYNEKLYIHAIGPLLANHGWSNAFFITDQGRSGKQPTGQQQWGDWCNVIGTGFGIRPSANTGDSLLDSFVWVKPGGECDGTSDSSAPRFDSHCALPDALQPAPQAGAWFQAYFVQLLTNANPSFL

SEQ ID NO: 16 - protein, 471 aa, Hypocrea koningii
MIVGILTTLATLATLAASVPLEERQACSSVWGQCGGQNWSGPTCCASGSTCVYSNDYYSQCLPGAASSSSSTRAASTTSRVSPTTSRSSSATPPPGSTTTRVPPVGSGTATYSGNPFVGVTPWANAYYASEVSSLAIPSLTGAMATAAAAVAKVPSFMWLDTLDKTPLMEQTLADIRTANKNGGNYAGQFVVYDLPDRDCAALASNGEYSIADGGVAKYKNYIDTIRQIVVEYSDIRTLLVIEPDSLANLVTNLGTPKCANAQSAYLECINYAVTQLNLPNVAMYLDAGHAGWLGWPANQDPAAQLFANVYKNASSPRALRGLATNVANYNGWNITSPPSYTQGNAVYNEKLYIHAIGRLLANHGWSNAFFITDQGRSGKQPTGQQQWGDWCNVIGTGFGIRPSANTGDSLLDSFVWVKPGGECDGTSDSSAPRFDSHCALPDALQPAPQAGAWFQAYFVQLLTNANPSFL

SEQ ID NO: 17 - protein, 470 aa, Hypocrea koningii
MIVGILTTLATLATLAASVPLEERQACSSAWGQCGGQNWSGPTCCASGSTCVYSNDYYSQCLPGAASSSSSTRASSTTARASSTTSRSSATPPPGSSTTRVPPVGSGTATYSGNPFVGVTPWANAYYASEVSSLAIPSLTGAMATAAAAVAKVPSFMWLDTFDKTPLMEQTLADIRTANKNGGNYAGQFVVYDLPDRDCAALASNGEYSIADGGVDKYKNYIDTIRQIVVEYSDIRTLLVIEPDSLANLVTNLGTPKCANAQSAYLECINYAVTQLNLPNVAMYLDAGHAGWLGWPANQDPAAQLFANVYKNASSPRALRGLATNVANYNGWNITSPPSYTQGNAVYNEQLYIHAIGPLLANHGWSNAFFITDQGRSGKQPTGQQQWGDWCNVIGTGFGIRPSANTGDSLLDSFVWIKPGGECDGTSDSSAPRFDSHCALPDALQPAPQAGAWFQAYFVQLLTNANPSFL

SEQ ID NO: 18 - protein, 470 aa, Trichoderma parceramosum
MIVGILTTLATLATLAASVPLEERQACSSVWGQCGGQNWSGPTCCAAGSTCVYSNDYYSQCPPGAASSSSSTRASSTTNRVSSTTSTSSATPPPGSTTTRVPPVGSGTATYSGNPFVGVTPWANAYYASEVSSLAIPSLTGAMATAAAAVAKVPSFMWLDTLDKTPLMEQTLADIRTANKNGGNYAGQFVVYDLPDRDCAALASNGEYSIADGGVAKYKNYIDTIRQIVVEYSDIRTILVIEPDSLANLVTNLGTPKCANAQSAYLECINYAITQLNLPNIAMYLDAGHAGWLGWPANQDPAAQLFANVYKNASSPSALRGLATNVANYNGWNITSPPSYTQGNAVYNEKLYIHAIGPLLANHGWSNAFFITDQGRSGKQPTGQQQWGDWCNVIGTGFGIRPSSNTGDSLLDSFVWVKPGGECDGTSDSSAPRFDSHCALPDALQPAPQAGAWFQAYFVQLLTNANPSFL

SEQ ID NO: 19 - protein, 471 aa, Trichoderma viride
MIVGILTTLATLATLAASVPLEERQACSSVWGQCGGQNWSGPTCCASGSTCVYSNDYYSQCLPGAASSSSSTRAASTTSRVSPTTSRSSSATPPPGSTTTRVPPVGSGTATYSGNPFVGVTPWANAYYASEVSSLAIPSLTGAMATAAAAVAKVPSFMWLDTLDKTPLMEQTLADIRTANKNGGNYAGQFVVYDLPDRDCAALASNGEYSIADGGVAKYKNYIDTIRQIVVEYSDIRTLLVIEPDSLANLVTNLGTPKCANAPSAYLECINYAVTQLNLPNVAMYLDAGHAGWLGWPANQDPAAQLFANVYKNASSPRALRGLATNVANYNGWNITSPPSYTQGNAVYNEKLYIHAIGPLLANHGWSNAFFITDQGRSGKQPTGQQQWGDWCNVIGTGFGIRPSANTGDSLLDSFVWVKPGGECDGTSDSSAPRFDSHCALPDALQPAPQAGAWFQAYFVQLLTNANPSFL

SEQ ID NO: 20 - protein, 471 aa, Trichoderma reesei
MIVGILTTLATLATLAASVPLEERQACSSVWGQCGGQNWSGPTCCASGSTCVYSNDYYSQCLPGAASSSSSTRAASTTSRVSPTTSRSSSATPPPGSTTTRVPPVGSGTATYSGNPFVGVTPWANAYYASEVSSLAIPSLTGAMATAAAAVAKVPSFMWLDTLDKTPLMEQTLADIRTANKNGGNYAGQFVVYDLPDRDCAALASNGEYSIADGGVAKYKNYIDTIRQIVVEYSDIRTLLVIEPDSLANLVTNLGTPKCANAQSAYLECINYAVTQLNLPNVAMYLDAGHAGWLGWPANQDPAAQLFANVYKNASSPRALRGLATNVANYNGWNITSPPSYTQGNAVYNEKLYIHAIGRLLANHGWSNAFFITDQGRSGKQPTGQQQWGDWCNVIGTGFGIRPSANTGDSLLDSFVWVKPGGECDGTSDSSAPRFDSHCALPDALQPAAQAGAWFQAYFVQLLTNANPSFL

SEQ ID NO: 21 - protein, 447 aa, Trichoderma reesei
QACSSVWGQCGGQNWSGPTCCASGSTCVYSNDYYSQCLPGAASSSSSTRAASTTSRVSPTTSRSSSATPPPGSTTTRVPPVGSGTATYSGNPFVGVTPWANAAYASEVSSLAIPSLTGAMATAAAAVAKVPSFMWLDTLDKTPLMEQTLADIRTANKNGGNYAGQFVVYDLPDRDCAALASNGEYSIADGGVAKYKNYIDTIRQIVVEYSDIRTLLVIEPDSLANLVTNLGTPKCANAQSAYLECINYAVTQLNLPNVAMYLDAGHAGWLGWPANQDPAAQLFANVYKNASSPRALRGLATNVANYNGWNITSPPSYTQGNAVYNEKLYIHAIGPLLANHGWSNAFFITDQGRSGKQPTGQQQWGDWCNVIGTGFGIRPSANTGDSLLDSFVWVKPGGECDGTSDSSAPRFDPHCALPDALQPAPQAGAWFQAYFVQLLTNANPSFL

SEQ ID NO: 22 - protein, 447 aa, Trichoderma reesei
QACSSVWGQCGGQNWSGPTCCASGSTCVYSNDYYSQCLPGAASSSSSTRAASTTSRVSPTTSRSSSATPPPGSTTTRVPPVGSGTATYSGNPFVGVTPWANAHYASEVSSLAIPSLTGAMATAAAAVAKVPSFMWLDTLDKTPLMEQTLADIRTANKNGGNYAGQFVVYDLPDRDCAALASNGEYSIADGGVAKYKNYIDTIRQIVVEYSDIRTLLVIEPDSLANLVTNLGTPKCANAQSAYLECINYAVTQLNLPNVAMYLDAGHAGWLGWPANQDPAAQLFANVYKNASSPRALRGLATNVANYNGWNITSPPSYTQGNAVYNEKLYIHAIGPLLANHGWSNAFFITDQGRSGKQPTGQQQWGDWCNVIGTGFGIRPSANTGDSLLDSFVWVKPGGECDGTSDSSAPRFDPHCALPDALQPAPQAGAWFQAYFVQLLTNANPSFL

SEQ ID NO: 23 - protein, 447 aa, Trichoderma reesei
QACSSVWGQCGGQNWSGPTCCASGSTCVYSNDYYSQCLPGAASSSSSTRAASTTSRVSPTTSRSSSATPPPGSTTTRVPPVGSGTATYSGNPFVGVTPWANAKYASEVSSLAIPSLTGAMATAAAAVAKVPSFMWLDTLDKTPLMEQTLADIRTANKNGGNYAGQFVVYDLPDRDCAALASNGEYSIADGGVAKYKNYIDTIRQIVVEYSDIRTLLVIEPDSLANLVTNLGTPKCANAQSAYLECINYAVTQLNLPNVAMYLDAGHAGWLGWPANQDPAAQLFANVYKNASSPRALRGLATNVANYNGWNITSPPSYTQGNAVYNEKLYIHAIGPLLANHGWSNAFFITDQGRSGKQPTGQQQWGDWCNVIGTGFGIRPSANTGDSLLDSFVWVKPGGECDGTSDSSAPRFDPHCALPDALQPAPQAGAWFQAYFVQLLTNANPSFL

SEQ ID NO: 24 - protein, 447 aa, Trichoderma reesei
QACSSVWGQCGGQNWSGPTCCASGSTCVYSNDYYSQCLPGAASSSSSTRAASTTSRVSPTTSRSSSATPPPGSTTTRVPPVGSGTATYSGNPFVGVTPWANALYASEVSSLAIPSLTGAMATAAAAVAKVPSFMWLDTLDKTPLMEQTLADIRTANKNGGNYAGQFVVYDLPDRDCAALASNGEYSIADGGVAKYKNYIDTIRQIVVEYSDIRTLLVIEPDSLANLVTNLGTPKCANAQSAYLECINYAVTQLNLPNVAMYLDAGHAGWLGWPANQDPAAQLFANVYKNASSPRALRGLATNVANYNGWNITSPPSYTQGNAVYNEKLYIHAIGPLLANHGWSNAFFITDQGRSGKQPTGQQQWGDWCNVIGTGFGIRPSANTGDSLLDSFVWVKPGGECDGTSDSSAPRFDPHCALPDALQPAPQAGAWFQAYFVQLLTNANPSFL

SEQ ID NO: 25 - protein, 447 aa, Trichoderma reesei
QACSSVWGQCGGQNWSGPTCCASGSTCVYSNDYYSQCLPGAASSSSSTRAASTTSRVSPTTSRSSSATPPPGSTTTRVPPVGSGTATYSGNPFVGVTPWANAMYASEVSSLAIPSLTGAMATAAAAVAKVPSFMWLDTLDKTPLMEQTLADIRTANKNGGNYAGQFVVYDLPDRDCAALASNGEYSIADGGVAKYKNYIDTIRQIVVEYSDIRTLLVIEPDSLANLVTNLGTPKCANAQSAYLECINYAVTQLNLPNVAMYLDAGHAGWLGWPANQDPAAQLFANVYKNASSPRALRGLATNVANYNGWNITSPPSYTQGNAVYNEKLYIHAIGPLLANHGWSNAFFITDQGRSGKQPTGQQQWGDWCNVIGTGFGIRPSANTGDSLLDSFVWVKPGGECDGTSDSSAPRFDPHCALPDALQPAPQAGAWFQAYFVQLLTNANPSFL

SEQ ID NO: 26 - protein, 447 aa, Trichoderma reesei
QACSSVWGQCGGQNWSGPTCCASGSTCVYSNDYYSQCLPGAASSSSSTRAASTTSRVSPTTSRSSSATPPPGSTTTRVPPVGSGTATYSGNPFVGVTPWANAPYASEVSSLAIPSLTGAMATAAAAVAKVPSFMWLDTLDKTPLMEQTLADIRTANKNGGNYAGQFVVYDLPDRDCAALASNGEYSIADGGVAKYKNYIDTIRQIVVEYSDIRTLLVIEPDSLANLVTNLGTPKCANAQSAYLECINYAVTQLNLPNVAMYLDAGHAGWLGWPANQDPAAQLFANVYKNASSPRALRGLATNVANYNGWNITSPPSYTQGNAVYNEKLYIHAIGPLLANHGWSNAFFITDQGRSGKQPTGQQQWGDWCNVIGTGFGIRPSANTGDSLLDSFVWVKPGGECDGTSDSSAPRFDPHCALPDALQPAPQAGAWFQAYFVQLLTNANPSFL

SEQ ID NO: 27 - protein, 447 aa, Trichoderma reesei
QACSSVWGQCGGQNWSGPTCCASGSTCVYSNDYYSQCLPGAASSSSSTRAASTTSRVSPTTSRSSSATPPPGSTTTRVPPVGSGTATYSGNPFVGVTPWANARYASEVSSLAIPSLTGAMATAAAAVAKVPSFMWLDTLDKTPLMEQTLADIRTANKNGGNYAGQFVVYDLPDRDCAALASNGEYSIADGGVAKYKNYIDTIRQIVVEYSDIRTLLVIEPDSLANLVTNLGTPKCANAQSAYLECINYAVTQLNLPNVAMYLDAGHAGWLGWPANQDPAAQLFANVYKNASSPRALRGLATNVANYNGWNITSPPSYTQGNAVYNEKLYIHAIGPLLANHGWSNAFFITDQGRSGKQPTGQQQWGDWCNVIGTGFGIRPSANTGDSLLDSFVWVKPGGECDGTSDSSAPRFDPHCALPDALQPAPQAGAWFQAYFVQLLTNANPSFL

SEQ ID NO: 28 - protein, 447 aa, Trichoderma reesei
QACSSVWGQCGGQNWSGPTCCASGSTCVYSNDYYSQCLPGAASSSSSTRAASTTSRVSPTTSRSSSATPPPGSTTTRVPPVGSGTATYSGNPFVGVTPWANAVYASEVSSLAIPSLTGAMATAAAAVAKVPSFMWLDTLDKTPLMEQTLADIRTANKNGGNYAGQFVVYDLPDRDCAALASNGEYSIADGGVAKYKNYIDTIRQIVVEYSDIRTLLVIEPDSLANLVTNLGTPKCANAQSAYLECINYAVTQLNLPNVAMYLDAGHAGWLGWPANQDPAAQLFANVYKNASSPRALRGLATNVANYNGWNITSPPSYTQGNAVYNEKLYIHAIGPLLANHGWSNAFFITDQGRSGKQPTGQQQWGDWCNVIGTGFGIRPSANTGDSLLDSFVWVKPGGECDGTSDSSAPRFDPHCALPDALQPAPQAGAWFQAYFVQLLTNANPSFL

SEQ ID NO: 29 - protein, 447 aa, Trichoderma reesei
QACSSVWGQCGGQNWSGPTCCASGSTCVYSNDYYSQCLPGAASSSSSTRAASTTSRVSPTTSRSSSATPPPGSTTTRVPPVGSGTATYSGNPFVGVTPWANAYYASEVSSLAIPSLTGAMATAAAAVAKVPSFMWIDTLDKTPLMEQTLADIRTANKNGGNYAGQFVVYDLPDRDCAALASNGEYSIADGGVAKYKNYIDTIRQIVVEYSDIRTLLVIEPDSLANLVTNLGTPKCANAQSAYLECINYAVTQLNLPNVAMYLDAGHAGWLGWPANQDPAAQLFANVYKNASSPRALRGLATNVANYNGWNITSPPSYTQGNAVYNEKLYIHAIGPLLANHGWSNAFFITDQGRSGKQPTGQQQWGDWCNVIGTGFGIRPSANTGDSLLDSFVWVKPGGECDGTSDSSAPRFDPHCALPDALQPAPQAGAWFQAYFVQLLTNANPSFL

SEQ ID NO: 30 - protein, 447 aa, Trichoderma reesei
QACSSVWGQCGGQNWSGPTCCASGSTCVYSNDYYSQCLPGAASSSSSTRAASTTSRVSPTTSRSSSATPPPGSTTTRVPPVGSGTATYSGNPFVGVTPWANAYYASEVSSLAIPSLTGAMATAAAAVAKVPSFMWVDTLDKTPLMEQTLADIRTANKNGGNYAGQFVVYDLPDRDCAALASNGEYSIADGGVAKYKNYIDTIRQIVVEYSDIRTLLVIEPDSLANLVTNLGTPKCANAQSAYLECINYAVTQLNLPNVAMYLDAGHAGWLGWPANQDPAAQLFANVYKNASSPRALRGLATNVANYNGWNITSPPSYTQGNAVYNEKLYIHAIGPLLANHGWSNAFFITDQGRSGKQPTGQQQWGDWCNVIGTGFGIRPSANTGDSLLDSFVWVKPGGECDGTSDSSAPRFDPHCALPDALQPAPQAGAWFQAYFVQLLTNANPSFL

SEQ ID NO: 31 - protein, 447 aa, Trichoderma reesei
QACSSVWGQCGGQNWSGPTCCASGSTCVYSNDYYSQCLPGAASSSSSTRAASTTSRVSPTTSRSSSATPPPGSTTTRVPPVGSGTATYSGNPFVGVTPWANAYYASEVSSLAIPSLTGAMATAAAAVAKVPSFMWLDTLDKTPLMEQTLADIRTANKNGGNYAGQFVVYDLPDRDCAALASNGEYKIADGGVAKYKNYIDTIRQIVVEYSDIRTLLVIEPDSLANLVTNLGTPKCANAQSAYLECINYAVTQLNLPNVAMYLDAGHAGWLGWPANQDPAAQLFANVYKNASSPRALRGLATNVANYNGWNITSPPSYTQGNAVYNEKLYIHAIGPLLANHGWSNAFFITDQGRSGKQPTGQQQWGDWCNVIGTGFGIRPSANTGDSLLDSFVWVKPGGECDGTSDSSAPRFDPHCALPDALQPAPQAGAWFQAYFVQLLTNANPSFL

SEQ ID NO: 32 - protein, 447 aa, Trichoderma reesei
QACSSVWGQCGGQNWSGPTCCASGSTCVYSNDYYSQCLPGAASSSSSTRAASTTSRVSPTTSRSSSATPPPGSTTTRVPPVGSGTATYSGNPFVGVTPWANAYYASEVSSLAIPSLTGAMATAAAAVAKVPSFMWLDTLDKTPLMEQTLADIRTANKNGGNYAGQFVVYDLPDRDCAALASNGEYYIADGGVAKYKNYIDTIRQIVVEYSDIRTLLVIEPDSLANLVTNLGTPKCANAQSAYLECINYAVTQLNLPNVAMYLDAGHAGWLGWPANQDPAAQLFANVYKNASSPRALRGLATNVANYNGWNITSPPSYTQGNAVYNEKLYIHAIGPLLANHGWSNAFFITDQGRSGKQPTGQQQWGDWCNVIGTGFGIRPSANTGDSLLDSFVWVKPGGECDGTSDSSAPRFDPHCALPDALQPAPQAGAWFQAYFVQLLTNANPSFL

SEQ ID NO: 33 - protein, 447 aa, Trichoderma reesei
QACSSVWGQCGGQNWSGPTCCASGSTCVYSNDYYSQCLPGAASSSSSTRAASTTSRVSPTTSRSSSATPPPGSTTTRVPPVGSGTATYSGNPFVGVTPWANAYYASEVSSLAIPSLTGAMATAAAAVAKVPSFMWLDTLDKTPLMEQTLADIRTANKNGGNYAGQFVVYDLPDRDCAALASNGEYSIADGGVAKYKNYIDTIRQIVVEYSDIRTLLVIEPDSLANLVTNLGTPKCANAQSAYLECINYAVTQLNLPNVAMYLDAGHAGWLGWPANQDPAAQLFANVYKNASSPRALRGLATNVANYNGWNITSPPSYTQGNAVYNEKLYIHAIGPLLANHGWSNAFFITDQGRSGKQPTGQQQWDDWCNVIGTGFGIRPSANTGDSLLDSFVWVKPGGECDGTSDSSAPRFDPHCALPDALQPAPQAGAWFQAYFVQLLTNANPSFL

SEQ ID NO: 34 - protein, 447 aa, Trichoderma reesei
QACSSVWGQCGGQNWSGPTCCASGSTCVYSNDYYSQCLPGAASSSSSTRAASTTSRVSPTTSRSSSATPPPGSTTTRVPPVGSGTATYSGNPFVGVTPWANAYYASEVSSLAIPSLTGAMATAAAAVAKVPSFMWLDTLDKTPLMEQTLADIRTANKNGGNYAGQFVVYDLPDRDCAALASNGEYSIADGGVAKYKNYIDTIRQIVVEYSDIRTLLVIEPDSLANLVTNLGTPKCANAQSAYLECINYAVTQLNLPNVAMYLDAGHAGWLGWPANQDPAAQLFANVYKNASSPRALRGLATNVANYNGWNITSPPSYTQGNAVYNEKLYIHAIGPLLANHGWSNAFFITDQGRSGKQPTGQQQWEDWCNVIGTGFGIRPSANTGDSLLDSFVWVKPGGECDGTSDSSAPRFDPHCALPDALQPAPQAGAWFQAYFVQLLTNANPSFL

SEQ ID NO: 35 - protein, 447 aa, Trichoderma reesei
QACSSVWGQCGGQNWSGPTCCASGSTCVYSNDYYSQCLPGAASSSSSTRAASTTSRVSPTTSRSSSATPPPGSTTTRVPPVGSGTATYSGNPFVGVTPWANAYYASEVSSLAIPSLTGAMATAAAAVAKVPSFMWLDTLDKTPLMEQTLADIRTANKNGGNYAGQFVVYDLPDRDCAALASNGEYSIADGGVAKYKNYIDTIRQIVVEYSDIRTLLVIEPDSLANLVTNLGTPKCANAQSAYLECINYAVTQLNLPNVAMYLDAGHAGWLGWPANQDPAAQLFANVYKNASSPRALRGLATNVANYNGWNITSPPSYTQGNAVYNEKLYIHAIGPLLANHGWSNAFFITDQGRSGKQPTGQQQWQDWCNVIGTGFGIRPSANTGDSLLDSFVWVKPGGECDGTSDSSAPRFDPHCALPDALQPAPQAGAWFQAYFVQLLTNANPSFL

SEQ ID NO: 36 - protein, 447 aa, Trichoderma reesei
QACSSVWGQCGGQNWSGPTCCASGSTCVYSNDYYSQCLPGAASSSSSTRAASTTSRVSPTTSRSSSATPPPGSTTTRVPPVGSGTATYSGNPFVGVTPWANAYYASEVSSLAIPSLTGAMATAAAAVAKVPSFMWLDTLDKTPLMEQTLADIRTANKNGGNYAGQFVVYDLPDRDCAALASNGEYSIADGGVAKYKNYIDTIRQIVVEYSDIRTLLVIEPDSLANLVTNLGTPKCANAQSAYLECINYAVTQLNLPNVAMYLDAGHAGWLGWPANQDPAAQLFANVYKNASSPRALRGLATNVANYNGWNITSPPSYTQGNAVYNEKLYIHAIGPLLANHGWSNAFFITDQGRSGKQPTGQQQWSDWCNVIGTGFGIRPSANTGDSLLDSFVWVKPGGECDGTSDSSAPRFDPHCALPDALQPAPQAGAWFQAYFVQLLTNANPSFL

SEQ ID NO: 37 - protein, 447 aa, Trichoderma reesei
QACSSVWGQCGGQNWSGPTCCASGSTCVYSNDYYSQCLPGAASSSSSTRAASTTSRVSPTTSRSSSATPPPGSTTTRVPPVGSGTATYSGNPFVGVTPWANAYYASEVSSLAIPSLTGAMATAAAAVAKVPSFMWLDTLDKTPLMEQTLADIRTANKNGGNYAGQFVVYDLPDRDCAALASNGEYSIADGGVAKYKNYIDTIRQIVVEYSDIRTLLVIEPDSLANLVTNLGTPKCANAQSAYLECINYAVTQLNLPNVAMYLDAGHAGWLGWPANQDPAAQLFANVYKNASSPRALRGLATNVANYNGWNITSPPSYTQGNAVYNEKLYIHAIGPLLANHGWSNAFFITDQGRSGKQPTGQQQWGDWCNVIGTGFGIRPSANTGDSLLDSFVWVKPGGECDGTSDSSAPAFDPHCALPDALQPAPQAGAWFQAYFVQLLTNANPSFL

SEQ ID NO: 38 - protein, 447 aa, Trichoderma reesei
QACSSVWGQCGGQNWSGPTCCASGSTCVYSNDYYSQCLPGAASSSSSTRAASTTSRVSPTTSRSSSATPPPGSTTTRVPPVGSGTATYSGNPFVGVTPWANAYYASEVSSLAIPSLTGAMATAAAAVAKVPSFMWLDTLDKTPLMEQTLADIRTANKNGGNYAGQFVVYDLPDRDCAALASNGEYSIADGGVAKYKNYIDTIRQIVVEYSDIRTLLVIEPDSLANLVTNLGTPKCANAQSAYLECINYAVTQLNLPNVAMYLDAGHAGWLGWPANQDPAAQLFANVYKNASSPRALRGLATNVANYNGWNITSPPSYTQGNAVYNEKLYIHAIGPLLANHGWSNAFFITDQGRSGKQPTGQQQWGDWCNVIGTGFGIRPSANTGDSLLDSFVWVKPGGECDGTSDSSAPFFDPHCALPDALQPAPQAGAWFQAYFVQLLTNANPSFL

SEQ ID NO: 39 - protein, 447 aa, Trichoderma reesei
QACSSVWGQCGGQNWSGPTCCASGSTCVYSNDYYSQCLPGAASSSSSTRAASTTSRVSPTTSRSSSATPPPGSTTTRVPPVGSGTATYSGNPFVGVTPWANAYYASEVSSLAIPSLTGAMATAAAAVAKVPSFMWLDTLDKTPLMEQTLADIRTANKNGGNYAGQFVVYDLPDRDCAALASNGEYSIADGGVAKYKNYIDTIRQIVVEYSDIRTLLVIEPDSLANLVTNLGTPKCANAQSAYLECINYAVTQLNLPNVAMYLDAGHAGWLGWPANQDPAAQLFANVYKNASSPRALRGLATNVANYNGWNITSPPSYTQGNAVYNEKLYIHAIGPLLANHGWSNAFFITDQGRSGKQPTGQQQWGDWCNVIGTGFGIRPSANTGDSLLDSFVWVKPGGECDGTSDSSAPLFDPHCALPDALQPAPQAGAWFQAYFVQLLTNANPSFL

SEQ ID NO: 40 - protein, 447 aa, Trichoderma reesei
QACSSVWGQCGGQNWSGPTCCASGSTCVYSNDYYSQCLPGAASSSSSTRAASTTSRVSPTTSRSSSATPPPGSTTTRVPPVGSGTATYSGNPFVGVTPWANAYYASEVSSLAIPSLTGAMATAAAAVAKVPSFMWLDTLDKTPLMEQTLADIRTANKNGGNYAGQFVVYDLPDRDCAALASNGEYSIADGGVAKYKNYIDTIRQIVVEYSDIRTLLVIEPDSLANLVTNLGTPKCANAQSAYLECINYAVTQLNLPNVAMYLDAGHAGWLGWPANQDPAAQLFANVYKNASSPRALRGLATNVANYNGWNITSPPSYTQGNAVYNEKLYIHAIGPLLANHGWSNAFFITDQGRSGKQPTGQQQWGDWCNVIGTGFGIRPSANTGDSLLDSFVWVKPGGECDGTSDSSAPSFDPHCALPDALQPAPQAGAWFQAYFVQLLTNANPSFL

SEQ ID NO: 41 - protein, 447 aa, Trichoderma reesei
QACSSVWGQCGGQNWSGPTCCASGSTCVYSNDYYSQCLPGAASSSSSTRAASTTSRVSPTTSRSSSATPPPGSTTTRVPPVGSGTATYSGNPFVGVTPWANALYASEVSSLAIPSLTGAMATAAAAVAKVPSFMWIDTLDKTPLMEQTLADIRTANKNGGNYAGQFVVYDLPDRDCAALASNGEYSIADGGVAKYKNYIDTIRQIVVEYSDIRTLLVIEPDSLANLVTNLGTPKCANAQSAYLECINYAVTQLNLPNVAMYLDAGHAGWLGWPANQDPAAQLFANVYKNASSPRALRGLATNVANYNGWNITSPPSYTQGNAVYNEKLYIHAIGPLLANHGWSNAFFITDQGRSGKQPTGQQQWGDWCNVIGTGFGIRPSANTGDSLLDSFVWVKPGGECDGTSDSSAPRFDPHCALPDALQPAPQAGAWFQAYFVQLLTNANPSFL

SEQ ID NO: 42 - protein, 447 aa, Trichoderma reesei
QACSSVWGQCGGQNWSGPTCCASGSTCVYSNDYYSQCLPGAASSSSSTRAASTTSRVSPTTSRSSSATPPPGSTTTRVPPVGSGTATYSGNPFVGVTPWANALYASEVSSLAIPSLTGAMATAAAAVAKVPSFMWLDTLDKTPLMEQTLADIRTANKNGGNYAGQFVVYDLPDRDCAALASNGEYSIADGGVAKYKNYIDTIRQIVVEYSDIRTLLVIEPDSLANLVTNLGTPKCANAQSAYLECINYAVTQLNLPNVAMYLDAGHAGWLGWPANQDPAAQLFANVYKNASSPRALRGLATNVANYNGWNITSPPSYTQGNAVYNEKLYIHAIGPLLANHGWSNAFFITDQGRSGKQPTGQQQWGDWCNVIGTGFGIRPSANTGDSLLDSFVWVKPGGECDGTSDSSAPQFDPHCALPDALQPAPQAGAWFQAYFVQLLTNANPSFL

SEQ ID NO: 43 - protein, 447 aa, Trichoderma reesei
QACSSVWGQCGGQNWSGPTCCASGSTCVYSNDYYSQCLPGAASSSSSTRAASTTSRVSPTTSRSSSATPPPGSTTTRVPPVGSGTATYSGNPFVGVTPWANAYYASEVSSLAIPSLTGAMATAAAAVAKVPSFVWIDTLDKTPLMEQTLADIRTANKNGGNYAGQFVVYDLPDRDCAALASNGEYSIADGGVAKYKNYIDTIRQIVVEYSDIRTLLVIEPDSLANLVTNLGTPKCANAQSAYLECINYAVTQLNLPNVAMYLDAGHAGWLGWPANQDPAAQLFANVYKNASSPRALRGLATNVANYNGWNITSPPSYTQGNAVYNEKLYIHAIGPLLANHGWSNAFFITDQGRSGKQPTGQQQWGDWCNVIGTGFGIRPSANTGDSLLDSFVWVKPGGECDGTSDSSAPRFDPHCALPDALQPAPQAGAWFQAYFVQLLTNANPSFL

SEQ ID NO: 44 - protein, 447 aa, Trichoderma reesei
QACSSVWGQCGGQNWSGPTCCASGSTCVYSNDYYSQCLPGAASSSSSTRAASTTSRVSPTTSRSSSATPPPGSTTTRVPPVGSGTATYSGNPFVGVTPWANAYYASEVSSLAIPSLTGAMATAAAAVAKVPSFMWIDTLDKTPLMEQTLADIRTANKNGGNYAGQFVVYDLPDRDCAALASNGEYSIADGGVAKYKNYIDTIRQIVVEYSDIRTILVIEPDSLANLVTNLGTPKCANAQSAYLECINYAVTQLNLPNVAMYLDAGHAGWLGWPANQDPAAQLFANVYKNASSPRALRGLATNVANYNGWNITSPPSYTQGNAVYNEKLYIHAIGPLLANHGWSNAFFITDQGRSGKQPTGQQQWGDWCNVIGTGFGIRPSANTGDSLLDSFVWVKPGGECDGTSDSSAPRFDPHCALPDALQPAPQAGAWFQAYFVQLLTNANPSFL

SEQ ID NO: 45 - protein, 447 aa, Trichoderma reesei
QACSSVWGQCGGQNWSGPTCCASGSTCVYSNDYYSQCLPGAASSSSSTRAASTTSRVSPTTSRSSSATPPPGSTTTRVPPVGSGTATYSGNPFVGVTPWANAYYASEVSSLAIPSLTGAMATAAAAVAKVPSFMWIDTLDKTPLMEQTLADIRTANKNGGNYAGQFVVYDLPDRDCAALASNGEYYIADGGVAKYKNYIDTIRQIVVEYSDIRTLLVIEPDSLANLVTNLGTPKCANAQSAYLECINYAVTQLNLPNVAMYLDAGHAGWLGWPANQDPAAQLFANVYKNASSPRALRGLATNVANYNGWNITSPPSYTQGNAVYNEKLYIHAIGPLLANHGWSNAFFITDQGRSGKQPTGQQQWGDWCNVIGTGFGIRPSANTGDSLLDSFVWVKPGGECDGTSDSSAPRFDPHCALPDALQPAPQAGAWFQAYFVQLLTNANPSFL

SEQ ID NO: 46 - protein, 447 aa, Trichoderma reesei
QACSSVWGQCGGQNWSGPTCCASGSTCVYSNDYYSQCLPGAASSSSSTRAASTTSRVSPTTSRSSSATPPPGSTTTRVPPVGSGTATYSGNPFVGVTPWANAYYASEVSSLAIPSLTGAMATAAAAVAKVPSFMWIDTLDKTPLMEQTLADIRTANKNGGNYAGQFVVYDLPDRDCAALASNGEYSIADGGVAKYKNYIDTIRQIVVEYSDIRTLLVIEPDSLANLVTNLGTPKCANAQSAYLECINYAVTQLNLPNVAMYLDAGHAGWLGWPANQDPAAQLFANVYKNASSPRALRGLATNVANYNGWNITSPPSYTQGNAVYNEKLYIHAIGPLLANHGWSNAFFITDQGRSGKQPTGQQQWGDWCNVIGTGFGIRPSANTGDSLLDSFVWVKPGGECDGTSDSSAPQFDPHCALPDALQPAPQAGAWFQAYFVQLLTNANPSFL

SEQ ID NO: 47 - protein, 447 aa, Trichoderma reesei
QACSSVWGQCGGQNWSGPTCCASGSTCVYSNDYYSQCLPGAASSSSSTRAASTTSRVSPTTSRSSSATPPPGSTTTRVPPVGSGTATYSGNPFVGVTPWANAYYASEVSSLAIPSLTGAMATAAAAVAKVPSFVWIDTLDKTPLMEQTLADIRTANKNGGNYAGQFVVYDLPDRDCAALASNGEYSIADGGVAKYKNYIDTIRQIVVEYSDIRTILVIEPDSLANLVTNLGTPKCANAQSAYLECINYAVTQLNLPNVAMYLDAGHAGWLGWPANQDPAAQLFANVYKNASSPRALRGLATNVANYNGWNITSPPSYTQGNAVYNEKLYIHAIGPLLANHGWSNAFFITDQGRSGKQPTGQQQWGDWCNVIGTGFGIRPSANTGDSLLDSFVWVKPGGECDGTSDSSAPRFDPHCALPDALQPAPQAGAWFQAYFVQLLTNANPSFL

SEQ ID NO: 48 - protein, 447 aa, Trichoderma reesei
QACSSVWGQCGGQNWSGPTCCASGSTCVYSNDYYSQCLPGAASSSSSTRAASTTSRVSPTTSRSSSATPPPGSTTTRVPPVGSGTATYSGNPFVGVTPWANAYYASEVSSLAIPSLTGAMATAAAAVAKVPSFIWLDTLDKTPLMEQTLADIRTANKNGGNYAGQFVVYDLPDRDCAALASNGEYSIADGGVAKYKNYIDTIRQIVVEYSDIRTLLVIEPDSLANLVTNLGTPKCANAQSAYLECINYAVTQLNLPNVAMYLDAGHAGWLGWPANQDPAAQLFANVYKNASSPRALRGLATNVANYNGWNITSPPSYTQGNAVYNEKLYIHAIGPLLANHGWSNAFFITDQGRSGKQPTGQQQWGDWCNVIGTGFGIRPSANTGDSLLDSFVWVKPGGECDGTSDSSAPRFDPHCALPDALQPAPQAGAWFQAYFVQLLTNANPSFL

SEQ ID NO: 49 - protein, 447 aa, Trichoderma reesei
QACSSVWGQCGGQNWSGPTCCASGSTCVYSNDYYSQCLPGAASSSSSTRAASTTSRVSPTTSRSSSATPPPGSTTTRVPPVGSGTATYSGNPFVGVTPWANAYYASEVSSLAIPSLTGAMATAAAAVAKVPSFQWLDTLDKTPLMEQTLADIRTANKNGGNYAGQFVVYDLPDRDCAALASNGEYSIADGGVAKYKNYIDTIRQIVVEYSDIRTLLVIEPDSLANLVTNLGTPKCANAQSAYLECINYAVTQLNLPNVAMYLDAGHAGWLGWPANQDPAAQLFANVYKNASSPRALRGLATNVANYNGWNITSPPSYTQGNAVYNEKLYIHAIGPLLANHGWSNAFFITDQGRSGKQPTGQQQWGDWCNVIGTGFGIRPSANTGDSLLDSFVWVKPGGECDGTSDSSAPRFDPHCALPDALQPAPQAGAWFQAYFVQLLTNANPSFL

SEQ ID NO: 50 - protein, 447 aa, Trichoderma reesei
QACSSVWGQCGGQNWSGPTCCASGSTCVYSNDYYSQCLPGAASSSSSTRAASTTSRVSPTTSRSSSATPPPGSTTTRVPPVGSGTATYSGNPFVGVTPWANAYYASEVSSLAIPSLTGAMATAAAAVAKVPSFTWLDTLDKTPLMEQTLADIRTANKNGGNYAGQFVVYDLPDRDCAALASNGEYSIADGGVAKYKNYIDTIRQIVVEYSDIRTLLVIEPDSLANLVTNLGTPKCANAQSAYLECINYAVTQLNLPNVAMYLDAGHAGWLGWPANQDPAAQLFANVYKNASSPRALRGLATNVANYNGWNITSPPSYTQGNAVYNEKLYIHAIGPLLANHGWSNAFFITDQGRSGKQPTGQQQWGDWCNVIGTGFGIRPSANTGDSLLDSFVWVKPGGECDGTSDSSAPRFDPHCALPDALQPAPQAGAWFQAYFVQLLTNANPSFL

SEQ ID NO: 51 - protein, 447 aa, Trichoderma reesei
QACSSVWGQCGGQNWSGPTCCASGSTCVYSNDYYSQCLPGAASSSSSTRAASTTSRVSPTTSRSSSATPPPGSTTTRVPPVGSGTATYSGNPFVGVTPWANAYYASEVSSLAIPSLTGAMATAAAAVAKVPSFVWLDTLDKTPLMEQTLADIRTANKNGGNYAGQFVVYDLPDRDCAALASNGEYSIADGGVAKYKNYIDTIRQIVVEYSDIRTLLVIEPDSLANLVTNLGTPKCANAQSAYLECINYAVTQLNLPNVAMYLDAGHAGWLGWPANQDPAAQLFANVYKNASSPRALRGLATNVANYNGWNITSPPSYTQGNAVYNEKLYIHAIGPLLANHGWSNAFFITDQGRSGKQPTGQQQWGDWCNVIGTGFGIRPSANTGDSLLDSFVWVKPGGECDGTSDSSAPRFDPHCALPDALQPAPQAGAWFQAYFVQLLTNANPSFL

SEQ ID NO: 52 - protein, 447 aa, Trichoderma reesei
QACSSVWGQCGGQNWSGPTCCASGSTCVYSNDYYSQCLPGAASSSSSTRAASTTSRVSPTTSRSSSATPPPGSTTTRVPPVGSGTATYSGNPFVGVTPWANAYYASEVSSLAIPSLTGAMATAAAAVAKVPSFYWLDTLDKTPLMEQTLADIRTANKNGGNYAGQFVVYDLPDRDCAALASNGEYSIADGGVAKYKNYIDTIRQIVVEYSDIRTLLVIEPDSLANLVTNLGTPKCANAQSAYLECINYAVTQLNLPNVAMYLDAGHAGWLGWPANQDPAAQLFANVYKNASSPRALRGLATNVANYNGWNITSPPSYTQGNAVYNEKLYIHAIGPLLANHGWSNAFFITDQGRSGKQPTGQQQWGDWCNVIGTGFGIRPSANTGDSLLDSFVWVKPGGECDGTSDSSAPRFDPHCALPDALQPAPQAGAWFQAYFVQLLTNANPSFL

SEQ ID NO: 53 - protein, 459 aa, Trichoderma reesei
DYKDDDDKEFLEASCSSVWGQCGGQNWSGPTCCASGSTCVYSNDYYSQCLPGAASSSSSTRAASTTSRVSPTTSRSSSATPPPGSTTTRVPPVGSGTATYSGNPFVGVTPWANAYYASEVSSLAIPSLTGAMATAAAAVAKVPSFMWLDTLDKTPLMEQTLADIRTANKNGGNYAGQFVVYDLPDRDCAALASNGEYSIADGGVAKYKNYIDTIRQIVVEYSDIRTLLVIEPDSLANLVTNLGTPKCANAQSAYLECINYAVTQLNLPNVAMYLDAGHAGWLGWPANQDPAAQLFANVYKNASSPRALRGLATNVANYNGWNITSPPSYTQGNAVYNEKLYIHAIGPLLANHGWSNAFFITDQGRSGKQPTGQQQWGDWCNVIGTGFGIRPSANTGDSLLDSFVWVKPGGECDGTSDSSAPRFDSHCALPDALQPAPQAGAWFQAYFVQLLTNANPSFL

SEQ ID NO: 54 - protein, 447 aa, Trichoderma reesei
QACSSVWGQCGGQNWSGPTCCASGSTCVYSNDYYSQCLPGAASSSSSTRAASTTSRVSPTTSRSSSATPPPGSTTTRVPPVGSGTATYSGNPFVGVTPWANAYYASEVSSLAIPSLTGAMATAAAAVAKVPSFMWLDTLDKTPLMEQTLADIRTANKNGGNYAGQFVVYDLPDRDCAALASNGEYSIADGGVAKYKNYIDTIRQIVVEYSDIRTLLVIEPDSLANLVTNLGTPKCANAQSAYLECINYAVTQLNLPNVAMYLDAGHAGWLGWPANQDPAAQLFANVYKNASSPRALRGLATNVANYNGWNITSPPSYTQGNAVYNEKLYIHAIGPLLANHGWSNAFFITDQGRSGKQPTGQQQWGDWCNVIGTGFGIRPSANTGDSLLDSFVWVKPGGECDGTSDSSAPRFDPHCALPDALQPAPQAGAWFQAYFVQLLTNANPSFL

SEQ ID NO: 55 - protein, 447 aa, Trichoderma reesei
QACSSVWGQCGGQNWSGPTCCASGSTCVYSNDYYSQCLPGAASSSSSTRAASTTSRVSPTTSRSSSATPPPGSTTTRVPPVGSGTATYSGNPFVGVTPWANAYYASEVSSLAIPSLTGAMATAAAAVAKVPSFMWLDTLDKTPLMEQTLADIRTANKNGGNYAGQFVVYDLPDRDCAALASNGEYSIADGGVAKYKNYIDTIRQIVVEYSDIRTLLVIEPDSLANLVTNLSTPKCANAQSAYLECINYAVTQLNLPNVAMYLDAGHAGWLGWPANQDPAAQLFANVYKNASSPRALRGLATNVANYNGWNITSPPSYTQGNAVYNEKLYIHAIGPLLANHGWSNAFFITDQGRSGKQPTGQQQWGDWCNVIGTGFGIRPSANTGDSLLDSFVWVKPGGECDGTSDSSAPRFDPHCALPDALQPAPQAGAWFQAYFVQLLTNANPSFL

SEQ ID NO: 56 - protein, 447 aa, Trichoderma reesei
QACSSVWGQCGGQNWSGPTCCASGSTCVYSNDYYSQCLPGAASSSSSTRAASTTSRVSPTTSRSSSATPPPGSTTTRVPPVGSGTATYSGNPFVGVTPWANAYYASEVSSLAIPSLTGAMATAAAAVAKVPSFMWLDTLDKTPLMEQTLADIRTANKNGGNYAGQFVVYDLPDRDCAALASNGEYSIADGGVAKYKNYIDTIRQIVVEYSDIRTLLVIEPDSLANLVTNLGTPKCANAQSAYLECINYAVTQLNLPNVAMYLDAGHAGWLGWPANQDPAAQLFANVYKNASSPRALRGLATNVASYNGWNITSPPSYTQGNAVYNEKLYIHAIGPLLANHGWSNAFFITDQGRSGKQPTGQQQWGDWCNVIGTGFGIRPSANTGDSLLDSFVWVKPGGECDGTSDSSAPRFDPHCALPDALQPAPQAGAWFQAYFVQLLTNANPSFL

SEQ ID NO: 57 - protein, 447 aa, Trichoderma reesei
QACSSVWGQCGGQNWSGPTCCASGSTCVYSNDYYSQCLPGAASSSSSTRAASTTSRVSPTTSRSSSATPPPGSTTTRVPPVGSGTATYSGNPFVGVTPWANAYYASEVSSLAIPSLTGAMATAAAAVAKVPSFMWLDTLDKTPLMEQTLADIRTANKNGGNYAGQFVVYDLPDRDCAALASNGEYSIADGGVAKYKNYIDTIRQIVVEYSDIRTLLVIEPDSLANLVTNLGTPKCANAQSAYLECINYAVTQLNLPNVAMYLDAGHAGWLGWPANQDPAAQLFANVYKNASSPRALRGLATNVANYNGWNITSPPSYTQGNAVYNEKLYIHAIGPLLANHGWSNAFFITDQGRSGKQPTGQQQWGDWCNVIGTGFGIRPSANTGDSLLDSFVWVKPGGECDGTSDSSAPQFDPHCALPDALQPAPQAGAWFQAYFVQLLTNANPSFL

SEQ ID NO: 58 - protein, 447 aa, Trichoderma reesei
QACSSVWGQCGGQNWSGPTCCASGSTCVYSNDYYSQCLPGAASSSSSTRAASTTSRVSPTTSRSSSATPPPGSTTTRVPPVGSGTATYSGNPFVGVTPWANAYYASEVSSLAIPSLTGAMATAAAAVAKVPSFMWLDTLDKTPLMEQTLADIRTANKNGGNYAGQFVVYDLPDRDCAALASNGEYSIADGGVAKYKNYIDTIRQIVVEYSDIRTLLVIEPDSLANLVTNLSTPKCANAQSAYLECINYAVTQLNLPNVAMYLDAGHAGWLGWPANQDPAAQLFANVYKNASSPRALRGLATNVASYNGWNITSPPSYTQGNAVYNEKLYIHAIGPLLANHGWSNAFFITDQGRSGKQPTGQQQWGDWCNVIGTGFGIRPSANTGDSLLDSFVWVKPGGECDGTSDSSAPRFDPHCALPDALQPAPQAGAWFQAYFVQLLTNANPSFL

SEQ ID NO: 59 - protein, 447 aa, Trichoderma reesei
QACSSVWGQCGGQNWSGPTCCASGSTCVYSNDYYSQCLPGAASSSSSTRAASTTSRVSPTTSRSSSATPPPGSTTTRVPPVGSGTATYSGNPFVGVTPWANAYYASEVSSLAIPSLTGAMATAAAAVAKVPSFMWLDTLDKTPLMEQTLADIRTANKNGGNYAGQFVVYDLPDRDCAALASNGEYSIADGGVAKYKNYIDTIRQIVVEYSDIRTLLVIEPDSLANLVTNLSTPKCANAQSAYLECINYAVTQLNLPNVAMYLDAGHAGWLGWPANQDPAAQLFANVYKNASSPRALRGLATNVASYNGWNITSPPSYTQGNAVYNEKLYIHAIGPLLANHGWSNAFFITDQGRSGKQPTGQQQWGDWCNVIGTGFGIRPSANTGDSLLDSFVWVKPGGECDGTSDSSAPQFDPHCALPDALQPAPQAGAWFQAYFVQLLTNANPSFL

SEQ ID NO: 60 - protein, 447 aa, Trichoderma reesei
QACSSVWGQCGGQNWSGPTCCASGSTCVYSNDYYSQCLPGAASSSSSTRAASTTSRVSPTTSRSSSATPPPGSTTTRVPPVGSGTATYSGNPFVGVTPWANAYYASEVSSLAIPSLTGAMATAAAAVAEVPSFMWLDTLDKTPLMEQTLADIRTANKNGGNYAGQFVVYDLPDRDCAALASNGEYSIADGGVAKYKNYIDTIRQIVVEYSDIRTLLVIEPDSLANLVTNLGTPKCANAQSAYLECINYAVTQLNLPNVAMYLDAGHAGWLGWPANQDPAAQLFANVYKNASSPRALRGLATNVANYNGWNITSPPSYTQGNAVYNEKLYIHAIGPLLANHGWSNAFFITDQGRSGKQPTGQQQWGDWCNVIGTGFGIRPSANTGDSLLDSFVWVKPGGECDGTSDSSAPRFDPHCALPDALQPAPQAGAWFQAYFVQLLTNANPSFL

SEQ ID NO: 61 - protein, 447 aa, Trichoderma reesei
QACSSVWGQCGGQNWSGPTCCASGSTCVYSNDYYSQCLPGAASSSSSTRAASTTSRVSPTTSRSSSATPPPGSTTTRVPPVGSGTATYSGNPFVGVTPWANAYYASEVSSLAIPSLTGAMATAAAAVAKVPSFMWLDTLDKTPLMEQTLADIRTANKNGGNYAGQFVVYDLPDRDCAALASNGEYTIADGGVAKYKNYIDTIRQIVVEYSDIRTLLVIEPDSLANLVTNLGTPKCANAQSAYLECINYAVTQLNLPNVAMYLDAGHAGWLGWPANQDPAAQLFANVYKNASSPRALRGLATNVANYNGWNITSPPSYTQGNAVYNEKLYIHAIGPLLANHGWSNAFFITDQGRSGKQPTGQQQWGDWCNVIGTGFGIRPSANTGDSLLDSFVWVKPGGECDGTSDSSAPRFDPHCALPDALQPAPQAGAWFQAYFVQLLTNANPSFL

SEQ ID NO: 62 - protein, 447 aa, Trichoderma reesei
QACSSVWGQCGGQNWSGPTCCASGSTCVYSNDYYSQCLPGAASSSSSTRAASTTSRVSPTTSRSSSATPPPGSTTTRVPPVGSGTATYSGNPFVGVTPWANAYYASEVSSLAIPSLTGAMATAAAAVAKVPSFMWLDTLDKTPLMEQTLADIRTANKNGGNYAGQFVVYDLPDRDCAALASNGEYSIADGGVAKYKNYIDTIRQIVVEYSDIRTLLVIEPDSLANLVTNLGTPKCANAQSAYLECINYAVTQLNLPNVAMYLDAGHAGWLGWPANQDPAAQLFANVYKNASSPRALRGLATNVANYNGWNITSPPSYTQGNDVYNEKLYIHAIGPLLANHGWSNAFFITDQGRSGKQPTGQQQWGDWCNVIGTGFGIRPSANTGDSLLDSFVWVKPGGECDGTSDSSAPRFDPHCALPDALQPAPQAGAWFQAYFVQLLTNANPSFL

SEQ ID NO: 63 - protein, 447 aa, Trichoderma reesei
QACSSVWGQCGGQNWSGPTCCASGSTCVYSNDYYSQCLPGAASSSSSTRAASTTSRVSPTTSRSSSATPPPGSTTTRVPPVGSGTATYSGNPFVGVTPWANAYYASEVSSLAIPSLTGAMATAAAAVAKVPSFMWLDTLDKTPLMEQTLADIRTANKNGGNYAGQFVVYDLPDRDCAALASNGEYSIADGGVAKYKNYIDTIRQIVVEYSDIRTLLVIEPDSLANLVTNLGTPKCANAQSAYLECINYAVTQLNLPNVAMYLDAGHAGWLGWPANQDPAAQLFANVYKNASSPRALRGLATNVANYNGWNITSPPSYTQGNAVYNEKLYIHAIGPLLANHGWSNAFFITDQGRSGKQPTGQQEWGDWCNVIGTGFGIRPSANTGDSLLDSFVWVKPGGECDGTSDSSAPRFDPHCALPDALQPAPQAGAWFQAYFVQLLTNANPSFL

SEQ ID NO: 64 - protein, 447 aa, Trichoderma reesei
QACSSVWGQCGGQNWSGPTCCASGSTCVYSNDYYSQCLPGAASSSSSTRAASTTSRVSPTTSRSSSATPPPGSTTTRVPPVGSGTATYSGNPFVGVTPWANAYYASEVSSLAIPSLTGAMATAAAAVAKVPSFMWLDTLDKTPLMEQTLADIRTANKNGGNYAGQFVVYDLPDRDCAALASNGEYSIADGGVAKYKNYIDTIRQIVVEYSDIRTLLVIEPDSLANLVTNLGTPKCANAQSAYLECINYAVTQLNLPNVAMYLDAGHAGWLGWPANQDPAAQLFANVYKNASSPRALRGLATNVANYNGWNITSPPSYTQGNAVYNEKLYIHAIGPLLANHGWSNAFFITDQGRSGKQPTGQQQWGDWCNVIGTGFGIRPSANTGDSLLDSFVWVKPGGECDGTSDSSAPGFDPHCALPDALQPAPQAGAWFQAYFVQLLTNANPSFL

SEQ ID NO: 65 - protein, 447 aa, Trichoderma reesei
ASCSSVWGQCGGQNWSGPTCCASGSTCVYSNDYYSQCLPGAASSSSSTRAASTTSRVSPTTSRSSSATPPPGSTTTRVPPVGSGTATYSGNPFVGVTPWANAYYASEVSSLAIPSLTGAMATAAAAVAKVPSFMWLDTLDKTPLMEQTLADIRTANKNGGNYAGQFVVYDLPDRDCAALASSGEYSIADGGVAKYKNYIDTIRQIVVEYSDIRTLLVIEPDSLANLVTNLGTPKCANAQSAYLECINYAVTQLNLPNVAMYLDAGHAGWLGWPANQDPAAQLFANVYKNASSPRALRGLATNVANYNGWNITSPPSYTQGNAVYNEKLYIHAIGPLLANHGWSNAFFITDQGRSGKQPTGQQQWGDWCNVIGTGFGIRPSANTGDSLLDSFVWVKPGGECDGTSDSSAPRFDPHCALPDALQPAPQAGAWFQAYFVQLLTNANPSFL

SEQ ID NO: 66 - protein, 447 aa, Trichoderma reesei
ASCSSVWGQCGGQNWSGPTCCASGSTCVYSNDYYSQCLPGAASSSSSTRAASTTSRVSPTTSRSSSATPPPGSTTTRVPPVGSGTATYSGNPFVGVTPWANAYYASEVSSLAIPSLTGAMATAAAAVAKVPSFMWLDTLDKTPLMEQTLADIRTANKNGGNYAGQFVVYDLPDRDCAALASGGEYSIADGGVAKYKNYIDTIRQIVVEYSDIRTLLVIEPDSLANLVTNLGTPKCANAQSAYLECINYAVTQLNLPNVAMYLDAGHAGWLGWPANQDPAAQLFANVYKNASSPRALRGLATNVANYNGWNITSPPSYTQGNAVYNEKLYIHAIGPLLANHGWSNAFFITDQGRSGKQPTGQQQWGDWCNVIGTGFGIRPSANTGDSLLDSFVWVKPGGECDGTSDSSAPRFDPHCALPDALQPAPQAGAWFQAYFVQLLTNANPSFL

SEQ ID NO: 67 - protein, 447 aa, Trichoderma reesei
ASCSSVWGQCGGQNWSGPTCCASGSTCVYSNDYYSQCLPGAASSSSSTRAASTTSRVSPTTSRSSSATPPPGSTTTRVPPVGSGTATYSGNPFVGVTPWANAYYASEVSSLAIPSLTGAMATAAAAVAKVPSFMWLDTLDKTPLMEQTLADIRTANKNGGNYAGQFVVYDLPDRDCAALASNGEYSIADGGVAKYKNYIDTIRQIVVEYSDIRTLLVIEPDSLANLVTNLGTPKCANAQSAYLECINYAVTQLNLPNVAMYLDAGHAGWLGWPANQDPAAQLFANVYKNASSPRALRGLATNVANYNGWNITSPPSYTQGNAVYNEKLYIHAIGPLLANHGWSNAFFITDQGRSGKQPTGQQQWGDWCNVIGTGFGIRPSANTGDSLLDSFVWVKPGGTCDGTSDSSAPRFDPHCALPDALQPAPQAGAWFQAYFVQLLTNANPSFL

SEQ ID NO: 68 - protein, 447 aa, Trichoderma reesei
ASCSSVWGQCGGQNWSGPTCCASGSTCVYSNDYYSQCLPGAASSSSSTRAASTTSRVSPTTSRSSSATPPPGSTTTRVPPVGSGTATYSGNPFVGVTPWANAYYASEVSSLAIPSLTGAMATAAAAVAKVPSFMWLDTLDKTPLMEQTLADIRTANKNGGNYAGQFVVYDLPDRDCAALASNGEYSIADGGVAKYKNYIDTIRQIVVEYSDIRTLLVIEPDSLANLVTNLGTPKCANAQSAYLECINYAVTQLNLPNVAMYLDAGHAGWLGWPANQDPAAQLFANVYKNASSPRALRGLATNVANYNGWNITSPPSYTQGNAVYNEKLYIHAIGPLLANHGWSNAFFITDQGRSGKQPTGQQQWGDWCNVIGTGFGIRPSANTGDSLLDSFVWVKPGGSCDGTSDSSAPRFDPHCALPDALQPAPQAGAWFQAYFVQLLTNANPSFL

SEQ ID NO: 69 - protein, 447 aa, Trichoderma reesei
ASCSSVWGQCGGQNWSGPTCCASGSTCVYSNDYYSQCLPGAASSSSSTRAASTTSRVSPTTSRSSSATPPPGSTTTRVPPVGSGTATYSGNPFVGVTPWANAYYASEVSSLAIPSLTGAMATAAAAVAKVPSFMWLDTLDKTPLMEQTLADIRTANKNGGNYAGQFVVYDLPDRDCAALASNGEYSIADGGVAKYKNYIDTIRQIVVEYSDIRTLLVIEPDSLANLVTNLGTPKCANAQSAYLECINYAVTQLNLPNVAMYLDAGHAGWLGWPANQDPAAQLFANVYKNASSPRALRGLATNVANYNGWNITSPPSYTQGNAVYNEKLYIHAIGPLLANHGWSNAFFITDQGRSGKQPTGQQQWGDWCNVIGTGFGIRPSANTGDSLLDSFVWVKPGGECDGTSDSSAPRFDPHCALPDALQPAPQVGAWFQAYFVQLLTNANPSFL

SEQ ID NO: 70 - protein, 447 aa, Trichoderma reesei
ASCSSVWGQCGGQNWSGPTCCASGSTCVYSNDYYSQCLPGAASSSSSTRAASTTSRVSPTTSRSSSATPPPGSTTTRVPPVGSGTATYSGNPFVGVTPWANAYYASEVSSLAIPSLTGAMATAAAAVAKVPSFMWLDTLDKTPLMEQTLADIRTANKNGGNYAGQFVVYDLPDRDCAALASNGEYSIADGGVAKYKNYIDTIRQIVVEYSDIRTLLVIEPDSLANLVTNLGTPKCANAQSAYLECINYAVTQLNLPNVAMYLDAGHAGWLGWPANQDPAAQLFANVYKNASSPRALRGLATNVANYNGWNITSPPSYTQGNAVYNEKLYIHAIGPLLANHGWSNAFFITDQGRSGKQPTGQQQWGDWCNVIGTGFGIRPSANTGDSLLDSFVWVKPGGECDGTSDSSAPRFDPHCALPDALQPAPQLGAWFQAYFVQLLTNANPSFL

SEQ ID NO: 71 - protein, 447 aa, Trichoderma reesei
ASCSSVWGQCGGQNWSGPTCCASGSTCVYSNDYYSQCLPGAASSSSSTRAASTTSRVSPTTSRSSSATPPPGSTTTRVPPVGSGTATYSGNPFVGVTPWANAYYASEVSSLAIPSLTGAMATAAAAVAKVPSFMWLDTLDKTPLMEQTLADIRTANKNGGNYAGQFVVYDLPDRDCAALASNGEYSIADGGVAKYKNYIDTIRQIVVEYSDIRTLLVIEPDSLANLVTNLGTPKCANAQSAYLECINYAVTQLNLPNVAMYLDAGHAGWLGWPANQDPAAQLFANVYKNASSPRALRGLATNVANYNGWNITSPPSYTQGNAVYNEKLYIHAIGPLLANHGWSNAFFITDQGRSGKQPTGQQQWGDWCNVIGTGFGIRPSANTGDSLLDSFVWVKPGGECDGTSDSSAPRFDPHCALPDALQPAPQSGAWFQAYFVQLLTNANPSFL

SEQ ID NO: 72 - protein, 447 aa, Trichoderma reesei
QACSSVWGQCGGQNWSGPTCCASGSTCVYSNDYYFQCLPGAASSSSSTRAASTTSRVSPTTSRSSSATPPPGSTTTRVPPVGSGTATYSGNPFVGVTPWANAYYASEVSSLAIPSLTGAMATAAAAVAKVPSFMWLDTLDKTPLMEQTLADIRTANKNGGNYAGQFVVYDLPDRDCAALASNGEYSIADGGVAKYKNYIDTIRQIVVEYSDIRTLLVIEPDSLANLVTNLGTPKCANAQSAYLECINYAVTQLNLPNVAMYLDAGHAGWLGWPANQDPAAQLFANVYKNASSPRALRGLATNVANYNGWNITSPPSYTQGNAVYNEKLYIHAIGPLLANHGWSNAFFITDQGRSGKQPTGQQQWGDWCNVIGTGFGIRPSANTGDSLLDSFVWVKPGGECDGTSDSSAPRFDPHCALPDALQPAPQAGAWFQAYFVQLLTNANPSFL

SEQ ID NO: 73 - protein, 447 aa, Trichoderma reesei; identical to SEQ ID NO: 72

SEQ ID NO: 74 - protein, 447 aa, Trichoderma reesei; identical to SEQ ID NO: 72

SEQ ID NO: 75 - protein, 447 aa, Trichoderma reesei; identical to SEQ ID NO: 72

SEQ ID NO: 76 - protein, 447 aa, Trichoderma reesei; identical to SEQ ID NO: 72

SEQ ID NO: 77 - protein, 447 aa, Trichoderma reesei; identical to SEQ ID NO: 72

SEQ ID NO: 78 - protein, 447 aa, Trichoderma reesei; identical to SEQ ID NO: 72

SEQ ID NO: 79 - protein, 447 aa, Hypocrea jecorina; identical to SEQ ID NO: 72

SEQ ID NO: 80 - protein, 447 aa, Artificial Sequence; identical to SEQ ID NO: 72
  Obtained from environmental sample

SEQ ID NO: 81 - protein, 447 aa, Trichoderma reesei; identical to SEQ ID NO: 72

SEQ ID NO: 82 - protein, 493 aa, Artificial Sequence
  Obtained from environmental sample
VPLEERQACSSVWGQCGGQNWSGPTCCASGSTCVYSNDYYSQCLPGAASSSSSTRAASTTSRVSPTTSRSSSATPPPGSTTTRVPPVGSGTATYSGNPFVGVTPWANAYYASEVSSLAIPSLTGAMATAAAAVAKVPSFMWLDTLDKTPLMEQTLADIRTANKNGGNYAGQFVVYDLPDRDCAALASNGEYSIADGGVAKYKNYIDTIRQIVVEYSDIRTLLVIEPDSLANLVTNLGTPKCANAQSAYLECINYAVTQLNLPNVAMYLDAGHAGWLGWPANQDPAAQLFANVYKNASSPRALRGLATNVANYNGWNITSPPSYTQGNAVYNEKLYIHAIGPLLANHGWSNAFFITDQGRSGKQPTGQQQWGDWCNVIGTGFGIRPSANTGDSLLDSFVWVKPGGECDGTSDSSAPRFDSHCALPDALQPAPQAGAWFQAYFVQLLTNANPSFLGSGGGGSGGGGSHHHHHHGGENLYFQGGGGGSGGGGSGSA

SEQ ID NO: 83 - protein, 457 aa, Gibberella zeae
MTAYKLFLAAAFAATALAAPVEERQSCSNGVWSQCGGQNWSGTPCCTSGNKCVKVNDFYSQCQPGSADPSPTSTIVSATTTKATTTGSGGSVTSPPPVATNNPFSGVDLWANNYYRSEVSTLAIPKLSGAMATAAAKVADVPSFQWMDTYDHISFMEDSLADIRKANKAGGNYAGQFVVYDLPDRDCAAAASNGEYSLDKDGKNKYKAYIADQGILQDYSDTRIILVIEPDSLANMVTNMNVPKCANAASAYKELTIHALKELNLPNVSMYIDAGHGGWLGWPANLPPAAQLYGQLYKDAGKPSRLRGLVTNVSNYNAWKLSSKPDYTESNPNYDEQKYIHALSPLLEQEGWPGAKFIVDQGRSGKQPTGQKAWGDWCNAPGTGFGLRPSANTGDALVDAFVWVKPGGESDGTSDTSAARYDYHCGIDGAVKPAPEAGTWFQAYFEQLLKNANPSFL

SEQ ID NO: 84 - protein, 462 aa, Fusarium oxysporum
MAYKLILAAFAATALAAPVEERQSCSNGVWAQCGGQNWSGTPCCTSGNKCVKLNDFYSQCQPGSAEPSSTAAGPSSTTATKTTATGGSSTTAGGSVTSAPPAASDNPYAGVDLWANNYYRSEVMNLAVPKLSGAKATAAAKVADVPSFQWMDTYDHISLMEDTLADIRKANKAGGKYAGQFVVYDLPNRDCAAAASNGEYSLDKDGANKYKAYIAKIKGILQNYSDTKVILVIEPDSLANLVTNLNVDKCAKAESAYKELTVYAIKELNLPNVSMYLDAGHGGWLGWPANIGPAAKLYAQIYKDAGKPSRVRGLVTNVSNYNGWKLSTKPDYTESNPNYDEQRYINAFAPLLAQEGWSNVKFIVDQGRSGKQPTGQKAQGDWCNAKGTGFGLRPSTNTGDALADAFVWVKPGGESDGTSDTSAARYDYHCGLDDALKPAPEAGTWFQAYFEQLLDNANPSFL

SEQ ID NO: 85 - protein, 471 aa, Trichoderma reesei
MIVGILTTLATLATLAASVPLEERQACSSVWGQCGGQNWSGPTCCASGSTCVYSNDYYSQCLPGAASSSSSTRAASTTSRVSPTTSRSSSATPPPGSTTTRVPPVGSGTATYSGNPFVGVTPWANAYYASEVSSLAIPSLTGAMATAAAAVAKVPSFMWLDTLDKTPLMEQTLADIRTANKNGGNYAGQFVVYDLPDRDCAALASNGEYSIADGGVAKYKNYIDTIRQIVVEYSDIRTLLVIEPDSLANLVTNLGTPKCANAQSAYLECINYAVTQLNLPNVAMYLDAGHAGWLGWPANQDPAAQLFANVYKNASSPRALRGLATNVANYNGWNITSPPSYTQGNAVYNEKLYIHAIGPLLANHGWSNAFFITDQGRSGKQPTGQQQWGDWCNVTGTGFGIRPSANTGDSLLDSFVWVKPGGECDGTSDSSAPRFDSHCALPDALQPAPQAGAWFQAYFVQLLTNANPSFL

SEQ ID NO: 86 - protein, 471 aa, Trichoderma reesei; identical to SEQ ID NO: 14

SEQ ID NO: 87 - protein, 471 aa, Hypocrea rufa; identical to SEQ ID NO: 19

SEQ ID NO: 88 - protein, 471 aa, Trichoderma reesei; identical to SEQ ID NO: 16

SEQ ID NO: 89 - protein, 471 aa, Trichoderma reesei; identical to SEQ ID NO: 20

SEQ ID NO: 90 - protein, 470 aa, Hypocrea koningii; identical to SEQ ID NO: 17

SEQ ID NO: 91 - protein, 470 aa, Trichoderma longibrachiatum
MIVGILTTLATLATLAASVPLEERQACSSVWGQCGGQNWSGPTCCASGSTCVYSNDYYSQCLPGAASSSSSTRASSTTARASSTTSRSSATPPPGSSTTRVPPVGSGTATYSGNPFVGVTPWANAYYASEVSSLAIPSLTGAMATAAAAVAKVPSFMWLDTFDKTPLMEQTLADIRTANKNGGNYAGQFVVYDLPDRDCAALASNGEYSIADGGVDKYKNYIDTIRQIVVEYSDIRTLLVIEPDSLANLVTNLGTPKCANAQSAYLECINYAVTQLNLPNVAMYLDAGHAGWLGWPANQDPAAQLFANVYKNASSPRALRGLATNVANYNGWNITSPPSYTQGNAVYNEQLYIHAIGPLLANHGWSNAFFITDQGRSGKQPTGQQQWGDWCNVIGTGFGIRPSANTGDSLLDSFVWIKPGGECDGTSDSSAPRFDSHCALPDALQPAPQAGAWFQAYFVQLLTNANPSFL

SEQ ID NO: 92 - protein, 470 aa, Hypocrea koningii
MIVGILTTLATLATLAASVPLEERQACSSVWGQCGGQNWSGPTCCASGSTCVYSNDYYSQCLPGAASSSSSTRASSTTARASSTTSRSSATPPPGSSTTRVPPVGSGTATYSGNPFVGVTPWANAYYASEVSSLAIPSLTGAMATAAAAVAKVPSSMWLDTFDKTPLMEQTLADIRTANKNGGNYAGQFVVYDLPDRDCAALASNGEYSIADGGVDKYKNYIDTIRQIVVEYSDIRTLLVIEPDSLANLVTNLGTPKCANAQSAYLECINYAVTQLNLPNVAMYLDAGHAGWLGWPANQDPAAQLFANVYKNASSPRALRGLATNVANYNGWNITSPPSYTQGNAVYNEQLYIHAIGPLLANHGWSNAFFITDQGRSGKQPTGQQQWGDWCNVIGTGFGIRPSANTGDSLLDSFVWIKPGGECDGTSDSSAPRFDSHCALPDALQPAPQAGAWFQAYFVQLLTNANPSFL

SEQ ID NO: 93 - protein, 470 aa, Trichoderma sp
MIVGILTTLATLATLAASVPLEERQACSSVWGQCGGQNWSGPTCCASGSTCVYSNDYYSQCLPGAASSSSSTRASSTTARASSTTSRSSATPPPGSSTTRVPPVGSGTATYSGNPFVGVTPWANAYYASEVSSLAIPSLTGAMATAAAAVAKVPSFMWLDTFDKTPLMEQTLADIRTANKNGGNYAGQFVVYDLPDRDCAALASNGEYSIADGGVDKYKNYIDTIRQIVVEYSDIRTLLVIEPDSLANLVTNLGTPKCANAQSAYLECINYAVTQLNLPNVAMYLDAGHAGWLGWPANQDPAAQLFANVYKNASSPRALRGLATNVANYNGWNITSPPSYTQGNAVYNEQLYIHAIGPLLANHGWSNAFFITDQGRSGKQPTGHQQWGDWCNVIGTGFGIRPSANTGDSLLDSFVWIKPGGECDGTSDSSAPRFDSHCALPDALQPAPQAGAWFQAYFVQLLTNANPSFL

SEQ ID NO: 94 - protein, 470 aa, Hypocrea rufa
MIVGILTTLATLATLAASVPLEERQACSSVWGQCGGQNWSGPTCCASGSTCVYSNDYYSQCLPGAASSSSSTRASSTTARASSTTSRSSATPPPGSSTTRVPPVGSGTATYSGNPFVGVTPWANAYYASEVSSLAIPSLTGAMATAAAAVAKVPSFMWLDTFDKTPLMEQTLADIRTANKNGGNYAGQFVVYDLPDRDCAALASNGEYSIADGGVDKYKNYIDTIRQIVVEYSDIRTLLVIEPDSLANLVTNLGTPKCANAQFAYLECINYAVTQLNLPNVAMYLDAGHAGWLGWPANQDPAAQLFANVYKNASSPRALRGLATNVANYNGWNITSPPSYTQGNAVYNEQLYIHAIGPLLANHGWSNAFFITDQGRSGKQPTGQQQWGDWCNVIGTGFGIRPSANTGDSLLDSFVWIKPGGECDGTSDSSAPRFDSHCALPDALQPAPQAGAWFQAYFVQFLTNANPSFL

SEQ ID NO: 95 - protein, 470 aa, Trichoderma parceramosum; identical to SEQ ID NO: 18

SEQ ID NO: 96 - protein, 481 aa, Thievlavia terrestris NRRL 8126
MAQKLLLAAALAASALAAPVVEERQNCGSVWSQCGGIGWSGATCCASGNTCVELNPYYSQCLPNSQVTTSTSKTTSTTTRSSTTSHSSGPTSTSTTTTSSPVVTTPPSTSIPGGASSTASWSGNPFSGVQMWANDYYASEVSSLAIPSMTGAMATKAAEVAKVPSFQWLDRNVTIDTLFAHTLSQIRAANQKGANPPYAGIFVVYDLPDRDCAAAASNGEFSIANNGAANYKTYIDAIRSLVIQYSDIRIIFVIEPDSLANMVTNLNVAKCANAESTYKELTVYALQQLNLPNVAMYLDAGHAGWLGWPANIQPAANLFAEIYTSAGKPAAVRGLATNVANYNGWSLATPPSYTQGDPNYDESHYVQALAPLLTANGFPAHFITDTGRNGKQPTGQRQWGDWCNVIGTGFGVRPTTNTGLDIEDAFVWVKPGGECDGTSNTTSPRYDYHCGLSDALQPAPEAGTWFQAYFEQLLTNANPPF

SEQ ID NO: 97 - protein, 473 aa, Artificial Sequence
  Obtained from environmental sample
MRVLLVALALLALAASATSVPLEERQSCSSVWGQCGGQNWAGPFCCASGSTCVYSNDYYSQCLPGTASSSSSTRASSTTSRVSSATSTRSSSSTPPPASSTTPAPPVGSGTATYSGNPFAGVTPWANSFYASEVSTLAIPSLTGAMATAAAAVAKVPSFMWLDTLDKTPLMSSTLSDIRAANKAGGNYAGQFVVYDLPDRDCAAAASNGEYSIADGGVAKYKNYIDTIRGIVTTFSDVRILLVIEPDSLANLVTNLATPKCSNAQSAYLECINYAITQLNLPNVAMYLDAGHAGWLGWPANQDPAAQLFANVYKNASSPRAVRGLATNVANYNAWNITTPPSYTQGNAVYNEKLYIHALGPLLANHGWSNAFFITDQGRSGKQPTGQLEWGNWCNAVGTGFGIRPSANTGDSLLDSFVWIKPGGECDGTSNSSAPRFDYHCASADALQPAPQAGSWFQAYFVQLLTNANPSFL

SEQ ID NO: 98 - protein, 482 aa, Myceliophthora thermophila
MAKKLFITAALAAAVLAAPVIEERQNCGAVWTQCGGNGWQGPTCCASGSTCVAQNEWYSQCLPNSQVTSSTTPSSTSTSQRSTSTSSSTTRSGSSSSSSTTPPPVSSPVTSIPGGATSTASYSGNPFSGVRLFANDYYRSEVHNLAIPSMTGTLAAKASAVAEVPSFQWLDRNVTIDTLMVQTLSQVRALNKAGANPPYAAQLVVYDLPDRDCAAAASNGEFSIANGGAANYRSYIDAIRKHIIEYSDIRIILVIEPDSMANMVTNMNVAKCSNAASTYHELTVYALKQLNLPNVAMYLDAGHAGWLGWPANIQPAAELFAGIYNDAGKPAAVRGLATNVANYNAWSIASAPSYTSPNPNYDEKHYIEAFSPLLNSAGFPARFIVDTGRNGKQPTGQQQWGDWCNVKGTGFGVRPTANTGHELVDAFVWVKPGGESDGTSDTSAARYDYHCGLSDALQPAPEAGQWFQAYFEQLLTNANPPF

SEQ ID NO: 99 - protein, 476 aa, Chaetomium thermophilum
MAKQLLLTAALAATSLAAPLLEERQSCSSVWGQCGGINYNGPTCCQSGSVCTYLNDWYSQCIPGQAQPGTTSTTARTTSTSTTSTSSVRPTTSNTPVTTAPPTTTIPGGASSTASYNGNPFSGVQLWANTYYSSEVHTLAIPSLSPELAAKAAKVAEVPSFQWLDRNVTVDTLFSGTLAEIRAANQRGANPPYAGIFVVYDLPDRDCAAAASNGEWSIANNGANNYKRYIDRIRELLIQYSDIRTILVIEPDSLANMVTNMNVQKCSNAASTYKELTVYALKQLNLPHVAMYMDAGHAGWLGWPANIQPAAELFAQIYRDAGRPAAVRGLATNVANYNAWSIASPPSYTSPNPNYDEKHYIEAFAPLLRNQGFDAKFIVDTGRNGKQPTGQLEWGHWCNVKGTGFGVRPTANTGHELVDAFVWVKPGGESDGTSDTSAARYDYHCGLSDALTPAPEAGQWFQAYFEQLLINANPPF

SEQ ID NO: 100 - protein, 484 aa, Podospora anserina S mat+
MAKRLLLTAALAATTLAAPVIEERQNCGSVWSQCGGQGWTGATCCASGSTCVAQNQWYSQCLPGSQVTTTAQAPSSTRTTTSSSSRPTSSSISTSAVNVPTTTTSAGASVTVPPGGGASSTASYSGNPFLGVQQWANSYYSSEVHTLAIPSLTGPMATKAAAVAKVPSFQWMDRNVTVDTLFSGTLADIRAANRAGANPPYAGIFVVYDLPDRDCAAAASNGEWAIADGGAAKYKAYIDRIRHHLVQYSDIRTILVIEPDSLANMVTNMNVPKCQGAANTYKELTVYALKQLNLPNVAMYLDAGHAGWLGWPANIGPAAELFAGIYKDAGRPTSLRGLATNVANYNGWSLSSAPSYTTPNPNFDEKRFVQAFSPLLTAAGFPAHFITDTGRSGKQPTGQLEWGHWCNAIGTGFGPRPTTDTGLDIEDAFVWIKPGGECDGTSDTTAARYDHHCGFADALKPAPEAGQWFQAYFEQLLTNANPPF

SEQ ID NO: 101 - protein, 476 aa, Chaetomium thermophilum
MAKQLLLTAALAAISLAAPLLEERQSCSSVWGQCGGINYNGPTCCQSGSVCAYLNDWYSQCIPGQAQPGTTSTTARTTSTSTTSTSSVRPTTSNTPVTTAPPTTTIPGGASSTASYNGNPFSGVQLWANTYYSSEVHTLAIPSLSPELAAKAAKVAEVPSFQWLDRNVTVDTLFSGTLAEIRAANQRGANPPYAGIFVVYDLPDRDCAAAASNGEWSIANNGANNLQRYIDRIRELLIQYSDIRTILVIEPDSLANMVTNMNVQKCSNAASTYKELTVYALKQLNLPHVAMYMDAGHAGWLGWPANIQPAAELFAQIYRDAGRPAAVRGLATNVANYNAWSIASPPSYTSPNPNYDEKHYIEAFAPLLRNQGFDAKFIVDTGRNGKQPTGQLEWGHWCNVKGTGFGVRPTANTGHELVDAFVWVKPGGESDGTSDTSAARYDYHCGLSDALTPAPEAGQWFQAYFEQLLINANPPF

SEQ ID NO: 102 - protein, 465 aa, Phialophora sp
MTAKHVFLAAALAATALAAPVSESQNCASEWGQCGGTGFTGASCCASGSTCTQQNEYYSQCVPGSQVTTGQIASTPAATVVGSATMGSSPSQMTAPAASASGTTSYSGNPFEGVQMWANAYYASEVLNLAVPSLSGDMVAKASAVAKVPSFQWLDTAAKVPTVMADTLADIAKANQAGASPAYAGLFVVYDLPDRDCAAAASNGEYSIADNGVANYKAYIDAIKAQLVANSDTRILLVVEPDSLANLVTNMNVAKCANAHDAYLECINYAVTQLNLPNVAMYLDAGHAGWLGWSANLQPAATLFANVYSNAGKPASLRGLATNVANYNAWTIASAPSYTQGDSNYDEKLYVQALSPLLSSAGWDAHFITDQSRSGKQPTGQNAWGDWCNVIGTGFGTRPTTDTGLDIEDAVWVKPGGECDGTSNTTAARYDYHCGLSDALQPSPEAGTWFQAYFVQLLTNANPAF

SEQ ID NO: 103 - protein, 484 aa, Neurospora crassa
MAAKKLLLAAALTASALAAPVLEDRQNCGSAWSQCGGIGWSGATCCSSGNSCVEINSYYSQCLPGAQVTTTAGASSTSPTSTSKVSSTTSKVTSSSAAQPITTTTAPSVPTTTIAGGASSTASFTGNPFLGVQGWANSYYSSEIYNHAIPSMTGSLAAQASAVAKVPTFQWLDRNVTVDTLMKSTLEEIRAANKAGANPPYAAHFVVYDLPDRDCAAAASNGEFSIANGGVANYKTYINAIRKLLIEYSDIRTILVIEPDSLANLVTNTNVAKCANAASAYRECTNYAITQLDLPHVAQYLDAGHGGWLGWPANIQPAATLFADIYKAAGKPKSVRGLVTNVSNYNGWSLSSAPSYTTPNPNYDEKKYIEAFSPLLNAAGFPAQFIVDTGRSGKQPTGQIEQGDWCNAIGTGFGVRPTTNTGSSLADAFVWVKPGGESDGTSDTSATRYDYHCGLSDALKPAPEAGQWFQAYFEQLLKNANPAF

SEQ ID NO: 104 - protein, 476 aa, Humicola insolens
MAKFFLTAAFAAAALAAPVVEERQNCAPTWGQCGGIGFNGPTCCQSGSTCVKQNDWYSQCLPGSQVTTTSTTSTSSSSTTSRATSTTRTGGVTSITTAPTRTVTIPGGATTTASYNGNPFEGVQLWANNYYRSEVHTLAIPQITDPALRAAASAVAEVPSFQWLDRNVTVDTLLVETLSEIRAANQAGANPPYAAQIVVYDLPDRDCAAAASNGEWAIANNGANNYKGYINRIREILISFSDVRTILVIEPDSLANMVTNMNVAKCSGAASTYRELTIYALKQLDLPHVAMYMDAGHAGWLGWPANIQPAAELFAKIYEDAGKPRAVRGLATNVANYNAWSISSPPPYTSPNPNYDEKHYIEAFRPLLEARGFPAQFIVDQGRSGKQPTGQKEWGHWCNAIGTGFGMRPTANTGHQYVDAFVWVKPGGECDGTSDTTAARYDYHCGLEDALKPAPEAGQWFQAYFEQLLRNANPPF

SEQ ID NO: 105 - protein, 455 aa, Emericella nidulans
MHYSASGLALAFLLPAIQAQQTLYGQCGGSGWTGATSCVAGAACSTLNQWYAQCLPAATTTSTTLTTTTSSVTTTSNPGSTTTTSSVTVTATASGNPFSGYQLYVNPYYSSEVQSIAIPSLTGTLSSLAPAATAAAKVPSFVWLDVAAKVPTMATYLADIRSQNAAGANPPIAGQFVVYDLPDRDCAALASNGEFAISDGGVQHYKDYIDSIREILVEYSDVHVILVIEPDSLANLVTNLNVAKCANAQSAYLECTNYAVTQLNLPNVAMYLDAGHAGWLGWPANLQPAANLYAGVYSDAGSPAALRGLATNVANYNAWAIDTCPSYTQGNSVCDEKDYINALAPLLRAQGFDAHFITDTGRNGKQPTGQQAWGDWCNVIGTGFGARPSTNTGDSLLDAFVWVKPGGESDGTSDTSAARYDAHCGYSDALQPAPEAGTWFQAYFVQLLQNANPSF

SEQ ID NO: 106 - protein, 459 aa, Aspergillus niger
MHYPLSLALAFLPFGIQAQQTLWGQCGGQGYSGATSCVAGATCATVNEYYAQCTPAAGTSSATTLKTTTSSTTAAVTTTTTTQSPTGSASPTTTASASGNPFSGYQLYVNPYYSSEVASLAIPSLTGSLSSLQAAATAAAKVPSFVWLDTAAKVPTMGDYLADIQSQNAAGANPPIAGQFVVYDLPDRDCAALASNGEYSIADNGVEHYKSYIDSIREILVQYSDVHTLLVIEPDSLANLVTNLNVAKCANAESAYLECTNYALTQLNLPNVAMYLDAGHAGWLGWPANQQPAADLFASVYKNASSPAAVRGLATNVANYNAWTISSCPSYTQGNSVCDEQQYINAIAPLLQAQGFDAHFIVDTGRNGKQPTGQQAWGDWCNVINTGFGERPTTDTGDALVDAFVWVKPGGESDGTSDSSATRYDAHCGYSDALQPAPEAGTWFQAYFVQLLTNANPAF

SEQ ID NO: 107 - protein, 391 aa, Penicillium chrysogenum
MRSFIPFVSLLATSAAAAAISSAASPTVTAAAAGNPFSGYQLYANSYYASEVSSLALPSMTGAAKAAASVAAKVPSFYWLDTAAKVPTMGEFLADIRAKNKAGASPPIAGQFVVYDLPDRDCAALASNGEYSIADGGVAKYKAYIDAIREILVEYSDIQTILVVEPDSLANLVTNMAVSKCANAHDAYLECTNYAVTQLNLDNVAMYLDAGHAGWLGWPANLGPAAELYANVYKTANKPASMRGLATNVANYNGWSLSTCPSYTSGNSNCDEKKYINALGPLLKTAGWDAHFITDTGRNGVQPTSQSAWGDWCNVKGTGFGVRPTTETGDALADAFVWVKPGGESDGTSDSSAARYDAHCGYSDALQPAPEAGTWFQAYFAQLVENANPSL

SEQ ID NO: 108 - protein, 400 aa, Aspergillus niger
MRAIWPLVSLFSAVKALPAASATASASVAASSSPAPTASATGNPFEGYQLYANPYYKSQVESSAIPSLSASSLVAQASAAADVPSFYWLDTADKVPTMGEYLEDIQTQNAAGASPPIAGIFVVYDLPDRDCSALASNGEYSISDGGVEKYKAYIDSIREQVETYSDVQTILIIEPDSLANLVTNLDVAKCANAESAYLECTNYALEQLNLPNVAMYLDAGHAGWLGWPANIGPAAQLYASVYKNASSPAAVRGLATNVANFNAWSIDSCPSYTSGNDVCDEKSYINAIAPELSSAGFDAHFITDTGRNGKQPTGQSAWGDWCNVKDTGFGAQPTTDTGDELADAFVWVKPGGESDGTSDTSSSRYDAHCGYSDALQPAPEAGTWFQAYFEQLLTNANPSL

SEQ ID NO: 109 - protein, 407 aa, Aspergillus oryzae
MHTLNMQALVALSPLLFSAATALPQASVTPSPSSSVPASSGPAPTATAGGNPFEGYDLYVNPYYKSEVESLAIPSMTGSLAEKASAAANVPSFHWLDTTDKVPQMGEFLEDIKTKNAAGANPPTAGIFVVYDLPDRDCAALASNGEFLISDGGVEKYKAYIDSIREQVEKYSDTQIILVIEPDSLANLVTNLNVQKCANAQDAYLECTNYALTQLNLPNVAMYLDAGHAGWLGWPANIGPAAELYASVYKNASSPAAVRGLATNVANYNAFSIDSCPSYTQGSTVCDEKTYINNFAPQLKSAGFDAHFIVDTGRNGNQPTGQSQWGDWCNVKNTGFGVRPTTDTGDELVDAFVWVKPGGESDGTSDTSAERYDAHCGYADALTPAPEAGTWFQAYFEQLVENANPSL

SEQ ID NO: 110 - protein, 459 aa, Botryotinia fuckeliana
MGFKNALLAAAAVAPTVYAQGAAYAQCGGQGWSGATTCVSGYTCVVNNAYYSQCLPGSAVTTTATTAPTATTPTTIITSTTKATTTTGGSSATTTAAVAGNPFSGKALYANPYYASEISASAIPSLTGAMATKAAAVAKVPTFYWLLSDTAAKVPLMGTYLANIRALNKAGANPPVAGTFVVYDLPDRDCAAAASNGEYSIADGGLVKYKAYIDSIVALLKTYSDVSVILVIEPDSLANLVTNLSVAKCSNAQAAYLEGTEYAIAQLNLPNVAMYLDAGHAGWLGWPANIGPAAQLFGQIYKAAGSPAAVRGLATNVANYNAWTSTTCPSYTSGDSNCNEKLYINALAPLLTAQGFPAHFIMDTSRNGVQPTAQQAWGDWCNLIGTGFGVRPTTNTGDALEDAFVWIKPGGEGDGTSDTTAARYDFHCGLADALKPAPEAGTWFQAYFAQLLTNANPSF

SEQ ID NO: 111 - protein, 459 aa, Rasamsonia emersonii
MRNLLALAPAALLVGAAEAQQSLWGQCGGSSWTGATSCAAGATCSTINPYYAQCVPATATPTTLTTTTKPTSTGGAAPTTPPPTTTGTTTSPVVTRPASASGNPFEGYQLYANPYYASEVISLAIPSLSSELVPKASEVAKVPSFVWLDQAAKVPSMGDYLKDIQSQNAAGADPPIAGIFVVYDLPDRDCAAAASNGEFSIANNGVALYKQYIDSIREQLTTYSDVHTILVIEPDSLANVVTNLNVPKCANAQDAYLECINYAITQLDLPNVAMYLDAGHAGWLGWQANLAPAAQLFASVYKNASSPASVRGLATNVANYNAWSISRCPSYTQGDANCDEEDYVNALGPLFQEQGFPAYFIIDTSRNGVRPTKQSQWGDWCNVIGTGFGVRPTTDTGNPLEDAFVWVKPGGESDGTSNTTSPRYDYHCGLSDALQPAPEAGTWFQAYFEQLLTNANPLF

SEQ ID NO: 112 - protein, 457 aa, Penicillium funiculosum
MLRYLSIVAATAILTGVEAQQSVWGQCGGQGWSGATSCAAGSTCSTLNPYYAQCIPGTATSTTLVKTTSSTSVGTTSPPTTTTTKASTTATTTAAASGNPFSGYQLYANPYYSSEVHTLAIPSLTGSLAAAATKAAEIPSFVWLDTAAKVPTMGTYLANIEAANKAGASPPIAGIFVVYDLPDRDCAAAASNGEYTVANNGVANYKAYIDSIVAQLKAYPDVHTILIIEPDSLANMVTNLSTAKCAEAQSAYYECVNYALIKPHLAHVAMYIDAGHAGWLGWSANLSPAAQLFATVYKNASAPASLRGLATNVANYNAWSISSPPSYTSGDSNYDEKLYINALSPLLTSNGWPDAHFIMDTSRNGVQPTKQQAWGDWCNVIGTGFGVQPTTNTGDPLEDAFVWVKPGGESDGTSNSSATRYDFHCGYSGALQPAPEAGTWFQAYFVQLLTNANPALV

SEQ ID NO: 113 - protein, 464 aa, Penicillium decumbens
MQRTSAWALLLLAQIATAQQTVWGQCGGIGYSGPTSCVAGSSCSTQNSYYAQCLPGSGNGGGGAATTTTTAGQTTKTTMATTTTTSTKTSAGSGGSTTTAPPASNSGNPFKGYQPYVNPYYASEVQSLAIPSLAASLAPKASAVAKVPSFVWLDTAAKVPTMGTYLADIKAKNAAGANPPIAGIFVVYDLPDRDCAALASNGEYSIANGGVANYKKYIDSIRAQLLKYPDVHTILVIEPDSLANLVTNMNVAKCSGAHDAYLECTDYALKQLNLPNVAMYLDAGHAGWLGWPANIGPAADLFASVYKNAGSPAAVRGLATNVANYNAWSISTCPSYTQGDQNCDEKRYINALAPLLRANGFDAHFIMDTSRNGVQPTKQQAWGDWCNVIGTGFGTPFTTDTGDALQDAFIWVKPGGECDGTSDTSSPRYDAHCGYSDALKPAPEAGTWFQAYFEQLLVNANPSF

SEQ ID NO: 114 - protein, 457 aa, Acremonium cellulolyticus Y-94
MLRYLSIVAATAILTGVEAQQSVWGQCGGQGWSGATSCAAGSTCSTLNPYYAQCIPGTATSTTLVKTTSSTSVGTTSPPTTTTTKASTTATTTAAASGNPFSGYQLYANPYYSSEVHTLAIPSLTGSLAAAATKAAEIPSFVWLDTAAKVPTMGTYLANIEAANKAGASPPIAGIFVVYDLPDRDCAAAASNGEYTVANNGVANYKAYIDSIVAQLKAYPDVHTILIIEPDSLANMVTNLSTAKCAEAQSAYYECVNYALINLNLANVAMYIDAGHAGWLGWSANLSPAAQLFATVYKNASAPASLRGLATNVANYNAWSISSPPSYTSGDSNYDEKLYINALSPLLTSNGWPNAHFIMDTSRNGVQPTKQQAWGDWCNVIGTGFGVQPTTNTGDPLEDAFVWVKPGGESDGTSNSSATRYDFHCGYSDALQPAPEAGTWFQAYFVQLLTNANPALV

SEQ ID NO: 115 - protein, 399 aa, Leptosphaeria maculans
MLNIFLTAAFAAGLSQALPQATSAPASSSQSSMTTMAPAATGNPFADKNFYANPYYSSEVHTLAMPSLPASLKPAATAAANVGSFVWMDTRAKVPTMDTYLADIKAKNAAGANLMGTFVVYNLPDRDCAALASNGELKIAEDGANIYKTDYIDKIAAIIQKYPDVKINLAIEPDSLANMVTNMGVAKCSNAAPYYRNLTSYALEKLNFDNVDMYLDGGHAGWLGWDANIGPAAKLYAEVYKAAGSPRGVRGLVTNVSNYNAFRAATCPAITSGNKNCDEERYINAFAPLLSAEGFPAHFIVDTGRSGKQPTDQAAWGEWCNVRGAGFGIRPTTTTDNALVDAFVWVKPGGESDGTSNTTSARYDGFCGRDSAFKPAPEAGTWFQAYFEMLLQNANPKLA

SEQ ID NO: 116 - protein, 389 aa, Cochliobolus heterostrophus
MLSNVFLTAALAAGLAQALPQATPTPTAAPSGNPFAGKNFYANPYYSSEVHTLAMPSLPASLKPAATAVAKVGSFVWMDTMAKVPLMDTYLADIKAKNAAGANLMGTFVVYDLPDRDCAALASNGELKIDEGGVEKYKTQYIDKIAAIIKKYPDVKINLAIEPDSLANMVTNMGVQKCSRAAPYYKELTAYALKTLNFNNVDMYMDGGHAGWLGWDANIGPTAKLFAEVYKAAGSPRGVRGIVTNVSNYNALRVSSCPSITQGNKNCDEERYINALAPLLKNEGFPAHFIVDQGRSGKVPTNQQEWGDWCNVSGAGFGTRPTTNTGNALIDAIVWVKPGGESDGTSDTSAARYDAHCGRNSAFKPAPEAGTWFQAYFEMLLKNANPALA

SEQ ID NO: 117 - protein, 438 aa, Agaricus bisporus
MFKFAALLALASLVPGFVQAQSPVWGQCGGNGWTGPTTCASGSTCVKQNDFYSQCLPNNQAPPSTTTQPGTTPPATTTSGGTGPTSGAGNPYTGKTVWLSPFYADEVAQAAADISNPSLATKAASVAKIPTFVWFDTVAKVPDLGGYLADARSKNQLVQIVVYDLPDRDCAALASNGEFSLANDGLNKYKNYVDQIAAQIKQFPDVSVVAVIEPDSLANLVTNLNVQKCANAQSAYKEGVIYAVQKLNAVGVTMYIDAGHAGWLGWPANLSPAAQLFAQIYRDAGSPRNLRGIATNVANFNALRASSPDPITQGNSNYDEIHYIEALAPMLSNAGFPAHFIVDQGRSGVQNIRDQWGDWCNVKGAGFGQRPTTNTGSSLIDAIVWVKPGGECDGTSDNSSPRFDSHCSLSDAHQPAPEAGTWFQAYFETLVANANPAL

SEQ ID NO: 118 - protein, 395 aa, Unknown
  Obtained from environmental sample
MKFVQSATLAFAATALAAPSRTTPQKPRQASAGCASAVTLDASTNVFQQYTLHPNNFYRAEVEAAAEAISDSALAEKARKVADVGTFLWLDTIENIGRLEPALEDVPCENIVGLVIYDLPGRDCAAKASNGELKVGELDRYKTEYIDKIAEILKAHSNTAFALVIEPDSLPNLVTNSDLQTCQQSASGYREGVAYALKQLNLPNVVMYIDAGHGGWLGWDANLKPGAQELASVYKSAGSPSQVRGISTNVAGWNAWDQEPGEFSDASDAQYNKCQNEKIYINTFGAELKSAGMPNHAIIDTGRNGVTGLRDEWGDWCNVNGAGFGVRPTANTGDELADAFVWVKPGGESDGTSDSSAARYDSFCGKPDAFKPSPEAGTWNQAYFEMLLKNANPSF

SEQ ID NO: 119 - protein, 454 aa, Coprinopsis cinerea
MLKGSKFFALSLALLPALVQAQRPLYAQCGGTGWTGETTCVSGAVCEVINQWYHQCLPGSNQPQPPVTTQPPVVVPTTSQPPVVVPTNPPGGTPVPSTGNPFEGYDIYLSPYYAEEVEAAAAMIDDPVLKAKALKVKEIPTFIWFDVVRKTPDLGRYLADATAIQQRTGRKQLVQIVVYDLPDRDCAAAASNGEFSLADGGMEKYKDYVDRLASEIRKYPDVRIVAVIEPDSLANMVTNMNVAKCRGAEAAYKEGVIYALRQLSALGVYSYVDAGHAGWLGWNANLAPSARLFAQIYKDAGRSAFIRGLATNVSNYNALSATTRDPVTQGNDNYDELRFINALAPLLRNEGWDAKFIVDQGRSGVQNIRQEWGNWCNVYGAGFGMRPTLNTPSSAIDAIVWIKPGGEADGTSDTSAPRYDTHCGKSDSHKPAPEAGTWFQEYFVNLVKNANPPL

SEQ ID NO: 120 - protein, 400 aa, Coprinopsis cinerea
MKYLNLLAALLAVAPLSLAAPSIEARQSNVNPYIGKSPLVIRSYAQKLEETVRTFQQRGDQLNAARTRTVQNVATFAWISDTNGIGAIRPLIQDALAQQARTGQKVIVQIVVYNLPDRDCSANASTGEFTVGNDGLNRYKNFVNTIARELSTADADKLHFALLLEPDALANLVTNANAPRCRIAAPAYKEGIAYTLATLSKPNVDVYIDAANGGWLGWNDNLRPSAELFKEVYDLARRINPNAKVRGLAVNVSNYNQYRAEVREPFTEWNDAWDESRYVNVLTPHLNAVGFPAHFIVDQGRGGKGGIRTEWGQWCNVRNAGFGIRPTADQGVLQNPNVDAIVWVKPGGESDGTSDLNSNRYDPTCRSPVAHVPAPEAGQWFNEYVVNLVLNANPPLEPTW

SEQ ID NO: 121 - protein, 403 aa, Coprinopsis cinerea
MKFLNLLAALVAVAPLSLAAPSASFERRQGSVNPYIGRSPLVIKSYAEKLEETIAYFEAQGDELNAARTRTVQGIPTFAWISDSATIDTIQPLIADAVAHQEASGEQVLVQLVIYNLPDRDCAAKASDGEFHLDDDGANKYRAYVDRIVAELSTADADKLHFSIVLEPDSLGNMVTNMHVPKCQGAATAYKEGIAYTIASLQKPNIDLYIDAAHGGWLGWNDNLRPSAEIFKETLDLARQITPNATVRGLAINVSNYNPYKTRAREDYTEWNNAYDEWNYVKTLTPHLQAVGFPAQFIVDQGRSGREGIRTEWGQWCNIRNAGFGIRPTTDQAIVDSANVDAIVWVKPGGESDGTSDVNAVRFDENCRSPASHVPAPEAGEWFNEFVVNLVINANPPLEPTYA

SEQ ID NO: 122 - protein, 348 aa, Humicola insolens
QSGNPFSGRTLLVNSDYSSKLDQTRQAFLSRGDQTNAAKVKYVQEKVGTFYWISNIFLLRDIDVAIQNARAAKARGENPIVGLVLYNLPDRDCSAGESSGELKLSQNGLNRYKNEYVNPFAQKLKAASDVQFAVILEPDAIGNMVTGTSAFCRNARGPQQEAIGYAISQLQASHIHLYLDVANGGWLGWADKLEPTAQEVATILQKAGNNAKIRGFSSNVSNYNPYSTSNPPPYTSGSPSPDESRYATNIANAMRQRGLPTQFIIDQSRVALSGARSEWGQWCNVNPAGFGQPFTTNTNNPNVDAIVWVKPGGESDGQCGMGGAPAAGMWFDAYAQMLTQNAHDEIAR

SEQ ID NO: 123 - protein, 452 aa, Irpex lacteus
MKSAAFLAALAAILPAYVAGQAQTWAQCGGIGFTGPTTCVAGSVCTKQNDYYSQCIPGSATTPTSAPTSAPTSQPSQPSSTSSAPSGPSSTPTPSANNPWTGYQIYLSPYYANEVAAAAKAITDPTLAAKAASVANIPNFTWLDSVSKIADLKTYLADASALGKSSGQKQLLQIVVYDLPDRDCAAKASNGEFSIADNGLANYQNYIDQIVAAVKQFPDVRVVAVIEPDSLANLVTNLNVQKCANAKSTYLTAVNYALKQLSSVGVYQYMDAGHAGWLGWPANLTPAAQLFAQVYSDAGKSPFIKGLATNVANYNALSAASPDPITQGDPNYDEIHYINALAPALQSAGFPATFIVDQGRSGQQNHRQQWGDWCNIKGAGFGTRPTTNTGSSLIDSIVWVKPGGESDGTSNSSSPRFDSTCSLSDATQPAPEAGTWFQAYFETLVSKANPPL

SEQ ID NO: 124 - protein, 443 aa, Lentinula edodes
MKITSTGLLALSSLLPFALGQSQLYAQCGGIGWSGATTCVSGATCTVVNAYYSQCLPGSASAPPTSTSSIGTGTTTSSAPGSTGTTTPAAGNPFTEQIYLSPYYANEIAAAVTQISDPTTAAAAAKVANIPTFIWLDQVAKVPDLGTYLADASAKQKSEGKNYLVQIVVYDLPDRDCAALASNGEFTIADNGEANYHDYIDQIVAQIKQYPDVHVVAVIEPDSLANLVTNLSVAKCANAQTTYLECVTYAMQQLSAVGVTMYLDAGHAGWLGWPANLSPAAQLFTSLYSNAGSPSGVRGLATNVANYNALVATTPDPITQGDPNYDEMLYIEALAPLLGSFPAHFIVDQGRSGVQDIRQQWGDWCNVLGAGFGTQPTTNTGSSLIDSIVWVKPGGECDGTSNTSSPRYDAHCGLPDATPNAPEAGTWFQAYFETLVEKANPPL

SEQ ID NO: 125 - protein, 390 aa, Malbranchea cinnamomea
MRDSLFTLLSLALGSASASPFLLPRQANSSNPFAGHTIYPNPYYSNEIDEFAIPALQETDPALVEKAALVKEVGTFFWIDVVAKVPDIGPYLQGIQEANAAGQNPPYIGAIVVYDLPNRDCAAAASNGEFSLEDGGEEKYRGYIDGIREQIEKYPDVRVALVIEPDSLANMVTNLNVPKCAESEQAYRDGVAYALKQLDLPNVWTYIDAGHSGWLGWPANIEPAAEIFVEVWNAAGRPKSTRGFATNVSNYNGYSLSTAPPYTEPNPNFDEVRYINAFRPLLEARGFPAYFIVDQGRSGVQPTAQIEQGHWCNVIDTGFGTRPTTDTGNEYVDSIVWVKPGGESDGTSDTSAERYDYHCGLEDALKPAPEAGQWFQAYFEQLLRNANPPF

SEQ ID NO: 126 - protein, 455 aa, Orpinomyces sp.
MKFLTIASLFIAGTLASQCHPNYPCCQNCGEVFYTDSDGQWGIENNDWCLIQPSKCNSNQSCKFNALGYSCCSHCNSVYSDNDGQWGIENGNWCGLKDSGFGNVTPTTTRNSNPTTSVNTNDPDNFFNNRIYCNDDRKKRVQSSINQLSGELRAKAEKIKDVPTALWLSWDRAPESVSGHLSQAGDQTAVFILYWIPTRDCNSYASQGGAQDMNRYQQYVQRIYNAFRSYPNSKIVVVIEPDTLGNMVTSQSNQHCRDVHDLHKQAIAYALNTLGSLNNVRAYIDAAHGRWLGPHTDEVAKIIKDIVSMAPQGKLRGLSTNVSNYQSTRDEYAYHQKLNSALENVGIRNMKFIVDTARNGVDVAESLVRTGTWCNVIGTGFGERPKGTPDPVNMPLLDAYMWLKPGGDSDGSSSGPYADPNCAHSDSLPGAGNAGDWFHEYFVQLIKNANPPIQA

SEQ ID NO: 127 - protein, 459 aa, Orpinomyces sp.
MKFSTVLATLFATGALASECHWQYPCCKDCTVYYTDTEGKWGVLNNDWCMIDNRRCSSNNNNCSSSITSQGYPCCSNNNCKVEYTDNDGKWGVENNNWCGISNSCGGGQQQQPTQPTQPTQPQQPTQPSSDNFFENEIYSNYKFQGEVDISIKKLNGDLKAKAEKVKYVPTAVWLAWDGAPQEVPRYLQEAGNKTVVFVLYMIPTRDCGANASAGGSATIDKYKGYINNIYNTSNQYKNSKIVMILEPDTIGNLVTNNNDNCRNVRNMHKQALSYAISKFGTQSHVKVYLDAAHGAWLNQYADQTANVIKEILNNAGSGKLRGISTNVSNYQSIESEYKYHQNLNRALESKGVRGLKFIVDTSRNGANVEGAFNASGTWCNFKGAGLGQRPKGNPNPGSMPLLDAYMWIKTPGEADGSSQGSRADPVCARGDSLQGAPDAGSWFHEYFTMLIQNANPPF

SEQ ID NO: 128 - protein, 449 aa, Orpinomyces sp.
MKFSALISTLFAAGAMASRCHPSYPCCNGCNVEYTDTEGNWGVENFDWCFIDESRCNPGYCKFEALGYSCCKGCEVVYSDEDGNWGVENQQWCGIRDNCTPNVPATSARTTTRTTTTTRTTTVNSLPTSDNFFENELYSNYKFQGEVDQSIQRLSGSLQEKAKKVKYVPTAAWLAWSGATNEVARYLNEAGSKTVVFVLYMIPTRDCNAGGSNGGADNLSTYQGYVNSIYNTINQYPNSRIVMIIEPDTIGNLVTANNANCRNVHDMHKQALSYAISKFGTQKNVRVYLDAAHGGWLNSSADRTAEVIAEILRNAGNGKIRGISTNVSNYQPVYSEYQYHQNLNRALESRGVRGMKFIVDTSRNGRNPSSATWCNLKGAGLGARPQANPDPNMPLLDAYVWIKTPGESDSASSADPVCRNSDSLQGAPAAGSWFHDYFVMLLENANPPF

SEQ ID NO: 129 - protein, 460 aa, Phanerochaete chrysosporium
MKSTAFFAALVTLLPAYVAGQASEWGQCGGIGWTGPTTCVSGTTCTVLNPYYSQCLPGSAVTTTSVITSHSSSVSSVSSHSGSSTSTSSPTGPTGTNPPPPPSANNPWTGFQIFLSPYYANEVAAAAKQITDPTLSSKAASVANIPTFTWLDSVAKIPDLGTYLASASALGKSTGTKQLVQIVIYDLPDRDCAAKASNGEFSIANNGQANYENYIDQIVAQIQQFPDVRVVAVIEPDSLANLVTNLNVQKCANAKTTYLACVNYALTNLAKVGVYMYMDAGHAGWLGWPANLSPAAQLFTQVWQNAGKSPFIKGLATNVANYNALQAASPDPITQGNPNYDEIHYINALAPLLQQAGWDATFIVDQGRSGVQNIRQQWGDWCNIKGAGFGTRPTTNTGSQFIDSIVWVKPGGECDGTSNSSSPRYDSTCSLPDAAQPAPEAGTWFQAYFQTLVSAANPPL

SEQ ID NO: 130 - protein, 486 aa, Piromyces equi
  misc_feature (114)..(114): Xaa can be any naturally occurring amino acid
MKTSIALTAVAALAAKASAACWSEKLGYKCCSSANAPVVYQDADGDWSVENNDWCGIPAATPIQSCWSEKLGYPCCKSTSAVVYQDADGDWGVENNDWCGISGDIKPIPTDDPXPGEQYTHVGNPFKGHKFFINPXYTDEVDKAIAQMSDSSLIKKAEKMKEFSNAIWLDNMENMNNWLERNLKTALAEQQSGSQTVLTVFVVYDLPGRDCHALASNGELLANDADFERYKTDYIDVIAEKLAYYKSQPVVLVIEPDSLANMVTNIESTPACAKSEKYYMDGHAYLIKKLGQFPHVAMYLDIGHAFXLGWDDNREKGGKVYSKVIKSGSPGKVRGFASNVANYTPWEDPELSRGPETEWNSCPDEKRYIQAMYKDFKAAGIESVYFIDDSSRNGVKNDRFHPGEWCNQTGSGIGARPEANPVSGMDYLDAFYWVKPYGESDGTSDESAKRYDGYCGHRTAMKPAPEAGQWFQAFFEEGLKNANPPL

SEQ ID NO: 131 - protein, 460 aa, Piromyces rhizinflatus
MKFSTLIGTLFATGALASSCHRDYPCCNDCNVVYQDWERDWGVLNGQEWCFIDKNRCNGGGYCKFESLGYPCCNGCDVYYTDNDGRWGVENGNWCGIRDDKCNGYQQPRTTTTTRTTTRTTTTQRPVQTNVSDNFFENTLYSNFKFQGEVQSSIQKLSGDMAKKAEKVKYVPTAVWLAWEGAPREVPQYLDDAGSKTVVFVLYMIPTRDCNANASVGGSATLEKYKGYIDNIYNTFNQYPNSKIVMILEPDTIGNLVTANNANCMNVQNLHKQGLAYAISKFGTQKNVRVYLDAAHGAWLSSHADKTAQVIKEILNNAGSGKLRGITTNVSNYQTVNDEYSYQMRLNSALQNLGVRDLHYIIDTSRNGANIAQQFNQSGTWCNFKGAGLGARPQANPDSSKPLLDAYMWIKTPGEADGSSSGSRADPVCGRWDSLQGAPDAGSWFHDYFVMLLQNANPPF

SEQ ID NO: 132 - protein, 491 aa, Piromyces sp. E2
MKASIALTAIAALAANASAACFSERLGYPCCRGNEVFYTDNDGDWGVENGNWCGIGGASATTCWSQALGYPCCTSTSDVAYVDGDGNWGVENGNWCGIIAGGNSSNNNSGSTINVGDVTIGNQYTHTGNPFAGHKFFINPYYTAEVDGAIAQISNASLRAKAEKMKEFSNAIWLDTIKNMNEWLEKNLKYALAEQNETGKTVLTVFVVYDLPGRDCHALASNGELLANDSDWARYQSEYIDVIEEKLKTYKSQPVVLVVEPDSLANMVTNLDSTPACRDSEKYYMDGHAYLIKKLGVLPHVAMYLDIGHAFWLGWDDNRLKAGKVYSKVIQSGAPGNVRGFASNVANYTPWEDPTLSRGPDTEWNPCPDEKRYIEAMYKDFKSAGIKSVYFIDDTSRNGHKTDRTHPGEWCNQTGVGIGARPQANPISGMDYLDAFYWVKPLGESDGYSDTTAVRYDGYCGHATAMKPAPEAGQWFQKHFEQGLENANPPL

SEQ ID NO: 133 - protein, 458 aa, Stilbella annulata
MAGRFFLSAAFLASAALAVPLEERQNCSPQWAQCGGNGWSGPTCCASGSNCQVTNEWYSQCVPGAAPPPPPVTTTRSTTTPPTTTTRTTADAPPPTGGATYTGNPFLGVNQWANNFYRSEIMNIAVPSLSGAMATAAAKVADVPTFQWIDKMDKLPLIDEALADVRAANARGGNYASILVVYNLPDRDCAAAASNGEFAIADGGVAKYKNYIDEIRKLVIKYNDLRIILVIEPDSLANMVTNMNVAKCQNAASAYRECTNYALTNLDLPNVAQYMDAGHAGWLGWPANITPAAQLFAEVYKQAGSPKSVRGLAINVSNYNAWSVSSPPPYTSPNPNYDERHFVEAFAPLLRQNGWDAKFIVDQGRSGRQPTGQQEWGHWCNAIGTGFGQRPTSNTGHADVDAFVWIKPGGECDGTSDTSAARYDHFCGNPDALKPAPEAGEWFQAYFEQLLRNANPAF

SEQ ID NO: 134 - DNA, 1407 bp, Unknown
  Obtained from environmental sample
atgactgtctatcaactcttgtttacggccgctttggctggtacagcacttgctgcccctcttgtcgaggaacgccaggcttgcgccagccagtgggcccagtgtggtggcttcagctggaatggtgctacttgctgccagtctggtagttactgtagcaagatcaatgactattactctcagtgtattcctggagagggtcccgccacttccaagacaagcacgcttcctgcttctaccaccaccagcaagccgacttccacttccactgctggtacttcttccactacgaagcctccacctgctggaagcggcactgccacttatagcggaaacccctactctggtgttaacctttgggccaacagctactatcgctcagaggttaccaacttggccatccccaagttgagcggtgccatggccacggctgctgccaaggtcgctgatgttccctcttatcagtggatggactctttcgagcacatctccctgatggaggatactcttgttgacattcgaaaggccaaccaggctggtggtaactacgccggccagtttgtcgtctatgatctccctgatcgtgactgcgctgctgccgcttccaacggagagtattcccttgacaaggatggtgccaacaagtacaagaactacatcaacactatcaagaagatcatccagagctactctgatatccgagtgctccttgttattgagcctgactccctggctaacctggtcaccaacatggatgttgccaagtgcgccaaggcccatgatgcgtacatcagcctgacgaactacgctgtcacggaactgaacctacccaacgtcgccatgtatcttgatgcaggccacgctggctggctcggctggcccaacaaccaaggccctgctgcgaagctctttgctagcatctacaaggatgccggcaagccagctgcgctccgtggactcgccaccaacgttgctaactacaacgcctggagcctcagcagtgctcccccttatacccaaggcgcctccatctacgacgagaagagtttcattcacgcaatgggtcctctcctggagcagaatggctggcctggcgctcacttcattaccgaccagggccgttctggcaagcagcccaccggccagatccagtggggtgactggtgcaactccaaaggcactggctttggtatccgtccctctgccaacactggtgacagcctcctcgatgcttttgtctgggtcaagcctggtggtgagtctgatggtacctcggacacgagtgctacccgttacgactaccactgcggtgcttctgccgctcttcagccggcacctgaggcaggaacctggttccaggcctacttcgagcagcttcttaccaatgccaacccttcgttcctgtaa

SEQ ID NO: 135 - DNA, 1407 bp, Unknown
  Obtained from environmental sample
atgactgtctatcaactcttgtttacggccgctttggctggtacagcacttgctgcccctcttgtcgaggaacgccaggcttgcgccagccagtgggcccagtgtggtggcttcagctggaatggtgctacttgctgccagtctggtagttactgtagcaagatcaatgactattactctcagtgtatttggggagagggtcccgccacttccaagacaagcacgcttcctgcttctaccaccaccagcaagccgacttccacttccactgctggtacttcttccactacgaagcctccacctgctggaagcggcactgccacttatagcggaaacccctactctggtgttaacctttgggccaacagctactatcgctcagaggttaccaacttggccatccccaagttgagcggtgccatggccacggctgctgccaaggtcgctgatgttccctcttatcagtggatggactctttcgagcacatctccctgatggaggatactcttgttgacattcgaaaggccaaccaggctggtggtaactacgccggccagtttgtcgtctatgatctccctgatcgtgactgcgctgctgccgcttccaacggagagtattcccttgacaaggatggtgccaacaagtacaagaactacatcaacactatcaagaagatcatccagagctactctgatatccgaatcctccttgttattgagcctgactccctggctaacctggtcaccaacatggatgttgccaagtgcgccaaggcccatgatgcgtacatcagcctgacgaactacgctgtcacggaactgaacctacccaacgtcgccatgtatcttgatgcaggccacgctggctggctcggctggcccaacaaccaaggccctgctgcgaagctctttgctagcatctacaaggatgccggcaagccagctgcgctccgtggactcgccaccaacgttgctaactacaacgcctggagcctcagcagtgctcccccttatacccaaggcgcctccatctacgacgagaagagtttcattcacgcaatgggtcctctcctggagcagaatggctggcctggcgctcacttcattaccgaccagggccgttctggcaagcagcccaccggccagatccagtggggtgactggtgcaactccaaaggcactggctttggtatccgtccctctgccaacactggtgacagcctcctcgatgcttttgtctgggtcaagcctggtggtgagtctgatggtacctcggacacgagtgctacccgttacgactaccactgcggtgcttctgccgctcttcagccggcacctgaggcaggaacctggttccaggcctacttcgagcagcttcttaccaatgccaacccttcgttcctgtaa

SEQ ID NO: 136 - DNA, 1407 bp, Unknown
  Obtained from environmental sample
atgactgtctatcaactcttgtttacggccgctttggctggtacagcacttgctgcccctcttgtcgaggaacgccaggcttgcgccagccagtgggcccagtgtggtggcttcagctggaatggtgctacttgctgccagtctggtagttactgtagcaagatcaatgactattactctcagtgtattgagggagagggtcccgccacttccaagacaagcacgcttcctgcttctaccaccaccagcaagccgacttccacttccactgctggtacttcttccactacgaagcctccacctgctggaagcggcactgccacttatagcggaaacccctactctggtgttaacctttgggccaacagctactatcgctcagaggttaccaacttggccatccccaagttgagcggtgccatggccacggctgctgccaaggtcgctgatgttccctcttatcagtggatggactctttcgagcacatctccctgatggaggatactcttgttgacattcgaaaggccaaccaggctggtggtaactacgccggccagtttgtcgtctatgatctccctgatcgtgactgcgctgctgccgcttccaacggagagtattcccttgacaaggatggtgccaacaagtacaagaactacatcaacactatcaagaagatcatccagagctactctgatatccgaatcctccttgttattgagcctgactccctggctaacctggtcaccaacatggatgttgccaagtgcgccaaggcccatgatgcgtacatcagcctgacgaactacgctgtcacggaactgaacctacccaacgtcgccatgtatcttgatgcaggccacgctggctggctcggctggcccaacaaccaaggccctgctgcgaagctctttgctagcatctacaaggatgccggcaagccagctgcgctccgtggactcgccaccaacgttgctaactacaacgcctggagcctcagcagtgctcccccttatacccaaggcgcctccatctacgacgagaagagtttcattcacgcaatgggtcctctcctggagcagaatggctggcctggcgctcacttcattaccgaccagggccgttctggcaagcagcccaccggccagatccagtggggtgactggtgcaactccaaaggcactggctttggtatccgtccctctgccaacactggtgacagcctcctcgatgcttttgtctgggtcaagcctggtggtgagtctgatggtacctcggacacgagtgctacccgttacgactaccactgcggtgcttctgccgctcttcagccggcacctgaggcaggaacctggttccaggcctacttcgagcagcttcttaccaatgccaacccttcgttcctgtaa

SEQ ID NO: 137 - DNA, 1407 bp, Unknown
  Obtained from environmental sample
atgactgtctatcaactcttgtttacggccgctttggctggtacagcacttgctgcccctagggtcgaggaacgccaggcttgcgccagccagtgggcccagtgtggtggcttcagctggaatggtgctacttgctgccagtctggtagttactgtagcaagatcaatgactattactctcagtgtattcctggagagggtcccgccacttccaagacaagcacgcttcctgcttctaccaccaccagcaagccgacttccacttccactgctggtacttcttccactacgaagcctccacctgctggaagcggcactgccacttatagcggaaacccctactctggtgttaacctttgggccaacagctactatcgctcagaggttaccaacttggccatccccaagttgagcggtgccatggccacggctgctgccaaggtcgctgatgttccctcttatcagtggatggactctttcgagcacatctccctgatggaggatactcttgttgacattcgaaaggccaaccaggctggtggtaactacgccggccagtttgtcgtctatgatctccctgatcgtgactgcgctgctgccgcttccaacggagagtattcccttgacaaggatggtgccaacaagtacaagaactacatcaacactatcaagaagatcatccagagctactctgatatccgaatcctccttgttattgagcctgactccctggctaacctggtcaccaacatggatgttgccaagtgcgccaaggcccatgatgcgtacatcagcctgacgaactacgctgtcacggaactgaacctacccaacgtcgccatgtatcttgatgcaggccacgctggctggctcggctggcccaacaaccaaggccctgctgcgaagctctttgctagcatctacaaggatgccggcaagccagctgcgctccgtggactcgccaccaacgttgctaactacaacgcctggagcctcagcagtgctcccccttatacccaaggcgcctccatctacgacgagaagagtttcattcacgcaatgggtcctctcctggagcagaatggctggcctggcgctcacttcattaccgaccagggccgttctggcaagcagcccaccggccagatccagtggggtgactggtgcaactccaaaggcactggctttggtatccgtccctctgccaacactggtgacagcctcctcgatgcttttgtctgggtcaagcctggtggtgagtctgatggtacctcggacacgagtgctacccgttacgactaccactgcggtgcttctgccgctcttcagccggcacctgaggcaggaacctggttccaggcctacttcgagcagcttcttaccaatgccaacccttcgttcctgtaa

SEQ ID NO: 138 - DNA, 1407 bp, Unknown
  Obtained from environmental sample
atgactgtctatcaactcttgtttacggccgctttggctggtacagcacttgctgcccctcttgtcgaggaacgccaggcttgcgccagccagtgggcccagtgtggtggcttcagctggaatggtgctacttgctgccagtctggtagttactgtagcaagatcaatgactattactctcagtgtattcctggagagggtcccgccacttccaagacaagcacgcttcctgcttctaccaccaccagcaagccgacttccacttccactgctggtacttcttccactacgaagcctccacctgctggagttggcactgccacttatagcggaaacccctactctggtgttaacctttgggccaacagctactatcgctcagaggttaccaacttggccatccccaagttgagcggtgccatggccacggctgctgccaaggtcgctgatgttccctcttatcagtggatggactctttcgagcacatctccctgatggaggatactcttgttgacattcgaaaggccaaccaggctggtggtaactacgccggccagtttgtcgtctatgatctccctgatcgtgactgcgctgctgccgcttccaacggagagtattcccttgacaaggatggtgccaacaagtacaagaactacatcaacactatcaagaagatcatccagagctactctgatatccgaatcctccttgttattgagcctgactccctggctaacctggtcaccaacatggatgttgccaagtgcgccaaggcccatgatgcgtacatcagcctgacgaactacgctgtcacggaactgaacctacccaacgtcgccatgtatcttgatgcaggccacgctggctggctcggctggcccaacaaccaaggccctgctgcgaagctctttgctagcatctacaaggatgccggcaagccagctgcgctccgtggactcgccaccaacgttgctaactacaacgcctggagcctcagcagtgctcccccttatacccaaggcgcctccatctacgacgagaagagtttcattcacgcaatgggtcctctcctggagcagaatggctggcctggcgctcacttcattaccgaccagggccgttctggcaagcagcccaccggccagatccagtggggtgactggtgcaactccaaaggcactggctttggtatccgtccctctgccaacactggtgacagcctcctcgatgcttttgtctgggtcaagcctggtggtgagtctgatggtacctcggacacgagtgctacccgttacgactaccactgcggtgcttctgccgctcttcagccggcacctgaggcaggaacctggttccaggcctacttcgagcagcttcttaccaatgccaacccttcgttcctgtaa

SEQ ID NO: 139 - DNA, 1407 bp, Unknown
  Obtained from environmental sample
atgactgtctatcaactcttgtttacggccgctttggctggtacagcacttgctgcccctcttgtcgaggaacgccaggcttgcgccagccagtgggcccagtgtggttcgttcagctggaatggtgctacttgctgccagtctggtagttactgtagcaagatcaatgactattactctcagtgtattcctggagagggtcccgccacttccaagacaagcacgcttcctgcttctaccaccaccagcaagccgacttccacttccactgctggtacttcttccactacgaagcctccacctgctggaagcggcactgccacttatagcggaaacccctactctggtgttaacctttgggccaacagctactatcgctcagaggttaccaacttggccatccccaagttgagcggtgccatggccacggctgctgccaaggtcgctgatgttccctcttatcagtggatggactctttcgagcacatctccctgatggaggatactcttgttgacattcgaaaggccaaccaggctggtggtaactacgccggccagtttgtcgtctatgatctccctgatcgtgactgcgctgctgccgcttccaacggagagtattcccttgacaaggatggtgccaacaagtacaagaactacatcaacactatcaagaagatcatccagagctactctgatatccgaatcctccttgttattgagcctgactccctggctaacctggtcaccaacatggatgttgccaagtgcgccaaggcccatgatgcgtacatcagcctgacgaactacgctgtcacggaactgaacctacccaacgtcgccatgtatcttgatgcaggccacgctggctggctcggctggcccaacaaccaaggccctgctgcgaagctctttgctagcatctacaaggatgccggcaagccagctgcgctccgtggactcgccaccaacgttgctaactacaacgcctggagcctcagcagtgctcccccttatacccaaggcgcctccatctacgacgagaagagtttcattcacgcaatgggtcctctcctggagcagaatggctggcctggcgctcacttcattaccgaccagggccgttctggcaagcagcccaccggccagatccagtggggtgactggtgcaactccaaaggcactggctttggtatccgtccctctgccaacactggtgacagcctcctcgatgcttttgtctgggtcaagcctggtggtgagtctgatggtacctcggacacgagtgctacccgttacgactaccactgcggtgcttctgccgctcttcagccggcacctgaggcaggaacctggttccaggcctacttcgagcagcttcttaccaatgccaacccttcgttcctgtaa

SEQ ID NO: 140 - DNA, 1407 bp, Unknown
  Obtained from environmental sample
atgactgtctatcaactcttgtttacggccgctttggctggtacagcacttgctgcccctcttgtcgaggaacgccaggcttgcgccagccagtgggcccagtgtggtggcttcagctggaatggtgctacttgctgccagtctggtagttactgtagcaagatcaatgactattactctcagtgtattcctcttgagggtcccgccacttccaagacaagcacgcttcctgcttctaccaccaccagcaagccgacttccacttccactgctggtacttcttccactacgaagcctccacctgctggaagcggcactgccacttatagcggaaacccctactctggtgttaacctttgggccaacagctactatcgctcagaggttaccaacttggccatccccaagttgagcggtgccatggccacggctgctgccaaggtcgctgatgttccctcttatcagtggatggactctttcgagcacatctccctgatggaggatactcttgttgacattcgaaaggccaaccaggctggtggtaactacgccggccagtttgtcgtctatgatctccctgatcgtgactgcgctgctgccgcttccaacggagagtattcccttgacaaggatggtgccaacaagtacaagaactacatcaacactatcaagaagatcatccagagctactctgatatccgaatcctccttgttattgagcctgactccctggctaacctggtcaccaacatggatgttgccaagtgcgccaaggcccatgatgcgtacatcagcctgacgaactacgctgtcacggaactgaacctacccaacgtcgccatgtatcttgatgcaggccacgctggctggctcggctggcccaacaaccaaggccctgctgcgaagctctttgctagcatctacaaggatgccggcaagccagctgcgctccgtggactcgccaccaacgttgctaactacaacgcctggagcctcagcagtgctcccccttatacccaaggcgcctccatctacgacgagaagagtttcattcacgcaatgggtcctctcctggagcagaatggctggcctggcgctcacttcattaccgaccagggccgttctggcaagcagcccaccggccagatccagtggggtgactggtgcaactccaaaggcactggctttggtatccgtccctctgccaacactggtgacagcctcctcgatgcttttgtctgggtcaagcctggtggtgagtctgatggtacctcggacacgagtgctacccgttacgactaccactgcggtgcttctgccgctcttcagccggcacctgaggcaggaacctggttccaggcctacttcgagcagcttcttaccaatgccaacccttcgttcctgtaa

SEQ ID NO: 141 - DNA, 1407 bp, Unknown
  Obtained from environmental sample
atgactgtctatcaactcttgtttacggccgctttggctggtacagcacttgctgcccctcttgtcgaggaacgccaggcttgcgccagccagtgggcccagtgtggtggcttcagctggaatggtgctacttgctgccagtctggtagttactgtagcaagatcaatgactattactctcagtgtattcctggagagggtcccgccacttccaagacaagcacgcttcctgcttctaccaccaccagcaagccgacttccacttccactgctggtacttcttccactacgaagcctccacctgctggaagcggcactgccacttatagcggaaacccctactctggtgttaacctttgggccaacagctactatcgctcagaggttaccaacttggccatccccaagttgagcggtgccatggccacggctgctgccaaggtcgctgatgttccctcttatcagtggatggactctttcgagcacatctccctgatggaggatactcttgttgacattcgaaaggccaaccaggctggtggtaactacgccggccagtttgtcgtctatgatctccctgatcgtgactgcgctgctgccgcttccaacggagagtattcccttgacaaggatggtgccaacaagtacaagaactacatcaacactatcaagaagatcatccagagctactctgatatccgaatcctccttgttattgagcctgactccctggctaacctggtcaccaacatggatgttgccaagtgcgccaaggcccatgatgcgtacatcagcctgacgaactacgctgtcacggaactgaacctacccaacgtcgccatgtatcttgatgcaggccacgctggctggctcggctggcccaacaaccaaggccctgctgcgaagctctttgctagcatctaccatgatgccggcaagccagctgcgctccgtggactcgccaccaacgttgctaactacaacgcctggagcctcagcagtgctcccccttatacccaaggcgcctccatctacgacgagaagagtttcattcacgcaatgggtcctctcctggagcagaatggctggcctggcgctcacttcattaccgaccagggccgttctggcaagcagcccaccggccagatccagtggggtgactggtgcaactccaaaggcactggctttggtatccgtccctctgccaacactggtgacagcctcctcgatgcttttgtctgggtcaagcctggtggtgagtctgatggtacctcggacacgagtgctacccgttacgactaccactgcggtgcttctgccgctcttcagccggcacctgaggcaggaacctggttccaggcctacttcgagcagcttcttaccaatgccaacccttcgttcctgtaa

SEQ ID NO: 142 - DNA, 1407 bp, Unknown
  Obtained from environmental sample
atgactgtctatcaactcttgtttacggccgctttggctggtacagcacttgctgcccctcttgtcgaggaacgccaggcttgcgccagccagtgggcccagtgtggtggcttcagctggaatggtgctacttgctgccagtctggtagttactgtagcaagatcaatgactattactctcagtgtattcctggacggggtcccgccacttccaagacaagcacgcttcctgcttctaccaccaccagcaagccgacttccacttccactgctggtacttcttccactacgaagcctccacctgctggaagcggcactgccacttatagcggaaacccctactctggtgttaacctttgggccaacagctactatcgctcagaggttaccaacttggccatccccaagttgagcggtgccatggccacggctgctgccaaggtcgctgatgttccctcttatcagtggatggactctttcgagcacatctccctgatggaggatactcttgttgacattcgaaaggccaaccaggctggtggtaactacgccggccagtttgtcgtctatgatctccctgatcgtgactgcgctgctgccgcttccaacggagagtattcccttgacaaggatggtgccaacaagtacaagaactacatcaacactatcaagaagatcatccagagctactctgatatccgaatcctccttgttattgagcctgactccctggctaacctggtcaccaacatggatgttgccaagtgcgccaaggcccatgatgcgtacatcagcctgacgaactacgctgtcacggaactgaacctacccaacgtcgccatgtatcttgatgcaggccacgctggctggctcggctggcccaacaaccaaggccctgctgcgaagctctttgctagcatctacaaggatgccggcaagccagctgcgctccgtggactcgccaccaacgttgctaactacaacgcctggagcctcagcagtgctcccccttatacccaaggcgcctccatctacgacgagaagagtttcattcacgcaatgggtcctctcctggagcagaatggctggcctggcgctcacttcattaccgaccagggccgttctggcaagcagcccaccggccagatccagtggggtgactggtgcaactccaaaggcactggctttggtatccgtccctctgccaacactggtgacagcctcctcgatgcttttgtctgggtcaagcctggtggtgagtctgatggtacctcggacacgagtgctacccgttacgactaccactgcggtgcttctgccgctcttcagccggcacctgaggcaggaacctggttccaggcctacttcgagcagcttcttaccaatgccaacccttcgttcctgtaa

SEQ ID NO: 143 - DNA, 1407 bp, Unknown
  Obtained from environmental sample
atgactgtctatcaactcttgtttacggccgctttggctggtacagcacttgctgcccctcttgtcgaggaacgccaggcttgcgccagccagtgggcccagtgtggtggcttcagctggaatggtgctacttgctgccagtctggtagttactgtagcaagatcaatgactattactctcagtgtattcctggagagggtcccgccacttccaagacaagcacgcttcctgcttctaccaccaccagcaagccgacttccacttccactgctggtacttcttccactacgaagcctccacctgctggaagcggcactgccacttatagcggaaacccctacgctggtgttaacctttgggccaacagctactatcgctcagaggttaccaacttggccatccccaagttgagcggtgccatggccacggctgctgccaaggtcgctgatgttccctcttatcagtggatggactctttcgagcacatctccctgatggaggatactcttgttgacattcgaaaggccaaccaggctggtggtaactacgccggccagtttgtcgtctatgatctccctgatcgtgactgcgctgctgccgcttccaacggagagtattcccttgacaaggatggtgccaacaagtacaagaactacatcaacactatcaagaagatcatccagagctactctgatatccgaatcctccttgttattgagcctgactccctggctaacctggtcaccaacatggatgttgccaagtgcgccaaggcccatgatgcgtacatcagcctgacgaactacgctgtcacggaactgaacctacccaacgtcgccatgtatcttgatgcaggccacgctggctggctcggctggcccaacaaccaaggccctgctgcgaagctctttgctagcatctacaaggatgccggcaagccagctgcgctccgtggactcgccaccaacgttgctaactacaacgcctggagcctcagcagtgctcccccttatacccaaggcgcctccatctacgacgagaagagtttcattcacgcaatgggtcctctcctggagcagaatggctggcctggcgctcacttcattaccgaccagggccgttctggcaagcagcccaccggccagatccagtggggtgactggtgcaactccaaaggcactggctttggtatccgtccctctgccaacactggtgacagcctcctcgatgcttttgtctgggtcaagcctggtggtgagtctgatggtacctcggacacgagtgctacccgttacgactaccactgcggtgcttctgccgctcttcagccggcacctgaggcaggaacctggttccaggcctacttcgagcagcttcttaccaatgccaacccttcgttcctgtaa

SEQ ID NO: 144 - DNA, 1407 bp, Unknown
  Obtained from environmental sample
atgactgtctatcaactcttgtttacggccgctttggctggtacagcacttgctgcccctcttgtcgaggaacgccaggcttgcgccagccagtgggcccagtgtggtggcttcagctggaatggtgctacttgctgccagtctggtagttactgtagcaagatcaatgactattactctcagtgtattcctggagagaagcccgccacttccaagacaagcacgcttcctgcttctaccaccaccagcaagccgacttccacttccactgctggtacttcttccactacgaagcctccacctgctggaagcggcactgccacttatagcggaaacccctactctggtgttaacctttgggccaacagctactatcgctcagaggttaccaacttggccatccccaagttgagcggtgccatggccacggctgctgccaaggtcgctgatgttccctcttatcagtggatggactctttcgagcacatctccctgatggaggatactcttgttgacattcgaaaggccaaccaggctggtggtaactacgccggccagtttgtcgtctatgatctccctgatcgtgactgcgctgctgccgcttccaacggagagtattcccttgacaaggatggtgccaacaagtacaagaactacatcaacactatcaagaagatcatccagagctactctgatatccgaatcctccttgttattgagcctgactccctggctaacctggtcaccaacatggatgttgccaagtgcgccaaggcccatgatgcgtacatcagcctgacgaactacgctgtcacggaactgaacctacccaacgtcgccatgtatcttgatgcaggccacgctggctggctcggctggcccaacaaccaaggccctgctgcgaagctctttgctagcatctacaaggatgccggcaagccagctgcgctccgtggactcgccaccaacgttgctaactacaacgcctggagcctcagcagtgctcccccttatacccaaggcgcctccatctacgacgagaagagtttcattcacgcaatgggtcctctcctggagcagaatggctggcctggcgctcacttcattaccgaccagggccgttctggcaagcagcccaccggccagatccagtggggtgactggtgcaactccaaaggcactggctttggtatccgtccctctgccaacactggtgacagcctcctcgatgcttttgtctgggtcaagcctggtggtgagtctgatggtacctcggacacgagtgctacccgttacgactaccactgcggtgcttctgccgctcttcagccggcacctgaggcaggaacctggttccaggcctacttcgagcagcttcttaccaatgccaacccttcgttcctgtaa

SEQ ID NO: 145 - DNA, 1407 bp, Unknown
  Obtained from environmental sample
atgactgtctatcaactcttgtttacggccgctttggctggtacagcacttgctgcccctcttgtcaaggaacgccaggcttgcgccagccagtgggcccagtgtggtggcttcagctggaatggtgctacttgctgccagtctggtagttactgtagcaagatcaatgactattactctcagtgtattcctggagagggtcccgccacttccaagacaagcacgcttcctgcttctaccaccaccagcaagccgacttccacttccactgctggtacttcttccactacgaagcctccacctgctggaagcggcactgccacttatagcggaaacccctactctggtgttaacctttgggccaacagctactatcgctcagaggttaccaacttggccatccccaagttgagcggtgccatggccacggctgctgccaaggtcgctgatgttccctcttatcagtggatggactctttcgagcacatctccctgatggaggatactcttgttgacattcgaaaggccaaccaggctggtggtaactacgccggccagtttgtcgtctatgatctccctgatcgtgactgcgctgctgccgcttccaacggagagtattcccttgacaaggatggtgccaacaagtacaagaactacatcaacactatcaagaagatcatccagagctactctgatatccgaatcctccttgttattgagcctgactccctggctaacctggtcaccaacatggatgttgccaagtgcgccaaggcccatgatgcgtacatcagcctgacgaactacgctgtcacggaactgaacctacccaacgtcgccatgtatcttgatgcaggccacgctggctggctcggctggcccaacaaccaaggccctgctgcgaagctctttgctagcatctacaaggatgccggcaagccagctgcgctccgtggactcgccaccaacgttgctaactacaacgcctggagcctcagcagtgctcccccttatacccaaggcgcctccatctacgacgagaagagtttcattcacgcaatgggtcctctcctggagcagaatggctggcctggcgctcacttcattaccgaccagggccgttctggcaagcagcccaccggccagatccagtggggtgactggtgcaactccaaaggcactggctttggtatccgtccctctgccaacactggtgacagcctcctcgatgcttttgtctgggtcaagcctggtggtgagtctgatggtacctcggacacgagtgctacccgttacgactaccactgcggtgcttctgccgctcttcagccggcacctgaggcaggaacctggttccaggcctacttcgagcagcttcttaccaatgccaacccttcgttcctgtaa

SEQ ID NO: 146 - DNA, 1407 bp, Unknown
  Obtained from environmental sample
atgactgtctatcaactcttgtttacggccgctttggctggtacagcacttgctgcccctcttgtcgaggaacgccaggcttgcgccagccagtgggcccagtgtggtggcttcagctggaatggtgctacttgctgccagtctggtagttactgtagcaagatcaatgactattactctcagtgtattcctggagagggtcccgccacttccaagacaagcacgcttcctgcttctaccaccaccagcaagccgacttccacttccactgctggtacttcttccactacgaagcctccacctgctggaagcggcactgccacttatagcggaaacccctacatgggtgttaacctttgggccaacagctactatcgctcagaggttaccaacttggccatccccaagttgagcggtgccatggccacggctgctgccaaggtcgctgatgttccctcttatcagtggatggactctttcgagcacatctccctgatggaggatactcttgttgacattcgaaaggccaaccaggctggtggtaactacgccggccagtttgtcgtctatgatctccctgatcgtgactgcgctgctgccgcttccaacggagagtattcccttgacaaggatggtgccaacaagtacaagaactacatcaacactatcaagaagatcatccagagctactctgatatccgaatcctccttgttattgagcctgactccctggctaacctggtcaccaacatggatgttgccaagtgcgccaaggcccatgatgcgtacatcagcctgacgaactacgctgtcacggaactgaacctacccaacgtcgccatgtatcttgatgcaggccacgctggctggctcggctggcccaacaaccaaggccctgctgcgaagctctttgctagcatctacaaggatgccggcaagccagctgcgctccgtggactcgccaccaacgttgctaactacaacgcctggagcctcagcagtgctcccccttatacccaaggcgcctccatctacgacgagaagagtttcattcacgcaatgggtcctctcctggagcagaatggctggcctggcgctcacttcattaccgaccagggccgttctggcaagcagcccaccggccagatccagtggggtgactggtgcaactccaaaggcactggctttggtatccgtccctctgccaacactggtgacagcctcctcgatgcttttgtctgggtcaagcctggtggtgagtctgatggtacctcggacacgagtgctacccgttacgactaccactgcggtgcttctgccgctcttcagccggcacctgaggcaggaacctggttccaggcctacttcgagcagcttcttaccaatgccaacccttcgttcctgtaa

SEQ ID NO: 147 - DNA, 1407 bp, Unknown
  Obtained from environmental sample
atgactgtctatcaactcttgtttacggccgctttggctggtacagcacttgctgcccctcttgtcgaggaacgccaggcttgcgccagccagtggaagcagtgtggtggcttcagctggaatggtgctacttgctgccagtctggtagttactgtagcaagatcaatgactattactctcagtgtattcctggagagggtcccgccacttccaagacaagcacgcttcctgcttctaccaccaccagcaagccgacttccacttccactgctggtacttcttccactacgaagcctccacctgctggaagcggcactgccacttatagcggaaacccctactctggtgttaacctttgggccaacagctactatcgctcagaggttaccaacttggccatccccaagttgagcggtgccatggccacggctgctgccaaggtcgctgatgttccctcttatcagtggatggactctttcgagcacatctccctgatggaggatactcttgttgacattcgaaaggccaaccaggctggtggtaactacgccggccagtttgtcgtctatgatctccctgatcgtgactgcgctgctgccgcttccaacggagagtattcccttgacaaggatggtgccaacaagtacaagaactacatcaacactatcaagaagatcatccagagctactctgatatccgaatcctccttgttattgagcctgactccctggctaacctggtcaccaacatggatgttgccaagtgcgccaaggcccatgatgcgtacatcagcctgacgaactacgctgtcacggaactgaacctacccaacgtcgccatgtatcttgatgcaggccacgctggctggctcggctggcccaacaaccaaggccctgctgcgaagctctttgctagcatctacaaggatgccggcaagccagctgcgctccgtggactcgccaccaacgttgctaactacaacgcctggagcctcagcagtgctcccccttatacccaaggcgcctccatctacgacgagaagagtttcattcacgcaatgggtcctctcctggagcagaatggctggcctggcgctcacttcattaccgaccagggccgttctggcaagcagcccaccggccagatccagtggggtgactggtgcaactccaaaggcactggctttggtatccgtccctctgccaacactggtgacagcctcctcgatgcttttgtctgggtcaagcctggtggtgagtctgatggtacctcggacacgagtgctacccgttacgactaccactgcggtgcttctgccgctcttcagccggcacctgaggcaggaacctggttccaggcctacttcgagcagcttcttaccaatgccaacccttcgttcctgtaa

SEQ ID NO: 148 - DNA, 1407 bp, Unknown
  Obtained from environmental sample
atgactgtctatcaactcttgtttacggccgctttggctggtacagcacttgctgcccctcttgtcaatgaacgccaggcttgcgccagccagtgggcccagtgtggtggcttcagctggaatggtgctacttgctgccagtctggtagttactgtagcaagatcaatgactattactctcagtgtattcctggagagggtcccgccacttccaagacaagcacgcttcctgcttctaccaccaccagcaagccgacttccacttccactgctggtacttcttccactacgaagcctccacctgctggaagcggcactgccacttatagcggaaacccctactctggtgttaacctttgggccaacagctactatcgctcagaggttaccaacttggccatccccaagttgagcggtgccatggccacggctgctgccaaggtcgctgatgttccctcttatcagtggatggactctttcgagcacatctccctgatggaggatactcttgttgacattcgaaaggccaaccaggctggtggtaactacgccggccagtttgtcgtctatgatctccctgatcgtgactgcgctgctgccgcttccaacggagagtattcccttgacaaggatggtgccaacaagtacaagaactacatcaacactatcaagaagatcatccagagctactctgatatccgaatcctccttgttattgagcctgactccctggctaacctggtcaccaacatggatgttgccaagtgcgccaaggcccatgatgcgtacatcagcctgacgaactacgctgtcacggaactgaacctacccaacgtcgccatgtatcttgatgcaggccacgctggctggctcggctggcccaacaaccaaggccctgctgcgaagctctttgctagcatctacaaggatgccggcaagccagctgcgctccgtggactcgccaccaacgttgctaactacaacgcctggagcctcagcagtgctcccccttatacccaaggcgcctccatctacgacgagaagagtttcattcacgcaatgggtcctctcctggagcagaatggctggcctggcgctcacttcattaccgaccagggccgttctggcaagcagcccaccggccagatccagtggggtgactggtgcaactccaaaggcactggctttggtatccgtccctctgccaacactggtgacagcctcctcgatgcttttgtctgggtcaagcctggtggtgagtctgatggtacctcggacacgagtgctacccgttacgactaccactgcggtgcttctgccgctcttcagccggcacctgaggcaggaacctggttccaggcctacttcgagcagcttcttaccaatgccaacccttcgttcctgtaa

SEQ ID NO: 149 - DNA, 45 bp, Artificial Sequence
  Catalytic site in CBH II
tatgatctccctgatcgtgactgcgctgctgccgcttccaacgga

SEQ ID NO: 150 - protein, 15 aa, Artificial Sequence
  Catalytic site in CBH II
YDLPDRDCAAAASNG

SEQ ID NO: 151 - DNA, 45 bp, Artificial Sequence
  Catalytic site in CBH II
tacgatctgcccaaccgcgattgcgccgccctggcctccaacggc

SEQ ID NO: 152 - protein, 15 aa, Artificial Sequence
  Catalytic site in CBH II
YDLPNRDCAALASNG

SEQ ID NO: 153 - DNA, 99 bp, Artificial Sequence
  Catalytic site in CBH II
cctggtggtgagtctgatggtacctcggacacgagtgctacccgttacgactaccactgcggtgcttctgccgctcttcagccggcacctgaggcagga

SEQ ID NO: 154 - protein, 33 aa, Artificial Sequence
  Catalytic site in CBH II
PGGESDGTSDTSATRYDYHCGASAALQPAPEAG

SEQ ID NO: 155 - DNA, 99 bp, Artificial Sequence
  Catalytic site in CBH II
cccggcggcgagtgcgatggcacctccaacacctcctcccctcgctacgatgcccactgcggcctgcccgatgccacccctaacgcccctgaggccggc

SEQ ID NO: 156 - protein, 33 aa, Artificial Sequence
  Catalytic site in CBH II
PGGECDGTSNTSSPRYDAHCGLPDATPNAPEAG

(19) United States
(12) Patent Application Publication
(10) Pub. No.: US 2015/0376589 A1
(43) Pub. Date: Dec. 31, 2015

(54) VARIANT CBHII POLYPEPTIDES WITH IMPROVED SPECIFIC ACTIVITY

(71) Applicants: Christopher S. LYON, San Diego, CA (US); Peter LUGINBUHL, San Diego, CA (US); Justin Trent STEGE, San Diego, CA (US)

(72) Inventors: Christopher S. LYON, San Diego, CA (US); Peter LUGINBUHL, San Diego, CA (US); Justin Trent STEGE, San Diego, CA (US)

(73) Assignee: BP Corporation North America Inc., Naperville, IL (US)

(21) Appl. No.: 14/441,670

(22) PCT Filed: Nov. 14, 2013
(86) PCT No.: PCT/US13/70116

§ 371 Date: May 8, 2015

Related U.S. Application Data
(60) Provisional application No. 61/726,712, filed on Nov. 15, 2012.

(51) Int. Cl. C12N 9/42; C12P 7/14; C12P 19/02; C12P 19/14

(52) U.S. Cl. CPC C12N 9/2434; C12P 19/14; C12P 7/14; C12P 19/02; C12Y 302/01091; C12P 2203/00

(57) ABSTRACT

The present disclosure relates to variant CBH II polypeptides that have improved specific activity, and compositions, e.g., cellulase compositions, comprising variant CBH II polypeptides. The variant CBH II polypeptides and related compositions can be used in variety of agricultural and industrial applications. The present disclosure further relates to nucleic acids encoding variant CBH II polypeptides and host cells that recombinantly express the variant CBH II polypeptides.

### REFERENCE TO SEQUENCE LISTING SUBMITTED VIA EFS-WEB

This application is being transmitted by EFS-Web, as authorized and set forth in MPEP §502.05, including a sequence listing submitted under 37 C.F.R. §1.821 in ASCII text file (.txt) format.

### BACKGROUND

Cellulose is an unbranched polymer of glucose linked by β(1→4)-glycosidic bonds. Cellulose chains can interact with each other via hydrogen bonding to form a crystalline solid of high mechanical strength and chemical stability. The cellulose chains are depolymerized into glucose and short oligosaccharides before organisms, such as the fermenting microbes used in ethanol production, can use them as metabolic fuel. Cellulase enzymes catalyze the hydrolysis of the cellulose (hydrolysis of β-1,4-D-glucan linkages) in the biomass into products such as glucose, cellobiose, and other cellooligosaccharides. Cellulase is a generic term denoting a multienzyme mixture comprising exo-acting cellobiohydrolases (CBHs), endoglucanases (EGs) and β-glucosidases (BGs) that can be produced by a number of plants and microorganisms. Enzymes in the cellulase of Trichoderma reesei include CBH I (more generally, Cel7A), CBH2 (Cel6A), EG1 (Cel7B), EG2 (Cel5), EG3 (Cel12), EG4 (Cel61A), EG5 (Cel45A), EG6 (Cel74A), Cip1, Cip2, β-glucosidases (including, e.g., Cel3A), acetyl xylan esterase, β-mannanase, and swollenin.

Cellulase enzymes work synergistically to hydrolyze cellulose to glucose. CBH I and CBH II act on opposing ends of cellulose chains (Barr et al., 1996, Biochemistry 35:586-92), while the endoglucanases act at internal locations in the cellulose. The primary product of these enzymes is cellobiose, which is further hydrolyzed to glucose by one or more (3-glucosidases.

There is a need for new and improved cellobiohyrolases with improved specific activity, for use in the conversion of cellulose into fermentable sugars and for related fields of cellulosic material processing such as pulp and paper, textiles and animal feeds.

### SUMMARY

The present disclosure relates to variant CBH II polypeptides engineered to include at least one amino acid substitution that increases specific activity as compared to a wild-type CBH II, for example the CBH II polypeptide of SEQ ID NO:2 (BD23134). The variant CBH II polypeptides of the present disclosure have at least one or more substitutions at the amino acid positions corresponding to I235, P64, L21, S104, G37, G65, K309, E66, S115, G67, E23, or A33 and/or the catalytic loop reassembly substitutions at amino acid positions corresponding to D194, A200, S421, D426, A429, T430, Y434, A438, S439, A440, L442, Q443, and P444 of SEQ ID NO:2. Such substitutions increase specific activity towards a CBH II substrate, e.g., cellulose, as compared to wild-type. The amino acid sequences of exemplary CBH II polypeptides into which a substitution at I235, P64, L21, S104, G37, G65, K309, E66, S115, G67, E23, or A33, and/or substitutions at D194, A200, S421, D426, A429, T430, Y434, A438, S439, A440, L442, Q443, and P444 can be introduced are shown in Table 1.

Accordingly, the present invention provides polypeptides (variant CBH II polypeptides) in which the CBH II has been engineered to incorporate an amino acid substitution that results in increased specific activity. Exemplary substitutions of the CBH II polypeptide include an I235V substitution, a P64W substitution, a P64E substitution, a L21R substitution, a S104V substitution, a G37S substitution, a G65L substitution, a K309H substitution, an E66R substitution, a S115A substitution, a G67K substitution, an E23K substitution, a S115M substitution, an A33K substitution, or an E23N substitution, and/or the loop reassembly substitutions D194N, A200L, S421C, D426N, A429S, T430P, Y434A, A438L, S439P, A440D, L442T, Q443P, and P444N. As used herein, an “I235V substitution” refers to a substitution of the isoleucine at the amino acid position corresponding to amino acid 235 of SEQ ID NO:2 with a valine. A “P64W substitution” refers to a substitution of the proline at the amino acid position corresponding to amino acid 64 of SEQ ID NO:2 with a tryptophan. “P64E substitution” refers to a substitution of the proline at the amino acid position corresponding to amino acid 64 of SEQ ID NO:2 with a glutamic acid, and so on.

One or more amino acid substitutions increase specific activity of the variant polypeptides of the disclosure by at least 2%, at least 3%, at least 4%, at least 5%, at least 10%, at least 15%, at least 20%, at least 25%, or at least 30% as compared to a CBH II which does not have the corresponding substitution(s). Specific activity can suitably be determined by assaying the amount of cellulose conversion to glucose in the presence of an amount of the polypeptide.

The variant CBH II polypeptides of the disclosure typically include a CD comprising an amino acid sequence having at least 50%, at least 60%, at least 70%, at least 80%, at least 90%, or at least 95% sequence identity to a CD of a reference CBH II exemplified in Table 1. The CD portion of the CBH II polypeptide having an amino acid sequence as shown in SEQ ID NO:2 is delineated in FIG. 1. The variant CBH II polypeptides can have a cellulose binding domain (“CBD”) sequence in addition to the catalytic domain (“CD”) sequence. The CBD can be N- or C-terminal to the CD, and the CBD and CD are optionally connected via a CBD-CD linker sequence. As used herein, a “CBD-CD linker” or “CBD-CD linker sequence” is an amino acid sequence that can be used to connect a CBD to a CD.

The variant CBH II polypeptides can be mature polypeptides or they may further comprise a signal sequence (“SS”). The SS is optionally connected to the CBD or CD via a SS linker sequence. As used herein, a “SS linker” or “SS linker sequence” is an amino acid sequence that can be used to connect a SS to a CBD or CD. Additional embodiments of the variant CBH II polypeptides are provided in Section 1.1.

The present disclosure further provides compositions (including cellulase compositions, e.g., whole cellulase compositions, and fermentation broths) comprising variant CBH II polypeptides. Additional embodiments of compositions comprising variant CBH II polypeptides are provided in Section 1.3. The variant CBH II polypeptides and compositions comprising them can be used, inter alia, in processes for saccharifying biomass. Additional details of saccharification reactions, and additional applications of the variant CBH II polypeptides, are provided in Section 1.4.

The present disclosure further provides nucleic acids (e.g., vectors) comprising nucleotide sequences encoding variant CBH II polypeptides as described herein, and recombinant cells engineered to express the variant CBH II polypeptides. The recombinant cell can be a prokaryotic (e.g., bacterial) or eukaryotic (e.g., yeast or filamentous fungal) cell. Further provided are methods of producing and optionally recovering the variant CBH II polypeptides. Additional embodiments of the recombinant expression system suitable for expression and production of the variant CBH II polypeptides are provided in Section 1.2.

### BRIEF DESCRIPTION OF THE FIGURES AND TABLES

FIG. 1A-1B: BD23134 wild-type CBH II. FIG. 1A shows the nucleotide coding sequence (SEQ ID NO:1) and FIG. 1B shows the amino acid sequence (SEQ ID NO:2). Signal sequences are shown with underlining only, SS linker sequences are shown with bold text only, carbohydrate binding domains are shown with italics only, CBD-CD linker sequences are shown with bold text and double underlining, and catalytic domains are shown with italics and underlining.

FIG. 2A-2B: Nucleotide and amino acid sequence alignment of amino acids 190-204 (FIG. 2A) and 417-449 (FIG. 2B) of BD23134 with the corresponding sequences of an exemplary CBHII variant polypeptide of the present disclosure having amino acid substitutions at positions 194, 200, 421, 426, 429, 430, 434, 438, 439, 440, 442, 443, and 444. BD23134 sequences are shown by SEQ ID NOS: 149-150 and 153-154. The variant polypeptide sequences are shown by SEQ ID NOS: 151-152 and 155-156. Codon substitutions resulting in an amino acid substitution are shown in bold and silent codon substitutions are shown underlined.

FIG. 3: High throughput screening work flow used for assessing variant specific activity improvements over wild-type (WT).

FIG. 4A-4B: Plots of CBH II activity versus CBH II polypeptide quantity for CBH II variant polypeptides in primary (FIG. 4A) and secondary (FIG. 4B) screens.

FIG. 5A-5B: CBH II variant polypeptide tertiary screen saccharification results. FIG. 5A identifies CBH II variants having a specific activity at least 2% greater than the specific activity of the corresponding wild-type CBH II. FIG. 5B identifies CBH II variants having a specific activity at least 5% greater than the specific activity of the corresponding wild-type CBH II.

TABLE 1: Amino acid sequences of exemplary “reference” CBH II polypeptides that can be modified at positions corresponding to I235, P64, L21, S104, G37, G65, K309, E66, S115, G67, E23, A33, D194, A200, S421, D426, A429, T430, Y434, A438, S439, A440, L442, Q443, and/or P444 in BD23134 (SEQ ID NO:2). The database accession number or patent document disclosing each reference CBH II polypeptide is indicated in the second column. For sequences disclosed in a patent document, the number following the “−” indicates the SEQ ID NO. of the sequence in the patent document. Unless indicated otherwise, the accession numbers refer to the Genbank database. “*” indicates a nonpublic database.

TABLE 2A-2B: Exemplary variant polypeptides of BD23134 having improved specific activity compared to wild-type BD23134. Codon numbering corresponds to the amino acid numbering of sequence of SEQ ID NO:2.

TABLE 3: Nucleic acid sequences for exemplary CBH II polypeptides of the invention having single amino acid substitutions as compared to BD23134. Codon substitutions are shown by underlining.

TABLE 4A-4B: Amino acid positions of the exemplary reference CBH II polypeptides that correspond to positions 21, 23, 33, 37, 64, 65, 66, 67, 104, 115, 235, and 309 (Table 4A) and the loop reassembly positions 194, 200, 421, 426, 429, 430, 434, 438, 439, 440, 442, 443, and 444 (Table 4B) of BD23134. Database descriptors are as for Table 1.

TABLE 5: Approximate amino acid positions of CBH II polypeptide domains. Abbreviations used: SS is signal sequence; CD is catalytic domain; CBD is cellulose binding domain; and CBD-CD linker is the amino acid sequence that connects the CBD to the CD. Database descriptors are as for Table 1.

### DETAILED DESCRIPTION

The present disclosure relates to variant CBH II polypeptides engineered to include amino acid substitutions that increase specific activity as compared to a wild-type CBH II, for example the CBH II polypeptide of SEQ ID NO:2 (BD23134). The variant CBH II polypeptides of the present disclosure have one or more substitutions at an amino acid corresponding to I235, P64, L21, S104, G37, G65, K309, E66, S115, G67, E23, or A33 and/or substitutions at the amino acid positions corresponding to D194, A200, S421, D426, A429, T430, Y434, A438, S439, A440, L442, Q443, and P444 of SEQ ID NO:2. Such substitutions increase specific activity towards a CBH II substrate as compared to wild-type. The following subsections describe in greater detail the variant CBH II polypeptides and exemplary methods of their production, exemplary cellulase compositions comprising them, and some industrial applications of the polypeptides and cellulase compositions.

1.1. Variant CBH II Polypeptides

The present disclosure provides variant CBH II polypeptides comprising at least one amino acid substitution that result in increased specific activity. “Variant” means a polypeptide which differs in sequence from a reference polypeptide by substitution of one or more amino acids at one or a number of different sites in the amino acid sequence. Exemplary reference CBH II polypeptides are shown in Table 1.

The variant CBH II polypeptides of the disclosure include one or more of an I235V substitution, a P64W substitution, a P64E substitution, a L21R substitution, a S104V substitution, a G37S substitution, a G65L substitution, a K309H substitution, an E66R substitution, a S115A substitution, a G67K substitution, an E23K substitution, a S115M substitution, an A33K substitution, or an E23N substitution, and/or a D194N substitution, an A200L substitution, a S421C substitution, a D426N substitution, an A429S substitution, a T430P substitution, a Y434A substitution, an A438L substitution, a S439P substitution, an A440D substitution, a L442T substitution, a Q443P substitution, and a P444N substitution. It is noted that the amino acid numbering is made by reference to the full length BD23134 CBH II (SEQ ID NO:2), which includes a signal sequence that is generally absent from the mature enzyme.

Accordingly, an “I235V substitution” is a substitution of the isoleucine at the amino acid position corresponding to amino acid 235 of SEQ ID NO:2 with a valine. A “P64W substitution” is a substitution of the proline at the amino acid position corresponding to amino acid 64 of SEQ ID NO:2 with a tryptophan. “P64E substitution” is a substitution of the proline at the amino acid position corresponding to amino acid 64 of SEQ ID NO:2 with a glutamic acid. A “L21R substitution” is a substitution of the leucine at the amino acid position corresponding to amino acid 21 of SEQ ID NO:2 with an arginine. A “S104V substitution” is a substitution of the serine at the amino acid position corresponding to amino acid 104 of SEQ ID NO:2 with a valine. A “G375 substitution” is a substitution of the glycine at the amino acid position corresponding to amino acid 37 of SEQ ID NO:2 with a serine. A “G65L substitution” is a substitution of the glycine at the amino acid position corresponding to amino acid 65 of SEQ ID NO:2 with a leucine. A “K309H substitution” is a substitution of the lysine at the amino acid position corresponding to amino acid 309 of SEQ ID NO:2 with a histidine. An “E66R substitution” is a substitution of the glutamic acid at the amino acid position corresponding to amino acid 66 of SEQ ID NO:2 with an arginine. A “S115A substitution” is a substitution of the serine at the amino acid position corresponding to amino acid 115 of SEQ ID NO:2 with an alanine. A “G67K substitution” is a substitution of the glycine at the amino acid position corresponding to amino acid 67 of SEQ ID NO:2 with a lysine. An “E23K substitution” is a substitution of the glutamic acid at the amino acid position corresponding to amino acid 23 of SEQ ID NO:2 with a lysine. A “S115M substitution” is a substitution of the serine at the amino acid position corresponding to amino acid 115 of SEQ ID NO:2 with a methionine. An “A33K substitution” is a substitution of the alanine at the amino acid position corresponding to amino acid 33 of SEQ ID NO:2 with a lysine. An “E23N substitution” is a substitution of the glutamic acid at the amino acid position corresponding to amino acid 23 of SEQ ID NO:2 with an asparagine.

A “D194N substitution” is a substitution of the aspartic acid at the amino acid position corresponding to amino acid 194 of SEQ ID NO:2 with an asparagine. An “A200L substitution” is a substitution of the alanine at the amino acid position corresponding to amino acid 200 of SEQ ID NO:2 with a leucine. A “S421C substitution” is a substitution of the serine at the amino acid position corresponding to amino acid 421 of SEQ ID NO:2 with a cysteine. A “D426N substitution” is a substitution of the aspartic acid at the amino acid position corresponding to amino acid 426 of SEQ ID NO:2 with an asparagine. An “A429S substitution” is a substitution of the alanine at the amino acid position corresponding to amino acid 429 of SEQ ID NO:2 with a serine. A “T430P substitution” is a substitution of the threonine at the amino acid position corresponding to amino acid 430 of SEQ ID NO:2 with a proline. A “Y434A substitution” is a substitution of the tyrosine at the amino acid position corresponding to amino acid 434 of SEQ ID NO:2 with an alanine. An “A438L substitution” is a substitution of the alanine at the amino acid position corresponding to amino acid 438 of SEQ ID NO:2 with a leucine. A “S439P substitution” is a substitution of the serine at the amino acid position corresponding to amino acid 439 of SEQ ID NO:2 with a proline. An “A440D substitution” is a substitution of the alanine at the amino acid position corresponding to amino acid 440 of SEQ ID NO:2 with a aspartic acid. A “L442T substitution” is a substitution of the leucine at the amino acid position corresponding to amino acid 442 of SEQ ID NO:2 with a threonine. A “Q443P substitution” is a substitution of the glutamine at the amino acid position corresponding to amino acid 443 of SEQ ID NO:2 with a proline. A “P444N substitution” is a substitution of the proline at the amino acid position corresponding to amino acid 444 of SEQ ID NO:2 with an asparagine.

Amino acid positions in CBH II polypeptides that correspond to I235, P64, L201, S104, G37, G65, K309, E66, S115, G67, E23, A33, D194, A200, S421, D426, A429, T430, Y434, A438, S439, A440, L442, Q443, and P444 of SEQ ID NO:2 can be identified through alignment of their sequences with SEQ ID NO:2 using a sequence comparison algorithm. Optimal alignment of sequences for comparison can be conducted, e.g., by the local homology algorithm of Smith & Waterman, 1981, Adv. Appl. Math. 2:482-89; by the homology alignment algorithm of Needleman & Wunsch, 1970, J. Mol. Biol. 48:443-53; by the search for similarity method of Pearson & Lipman, 1988, Proc. Nat'l Acad. Sci. USA 85:2444-48, by computerized implementations of these algorithms (GAP, BESTFIT, FASTA, and TFASTA in the Wisconsin Genetics Software Package, Genetics Computer Group, 575 Science Dr., Madison, Wis.), or by visual inspection.

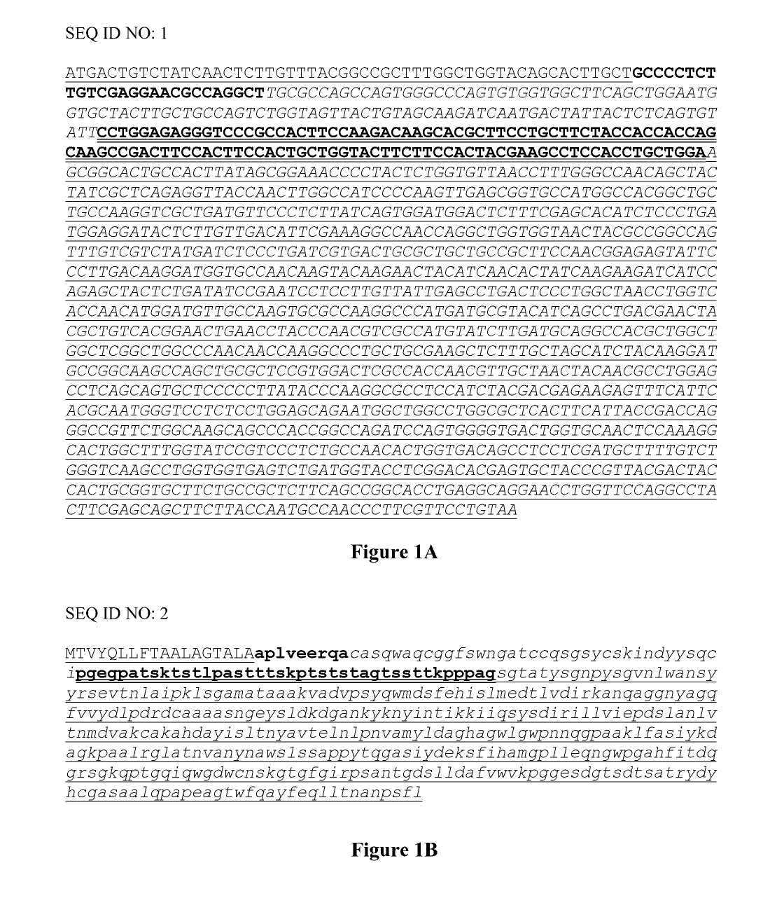

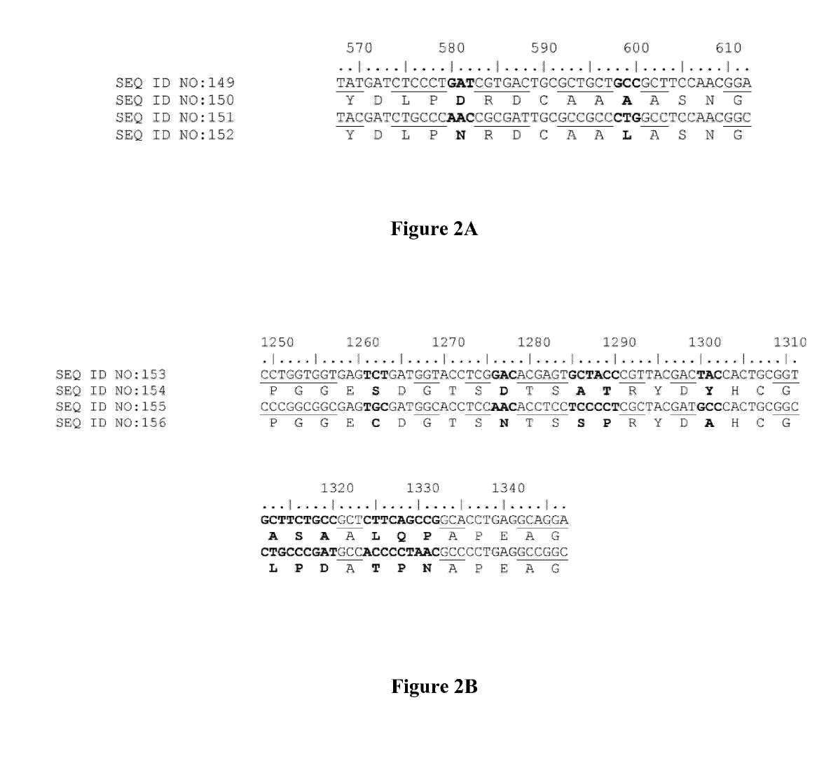

A variant CBH II can include only the CD “core” of CBH II. An exemplary reference CD comprises an amino acid sequence corresponding to positions 104 to 468 of SEQ ID NO:2 (FIG. 1B). The CDs of other exemplary CBH II polypeptides are delineated in Table 5.

The CBDs are particularly involved in the hydrolysis of crystalline cellulose. It has been shown that the ability of cellobiohydrolases to degrade crystalline cellulose decreases when the CBD is absent (Linder and Teeri, 1997, Journal of Biotechnol. 57:15-28). The variant CBH II polypeptides of the disclosure can further include a CBD. An exemplary CBD comprises an amino acid sequence corresponding to positions 28 to 63 of SEQ ID NO:2 (FIG. 1B). The CBDs of other exemplary CBH II polypeptides are delineated in Table 5.

The CD and CBD are often connected via a CBD-CD linker. The variant CBH II polypeptides of the disclosure can further include a CBD-CD linker. An exemplary CBD-CD linker sequence corresponds to positions 64 to 103 of SEQ ID NO:2 (FIG. 1B). Other exemplary CBH II CBD-CD linkers are delineated in Table 5.

The SS and CBD are often connected via a SS linker. The variant CBH II polypeptides of the disclosure can further include a SS linker. An exemplary SS linker corresponds to positions 19 to 27 of SEQ ID NO:2 (FIG. 1B).

Because CBH II polypeptides are modular, the CBDs, CDs and CBD-CD linkers of different CBH II polypeptides, such as the exemplary CBH II polypeptides of Table 1, can be used interchangeably. However, in a preferred embodiment, the CBDs, CDs and CBD-CD linkers of a variant CBH II of the disclosure originate from the same polypeptide.

The variant CBH II polypeptides of the disclosure preferably have a cellobiohydrolase activity that is at least 2%, at least 3%, at least 4%, at least 5%, at least 10%, at least 15%, at least 20%, at least 25%, or at least 30% greater than the cellobiohydrolase activity of the corresponding reference CBH II, e.g., CBH II lacking a substitution at I235, P64, L21, S104, G37, G65, K309, E66, S115, G67, E23, A33, D194, A200, S421, D426, A429, T430, Y434, A438, S439, A440, L442, Q443, or P444. Assays for cellobiohydrolase activity are described, for example, in Nidetzky et al., 1994, Biochem. J. 303:817-823. The ability of CBH II to hydrolyze isolated soluble and insoluble substrates can also be measured using assays described in Jager et al., 2010, Biotech. Biofuels 3:18:1-12 and Nidetzky and Claeyssens, 1994, Biotech. Bioeng. 44:961-966. Substrates useful for assaying cellobiohydrolase activity include crystalline cellulose, filter paper, phosphoric acid swollen cellulose, cellooligosaccharides, methylumbelliferyl lactoside, methylumbelliferyl cellobioside, orthonitrophenyl lactoside, paranitrophenyl lactoside, orthonitrophenyl cellobioside, paranitrophenyl cellobioside. Cellobiohydrolase activity can be measured in an assay utilizing PASC as the substrate and a calcofluor white detection method (Du et al., 2010, Applied Biochemistry and Biotechnology 161:313-317). PASC can be prepared as described by Walseth, 1952, TAPPI 35:228-235 and Wood, 1971, Biochem. J. 121:353-362.

Other than the I235, P64, L21, S104, G37, G65, K309, E66, S115, G67, E23, A33, or loop reassembly (D194, A200, S421, D426, A429, T430, Y434, A438, S439, A440, L442, Q443, and P444) substitutions, the variant CBH II polypeptides of the disclosure preferably:
comprise an amino acid sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more, or complete (100%) sequence identity to a CD of a reference CBH II exemplified in Table 1 (e.g., a CD comprising an amino acid sequence corresponding to positions 104 to 468 of SEQ ID NO:2); and/or
comprise an amino acid sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more, or complete (100%) sequence identity to a mature polypeptide of a reference CBH II exemplified in Table 1.

An example of an algorithm that is suitable for determining sequence similarity is the BLAST algorithm, which is described in Altschul et al., 1990, J. Mol. Biol. 215:403-410. Software for performing BLAST analyses is publicly available through the National Center for Biotechnology Information. This algorithm involves first identifying high scoring sequence pairs (HSPs) by identifying short words of length W in the query sequence that either match or satisfy some positive-valued threshold score T when aligned with a word of the same length in a database sequence. These initial neighborhood word hits act as starting points to find longer HSPs containing them. The word hits are expanded in both directions along each of the two sequences being compared for as far as the cumulative alignment score can be increased. Extension of the word hits is stopped when: the cumulative alignment score falls off by the quantity X from a maximum achieved value; the cumulative score goes to zero or below; or the end of either sequence is reached. The BLAST algorithm parameters W, T, and X determine the sensitivity and speed of the alignment. The BLAST program uses as defaults a word length (W) of 11, the BLOSUM62 scoring matrix (see Henikoff & Henikoff, 1992, Proc. Nat'l. Acad. Sci. USA 89:10915-10919) alignments (B) of 50, expectation (E) of 10, M′5, N′-4, and a comparison of both strands.

Most CBH II polypeptides are secreted and are therefore expressed with a signal sequence that is cleaved upon secretion of the polypeptide from the cell. Accordingly, in certain aspects, the variant CBH II polypeptides of the disclosure further include a signal sequence. An exemplary signal sequences comprises an amino acid sequence corresponding to positions 1 to 18 of SEQ ID NO:2 (FIG. 1B). Other exemplary signal sequences are delineated in Table 5.

1.2. Recombinant Expression of Variant CBH II Polypeptides

1.2.1. Cell Culture Systems

The disclosure also provides recombinant cells engineered to express variant CBH II polypeptides. Suitably, the variant CBH II polypeptide is encoded by a nucleic acid operably linked to a promoter. The promoters can be homologous or heterologous, and constitutive or inducible.

Suitable host cells include cells of any microorganism (e.g., cells of a bacterium, a protist, an alga, a fungus (e.g., a yeast or filamentous fungus), or other microbe), and are preferably cells of a bacterium, a yeast, or a filamentous fungus.

Where recombinant expression in a filamentous fungal host is desired, the promoter can be a fungal promoter (including but not limited to a filamentous fungal promoter), a promoter operable in plant cells, or a promoter operable in mammalian cells.

As described in U.S. provisional application No. 61/553,901, filed Oct. 31, 2011, the contents of which are hereby incorporated in their entireties, promoters that are constitutively active in mammalian cells (which can derived from a mammalian genome or the genome of a mammalian virus) are capable of eliciting high expression levels in filamentous fungi such as Trichoderma reesei. An exemplary promoter is the cytomegalovirus (“CMV”) promoter.

As described in U.S. provisional application No. 61/553,897, filed Oct. 31, 2011, the contents of which are hereby incorporated in their entireties, promoters that are constitutively active in plant cells (which can derived from a plant genome or the genome of a plant virus) are capable of eliciting high expression levels in filamentous fungi such as Trichoderma reesei. Exemplary promoters are the cauliflower mosaic virus (“CaMV”) 35S promoter or the Commelina yellow mottle virus (“CoYMV”) promoter.

Mammalian, mammalian viral, plant and plant viral promoters can drive particularly high expression when the associated 5′ UTR sequence (i.e., the sequence which begins at the transcription start site and ends one nucleotide (nt) before the start codon) normally associated with the mammalian or mammalian viral promoter is replaced by a fungal 5′ UTR sequence.

The source of the 5′ UTR can vary provided it is operable in the filamentous fungal cell. In various embodiments, the 5′ UTR can be derived from a yeast gene or a filamentous fungal gene. The 5′ UTR can be from the same species as one other component in the expression cassette (e.g., the promoter or the CBH II coding sequence), or from a different species. The 5′ UTR can be from the same species as the filamentous fungal cell that the expression construct is intended to operate in. In an exemplary embodiment, the 5′ UTR comprises a sequence corresponding to a fragment of a 5′ UTR from a T. reesei glyceraldehyde-3-phosphate dehydrogenase (gpd). In a specific embodiment, the 5′ UTR is not naturally associated with the CMV promoter

Examples of other promoters that can be used include, but are not limited to, a cellulase promoter, a xylanase promoter, the 1818 promoter (previously identified as a highly expressed protein by EST mapping Trichoderma). For example, the promoter can suitably be a cellobiohydrolase, endoglucanase, or β-glucosidase promoter. A particularly suitable promoter can be, for example, a T. reesei cellobiohydrolase, endoglucanase, or β-glucosidase promoter. Non-limiting examples of promoters include a cbh1, cbh2, egl1, egl2, egl3, egl4, egl5, pki1, gpd1, xyn1, or xyn2 promoter.

Suitable host cells of the bacterial genera include, but are not limited to, cells of Escherichia, Bacillus, Lactobacillus, Pseudomonas, and Streptomyces. Suitable cells of bacterial species include, but are not limited to, cells of Escherichia coli, Bacillus subtilis, Bacillus licheniformis, Lactobacillus brevis, Pseudomonas aeruginosa, and Streptomyces lividans.

Suitable host cells of the genera of yeast include, but are not limited to, cells of Saccharomyces, Schizosaccharomyces, Candida, Hansenula, Pichia, Kluyveromyces, and Phaffia. Suitable cells of yeast species include, but are not limited to, cells of Saccharomyces cerevisiae, Schizosaccharomyces pombe, Candida albicans, Hansenula polymorpha, Pichia pastoris, P. canadensis, Kluyveromyces marxianus, and Phaffia rhodozyma.

Suitable host cells of filamentous fungi include all filamentous forms of the subdivision Eumycotina. Suitable cells of filamentous fungal genera include, but are not limited to, cells of Acremonium, Aspergillus, Aureobasidium, Bjerkandera, Ceriporiopsis, Chrysoporium, Coprinus, Coriolus, Corynascus, Chaetomium, Cryptococcus, Filobasidium, Fusarium, Gibberella, Humicola, Hypocrea, Magnaporthe, Mucor, Myceliophthora, Mucor, Neocallimastix, Neurospora, Paecilomyces, Penicillium, Phanerochaete, Phlebia, Piromyces, Pleurotus, Scytaldium, Schizophyllum, Sporotrichum, Talaromyces, Thermoascus, Thielavia, Tolypocladium, Trametes, and Trichoderma. More preferably, the recombinant cell is a Trichoderma sp. (e.g., Trichoderma reesei), Penicillium sp., Humicola sp. (e.g., Humicola insolens); Aspergillus sp. (e.g., Aspergillus niger), Chrysosporium sp., Fusarium sp., or Hypocrea sp. Suitable cells can also include cells of various anamorph and teleomorph forms of these filamentous fungal genera.

Suitable cells of filamentous fungal species include, but are not limited to, cells of Aspergillus awamori, Aspergillus fumigatus, Aspergillus foetidus, Aspergillus japonicus, Aspergillus nidulans, Aspergillus niger, Aspergillus oryzae, Chrysosporium lucknowense, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Bjerkandera adusta, Ceriporiopsis aneirina, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Coprinus cinereus, Coriolus hirsutus, Humicola insolens, Humicola lanuginosa, Mucor miehei, Myceliophthora thermophila, Neurospora crassa, Neurospora intermedia, Penicillium purpurogenum, Penicillium canescens, Penicillium solitum, Penicillium funiculosum, Phanerochaete chrysosporium, Phlebia radiate, Pleurotus eryngii, Talaromyces flavus, Thielavia terrestris, Trametes villosa, Trametes versicolor, Trichoderma harzianum, Trichoderma koningii, Trichoderma longibrachiatum, Trichoderma reesei, and Trichoderma viride.

The engineered host cells can be cultured in conventional nutrient media modified as appropriate for activating promoters, selecting transformants, or amplifying the nucleic acid sequence encoding the variant CBH II polypeptide. Culture conditions, such as temperature, pH and the like, are those previously used with the host cell selected for expression, and will be apparent to those skilled in the art. As noted, many references are available for the culture and production of many cells, including cells of bacterial and fungal origin. Cell culture media in general are set forth in Atlas and Parks (eds.), 1993, The Handbook of Microbiological Media, CRC Press, Boca Raton, Fla., which is incorporated herein by reference. For recombinant expression in filamentous fungal cells, the cells are cultured in a standard medium containing physiological salts and nutrients, such as described in Pourquie et al., 1988, Biochemistry and Genetics of Cellulose Degradation, eds. Aubert, et al., Academic Press, pp. 71-86; and Ilmen et al., 1997, Appl. Environ. Microbiol. 63:1298-1306. Culture conditions are also standard, e.g., cultures are incubated at 28° C. in shaker cultures or fermenters until desired levels of variant CBH II expression are achieved. Preferred culture conditions for a given filamentous fungus may be found in the scientific literature and/or from the source of the fungi such as the American Type Culture Collection (ATCC). After fungal growth has been established, the cells are exposed to conditions effective to cause or permit the expression of a variant CBH II.

In cases where a variant CBH II coding sequence is under the control of an inducible promoter, the inducing agent, e.g., a sugar, metal salt or antibiotics, is added to the medium at a concentration effective to induce variant CBH II expression.

In one embodiment, the recombinant cell is an Aspergillus niger, which is a useful strain for obtaining overexpressed polypeptide. For example A. niger var. awamori dgr246 is known to product elevated amounts of secreted cellulases (Goedegebuur et al., 2002, Curr. Genet. 41:89-98). Other strains of Aspergillus niger var awamori such as GCDAP3, GCDAP4 and GAP3-4 are known (Ward et al., 1993, Appl. Microbiol. Biotechnol. 39:738-743).

In another embodiment, the recombinant cell is a Trichoderma reesei, which is a useful strain for obtaining overexpressed polypeptide. For example, RL-P37, described by Sheir-Neiss et al., 1984, Appl. Microbiol. Biotechnol. 20:46-53, is known to secrete elevated amounts of cellulase enzymes. Functional equivalents of RL-P37 include Trichoderma reesei strain RUT-C30 (ATCC No. 56765) and strain QM9414 (ATCC No. 26921). It is contemplated that these strains would also be useful in overexpressing variant CBH II polypeptides.

Cells expressing the variant CBH II polypeptides of the disclosure can be grown under batch, fed-batch or continuous fermentations conditions. Classical batch fermentation is a closed system, wherein the compositions of the medium is set at the beginning of the fermentation and is not subject to artificial alternations during the fermentation. A variation of the batch system is a fed-batch fermentation in which the substrate is added in increments as the fermentation progresses. Fed-batch systems are useful when catabolite repression is likely to inhibit the metabolism of the cells and where it is desirable to have limited amounts of substrate in the medium. Batch and fed-batch fermentations are common and well known in the art. Continuous fermentation is an open system where a defined fermentation medium is added continuously to a bioreactor and an equal amount of conditioned medium is removed simultaneously for processing. Continuous fermentation generally maintains the cultures at a constant high density where cells are primarily in log phase growth. Continuous fermentation systems strive to maintain steady state growth conditions. Methods for modulating nutrients and growth factors for continuous fermentation processes as well as techniques for maximizing the rate of product formation are well known in the art of industrial microbiology.

1.2.2. Recombinant Expression in Plants

The disclosure provides transgenic plants and seeds that recombinantly express a variant CBH II polypeptide. The disclosure also provides plant products, e.g., oils, seeds, leaves, extracts and the like, comprising a variant CBH II polypeptide.

The transgenic plant can be dicotyledonous (a dicot) or monocotyledonous (a monocot). The disclosure also provides methods of making and using these transgenic plants and seeds. The transgenic plant or plant cell expressing a variant CBH II can be constructed in accordance with any method known in the art. See, for example, U.S. Pat. No. 6,309,872. T. reesei CBH I has been successfully expressed in transgenic tobacco (Nicotiana tabaccum) and potato (Solanum tuberosum). See Hooker et al., 2000, in Glycosyl Hydrolases for Biomass Conversion, ACS Symposium Series, Vol. 769, Chapter 4, pp. 55-90. It is contemplated that CBH II can be similarly expressed.

In a particular aspect, the present disclosure provides for the expression of CBH II variants in transgenic plants or plant organs and methods for the production thereof. DNA expression constructs are provided for the transformation of plants with a nucleic acid encoding the variant CBH II polypeptide, preferably under the control of regulatory sequences which are capable of directing expression of the variant CBH II polypeptide. These regulatory sequences include sequences capable of directing transcription in plants, either constitutively, or in stage and/or tissue specific manners.

The expression of variant CBH II polypeptides in plants can be achieved by a variety of means. Specifically, for example, technologies are available for transforming a large number of plant species, including dicotyledonous species (e.g., tobacco, potato, tomato, Petunia, Brassica) and monocot species. Additionally, for example, strategies for the expression of foreign genes in plants are available. Additionally still, regulatory sequences from plant genes have been identified that are serviceable for the construction of chimeric genes that can be functionally expressed in plants and in plant cells (e.g., Klee, 1987, Ann. Rev. of Plant Phys. 38:467-486; Clark et al., 1990, Virology 179(2):640-7; Smith et al., 1990, Mol. Gen. Genet. 224(3):477-81.

The introduction of nucleic acids into plants can be achieved using several technologies including transformation with Agrobacterium tumefaciens or Agrobacterium rhizogenes. Non-limiting examples of plant tissues that can be transformed include protoplasts, microspores or pollen, and explants such as leaves, stems, roots, hypocotyls, and cotyls. Furthermore, DNA encoding a variant CBH II can be introduced directly into protoplasts and plant cells or tissues by microinjection, electroporation, particle bombardment, and direct DNA uptake.

Variant CBH II polypeptides can be produced in plants by a variety of expression systems. For instance, the use of a constitutive promoter such as the 35S promoter of Cauliflower Mosaic Virus (Guilley et al., 1982, Cell 30:763-73) is serviceable for the accumulation of the expressed protein in virtually all organs of the transgenic plant. Alternatively, promoters that are tissue-specific and/or stage-specific can be used (Higgins, 1984, Annu. Rev. Plant Physiol. 35:191-221; Shotwell and Larkins, 1989, In: The Biochemistry of Plants Vol. 15 (Academic Press, San Diego: Stumpf and Conn, eds.), p. 297), permitting expression of variant CBH II polypeptides in a target tissue and/or during a desired stage of development.

1.3. Compositions Of Variant CBH II Polypeptides

In general, a variant CBH II polypeptide produced in cell culture is secreted into the medium and may be purified or isolated, e.g., by removing unwanted components from the cell culture medium. However, in some cases, a variant CBH II polypeptide may be produced in a cellular form necessitating recovery from a cell lysate. In such cases the variant CBH II polypeptide is purified from the cells in which it was produced using techniques routinely employed by those skilled in the art. Examples include, but are not limited to, affinity chromatography (Van Tilbeurgh et al., 1984, FEBS Lett. 169(2):215-218), ion-exchange chromatographic methods (Goyal et al., 1991, Bioresource Technology, 36:37-50; Fliess et al., 1983, Eur. J. Appl. Microbiol. Biotechnol. 17:314-318; Bhikhabhai et al., 1984, J. Appl. Biochem. 6:336-345; Ellouz et al., 1987, Journal of Chromatography, 396:307-317), including ion-exchange using materials with high resolution power (Medve et al., 1998, J. Chromatography A, 808:153-165), hydrophobic interaction chromatography (Tomaz and Queiroz, 1999, J. Chromatography A, 865:123-128), and two-phase partitioning (Brumbauer et al., 1999, Bioseparation 7:287-295).

The variant CBH II polypeptides of the disclosure are suitably used in cellulase compositions. Cellulases are known in the art as enzymes that hydrolyze cellulose (beta-1,4-glucan or beta D-glucosidic linkages) resulting in the formation of glucose, cellobiose, cellooligosaccharides, and the like. Cellulase enzymes have been traditionally divided into three major classes: endoglucanases (“EG”), exoglucanases or cellobiohydrolases (EC 3.2.1.91) (“CBH”) and beta-glucosidases (EC 3.2.1.21) (“BG”) (Knowles et al., 1987, TIBTECH 5:255-261; Schulein, 1988, Methods in Enzymology 160(25):234-243).

Certain fungi produce complete cellulase systems which include exo-cellobiohydrolases or CBH-type cellulases, endoglucanases or EG-type cellulases and (3-glucosidases or BG-type cellulases (Schulein, 1988, Methods in Enzymology 160(25):234-243). Such cellulase compositions are referred to herein as “whole” cellulases. However, sometimes these systems lack CBH-type cellulases and bacterial cellulases also typically include little or no CBH-type cellulases. In addition, it has been shown that the EG components and CBH components synergistically interact to more efficiently degrade cellulose. See, e.g., Wood, 1985, Biochemical Society Transactions 13(2):407-410.

The cellulase compositions of the disclosure typically include, in addition to a variant CBH II polypeptide, one or more cellobiohydrolases, endoglucanases and/or β-glucosidases. In their crudest form, cellulase compositions contain the microorganism culture that produced the enzyme components. “Cellulase compositions” also refers to a crude fermentation product of the microorganisms. A crude fermentation is preferably a fermentation broth that has been separated from the microorganism cells and/or cellular debris (e.g., by centrifugation and/or filtration). In some cases, the enzymes in the broth can be optionally diluted, concentrated, partially purified or purified and/or dried. The variant CBH II polypeptide can be co-expressed with one or more of the other components of the cellulase composition or it can be expressed separately, optionally purified and combined with a composition comprising one or more of the other cellulase components.

When employed in cellulase compositions, the variant CBH II is generally present in an amount sufficient to allow release of soluble sugars from the biomass. The amount of variant CBH II enzymes added depends upon the type of biomass to be saccharified which can be readily determined by the skilled artisan. In certain embodiments, the weight percent of variant CBH II polypeptide is suitably at least 1, at least 5, at least 10, or at least 20 weight percent of the total polypeptides in a cellulase composition. Exemplary cellulase compositions include a variant CBH II of the disclosure in an amount ranging from about 1 to about 20 weight percent, from about 1 to about 25 weight percent, from about 5 to about 20 weight percent, from about 5 to about 25 weight percent, from about 5 to about 30 weight percent, from about 5 to about 35 weight percent, from about 5 to about 40 weight percent, from about 5 to about 45 weight percent, from about 5 to about 50 weight percent, from about 10 to about 20 weight percent, from about 10 to about 25 weight percent, from about 10 to about 30 weight percent, from about 10 to about 35 weight percent, from about 10 to about 40 weight percent, from about 10 to about 45 weight percent, from about 10 to about 50 weight percent, from about 15 to about 20 weight percent, from about 15 to about 25 weight percent, from about 15 to about 30 weight percent, from about 15 to about 35 weight percent, from about 15 to about 30 weight percent, from about 15 to about 45 weight percent, or from about 15 to about 50 weight percent of the total polypeptides in the composition.

1.4. Utility of Variant CBH II Polypeptides

It can be appreciated that the variant CBH II polypeptides of the disclosure and compositions comprising the variant CBH II polypeptides find utility in a wide variety applications, for example detergent compositions that exhibit enhanced cleaning ability, function as a softening agent and/or improve the feel of cotton fabrics (e.g., “stone washing” or “biopolishing”), or in cellulase compositions for degrading wood pulp into sugars (e.g., for bio-ethanol production). Other applications include the treatment of mechanical pulp (Pere et al., 1996, Tappi Pulping Conference, pp. 693-696 (Nashville, Tenn., Oct. 27-31, 1996)), for use as a feed additive (see, e.g., WO 91/04673) and in grain wet milling.

1.4.1. Saccharification Reactions

Biofuels such as ethanol can be produced via saccharification and fermentation processes from cellulosic biomass such as trees, herbaceous plants, municipal solid waste and agricultural and forestry residues. However, the ratio of individual cellulase enzymes within a naturally occurring cellulase mixture produced by a microbe may not be the most efficient for rapid conversion of cellulose in biomass to glucose. It is known that endoglucanases act to produce new cellulose chain ends which themselves are substrates for the action of cellobiohydrolases and thereby improve the efficiency of hydrolysis of the entire cellulase system. The use of optimized cellobiohydrolase activity may greatly enhance the production of ethanol.

Cellulase compositions comprising one or more of the variant CBH II polypeptides of the disclosure can be used in saccharification reaction to produce simple sugars for fermentation. Accordingly, the present disclosure provides methods for saccharification comprising contacting biomass with a cellulase composition comprising a variant CBH II polypeptide of the disclosure and, optionally, subjecting the resulting sugars to fermentation by a microorganism.

The term “biomass,” as used herein, refers to any composition comprising cellulose (optionally also hemicellulose and/or lignin). As used herein, biomass includes, without limitation, seeds, grains, tubers, plant waste or byproducts of food processing or industrial processing (e.g., stalks), corn (including, e.g., cobs, stover, and the like), grasses (including, e.g., Indian grass, such as Sorghastrum nutans; or, switchgrass, e.g., Panicum species, such as Panicum virgatum), wood (including, e.g., wood chips, processing waste), paper, pulp, and recycled paper (including, e.g., newspaper, printer paper, and the like). Other biomass materials include, without limitation, potatoes, soybean (e.g., rapeseed), barley, rye, oats, wheat, beets, and sugar cane bagasse.

The saccharified biomass (e.g., lignocellulosic material processed by enzymes of the disclosure) can be made into a number of bio-based products, via processes such as, e.g., microbial fermentation and/or chemical synthesis. As used herein, “microbial fermentation” refers to a process of growing and harvesting fermenting microorganisms under suitable conditions. The fermenting microorganism can be any microorganism suitable for use in a desired fermentation process for the production of bio-based products. Suitable fermenting microorganisms include, without limitation, filamentous fungi, yeast, and bacteria. The saccharified biomass can, for example, be made into a fuel (e.g., a biofuel such as a bioethanol, biobutanol, biomethanol, a biopropanol, a biodiesel, a jet fuel, or the like) via fermentation and/or chemical synthesis. The saccharified biomass can, for example, also be made into a commodity chemical (e.g., ascorbic acid, isoprene, 1,3-propanediol), lipids, amino acids, polypeptides, and enzymes, via fermentation and/or chemical synthesis.

Thus, in certain aspects, the variant CBH II polypeptides of the disclosure find utility in the generation of biofuels such as ethanol from biomass in either separate or simultaneous saccharification and fermentation processes. Separate saccharification and fermentation is a process whereby cellulose present in biomass is saccharified into simple sugars (e.g., glucose) and the simple sugars subsequently fermented by microorganisms (e.g., yeast) into ethanol. Simultaneous saccharification and fermentation is a process whereby cellulose present in biomass is saccharified into simple sugars (e.g., glucose) and, at the same time and in the same reactor, microorganisms (e.g., yeast) ferment the simple sugars into ethanol.

Prior to saccharification, biomass is preferably subject to one or more pretreatment step(s) in order to render cellulose material more accessible or susceptible to enzymes and thus more amenable to hydrolysis by the variant CBH II polypeptides of the disclosure.

In an exemplary embodiment, the pretreatment entails subjecting biomass material to a catalyst comprising a dilute solution of a strong acid and a metal salt in a reactor. The biomass material can, e.g., be a raw material or a dried material. This pretreatment can lower the activation energy, or the temperature, of cellulose hydrolysis, ultimately allowing higher yields of fermentable sugars. See, e.g., U.S. Pat. Nos. 6,660,506; 6,423,145.

Another exemplary pretreatment method entails hydrolyzing biomass by subjecting the biomass material to a first hydrolysis step in an aqueous medium at a temperature and a pressure chosen to effectuate primarily depolymerization of hemicellulose without achieving significant depolymerization of cellulose into glucose. This step yields a slurry in which the liquid aqueous phase contains dissolved monosaccharides resulting from depolymerization of hemicellulose, and a solid phase containing cellulose and lignin. The slurry is then subject to a second hydrolysis step under conditions that allow a major portion of the cellulose to be depolymerized, yielding a liquid aqueous phase containing dissolved/soluble depolymerization products of cellulose. See, e.g., U.S. Pat. No. 5,536,325.

A further exemplary method involves processing a biomass material by one or more stages of dilute acid hydrolysis using about 0.4% to about 2% of a strong acid; followed by treating the unreacted solid lignocellulosic component of the acid hydrolyzed material with alkaline delignification. See, e.g., U.S. Pat. No. 6,409,841. Another exemplary pretreatment method comprises prehydrolyzing biomass (e.g., lignocellulosic materials) in a prehydrolysis reactor; adding an acidic liquid to the solid lignocellulosic material to make a mixture; heating the mixture to reaction temperature; maintaining reaction temperature for a period of time sufficient to fractionate the lignocellulosic material into a solubilized portion containing at least about 20% of the lignin from the lignocellulosic material, and a solid fraction containing cellulose; separating the solubilized portion from the solid fraction, and removing the solubilized portion while at or near reaction temperature; and recovering the solubilized portion. The cellulose in the solid fraction is rendered more amenable to enzymatic digestion. See, e.g., U.S. Pat. No. 5,705,369. Further pretreatment methods can involve the use of hydrogen peroxide H₂O₂. See Gould, 1984, Biotech, and Bioengr. 26:46-52.

Pretreatment can also comprise contacting a biomass material with stoichiometric amounts of sodium hydroxide and ammonium hydroxide at a very low concentration. See Teixeira et al., 1999, Appl. Biochem. and Biotech. 77-79:19-34. Pretreatment can also comprise contacting a lignocellulose with a chemical (e.g., a base, such as sodium carbonate or potassium hydroxide) at a pH of about 9 to about 14 at moderate temperature, pressure, and pH. See PCT Publication WO2004/081185.

Ammonia pretreatment can also be used. Such a pretreatment method comprises subjecting a biomass material to low ammonia concentration under conditions of high solids. See, e.g., U.S. Patent Publication No. 20070031918 and PCT publication WO 06/110901.

1.4.2. Detergent Compositions Comprising Variant CBH II Proteins

The present disclosure also provides detergent compositions comprising a variant CBH II polypeptide of the disclosure. The detergent compositions may employ besides the variant CBH II polypeptide one or more of a surfactant, including anionic, non-ionic and ampholytic surfactants; a hydrolase; a bleaching agents; a bluing agent; a caking inhibitors; a solubilizer; and a cationic surfactant. All of these components are known in the detergent art.

The variant CBH II polypeptide is preferably provided as part of cellulase composition. The cellulase composition can be employed from about 0.00005 weight percent to about 5 weight percent or from about 0.0002 weight percent to about 2 weight percent of the total detergent composition. The cellulase composition can be in the form of a liquid diluent, granule, emulsion, gel, paste, and the like. Such forms are known to the skilled artisan. When a solid detergent composition is employed, the cellulase composition is preferably formulated as granules.

### 2. Example 1

### Identification and Characterization of

Variants of CBH II Having Increased Specific Activity

2.1. Materials and Methods

2.1.1. CBH II Library Generation

The wild-type BD23134 CBH II gene was inserted into the pDC-A2 vector and variants were made using Gene Site Saturation Mutagenesis (GSSM) technology. In addition to making a library of single amino acid variants, a “loop reassembly” library was made to test the effect of mutations within selected loops on substrate binding. Representative loops were selected from a survey and phylogenetic analysis of surface loops across fungal and bacterial CBH II.

Overlapping DNA primers containing NNK degeneracy, where N represents any nucleotide (A, C, G, or T) and where K represents the keto group containing nucleotides (G or T), were used to create a library of variants for every amino acid position following the signal peptide in wild-type BD23134. The mutated residues included the SS linker region, the complete N-terminal CBM domain, the CBD-CD linker region, and the catalytic domain. The NNK degeneracy of the mutagenesis primers can potentially generate 32 different codons covering all 20 possible amino acids at each residue.

GSSM reactions were run in 96-well plates using methylated template DNA of the wild-type CBH II prepared from a standard laboratory dam+E. coli host strain. Paired forward and reverse NNK degenerate primers for each amino acid position were combined with the template DNA along with dNTPs, reaction buffer and high fidelity DNA polymerase. GSSM reactions were run under standard PCR conditions, with elongation times appropriate for amplification of the protein of interest and the replicating plasmid on which it was contained. Each GSSM reaction produced products consisting of a library of variants, potentially containing up to all 20 possible amino acids, for a single residue. The reaction products were treated with DpnI restriction enzyme to digest the methylated wild-type template DNA and leave the non-methylated variant DNA intact. After DpnI treatment the PCR products were run on a 1% agarose gel and stained with ethidium bromide to confirm amplification of the plasmid.

The pDC-A2 vector used in making the CBH II variants was a reconstruction of the vector pGBFin-5 (described, e.g., in U.S. Pat. No. 7,220,542), which was remade to reduce the total size of the vector. The 2.1 kb 3′ Gla region of pGBFin-5 was reduced to 0.54 kb, the gpd promoter remained the same, but the 2.24 kb amdS sequence was replaced by the 1.02 kb hygB gene encoding hygromycin phosphotransferase. The 2.3 kb 3′ Gla region of pGBFin-5 was reduced to a 1.1 kb fragment representing the 5′ end of the original sequence. The E. coli replicon for pDC-A2 was taken from pUC18.

After transformation of the vectors from the GSSM reactions into E. coli Stbl2, individual E. coli transformants were picked into 96-well plates and grown in liquid culture in 200 μl LB plus ampicillin (100 μg/ml) per well overnight at 30° C. The cells were then used to generate template for sequencing reactions by colony PCR. The sequence data from the library of clones was analyzed to identify unique CBH II variants. The E. coli transformants containing the selected variants were then rearrayed in 96-well format and used to prepare linear DNA of the entire expression cassette (the contents of pDC-A2 with the exception of the E. coli replicon) by PCR, using primers hybridizing to the ends of the 3′ and 3″ Gla regions. Approximately 1 μg of PCR product from each clone was then used to transform A. niger protoplasts in a PEG-mediated transformation in one well of a 96-well plate (i.e. one clone per well). Transformants were selected on regeneration agar (200 μl per well of PDA plus sucrose at 340 g/l and hygromycin at 200 μg/ml) in the same 96-well format. After 7 days incubation at 30° C., transformants were replicated to 96-well plates containing PDA plus hygromycin (200 μg/ml) using a pintool. Following incubation at 30° C. for a further 7 days, spores from each well were used to inoculate 200 μl liquid media per well of a 96-well plate.

2.1.2. Preparation of CBH II Polypeptides for Biochemical Characterization

For primary screening, protein expression was carried out in an Aspergillus niger host strain that had been transformed with expression constructs for BD23134 variants. Variants were grown in liquid growth media by transferring transformation spores from agar plates into 96 well Pall® filter plates. In columns 6 and 12 of each plate wild-type BD23134 and a “host only” control (containing the expression vector without the CBHII construct inserted) were grown. The growth media had the following composition: NaNO3, 3.0 g/l; KCl, 0.26 g/l; KH₂PO₄, 0.76 g/l; 4M KOH, 0.56 ml/l; D-Glucose, 5.0 g/l; Casamino Acids, 0.5 g/l; Trace Element Solution 0.5 ml/l; Vitamin Solution 5 ml/l; Penicillin-Streptomycin Solution (10,000 U/ml and 10,000 m/ml, respectively) 5.0 ml/l; Maltose, 66.0 g/l; Soytone, 26.4 g/l; (NH₄)₂SO₄, 6.6 g/l; NaH₂PO₄.H₂O, 0.44 g/l; MgSO₄.7H₂O, 0.44 g/l; Arginine, 0.44 g/l; Tween-80, 0.035 ml/l; Pleuronic Acid Antifoam, 0.0088 ml/l; MES, 18.0 g/l. The Trace Element Solution had the following composition in 100 ml: ZnSO₄.7H₂O, 2.2 g; H₃BO₃, 1.1 g; FeSO₄.7H₂O, 0.5 g; CoCl₂.6H₂O, 0.17 g; CuSO₄.5H₂O, 0.16; MnCl₂.4H₂O, 0.5 g/l; NaMoO₄.2H₂O, 0.15 g/l; EDTA, 5 g/l. The Vitamin Solution had the following composition in 500 ml: Riboflavin, 100 mg; Thiamine HCl, 100 mg; Nicotinamide, 100 mg; Pyridoxine HCl, 50 mg; Panthotenic Acid, 10 mg; Biotin 0.2 mg.

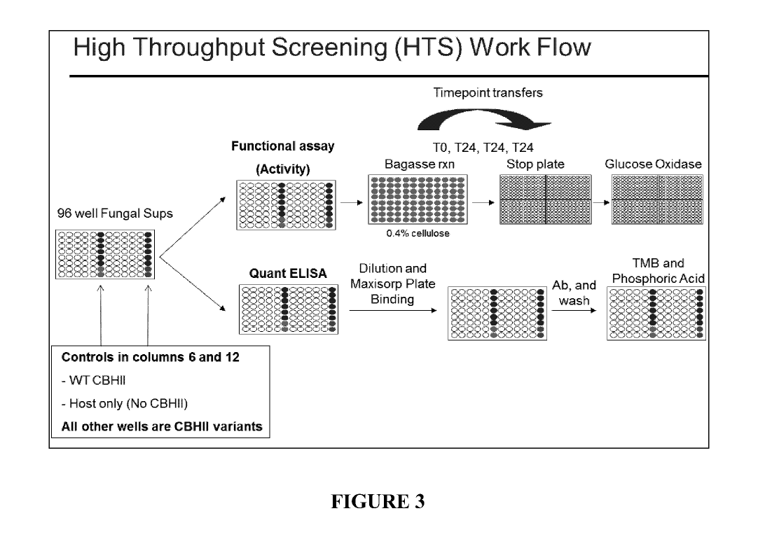

After 5-7 days of growth at 30° C., the A. niger liquid culture supernatants were filtered into a new 96-well plate to remove the fungal biomass prior to screening. Supernatants were then split into two streams for a high throughput glucose oxidase assay and a high throughput ELISA assay (FIG. 3).

For secondary screening, spores expressing CBH II variants identified as hits in the primary screens were picked from frozen archived fungal spore plates and grown in a liquid fungal media culture in quadruplicate. The growth media had the same composition as described above.

For tertiary screening, spores expressing CBH II variants identified as hits in the secondary screen were grown in shake flasks with 1 L of liquid fungal media culture as described above. 1 L samples were harvested and processed for larger scale enzyme activity screening. 1 L harvested samples were processed by hollow fiber dia-filtration, allowing for a 5-fold buffer exchange with 50 mM sodium citrate and sample concentration to about 200 ml. Concentrated supernatants were then frozen at −80° C. and lyophilized into a powder. For samples still containing residual glucose upon re-suspension, a PD10 de-salting column was used to remove the excess sugars. After harvesting and recovery was complete, protein concentrations for CBH II variants were determined using a standardized quantification method that involved running an SDS gel and using a purified CBH II protein standard to determine precise concentrations.

2.1.3. CBH II Assays

Glucose Oxidase Functional Assay:

This assay measures the digestibility of bagasse as a substrate and was used for primary and secondary screening. Acid-pretreated and steam-exploded bagasse was washed, dried and milled to 40 mesh with roughly 60% glucan content. This substrate was mixed with 50 mM sodium acetate buffer to a final concentration of 0.4% cellulose and added to 96-well plates. For secondary screens, rows A and H were left blank on the 96-well plates to minimize edge well evaporation effects. The A. niger-expressed CBH II supernatants were added to the 96-well plates to initiate the reaction. Samples were mixed and then centrifuged. An aliquot from each well was then transferred into a pH 10 100 mM sodium carbonate buffer to stop the reaction and generate an initial time point. The initial time point was used to monitor any potential residual glucose from fungal growth media. Samples were then mixed in a shaking incubator at 37° C. for 24 hours. After 24 hours, three aliquots from each sample were transferred into the pH 10 stop buffer. Stop buffer plates containing initial and 24 hr time points were sealed and stored at 4° C. overnight. The following day a glucose oxidase detection assay was done. Each stop plate was mixed with 50 mM pH 7.4 Sodium Phosphate buffer, a Glucose Oxidase (Sigma #G7141-50KU) and Horseradish Peroxidase (Sigma #P2088-5KU) mix, and Amplex red (Invitrogen No. 22177). The plates were incubated at 25° C. for 30 minutes and fluorescence was read at 560 Ex/610 Em.

ELISA Assay:

The ELISA assay measures the concentration of protein expressed with enzyme specific polyclonal antibodies and was used for primary and secondary screening. Enzymes were purified and polyclonal antibodies were produced in rabbits. The A. niger-expressed CBH II supernatants were diluted in PBS and transferred to NUNC Immuno maxisorp plates. For secondary screens, rows A and H were left blank on the 96-well plates to minimize edge well effects. The plates were left overnight to bind proteins. The next day, blocking reagent was added to the samples, followed by subsequent incubations with the optimized dilutions of 1° antibody produced in rabbits and 2° antibody (Sigma anti-rabbit whole molecule grown in goat with peroxidase). A wash step with PBS was performed between each incubation. Finally, a SureBlue™ TMB detection reagent was added followed by a stop reagent (1M phosphoric acid) and absorbance at 450 nm was read.

Saccharification Assay:

The saccharification assay measures cellulose conversion to glucose and was used for tertiary screening of CBH II variants. Reactions were performed in 10 ml vials in duplicate at 35° C. Reaction volume was 5.4 ml. A. niger expressed lyophilized CBH II, CBH I, and EG were dosed at 1:1:1 ratio to give a total dose of 10 mg enzyme/g of cellulose. Bagasse was loaded to give a concentration of 5% solids in each vial. The reaction buffer was 50 mM, pH5.2 sodium acetate. 1 mM sodium azide was present in reactions to prevent contamination. CBH II hits were compared to wild-type BD23134 (both grown up in flask as well as from lyophilized powder) in the presence of CBH I and EG because CBH II, CBH I, and EG act synergistically to digest bagasse. A saturating dose of Cochliobolus β-glucosidase expressed in Pichia was also added to the reactions to account for variable endogenous expression of β-glucosidase. Adding a saturating dose of β-glucosidase normalizes the activity between the samples being tested. Once the T=0 time point was taken, vials were placed in a hybridization oven and subsequent time points were taken at 24, 48, and 72 hours. The hybridization oven was used to provide gentle mixing via a tumbling motion at 8 RPM. HPLC was used to analyze samples. Refractive index detection (RID) was used to measure sugar products (glucose, cellobiose, etc.). Hits were considered “confirmed” if they showed at least a 2% improvement in specific activity over the WT average at the 72 hr time point.

2.2. Results

Primary Screen:

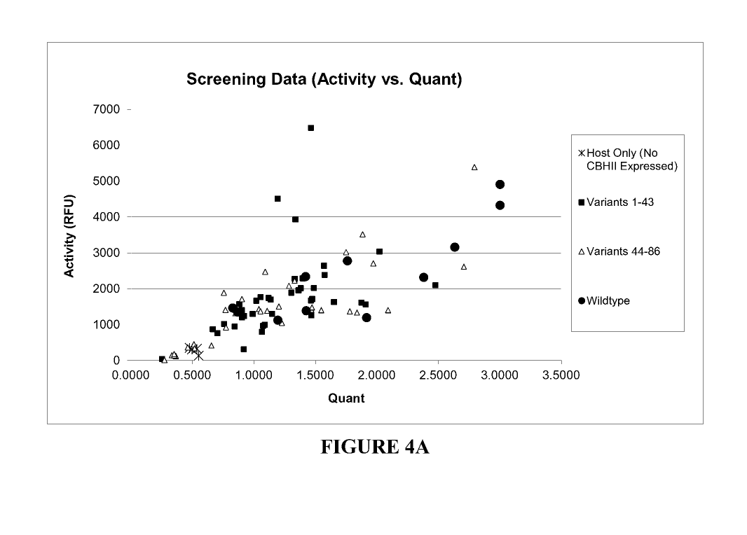

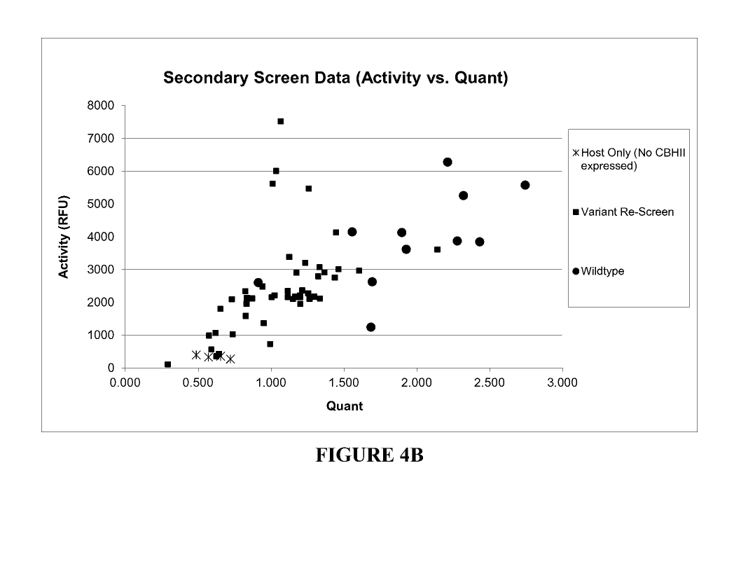

The results of one set of primary screening data from a 96-well plate are shown in FIG. 4A. Activity values obtained from the glucose oxidase functional assay are plotted on the Y-axis and protein concentration values obtained from the ELISA assay are plotted on the X-axis. Two amino acid locations were targeted for mutation per plate and are represented with squares or triangles. The wild-type protein is represented with circles. A host only control, expressing no CBH II, is shown as an asterisk. The host only control measures the endogenous A. niger enzyme activity from the screening strain. Samples that stood out above the wild-type controls were selected for secondary screening. In this plate, three wells represented as squares are shown as hits with improved activity, rising above the trend of the wild-type.

Secondary Screen:

The results of one set of secondary screen data are shown in FIG. 4B. Activity values obtained from the glucose oxidase functional assay are plotted on the Y-axis and protein concentration values obtained from the ELISA assay are plotted on the X-axis. Variant CBH II polypeptides are shown by squares. Wild-type CBH II is represented by circles. The host only control is represented by asterisks. Samples that stood out above the wild-type controls were selected for tertiary screening. In this set, a variant was reconfirmed as having increased specific activity as compared to the wild-type CBH II and was selected for tertiary screening.

Tertiary Screen:

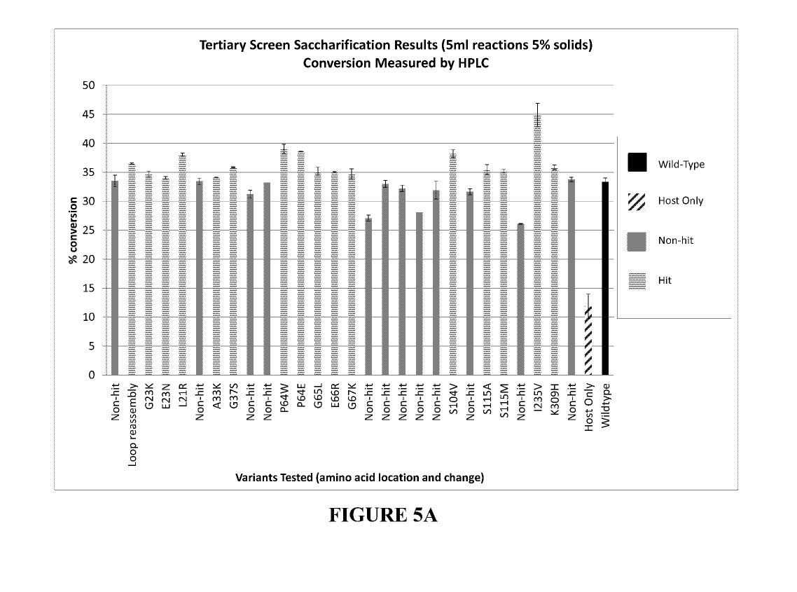

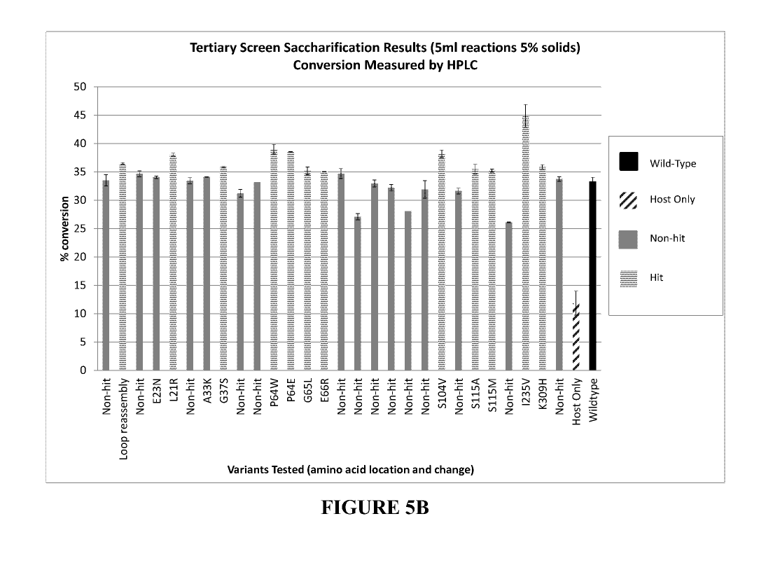

Tertiary screening results are shown in FIGS. 5A and 5B. FIG. 5A identifies sixteen CBH II variants having a specific activity at least 2% greater than the specific activity of the wild-type BD23134 at the 72 hour time point. FIG. 5B identifies twelve CBH II variants having a specific activity at least 5% greater than the specific activity of the wild-type BD23134 at the 72 hour time point. Single amino acid substitutions found to increase the specific activity of BD23134 are I235V, P64W, P64E, L21R, S104V, G37S, G65L, K309H, E66R, S115A, G67K, E23K, S115M, A33K, and E23N. Nucleic acid sequences coding for BD23134 polypeptides having each of these single amino acid substitutions are shown in Table 3. A CBH II variant designed to test the effect of modifying the catalytic loops involved in substrate binding having the following combination of amino acid substitutions was found have increased specific activity compared to BD23134: D194N, A200L, S421C, D426N, A429S, T430P, Y434A, A438L, S439P, A440D, L442T, Q443P, and P444N.

### 3. Specific Embodiments and Incorporation by Reference

All publications, patents, patent applications and other documents cited in this application are hereby incorporated by reference in their entireties for all purposes to the same extent as if each individual publication, patent, patent application or other document were individually indicated to be incorporated by reference for all purposes.

While various specific embodiments have been illustrated and described, it will be appreciated that various changes can be made without departing from the spirit and scope of the invention(s).

TABLE 1

| Sequence | Database Accession | | |
|---|---|---|---|
| Identifier | Number or Patent | | |
| (SEQ ID NO:) | Document Number | Species/source | Amino acid sequence |
| SEQ ID NO: 2 | W02008/095033-0282 | Fungal | MTVYQLLFTA ALAGTALAAP LVEERQACAS QWAQCGGFSW NGATCCQSGS YCSKINDYYS QCIPGEGPAT |
| | | | SKTSTLPAST TTSKPTSTST AGTSSTTKPP PAGSGTATYS GNPYSGVNLW ANSYYRSEVT NLAIPKLSGA |
| | | | MATAAAKVAD VPSYQWMDSF EHISLMEDTL VDIRKANQAG GNYAGQFVVY DLPDRDCAAA ASNGEYSLDK |
| | | | DGANKYKNYI NTIKKIIQSY SDIRILLVIE PDSLANLVTN MDVAKCAKAH DAYISLTNYA VTELNLPNVA |
| | | | MYLDAGHAGW LGWPNNQGPA AKLFASIYKD AGKPAALRGL ATNVANYNAW SLSSAPPYTQ GASIYDEKSF |
| | | | IHAMGPLLEQ NGWPGAHFIT DQGRSGKQPT GQIQWGDWCN SKGTGFGIRP SANTGDSLLD AFVWVKPGGE |
| | | | SDGTSDTSAT RYDYHCGASA ALQPAPEAGT WFQAYFEQLL TNANPSFL |
| SEQ ID NO: 3 | W02008/095033-0522 | Fungus | MRYTWSVAAA LLPCAIQAQQ TLYGQCGGQG YSGLTSCVAG ATCSTVNEYY AQCTPAAGSA TSTTLKTTTT |
| | | | TAGATTTTTS KTSASQTSTT KTSTSTASTT TATTTASASG NPFSGYQLYV NPYYSSEVAS LAIPSLTGTL |
| | | | SSLQAAATAA AKVPSFVWLD VAAKVPTMAT YLADIKAQNA AGANPPVAGQ FVVYDLPDRD CAALASNGEY |
| | | | SIANNGVANY KAYIDSIRKV LVQYSDVHTI LVIEPDSLAN LVTNLNVAKC ANAQSAYLEC TNYALEQLNL |
| | | | PNVAMYLDAG HAGWLGWPAN QQPAANLYAS VYKNASSPAA VRGLATNVAN YNAFTIASCP SYTQGNSVCD |
| | | | EQQYINAIAP LLSAQGFNAH FIVDTGRNGK QPTGQQAWGD WCNVINTGFG VRPTTNTGDA LVDAFVWVKP |
| | | | GGESDGTSDS SATRYDAHCG YSDALQPAPE AGTWFQAYFV QLLSNANPAF |
| SEQ ID NO: 4 | W02008/095033-0358 | Unknown | MVVGILATLA TLATLAASVP LEERQSCSSV WGQCGGQNWA GPFCCASGST CVYSNDYYSQ CLPGTASSSS |
| | | | STRASSTTSR VSSATSTRSS SSTPPPASST TPAPPVGSGT ATYSGNPFAG VTPWANSFYA SEVSTLAIPS |
| | | | LTGAMATAAA AVAKVPSFMW LDTLDKTPLM SSTLSDIRAA NKAGGNYAGQ FVVYDLPDRD CAALASNGEY |
| | | | SIADGGVAKY KNYIDTIRGI VTTFSDVRIL LVIEPDSLAN LVTNLATPKC SNAQSAYLEC INYAITQLNL |
| | | | PNVAMYLDAG HAGWLGWPAN QDPAAQLFAN VYKNASSPRA VRGLATNVAN YNAWNITTPP SYTQGNAVYN |
| | | | EKLYIHALGP LLANHGWSNA FFITDQGRSG KQPTGQLEWG NWCNAVGTGF GIRPSANTGD SLLDSFVWIK |
| | | | PGGECDGTSN SSAPRFDYHC ASADALQPAP QAGSWFQAYF VQLLTNANPS FL |
| SEQ ID NO: 5 | US8101393-0098 | Unknown | MRYTWSVAAA LLPCAIQAQQ TLYGQCGGQG YSGLTSCVAG ATCSTVNEYY AQCTPAAGSA TSTTLKTTTT |
| | | | TAGATTTTTS KTSASQTSTT KTSTSTASTT TATTTASASG NPFSGYQLYV NPYYSSEVAS LAIPSLTGTL |
| | | | SSLQAAATAA AKVPSFVWLD VAAKVPTMAT YLADIKAQNA AGANPPVAGQ FVVYDLPDRD CAALASNGEY |
| | | | SIANNGVANY KAYIDSIRKV LVQYSDVHTI LVIEPDSLAN LVTNLNVAKC ANAQSAYLEC TNYALEQLNL |
| | | | PNVAMYLDAG HAGWLGWPAN QQPAANLYAS VYKNASSPAA VRGLATNVAN YNAFTIASCP SYTQGNSVCD |
| | | | EQQYINAIAP LLSAQGFNAH FIVDTGRNGK QPTGQQAWGD WCNVINTGFG VRPTTNTGDA LVDAFVWVKP |
| | | | GGESDGTSDS SATRYDAHCG YSDALQPAPE AGTWFQAYFV QLLSNANPAF |
| SEQ ID NO: 6 | W02011059740-0002 | Aspergillus | MRYTWSVAAA LLPCAIQAQQ TLYGQCGGQG YSGLTSCVAG ATCSTVNEYY AQCTPAAGAT STTLKTTTTT |
| | | aculeatus | AGATTTTTTK SSASQTSTTK TSTGTVSTTT ATTTASASGN PFSGYQLYVN PYYSSEVASL AIPSLTGTLS |
| | | | SLQAAATAAA KVPSFVWLDV AAKVPTMATY LADIKAQNAA GANPPIAGQF VVYDLPDRDC AALASNGEYS |
| | | | IANNGVANYK AYIDSIRKVL VQYSDVHTIL VIEPDSLANL VTNLNVAKCA NAQSAYLECT NYALEQLNLP |
| | | | NVAMYLDAGH AGWLGWPANQ QPAANLYASV YKNASSPAAV RGLATNVANY NAFTISSCPS YTQGNSVCDE |
| | | | QQYINAIAPL LSAQGFDAHF IVDTGRNGKQ PTGQQAWGDW CNVINTGFGV RPTTSTGDAL VDAFVWVKPG |
| | | | GESDGTSDSS ATRYDAHCGY SDALQPAPEA GTWFQAYFVQ LLTNANPAF |
| SEQ ID NO: 7 | US8168863-0013 | Artificial | MVPLEERQAC SSVWGQCGGQ NWAGPFCCAS GSTCVYSNDY YSQCLPGAAS SSSSTRAAST TSRVSSATST |
| | | Sequence | RSSSSTPPPA SSTTPAPPVG SGTATYSGNP FAGVTPWANS FYASEVSTLA IPSLTGAMAT AAAAVAKVPS |
| | | | FMWLDTLDKT PLMSSTLSDI RAANKAGGNY AGQFVVYDLP DRDCAAAASN GEYSIADGGV AKYKNYIDTI |
| | | | RGIVTTFSDV RILLVIEPDS LANLVTNLAT PKCSNAQSAY LECINYAITQ LNLPNVAMYL DAGHAGWLGW |
| | | | PANQDPAAQL FANVYKNASS PRAVRGLATN VANYNAWNIT TPPSYTQGNA VYNEKLYIHA LGPLLANHGW |
| | | | SNAFFITDQG RSGKQPTGQL EWGNWCNAVG TGFGIRPSAN TGDSLLDSFV WIKPGGECDG TSNSSAPRFD |
| | | | YHCASADALQ PAPQAGSWFQ AYFVQLLTNA NPSFL |
| SEQ ID NO: 8 | US20090320831-0082 | Trichoderma | MVPLEERQAC SSVWGQCGGQ NWSGPTCCAS GSTCVYSNDY YSQCLPGAAS SSSSTRAAST TSRVSPTTSR |
| | | reesei | SSSATPPPGS TTTRVPPVGS GTATYSGNPF VGVTPWANAY YASEVSSLAI PSLTGAMATA AAAVAKVPSF |
| | | | MWLDTLDKTP LMEQTLADIR TANKNGGNYA GQFVVYDLPD RDCAALASNG EYSIADGGVA KYKNYIDTIR |
| | | | QIVVEYSDIR TLLVIEPDSL ANLVTNLGTP KCANAQSAYL ECINYAVTQL NLPNVAMYLD AGHAGWLGWP |
| | | | ANQDPAAQLF ANVYKNASSP RALRGLATNV ANYNGWNITS PPSYTQGNAV YNEKLYIHAI GPLLANHGWS |
| | | | NAFFITDQGR SGKQPTGQQQ WGDWCNVIGT GFGIRPSANT GDSLLDSFVW VKPGGECDGT SDSSAPRFDS |
| | | | HCALPDALQP APQAGAWFQA YFVQLLTNAN PSFL |
| SEQ ID NO: 9 | US20120142046-0060 | Trichoderma | MVSFTSLLAG VAAISGVLAA PAAEVEPVAV EKREAEAEAV PLEERQACSS VWGQCGGQNW SGPTCCASGS |
| | | reesei | TCVYSNDYYS QCLPGAASSS SSTRAASTTS RVSPTTSRSS SATPPPGSTT TRVPPVGSGT ATYSGNPFVG |
| | | | VTPWANAYYA SEVSSLAIPS LTGAMATAAA AVAKVPSFMW LDTLDKTPLM EQTLADIRTA NKNGGNYAGQ |
| | | | FVVYDLPDRD CAALASNGEY SIADGGVAKY KNYIDTIRQI VVEYSDIRTL LVIEPDSLAN LVTNLGTPKC |
| | | | ANAQSAYLEC INYAVTQLNL PNVAMYLDAG HAGWLGWPAN QDPAAQLFAN VYKNASSPRA LRGLATNVAN |
| | | | YNGWNITSPP SYTQGNAVYN EKLYIHAIGP LLANHGWSNA FFITDQGRSG KQPTGQQQWG DWCNVIGTGF |
| | | | GIRPSANTGDSLLDSFVWVKP GGECDGTSDS SAPRFDSHCA LPDALQPAPQ AGAWFQAYFV QLLTNANPSF |
| | | | L |
| SEQ ID NO: 10 | US20100313307-0046 | Artificial | MKTNLFLFLI FSLLLSLSSA EQACSSVWGQ CGGQNWSGPT CCASGSTCVY SNDYYSQCLP GAASSSSSTR |
| | | Sequence | AASTTSRVSP TTSRSSSATP PPGSTTTRVP PVGSGTATYS GNPFVGVTPW ANAYYASEVS SLAIPSLTGA |
| | | | MATAAAAVAK VPSFMWLDTL DKTPLMEQTL ADIRTANKNG GNYAGQFVVY DLPDRDCAAL ASNGEYSIAD |
| | | | GGVAKYKNYI DTIRQIVVEY SDIRILLVIE PDSLANLVTN LGTPKCANAQ SAYLECINYA VTQLNLPNVA |
| | | | MYLDAGHAGW LGWPANQDPA AQLFANVYKN ASSPRALRGL ATNVANYNGW NITSPPSYTQ GNAVYNEKLY |
| | | | IHAIGPLLAN HGWSNAFFIT DQGRSGKQPT GQQQWGDWCN VIGTGFGIRP SANTGDSLLD SFVWVKPGGE |
| | | | CDGTSDSSAP RFDSHCALPD ALQPAPQAGA WFQAYFVQLL TNANPSFLKD EL |
| SEQ ID NO: 11 | US20100189706-0282 | unknown | MTVYQLLFTA ALAGTALAAP LVEERQACAS QWAQCGGFSW NGATCCQSGS YCSKINDYYS QCIPGEGPAT |
| | | | SKTSTLPAST TTSKPTSTST AGTSSTTKPP PAGSGTATYS GNPYSGVNLW ANSYYRSEVT NLAIPKLSGA |
| | | | MATAAAKVAD VPSYQWMDSF EHISLMEDTL VDIRKANQAG GNYAGQFVVY DLPDRDCAAA ASNGEYSLDK |
| | | | DGANKYKNYI NTIKKIIQSY SDIRILLVIE PDSLANLVTN MDVAKCAKAH DAYISLTNYA VTELNLPNVA |
| | | | MYLDAGHAGW LGWPNNQGPA AKLFASIYKD AGKPAALRGL ATNVANYNGW SLSSAPPYTQ GASIYDEKSF |
| | | | IHAMGPLLEQ NGWPGAHFIT DQGRSGKQPT GQIQWGDWCN SKGTGFGIRP SANTGDSLLD AFVWVKPGGE |
| | | | SDGTSDTSAT RYDYHCGASA ALQPAPEAGT WFQAYFEQLL TNANPSFL |
| SEQ ID NO: 12 | US20100189706-0358 | unknown | MIVGILTTLA TLATLAASVP LEERQSCSSV WGQCGGQNWA GPTCCASGST CVYSNDYYSQ CLPGAASSSS |
| | | | STRAASTTSR VSSATSTRSS SSTPPPASST TPAPPVGSGT ATYSGNPFAG VTPWANSFYA SEVSTLAIPS |
| | | | LTGAMATAAA AVAKVPSFMW LDTLDKTPLM SSTLSDIRAA NKAGGNYAGQ FVVYDLPDRD CAAAASNGEY |
| | | | SIADGGVAKY KNYIDTIRGI VTTFSDVRIL LVIEPDSLAN LVTNLATPKC SNAQSAYLEC INYAITQLNL |
| | | | PNVAMYLDAG HAGWLGWPAN QDPAAQLFAN VYKNASSPRA VRGLATNVAN YNAWNITTPP SYTQGNAVYN |
| | | | EKLYIHALGP LLANHGWSNA FFITDQGRSG KQPTGQLEWG NWCNAVGTGF GIRPSANTGD SLLDSFVWIK |
| | | | PGGECDGTSN SSAPRFDYHC ASADALQPAP QAGSWFQAYF VQLLTNANPS FL |
| SEQ ID NO: 13 | US20100189706-0401 | unknown | MGVHKLFLAT ALFGLAVAAP IVEERESCAT QGQCGGINWN GVTCCESGSY CSKINDYYFQ CLSGSNPTTS |
| | | | KTSSLPISTT TSLITKSSST ASSTKTSAGS STTASPTVGA GTATYSGNPY SGVNLWANGY YRSEVSTLAI |
| | | | PSLSGAMATA AAKVAEVPSF QWMDSYAHIS LMEDTLADIR KANQAGGNYA GQFVVYDLPE RDCAAAASNG |
| | | | EYSLDNDGAN KYKNYINRVK TIIQSYSDIR IILVIEPDSL ANLVTNMNVA KCSKAHDAYL SLTNYAVTAL |
| | | | NLPNVAMYLD AGHAGWLGWP ANQSPAAQLF AGVYKDAGKP SSLRGLVTNV ANYNGWSLST APSYTQGNSI |
| | | | YDEKSFIHAM GPLLEQNGWA GAQFITDQGR SGKQPTGQAQ WGDWCNAKGT GFGIRPSANT GDSLLDAFVW |
| | | | VKPGGESDGT SDTTAARYDY HCGYSDALQP APEAGTWFQA YFVQLLTNAN PSFL |
| SEQ ID NO: 14 | US4894338-0002 | unknown | MIVGILTTLA TLATLAASVP LEERQACSSV WGQCGGQNWS GPTCCASGST CVYSNDYYSQ CLPGAASSSS |
| | | | STRAASTTSR VSPTTSRSSS ATPPPGSTTT RVPPVGSGTA TYSGNPFVGV TPWANAYYAS EVSSLAIPSL |
| | | | TGAMATAAAA VAKVPSFMWL DTLDKTPLME QTLADIRTAN KNGGNYAGQF VVYDLPDRDC AALASNGEYS |
| | | | IADGGVAKYK NYIDTIRQIV VEYSDIRTLL VIEPDSLANL VTNLGTPKCA NAQSAYLECI NYAVTQLNLP |
| | | | NVAMYLDAGH AGWLGWPANQ DPAAQLFANV YKNASSPRAL RGLATNVANY NGWNITSPPS YTQGNAVYNE |
| | | | KLYIHAIGPL LANHGWSNAF FITDQGRSGK QPTGQQQWGD WCNVIGTGFG IRPSANTGDS LLDSFVWVKP |
| | | | GGECDGTSDS SAPRFDSHCA LPDALQPAPQ AGAWFQAYFV QLLTNANPSF L |
| SEQ ID NO: 15 | US6114158-0008 | Trichoderma | MIVGILTTLA TLATLAASVP LEERQACSSV WGQCGGQNWS GPTCCASGST CVYSNDYYSQ CLPGAASSSS |
| | | reesei | STRAASTTRV SPTTSRSSSA TPPPGSTTTR VPPVGSGTAT YSGNPFVGVT PWANAYYASE VSSLAIPSLT |
| | | | GAMATAAAAV AKVPSFMWLD TLDKTPLMEQ TLADIRTANK NGGNYAGQFV VYDLPDRDCA ALASNGEYSI |
| | | | ADGGVAKYKN YIDTIRQIVV EYSDIRTLLV IEPDSLANLV TNLGTPKCAN AQSAYLECIN YAVTQLNLPN |
| | | | VAMYLDAGHA GWLGWPANQD PAAQLFANVY KNASSPRALR GLATNVANYN GWNITSPPSY TQGNAVYNEK |
| | | | LYIHAIGPLL ANHGWSNAFF ITDQGRSGKQ PTGQQQWGDW CNVIGTGFGI RPSANTGDSL LDSFVWVKPG |
| | | | GECDGTSDSS APRFDSHCAL PDALQPAPQA GAWFQAYFVQ LLTNANPSFL |
| SEQ ID NO: 16 | US8008056-0089 | Hypocrea | MIVGILTTLA TLATLAASVP LEERQACSSV WGQCGGQNWS GPTCCASGST CVYSNDYYSQ CLPGAASSSS |
| | | koningii | STRAASTTSR VSPTTSRSSS ATPPPGSTTT RVPPVGSGTA TYSGNPFVGV TPWANAYYAS EVSSLAIPSL |
| | | | TGAMATAAAA VAKVPSFMWL DTLDKTPLME QTLADIRTAN KNGGNYAGQF VVYDLPDRDC AALASNGEYS |
| | | | IADGGVAKYK NYIDTIRQIV VEYSDIRTLL VIEPDSLANL VTNLGTPKCA NAQSAYLECI NYAVTQLNLP |
| | | | NVAMYLDAGH AGWLGWPANQ DPAAQLFANV YKNASSPRAL RGLATNVANY NGWNITSPPS YTQGNAVYNE |
| | | | KLYIHAIGRL LANHGWSNAF FITDQGRSGK QPTGQQQWGD WCNVIGTGFG IRPSANTGDS LLDSFVWVKP |
| | | | GGECDGTSDS SAPRFDSHCA LPDALQPAPQ AGAWFQAYFV QLLTNANPSF L |
| SEQ ID NO: 17 | US20090325240-0923 | Hypocrea | MIVGILTTLA TLATLAASVP LEERQACSSA WGQCGGQNWS GPTCCASGST CVYSNDYYSQ CLPGAASSSS |
| | | koningii | STRASSTTAR ASSTTSRSSA TPPPGSSTTR VPPVGSGTAT YSGNPFVGVT PWANAYYASE VSSLAIPSLT |
| | | strain | GAMATAAAAV AKVPSFMWLD TFDKTPLMEQ TLADIRTANK NGGNYAGQFV VYDLPDRDCA ALASNGEYSI |
| | | 3.2774 | ADGGVDKYKN YIDTIRQIVV EYSDIRTLLV IEPDSLANLV TNLGTPKCAN AQSAYLECIN YAVTQLNLPN |
| | | | VAMYLDAGHA GWLGWPANQD PAAQLFANVY KNASSPRALR GLATNVANYN GWNITSPPSY TQGNAVYNEQ |
| | | | LYIHAIGPLL ANHGWSNAFF ITDQGRSGKQ PTGQQQWGDW CNVIGTGFGI RPSANTGDSL LDSFVWIKPG |
| | | | GECDGTSDSS APRFDSHCAL PDALQPAPQA GAWFQAYFVQ LLTNANPSFL |
| SEQ ID NO: 18 | US20090325240-0946 | Trichoderma | MIVGILTTLA TLATLAASVP LEERQACSSV WGQCGGQNWS GPTCCAAGST CVYSNDYYSQ CPPGAASSSS |
| | | parceramosum | STRASSTTNR VSSTTSTSSA TPPPGSTTTR VPPVGSGTAT YSGNPFVGVT PWANAYYASE VSSLAIPSLT |
| | | | GAMATAAAAV AKVPSFMWLD TLDKTPLMEQ TLADIRTANK NGGNYAGQFV VYDLPDRDCA ALASNGEYSI |
| | | | ADGGVAKYKN YIDTIRQIVV EYSDIRTLLV IEPDSLANLV TNLGTPKCAN AQSAYLECIN YAITQLNLPN |
| | | | IAMYLDAGHA GWLGWPANQD PAAQLFANVY KNASSPSALR GLATNVANYN GWNITSPPSY TQGNAVYNEK |
| | | | LYIHAIGPLL ANHGWSNAFF ITDQGRSGKQ PTGQQQWGDW CNVIGTGFGI RPSSNTGDSL LDSFVWVKPG |
| | | | GECDGTSDSS APRFDSHCAL PDALQPAPQA GAWFQAYFVQ LLTNANPSFL |
| SEQ ID NO: 19 | US20090325240-0970 | Trichoderma | MIVGILTTLA TLATLAASVP LEERQACSSV WGQCGGQNWS GPTCCASGST CVYSNDYYSQ CLPGAASSSS |
| | | viride strain | STRAASTTSR VSPTTSRSSS ATPPPGSTTT RVPPVGSGTA TYSGNPFVGV TPWANAYYAS EVSSLAIPSL |
| | | CICC 13038 | TGAMATAAAA VAKVPSFMWL DTLDKTPLME QTLADIRTAN KNGGNYAGQF VVYDLPDRDC AALASNGEYS |
| | | | IADGGVAKYK NYIDTIRQIV VEYSDIRTLL VIEPDSLANL VTNLGTPKCA NAPSAYLECI NYAVTQLNLP |
| | | | NVAMYLDAGH AGWLGWPANQ DPAAQLFANV YKNASSPRAL RGLATNVANY NGWNITSPPS YTQGNAVYNE |
| | | | KLYIHAIGPL LANHGWSNAF FITDQGRSGK QPTGQQQWGD WCNVIGTGFG IRPSANTGDS LLDSFVWVKP |
| | | | GGECDGTSDS SAPRFDSHCA LPDALQPAPQ AGAWFQAYFV QLLTNANPSF L |
| SEQ ID NO: 20 | US20120129229-0028 | Trichoderma | MIVGILTTLA TLATLAASVP LEERQACSSV WGQCGGQNWS GPTCCASGST CVYSNDYYSQ CLPGAASSSS |
| | | reesei | STRAASTTSR VSPTTSRSSS ATPPPGSTTT RVPPVGSGTA TYSGNPFVGV TPWANAYYAS EVSSLAIPSL |
| | | | TGAMATAAAA VAKVPSFMWL DTLDKTPLME QTLADIRTAN KNGGNYAGQF VVYDLPDRDC AALASNGEYS |
| | | | IADGGVAKYK NYIDTIRQIV VEYSDIRTLL VIEPDSLANL VTNLGTPKCA NAQSAYLECI NYAVTQLNLP |
| | | | NVAMYLDAGH AGWLGWPANQ DPAAQLFANV YKNASSPRAL RGLATNVANY NGWNITSPPS YTQGNAVYNE |
| | | | KLYIHAIGRLLANHGWSNAFF ITDQGRSGKQ PTGQQQWGDW CNVIGTGFGI RPSANTGDSL LDSFVWVKPG |
| | | | GECDGTSDSS APRFDSHCAL PDALQPAAQA GAWFQAYFVQ LLTNANPSFL |
| SEQ ID NO: 21 | US8012734-0037 | Trichoderma | QACSSVWGQC GGQNWSGPTC CASGSTCVYS NDYYSQCLPG AASSSSSTRA ASTTSRVSPT TSRSSSATPP |
| | | reesei | PGSTTTRVPP VGSGTATYSG NPFVGVTPWA NAAYASEVSS LAIPSLTGAM ATAAAAVAKV PSFMWLDTLD |
| | | | KTPLMEQTLA DIRTANKNGG NYAGQFVVYD LPDRDCAALA SNGEYSIADG GVAKYKNYID TIRQIVVEYS |
| | | | DIRTLLVIEP DSLANLVTNL GTPKCANAQS AYLECINYAV TQLNLPNVAM YLDAGHAGWL GWPANQDPAA |
| | | | QLFANVYKNA SSPRALRGLA TNVANYNGWN ITSPPSYTQG NAVYNEKLYI HAIGPLLANH GWSNAFFITD |
| | | | QGRSGKQPTG QQQWGDWCNV IGTGFGIRPS ANTGDSLLDS FVWVKPGGEC DGTSDSSAPR FDPHCALPDA |
| | | | LQPAPQAGAW FQAYFVQLLT NANPSFL |
| SEQ ID NO: 22 | US8012734-0038 | Trichoderma | QACSSVWGQC GGQNWSGPTC CASGSTCVYS NDYYSQCLPG AASSSSSTRA ASTTSRVSPT TSRSSSATPP |
| | | reesei | PGSTTTRVPP VGSGTATYSG NPFVGVTPWA NAHYASEVSS LAIPSLTGAM ATAAAAVAKV PSFMWLDTLD |
| | | | KTPLMEQTLA DIRTANKNGG NYAGQFVVYD LPDRDCAALA SNGEYSIADG GVAKYKNYID TIRQIVVEYS |
| | | | DIRTLLVIEP DSLANLVTNL GTPKCANAQS AYLECINYAV TQLNLPNVAM YLDAGHAGWL GWPANQDPAA |
| | | | QLFANVYKNA SSPRALRGLA TNVANYNGWN ITSPPSYTQG NAVYNEKLYI HAIGPLLANH GWSNAFFITD |
| | | | QGRSGKQPTG QQQWGDWCNV IGTGFGIRPS ANTGDSLLDS FVWVKPGGEC DGTSDSSAPR FDPHCALPDA |
| | | | LQPAPQAGAW FQAYFVQLLT NANPSFL |
| SEQ ID NO: 23 | US8012734-0039 | Trichoderma | QACSSVWGQC GGQNWSGPTC CASGSTCVYS NDYYSQCLPG AASSSSSTRA ASTTSRVSPT TSRSSSATPP |
| | | reesei | PGSTTTRVPP VGSGTATYSG NPFVGVTPWA NAKYASEVSS LAIPSLTGAM ATAAAAVAKV PSFMWLDTLD |
| | | | KTPLMEQTLA DIRTANKNGG NYAGQFVVYD LPDRDCAALA SNGEYSIADG GVAKYKNYID TIRQIVVEYS |
| | | | DIRTLLVIEP DSLANLVTNL GTPKCANAQS AYLECINYAV TQLNLPNVAM YLDAGHAGWL GWPANQDPAA |
| | | | QLFANVYKNA SSPRALRGLA TNVANYNGWN ITSPPSYTQG NAVYNEKLYI HAIGPLLANH GWSNAFFITD |
| | | | QGRSGKQPTG QQQWGDWCNV IGTGFGIRPS ANTGDSLLDS FVWVKPGGEC DGTSDSSAPR FDPHCALPDA |
| | | | LQPAPQAGAW FQAYFVQLLT NANPSFL |
| SEQ ID NO: 24 | US8012734-0040 | Trichoderma | QACSSVWGQC GGQNWSGPTC CASGSTCVYS NDYYSQCLPG AASSSSSTRA ASTTSRVSPT TSRSSSATPP |
| | | reesei | PGSTTTRVPP VGSGTATYSG NPFVGVTPWA NALYASEVSS LAIPSLTGAM ATAAAAVAKV PSFMWLDTLD |
| | | | KTPLMEQTLA DIRTANKNGG NYAGQFVVYD LPDRDCAALA SNGEYSIADG GVAKYKNYID TIRQIVVEYS |
| | | | DIRTLLVIEP DSLANLVTNL GTPKCANAQS AYLECINYAV TQLNLPNVAM YLDAGHAGWL GWPANQDPAA |
| | | | QLFANVYKNA SSPRALRGLA TNVANYNGWN ITSPPSYTQG NAVYNEKLYI HAIGPLLANH GWSNAFFITD |
| | | | QGRSGKQPTG QQQWGDWCNV IGTGFGIRPS ANTGDSLLDS FVWVKPGGEC DGTSDSSAPR FDPHCALPDA |
| | | | LQPAPQAGAW FQAYFVQLLT NANPSFL |
| SEQ ID NO: 25 | US8012734-0041 | Trichoderma | QACSSVWGQC GGQNWSGPTC CASGSTCVYS NDYYSQCLPG AASSSSSTRA ASTTSRVSPT TSRSSSATPP |
| | | reesei | PGSTTTRVPP VGSGTATYSG NPFVGVTPWA NAMYASEVSS LAIPSLTGAM ATAAAAVAKV PSFMWLDTLD |
| | | | KTPLMEQTLA DIRTANKNGG NYAGQFVVYD LPDRDCAALA SNGEYSIADG GVAKYKNYID TIRQIVVEYS |
| | | | DIRTLLVIEP DSLANLVTNL GTPKCANAQS AYLECINYAV TQLNLPNVAM YLDAGHAGWL GWPANQDPAA |
| | | | QLFANVYKNA SSPRALRGLA TNVANYNGWN ITSPPSYTQG NAVYNEKLYI HAIGPLLANH GWSNAFFITD |
| | | | QGRSGKQPTG QQQWGDWCNV IGTGFGIRPS ANTGDSLLDS FVWVKPGGEC DGTSDSSAPR FDPHCALPDA |
| | | | LQPAPQAGAW FQAYFVQLLT NANPSFL |
| SEQ ID NO: 26 | US8012734-0042 | Trichoderma | QACSSVWGQC GGQNWSGPTC CASGSTCVYS NDYYSQCLPG AASSSSSTRA ASTTSRVSPT TSRSSSATPP |
| | | reesei | PGSTTTRVPP VGSGTATYSG NPFVGVTPWA NAPYASEVSS LAIPSLTGAM ATAAAAVAKV PSFMWLDTLD |
| | | | KTPLMEQTLA DIRTANKNGG NYAGQFVVYD LPDRDCAALA SNGEYSIADG GVAKYKNYID TIRQIVVEYS |
| | | | DIRTLLVIEP DSLANLVTNL GTPKCANAQS AYLECINYAV TQLNLPNVAM YLDAGHAGWL GWPANQDPAA |
| | | | QLFANVYKNA SSPRALRGLA TNVANYNGWN ITSPPSYTQG NAVYNEKLYI HAIGPLLANH GWSNAFFITD |
| | | | QGRSGKQPTG QQQWGDWCNV IGTGFGIRPS ANTGDSLLDS FVWVKPGGEC DGTSDSSAPR FDPHCALPDA |
| | | | LQPAPQAGAW FQAYFVQLLT NANPSFL |
| SEQ ID NO: 27 | US8012734-0043 | Trichoderma | QACSSVWGQC GGQNWSGPTC CASGSTCVYS NDYYSQCLPG AASSSSSTRA ASTTSRVSPT TSRSSSATPP |
| | | reesei | PGSTTTRVPP VGSGTATYSG NPFVGVTPWA NARYASEVSS LAIPSLTGAM ATAAAAVAKV PSFMWLDTLD |
| | | | KTPLMEQTLA DIRTANKNGG NYAGQFVVYD LPDRDCAALA SNGEYSIADG GVAKYKNYID TIRQIVVEYS |
| | | | DIRTLLVIEP DSLANLVTNL GTPKCANAQS AYLECINYAV TQLNLPNVAM YLDAGHAGWL GWPANQDPAA |
| | | | QLFANVYKNA SSPRALRGLA TNVANYNGWN ITSPPSYTQG NAVYNEKLYI HAIGPLLANH GWSNAFFITD |
| | | | QGRSGKQPTG QQQWGDWCNV IGTGFGIRPS ANTGDSLLDS FVWVKPGGEC DGTSDSSAPR FDPHCALPDA |
| | | | LQPAPQAGAW FQAYFVQLLT NANPSFL |
| SEQ ID NO: 28 | US8012734-0044 | Trichoderma | QACSSVWGQC GGQNWSGPTC CASGSTCVYS NDYYSQCLPG AASSSSSTRA ASTTSRVSPT TSRSSSATPP |
| | | reesei | PGSTTTRVPP VGSGTATYSG NPFVGVTPWA NARYASEVSS LAIPSLTGAM ATAAAAVAKV PSFMWLDTLD |
| | | | KTPLMEQTLA DIRTANKNGG NYAGQFVVYD LPDRDCAALA SNGEYSIADG GVAKYKNYID TIRQIVVEYS |
| | | | DIRTLLVIEP DSLANLVTNL GTPKCANAQS AYLECINYAV TQLNLPNVAM YLDAGHAGWL GWPANQDPAA |
| | | | QLFANVYKNA SSPRALRGLA TNVANYNGWN ITSPPSYTQG NAVYNEKLYI HAIGPLLANH GWSNAFFITD |
| | | | QGRSGKQPTG QQQWGDWCNV IGTGFGIRPS ANTGDSLLDS FVWVKPGGEC DGTSDSSAPR FDPHCALPDA |
| | | | LQPAPQAGAW FQAYFVQLLT NANPSFL |
| SEQ ID NO: 29 | US8012734-0045 | Trichoderma | QACSSVWGQC GGQNWSGPTC CASGSTCVYS NDYYSQCLPG AASSSSSTRA ASTTSRVSPT TSRSSSATPP |
| | | reesei | PGSTTTRVPP VGSGTATYSG NPFVGVTPWA NAYYASEVSS LAIPSLTGAM ATAAAAVAKV PSFMWLDTLD |
| | | | KTPLMEQTLA DIRTANKNGG NYAGQFVVYD LPDRDCAALA SNGEYSIADG GVAKYKNYID TIRQIVVEYS |
| | | | DIRTLLVIEP DSLANLVTNL GTPKCANAQS AYLECINYAV TQLNLPNVAM YLDAGHAGWL GWPANQDPAA |
| | | | QLFANVYKNA SSPRALRGLA TNVANYNGWN ITSPPSYTQG NAVYNEKLYI HAIGPLLANH GWSNAFFITD |
| | | | QGRSGKQPTG QQQWGDWCNV IGTGFGIRPS ANTGDSLLDS FVWVKPGGEC DGTSDSSAPR FDPHCALPDA |
| | | | LQPAPQAGAW FQAYFVQLLT NANPSFL |
| SEQ ID NO: 30 | US8012734-0046 | Trichoderma | QACSSVWGQC GGQNWSGPTC CASGSTCVYS NDYYSQCLPG AASSSSSTRA ASTTSRVSPT TSRSSSATPP |
| | | reesei | PGSTTTRVPP VGSGTATYSG NPFVGVTPWA NAYYASEVSS LAIPSLTGAM ATAAAAVAKV PSFMWLDTLD |
| | | | KTPLMEQTLA DIRTANKNGG NYAGQFVVYD LPDRDCAALA SNGEYSIADG GVAKYKNYID TIRQIVVEYS |
| | | | DIRTLLVIEP DSLANLVTNL GTPKCANAQS AYLECINYAV TQLNLPNVAM YLDAGHAGWL GWPANQDPAA |
| | | | QLFANVYKNA SSPRALRGLA TNVANYNGWN ITSPPSYTQG NAVYNEKLYI HAIGPLLANH GWSNAFFITD |
| | | | QGRSGKQPTG QQQWGDWCNV IGTGFGIRPS ANTGDSLLDS FVWVKPGGEC DGTSDSSAPR FDPHCALPDA |
| | | | LQPAPQAGAW FQAYFVQLLT NANPSFL |
| SEQ ID NO: 31 | US8012734-0047 | Trichoderma | QACSSVWGQC GGQNWSGPTC CASGSTCVYS NDYYSQCLPG AASSSSSTRA ASTTSRVSPT TSRSSSATPP |
| | | reesei | PGSTTTRVPP VGSGTATYSG NPFVGVTPWA NAYYASEVSS LAIPSLTGAM ATAAAAVAKV PSFMWLDTLD |
| | | | KTPLMEQTLA DIRTANKNGG NYAGQFVVYD LPDRDCAALA SNGEYKIADG GVAKYKNYID TIRQIVVEYS |
| | | | DIRTLLVIEP DSLANLVTNL GTPKCANAQS AYLECINYAV TQLNLPNVAM YLDAGHAGWL GWPANQDPAA |
| | | | QLFANVYKNA SSPRALRGLA TNVANYNGWN ITSPPSYTQG NAVYNEKLYI HAIGPLLANH GWSNAFFITD |
| | | | QGRSGKQPTG QQQWGDWCNV IGTGFGIRPS ANTGDSLLDS FVWVKPGGEC DGTSDSSAPR FDPHCALPDA |
| | | | LQPAPQAGAW FQAYFVQLLT NANPSFL |
| SEQ ID NO: 32 | US8012734-0048 | Trichoderma | QACSSVWGQC GGQNWSGPTC CASGSTCVYS NDYYSQCLPG AASSSSSTRA ASTTSRVSPT TSRSSSATPP |
| | | reesei | PGSTTTRVPP VGSGTATYSG NPFVGVTPWA NAYYASEVSS LAIPSLTGAM ATAAAAVAKV PSFMWLDTLD |
| | | | KTPLMEQTLA DIRTANKNGG NYAGQFVVYD LPDRDCAALA SNGEYYIADG GVAKYKNYID TIRQIVVEYS |
| | | | DIRTLLVIEP DSLANLVTNL GTPKCANAQS AYLECINYAV TQLNLPNVAM YLDAGHAGWL GWPANQDPAA |
| | | | QLFANVYKNA SSPRALRGLA TNVANYNGWN ITSPPSYTQG NAVYNEKLYI HAIGPLLANH GWSNAFFITD |
| | | | QGRSGKQPTG QQQWGDWCNV IGTGFGIRPS ANTGDSLLDS FVWVKPGGEC DGTSDSSAPR FDPHCALPDA |
| | | | LQPAPQAGAW FQAYFVQLLT NANPSFL |
| SEQ ID NO: 33 | US8012734-0049 | Trichoderma | QACSSVWGQC GGQNWSGPTC CASGSTCVYS NDYYSQCLPG AASSSSSTRA ASTTSRVSPT TSRSSSATPP |
| | | reesei | PGSTTTRVPP VGSGTATYSG NPFVGVTPWA NAYYASEVSS LAIPSLTGAM ATAAAAVAKV PSFMWLDTLD |
| | | | KTPLMEQTLA DIRTANKNGG NYAGQFVVYD LPDRDCAALA SNGEYSIADG GVAKYKNYID TIRQIVVEYS |
| | | | DIRTLLVIEP DSLANLVTNL GTPKCANAQS AYLECINYAV TQLNLPNVAM YLDAGHAGWL GWPANQDPAA |
| | | | QLFANVYKNA SSPRALRGLA TNVANYNGWN ITSPPSYTQG NAVYNEKLYI HAIGPLLANH GWSNAFFITD |
| | | | QGRSGKQPTG QQQWDDWCNV IGTGFGIRPS ANTGDSLLDS FVWVKPGGEC DGTSDSSAPR FDPHCALPDA |
| | | | LQPAPQAGAW FQAYFVQLLT NANPSFL |
| SEQ ID NO: 34 | US8012734-0050 | Trichoderma | QACSSVWGQC GGQNWSGPTC CASGSTCVYS NDYYSQCLPG AASSSSSTRA ASTTSRVSPT TSRSSSATPP |
| | | reesei | PGSTTTRVPP VGSGTATYSG NPFVGVTPWA NAYYASEVSS LAIPSLTGAM ATAAAAVAKV PSFMWLDTLD |
| | | | KTPLMEQTLA DIRTANKNGG NYAGQFVVYD LPDRDCAALA SNGEYSIADG GVAKYKNYID TIRQIVVEYS |
| | | | DIRTLLVIEP DSLANLVTNL GTPKCANAQS AYLECINYAV TQLNLPNVAM YLDAGHAGWL GWPANQDPAA |
| | | | QLFANVYKNA SSPRALRGLA TNVANYNGWN ITSPPSYTQG NAVYNEKLYI HAIGPLLANH GWSNAFFITD |
| | | | QGRSGKQPTG QQQWEDWCNV IGTGFGIRPS ANTGDSLLDS FVWVKPGGEC DGTSDSSAPR FDPHCALPDA |
| | | | LQPAPQAGAW FQAYFVQLLT NANPSFL |
| SEQ ID NO: 35 | US8012734-0051 | Trichoderma | QACSSVWGQC GGQNWSGPTC CASGSTCVYS NDYYSQCLPG AASSSSSTRA ASTTSRVSPT TSRSSSATPP |
| | | reesei | PGSTTTRVPP VGSGTATYSG NPFVGVTPWA NAYYASEVSS LAIPSLTGAM ATAAAAVAKV PSFMWLDTLD |
| | | | KTPLMEQTLA DIRTANKNGG NYAGQFVVYD LPDRDCAALA SNGEYSIADG GVAKYKNYID TIRQIVVEYS |
| | | | DIRTLLVIEP DSLANLVTNL GTPKCANAQS AYLECINYAV TQLNLPNVAM YLDAGHAGWL GWPANQDPAA |
| | | | QLFANVYKNA SSPRALRGLA TNVANYNGWN ITSPPSYTQG NAVYNEKLYI HAIGPLLANH GWSNAFFITD |
| | | | QGRSGKQPTG QQQWQDWCNV IGTGFGIRPS ANTGDSLLDS FVWVKPGGEC DGTSDSSAPR FDPHCALPDA |
| | | | LQPAPQAGAW FQAYFVQLLT NANPSFL |
| SEQ ID NO: 36 | US8012734-0052 | Trichoderma | QACSSVWGQC GGQNWSGPTC CASGSTCVYS NDYYSQCLPG AASSSSSTRA ASTTSRVSPT TSRSSSATPP |
| | | reesei | PGSTTTRVPP VGSGTATYSG NPFVGVTPWA NAYYASEVSS LAIPSLTGAM ATAAAAVAKV PSFMWLDTLD |
| | | | KTPLMEQTLA DIRTANKNGG NYAGQFVVYD LPDRDCAALA SNGEYSIADG GVAKYKNYID TIRQIVVEYS |
| | | | DIRTLLVIEP DSLANLVTNL GTPKCANAQS AYLECINYAV TQLNLPNVAM YLDAGHAGWL GWPANQDPAA |
| | | | QLFANVYKNA SSPRALRGLA TNVANYNGWN ITSPPSYTQG NAVYNEKLYI HAIGPLLANH GWSNAFFITD |
| | | | QGRSGKQPTG QQQWSDWCNV IGTGFGIRPS ANTGDSLLDS FVWVKPGGEC DGTSDSSAPR FDPHCALPDA |
| | | | LQPAPQAGAW FQAYFVQLLT NANPSFL |
| SEQ ID NO: 37 | US8012734-0053 | Trichoderma | QACSSVWGQC GGQNWSGPTC CASGSTCVYS NDYYSQCLPG AASSSSSTRA ASTTSRVSPT TSRSSSATPP |
| | | reesei | PGSTTTRVPP VGSGTATYSG NPFVGVTPWA NAYYASEVSS LAIPSLTGAM ATAAAAVAKV PSFMWLDTLD |
| | | | KTPLMEQTLA DIRTANKNGG NYAGQFVVYD LPDRDCAALA SNGEYSIADG GVAKYKNYID TIRQIVVEYS |
| | | | DIRTLLVIEP DSLANLVTNL GTPKCANAQS AYLECINYAV TQLNLPNVAM YLDAGHAGWL GWPANQDPAA |
| | | | QLFANVYKNA SSPRALRGLA TNVANYNGWN ITSPPSYTQG NAVYNEKLYI HAIGPLLANH GWSNAFFITD |
| | | | QGRSGKQPTG QQQWGDWCNV IGTGFGIRPS ANTGDSLLDS FVWVKPGGEC DGTSDSSAPA FDPHCALPDA |
| | | | LQPAPQAGAW FQAYFVQLLT NANPSFL |
| SEQ ID NO: 38 | US8012734-0054 | Trichoderma | QACSSVWGQC GGQNWSGPTC CASGSTCVYS NDYYSQCLPG AASSSSSTRA ASTTSRVSPT TSRSSSATPP |
| | | reesei | PGSTTTRVPP VGSGTATYSG NPFVGVTPWA NAYYASEVSS LAIPSLTGAM ATAAAAVAKV PSFMWLDTLD |
| | | | KTPLMEQTLA DIRTANKNGG NYAGQFVVYD LPDRDCAALA SNGEYSIADG GVAKYKNYID TIRQIVVEYS |
| | | | DIRTLLVIEP DSLANLVTNL GTPKCANAQS AYLECINYAV TQLNLPNVAM YLDAGHAGWL GWPANQDPAA |
| | | | QLFANVYKNA SSPRALRGLA TNVANYNGWN ITSPPSYTQG NAVYNEKLYI HAIGPLLANH GWSNAFFITD |
| | | | QGRSGKQPTG QQQWGDWCNV IGTGFGIRPS ANTGDSLLDS FVWVKPGGEC DGTSDSSAPF FDPHCALPDA |
| | | | LQPAPQAGAW FQAYFVQLLT NANPSFL |
| SEQ ID NO: 39 | US8012734-0055 | Trichoderma | QACSSVWGQC GGQNWSGPTC CASGSTCVYS NDYYSQCLPG AASSSSSTRA ASTTSRVSPT TSRSSSATP |
| | | reesei | PPGSTTTRVPP VGSGTATYSG NPFVGVTPWA NAYYASEVSS LAIPSLTGAM ATAAAAVAKV PSFMWLDTLD |
| | | | KTPLMEQTL ADIRTANKNGG NYAGQFVVYD LPDRDCAALA SNGEYSIADG GVAKYKNYID TIRQIVVEYS |
| | | | DIRTLLVIEP DSLANLVTN LGTPKCANAQ SAYLECINYA VTQLNLPNVA MYLDAGHAGW LGWPANQDPA |
| | | | AQLFANVYKN ASSPRALRGL ATNVANYNG WNITSPPSYT QGNAVYNEKL YIHAIGPLLA NHGWSNAFFI |
| | | | TDQGRSGKQP TGQQQWGDWC NVIGTGFGIR PSANTGDSL LDSFVWVKPG GECDGTSDSS APLFDPHCAL |
| | | | PDALQPAPQA GAWFQAYFVQ LLTNANPSFL |
| SEQ ID NO: 40 | US8012734-0057 | Trichoderma | QACSSVWGQC GGQNWSGPTC CASGSTCVYS NDYYSQCLPG AASSSSSTRA ASTTSRVSPT TSRSSSATPP |
| | | reesei | PGSTTTRVPP VGSGTATYSG NPFVGVTPWA NAYYASEVSS LAIPSLTGAM ATAAAAVAKV PSFMWLDTLD |
| | | | KTPLMEQTLA DIRTANKNGG NYAGQFVVYD LPDRDCAALA SNGEYSIADG GVAKYKNYID TIRQIVVEYS |
| | | | DIRTLLVIEP DSLANLVTNL GTPKCANAQS AYLECINYAV TQLNLPNVAM YLDAGHAGWL GWPANQDPAA |
| | | | QLFANVYKNA SSPRALRGLA TNVANYNGWN ITSPPSYTQG NAVYNEKLYI HAIGPLLANH GWSNAFFITD |
| | | | QGRSGKQPTG QQQWGDWCNV IGTGFGIRPS ANTGDSLLDS FVWVKPGGEC DGTSDSSAPS FDPHCALPDA |
| | | | LQPAPQAGAW FQAYFVQLLT NANPSFL |
| SEQ ID NO: 41 | US8012734-0058 | Trichoderma | QACSSVWGQC GGQNWSGPTC CASGSTCVYS NDYYSQCLPG AASSSSSTRA ASTTSRVSPT TSRSSSATPP |
| | | reesei | PGSTTTRVPP VGSGTATYSG NPFVGVTPWA NALYASEVSS LAIPSLTGAM ATAAAAVAKV PSFMWLDTLD |
| | | | KTPLMEQTLA DIRTANKNGG NYAGQFVVYD LPDRDCAALA SNGEYSIADG GVAKYKNYID TIRQIVVEYS |
| | | | DIRTLLVIEP DSLANLVTNL GTPKCANAQS AYLECINYAV TQLNLPNVAM YLDAGHAGWL GWPANQDPAA |
| | | | QLFANVYKNA SSPRALRGLA TNVANYNGWN ITSPPSYTQG NAVYNEKLYI HAIGPLLANH GWSNAFFITD |
| | | | QGRSGKQPTG QQQWGDWCNV IGTGFGIRPS ANTGDSLLDS FVWVKPGGEC DGTSDSSAPR FDPHCALPDA |
| | | | LQPAPQAGAW FQAYFVQLLT NANPSFL |
| SEQ ID NO: 42 | US8012734-0061 | Trichoderma | QACSSVWGQC GGQNWSGPTC CASGSTCVYS NDYYSQCLPG AASSSSSTRA ASTTSRVSPT TSRSSSATPP |
| | | reesei | PGSTTTRVPP VGSGTATYSG NPFVGVTPWA NALYASEVSS LAIPSLTGAM ATAAAAVAKV PSFMWLDTLD |
| | | | KTPLMEQTLA DIRTANKNGG NYAGQFVVYD LPDRDCAALA SNGEYSIADG GVAKYKNYID TIRQIVVEYS |
| | | | DIRTLLVIEP DSLANLVTNL GTPKCANAQS AYLECINYAV TQLNLPNVAM YLDAGHAGWL GWPANQDPAA |
| | | | QLFANVYKNA SSPRALRGLA TNVANYNGWN ITSPPSYTQG NAVYNEKLYI HAIGPLLANH GWSNAFFITD |
| | | | QGRSGKQPTG QQQWGDWCNV IGTGFGIRPS ANTGDSLLDS FVWVKPGGEC DGTSDSSAPQ FDPHCALPDA |
| | | | LQPAPQAGAW FQAYFVQLLT NANPSFL |
| SEQ ID NO: 43 | US8012734-0062 | Trichoderma | QACSSVWGQC GGQNWSGPTC CASGSTCVYS NDYYSQCLPG AASSSSSTRA ASTTSRVSPT TSRSSSATPP |
| | | reesei | PGSTTTRVPP VGSGTATYSG NPFVGVTPWA NAYYASEVSS LAIPSLTGAM ATAAAAVAKV PSFMWIDTLD |
| | | | KTPLMEQTLA DIRTANKNGG NYAGQFVVYD LPDRDCAALA SNGEYSIADG GVAKYKNYID TIRQIVVEYS |
| | | | DIRTLLVIEP DSLANLVTNL GTPKCANAQS AYLECINYAV TQLNLPNVAM YLDAGHAGWL GWPANQDPAA |
| | | | QLFANVYKNA SSPRALRGLA TNVANYNGWN ITSPPSYTQG NAVYNEKLYI HAIGPLLANH GWSNAFFITD |
| | | | QGRSGKQPTG QQQWGDWCNV IGTGFGIRP SANTGDSLLD SFVWVKPGGE CDGTSDSSAP RFDPHCALPD |
| | | | ALQPAPQAGA WFQAYFVQLL TNANPSFL |
| SEQ ID NO: 44 | US8012734-0063 | Trichoderma | QACSSVWGQC GGQNWSGPTC CASGSTCVYS NDYYSQCLPG AASSSSSTRA ASTTSRVSPT TSRSSSATPP |
| | | reesei | PGSTTTRVPP VGSGTATYSG NPFVGVTPWA NAYYASEVSS LAIPSLTGAM ATAAAAVAKV PSFMWIDTLD |
| | | | KTPLMEQTLA DIRTANKNGG NYAGQFVVYD LPDRDCAALA SNGEYSIADG GVAKYKNYID TIRQIVVEYS |
| | | | DIRTILVIEP DSLANLVTNL GTPKCANAQS AYLECINYAV TQLNLPNVAM YLDAGHAGWL GWPANQDPAA |
| | | | QLFANVYKNA SSPRALRGLA TNVANYNGWN ITSPPSYTQG NAVYNEKLYI HAIGPLLANH GWSNAFFITD |
| | | | QGRSGKQPTG QQQWGDWCNV IGTGFGIRPS ANTGDSLLDS FVWVKPGGEC DGTSDSSAPR FDPHCALPDA |
| | | | LQPAPQAGAW FQAYFVQLLT NANPSFL |
| SEQ ID NO: 45 | US8012734-0064 | Trichoderma | QACSSVWGQC GGQNWSGPTC CASGSTCVYS NDYYSQCLPG AASSSSSTRA ASTTSRVSPT TSRSSSATPP |
| | | reesei | PGSTTTRVPP VGSGTATYSG NPFVGVTPWA NAYYASEVSS LAIPSLTGAM ATAAAAVAKV PSFMWIDTLD |
| | | | KTPLMEQTLA DIRTANKNGG NYAGQFVVYD LPDRDCAALA SNGEYYIADG GVAKYKNYID TIRQIVVEYS |
| | | | DIRTLLVIEP DSLANLVTNL GTPKCANAQS AYLECINYAV TQLNLPNVAM YLDAGHAGWL GWPANQDPAA |
| | | | QLFANVYKNA SSPRALRGLA TNVANYNGWN ITSPPSYTQG NAVYNEKLYI HAIGPLLANH GWSNAFFITD |
| | | | QGRSGKQPTG QQQWGDWCNV IGTGFGIRPS ANTGDSLLDS FVWVKPGGEC DGTSDSSAPR FDPHCALPDA |
| | | | LQPAPQAGAW FQAYFVQLLT NANPSFL |
| SEQ ID NO: 46 | US8012734-0066 | Trichoderma | QACSSVWGQC GGQNWSGPTC CASGSTCVYS NDYYSQCLPG AASSSSSTRA ASTTSRVSPT TSRSSSATPP |
| | | reesei | PGSTTTRVPP VGSGTATYSG NPFVGVTPWA NAYYASEVSS LAIPSLTGAM ATAAAAVAKV PSFMWIDTLD |
| | | | KTPLMEQTLA DIRTANKNGG NYAGQFVVYD LPDRDCAALA SNGEYSIADG GVAKYKNYID TIRQIVVEYS |
| | | | DIRTLLVIEP DSLANLVTNL GTPKCANAQS AYLECINYAV TQLNLPNVAM YLDAGHAGWL GWPANQDPAA |
| | | | QLFANVYKNA SSPRALRGLA TNVANYNGWN ITSPPSYTQG NAVYNEKLYI HAIGPLLANH GWSNAFFITD |
| | | | QGRSGKQPTG QQQWGDWCNV IGTGFGIRPS ANTGDSLLDS FVWVKPGGEC DGTSDSSAPQ FDPHCALPDA |
| | | | LQPAPQAGAW FQAYFVQLLT NANPSFL |
| SEQ ID NO: 47 | US8012734-0071 | Trichoderma | QACSSVWGQC GGQNWSGPTC CASGSTCVYS NDYYSQCLPG AASSSSSTRA ASTTSRVSPT TSRSSSATPP |
| | | reesei | PGSTTTRVPP VGSGTATYSG NPFVGVTPWA NAYYASEVSS LAIPSLTGAM ATAAAAVAKV PSFVWIDTLD |
| | | | KTPLMEQTLA DIRTANKNGG NYAGQFVVYD LPDRDCAALA SNGEYSIADG GVAKYKNYID TIRQIVVEYS |
| | | | DIRTILVIEP DSLANLVTNL GTPKCANAQS AYLECINYAV TQLNLPNVAM YLDAGHAGWL GWPANQDPAA |
| | | | QLFANVYKNA SSPRALRGLA TNVANYNGWN ITSPPSYTQG NAVYNEKLYI HAIGPLLANH GWSNAFFITD |
| | | | QGRSGKQPTG QQQWGDWCNV IGTGFGIRPS ANTGDSLLDS FVWVKPGGEC DGTSDSSAPR FDPHCALPDA |
| | | | LQPAPQAGAW FQAYFVQLLT NANPSFL |
| SEQ ID NO: 48 | US8012734-0157 | Trichoderma | QACSSVWGQC GGQNWSGPTC CASGSTCVYS NDYYSQCLPG AASSSSSTRA ASTTSRVSPT TSRSSSATPP |
| | | reesei | PGSTTTRVPP VGSGTATYSG NPFVGVTPWA NAYYASEVSS LAIPSLTGAM ATAAAAVAKV PSFQWLDTLD |
| | | | KTPLMEQTLA DIRTANKNGG NYAGQFVVYD LPDRDCAALA SNGEYSIADG GVAKYKNYID TIRQIVVEYS |
| | | | DIRTLLVIEP DSLANLVTNL GTPKCANAQS AYLECINYAV TQLNLPNVAM YLDAGHAGWL GWPANQDPAA |
| | | | QLFANVYKNA SSPRALRGLA TNVANYNGWN ITSPPSYTQG NAVYNEKLYI HAIGPLLANH GWSNAFFITD |
| | | | QGRSGKQPTG QQQWGDWCNV IGTGFGIRPS ANTGDSLLDS FVWVKPGGEC DGTSDSSAPR FDPHCALPDA |
| | | | LQPAPQAGAW FQAYFVQLLT NANPSFL |
| SEQ ID NO: 49 | US8012734-0158 | Trichoderma | QACSSVWGQC GGQNWSGPTC CASGSTCVYS NDYYSQCLPG AASSSSSTRA ASTTSRVSPT TSRSSSATPP |
| | | reesei | PGSTTTRVPP VGSGTATYSG NPFVGVTPWA NAYYASEVSS LAIPSLTGAM ATAAAAVAKV PSFQWLDTLD |
| | | | KTPLMEQTLA DIRTANKNGG NYAGQFVVYD LPDRDCAALA SNGEYSIADG GVAKYKNYID TIRQIVVEYS |
| | | | DIRTLLVIEP DSLANLVTNL GTPKCANAQS AYLECINYAV TQLNLPNVAM YLDAGHAGWL GWPANQDPAA |
| | | | QLFANVYKNA SSPRALRGLA TNVANYNGWN ITSPPSYTQG NAVYNEKLYI HAIGPLLANH GWSNAFFITD |
| | | | QGRSGKQPTG QQQWGDWCNV IGTGFGIRPS ANTGDSLLDS FVWVKPGGEC DGTSDSSAPR FDPHCALPDA |
| | | | LQPAPQAGAW FQAYFVQLLT NANPSFL |
| SEQ ID NO: 50 | US8012734-0159 | Trichoderma | QACSSVWGQC GGQNWSGPTC CASGSTCVYS NDYYSQCLPG AASSSSSTRA ASTTSRVSPT TSRSSSATPP |
| | | reesei | PGSTTTRVPP VGSGTATYSG NPFVGVTPWA NAYYASEVSS LAIPSLTGAM ATAAAAVAKV PSFQWLDTLD |
| | | | KTPLMEQTLA DIRTANKNGG NYAGQFVVYD LPDRDCAALA SNGEYSIADG GVAKYKNYID TIRQIVVEYS |
| | | | DIRTLLVIEP DSLANLVTNL GTPKCANAQS AYLECINYAV TQLNLPNVAM YLDAGHAGWL GWPANQDPAA |
| | | | QLFANVYKNA SSPRALRGLA TNVANYNGWN ITSPPSYTQG NAVYNEKLYI HAIGPLLANH GWSNAFFITD |
| | | | QGRSGKQPTG QQQWGDWCNV IGTGFGIRPS ANTGDSLLDS FVWVKPGGEC DGTSDSSAPR FDPHCALPDA |
| | | | LQPAPQAGAW FQAYFVQLLT NANPSFL |
| SEQ ID NO: 51 | US8012734-0160 | Trichoderma | QACSSVWGQC GGQNWSGPTC CASGSTCVYS NDYYSQCLPG AASSSSSTRA ASTTSRVSPT TSRSSSATPP |
| | | reesei | PGSTTTRVPP VGSGTATYSG NPFVGVTPWA NAYYASEVSS LAIPSLTGAM ATAAAAVAKV PSFVWLDTLD |
| | | | KTPLMEQTLA DIRTANKNGG NYAGQFVVYD LPDRDCAALA SNGEYSIADG GVAKYKNYID TIRQIVVEYS |
| | | | DIRTLLVIEP DSLANLVTNL GTPKCANAQS AYLECINYAV TQLNLPNVAM YLDAGHAGWL GWPANQDPAA |
| | | | QLFANVYKNA SSPRALRGLA TNVANYNGWN ITSPPSYTQG NAVYNEKLYI HAIGPLLANH GWSNAFFITD |
| | | | QGRSGKQPTG QQQWGDWCNV IGTGFGIRPS ANTGDSLLDS FVWVKPGGEC DGTSDSSAPR FDPHCALPDA |
| | | | LQPAPQAGAW FQAYFVQLLT NANPSFL |
| SEQ ID NO: 52 | US8012734-0161 | Trichoderma | QACSSVWGQC GGQNWSGPTC CASGSTCVYS NDYYSQCLPG AASSSSSTRA ASTTSRVSPT TSRSSSATPP |
| | | reesei | PGSTTTRVPP VGSGTATYSG NPFVGVTPWA NAYYASEVSS LAIPSLTGAM ATAAAAVAKV PSFYWLDTLD |
| | | | KTPLMEQTLA DIRTANKNGG NYAGQFVVYD LPDRDCAALA SNGEYSIADG GVAKYKNYID TIRQIVVEYS |
| | | | DIRTLLVIEP DSLANLVTNL GTPKCANAQS AYLECINYAV TQLNLPNVAM YLDAGHAGWL GWPANQDPAA |
| | | | QLFANVYKNA SSPRALRGLA TNVANYNGWN ITSPPSYTQG NAVYNEKLYI HAIGPLLANH GWSNAFFITD |
| | | | QGRSGKQPTG QQQWGDWCNV IGTGFGIRPS ANTGDSLLDS FVWVKPGGEC DGTSDSSAPR FDPHCALPDA |
| | | | LQPAPQAGAW FQAYFVQLLT NANPSFL |
| SEQ ID NO: 53 | US7785854-0023 | Trichoderma | DYKDDDDKEF LEASCSSVWG QCGGQNWSGP TCCASGSTCV YSNDYYSQCL PGAASSSSST RAASTTSRVS |
| | | reesei | PTTSRSSSAT PPPGSTTTRV PPVGSGTATY SGNPFVGVTP WANAYYASEV SSLAIPSLTG AMATAAAAVA |
| | | | KVPSFMWLDT LDKTPLMEQT LADIRTANKN GGNYAGQFVV YDLPDRDCAA LASNGEYSIA DGGVAKYKNY |
| | | | IDTIRQIVVE YSDIRTLLVI EPDSLANLVT NLGTPKCANA QSAYLECINY AVTQLNLPNV AMYLDAGHAG |
| | | | WLGWPANQDP AAQLFANVYK NASSPRALRG LATNVANYNG WNITSPPSYT QGNAVYNEKL YIHAIGPLLA |
| | | | NHGWSNAFFI TDQGRSGKQP TGQQQWGDWC NVIGTGFGIR PSANTGDSLL DSFVWVKPGG ECDGTSDSSA |
| | | | PRFDSHCALP DALQPAPQAG AWFQAYFVQL LTNANPSFL |
| SEQ ID NO: 54 | US8101398-0012 | Trichoderma | QACSSVWGQC GGQNWSGPTC CASGSTCVYS NDYYSQCLPG AASSSSSTRA ASTTSRVSPT TSRSSSATPP |
| | | reesei | PGSTTTRVPP VGSGTATYSG NPFVGVTPWA NAYYASEVSS LAIPSLTGAM ATAAAAVAKV PSFMWLDTLD |
| | | | KTPLMEQTLA DIRTANKNGG NYAGQFVVYD LPDRDCAALA SNGEYSIADG GVAKYKNYID TIRQIVVEYS |
| | | | DIRTLLVIEP DSLANLVTNL GTPKCANAQS AYLECINYAV TQLNLPNVAM YLDAGHAGWL GWPANQDPAA |
| | | | QLFANVYKNA SSPRALRGLA TNVANYNGWN ITSPPSYTQG NAVYNEKLYI HAIGPLLANH GWSNAFFITD |
| | | | QGRSGKQPTG QQQWGDWCNV IGTGFGIRPS ANTGDSLLDS FVWVKPGGEC DGTSDSSAPR FDPHCALPDA |
| | | | LQPAPQAGAW FQAYFVQLLT NANPSFL |
| SEQ ID NO: 55 | US8101398-0014 | Trichoderma | QACSSVWGQC GGQNWSGPTC CASGSTCVYS NDYYSQCLPG AASSSSSTRA ASTTSRVSPT TSRSSSATPP |
| | | reesei | PGSTTTRVPP VGSGTATYSG NPFVGVTPWA NAYYASEVSS LAIPSLTGAM ATAAAAVAKV PSFMWLDTLD |
| | | | KTPLMEQTLA DIRTANKNGG NYAGQFVVYD LPDRDCAALA SNGEYSIADG GVAKYKNYID TIRQIVVEYS |
| | | | DIRTLLVIEP DSLANLVTNL STPKCANAQS AYLECINYAV TQLNLPNVAM YLDAGHAGWL GWPANQDPAA |
| | | | QLFANVYKNA SSPRALRGLA TNVANYNGWN ITSPPSYTQG NAVYNEKLYI HAIGPLLANH GWSNAFFITD |
| | | | QGRSGKQPTG QQQWGDWCNV IGTGFGIRPS ANTGDSLLDS FVWVKPGGEC DGTSDSSAPR FDPHCALPDA |
| | | | LQPAPQAGAW FQAYFVQLLT NANPSFL |
| SEQ ID NO: 56 | US8101398-0015 | Trichoderma | QACSSVWGQC GGQNWSGPTC CASGSTCVYS NDYYSQCLPG AASSSSSTRA ASTTSRVSPT TSRSSSATPP |
| | | reesei | PGSTTTRVPP VGSGTATYSG NPFVGVTPWA NAYYASEVSS LAIPSLTGAM ATAAAAVAKV PSFMWLDTLD |
| | | | KTPLMEQTLA DIRTANKNGG NYAGQFVVYD LPDRDCAALA SNGEYSIADG GVAKYKNYID TIRQIVVEYS |
| | | | DIRTLLVIEP DSLANLVTNL GTPKCANAQS AYLECINYAV TQLNLPNVAM YLDAGHAGWL GWPANQDPAA |
| | | | QLFANVYKNA SSPRALRGLA TNVASYNGWN ITSPPSYTQG NAVYNEKLYI HAIGPLLANH GWSNAFFITD |
| | | | QGRSGKQPTG QQQWGDWCNV IGTGFGIRPS ANTGDSLLDS FVWVKPGGEC DGTSDSSAPR FDPHCALPDA |
| | | | LQPAPQAGAW FQAYFVQLLT NANPSFL |
| SEQ ID NO: 57 | US8101398-0016 | Trichoderma | QACSSVWGQC GGQNWSGPTC CASGSTCVYS NDYYSQCLPG AASSSSSTRA ASTTSRVSPT TSRSSSATPP |
| | | reesei | PGSTTTRVPP VGSGTATYSG NPFVGVTPWA NAYYASEVSS LAIPSLTGAM ATAAAAVAKV PSFMWLDTLD |
| | | | KTPLMEQTLA DIRTANKNGG NYAGQFVVYD LPDRDCAALA SNGEYSIADG GVAKYKNYID TIRQIVVEYS |
| | | | DIRTLLVIEP DSLANLVTNL GTPKCANAQS AYLECINYAV TQLNLPNVAM YLDAGHAGWL GWPANQDPAA |
| | | | QLFANVYKNA SSPRALRGLA TNVANYNGWN ITSPPSYTQG NAVYNEKLYI HAIGPLLANH GWSNAFFITD |
| | | | QGRSGKQPTG QQQWGDWCNV IGTGFGIRPS ANTGDSLLDS FVWVKPGGEC DGTSDSSAPQ FDPHCALPDA |
| | | | LQPAPQAGAW FQAYFVQLLT NANPSFL |
| SEQ ID NO: 58 | US8101398-0017 | Trichoderma | QACSSVWGQC GGQNWSGPTC CASGSTCVYS NDYYSQCLPG AASSSSSTRA ASTTSRVSPT TSRSSSATPP |
| | | reesei | PGSTTTRVPP VGSGTATYSG NPFVGVTPWA NAYYASEVSS LAIPSLTGAM ATAAAAVAKV PSFMWLDTLD |
| | | | KTPLMEQTLA DIRTANKNGG NYAGQFVVYD LPDRDCAALA SNGEYSIADG GVAKYKNYID TIRQIVVEYS |
| | | | DIRTLLVIEP DSLANLVTNL STPKCANAQS AYLECINYAV TQLNLPNVAM YLDAGHAGWL GWPANQDPAA |
| | | | QLFANVYKNA SSPRALRGLA TNVASYNGWN ITSPPSYTQG NAVYNEKLYI HAIGPLLANH GWSNAFFITD |
| | | | QGRSGKQPTG QQQWGDWCNV IGTGFGIRPS ANTGDSLLDS FVWVKPGGEC DGTSDSSAPR FDPHCALPDA |
| | | | LQPAPQAGAW FQAYFVQLLT NANPSFL |
| SEQ ID NO: 59 | US8101398-0020 | Trichoderma | QACSSVWGQC GGQNWSGPTC CASGSTCVYS NDYYSQCLPG AASSSSSTRA ASTTSRVSPT TSRSSSATPP |
| | | reesei | PGSTTTRVPP VGSGTATYSG NPFVGVTPWA NAYYASEVSS LAIPSLTGAM ATAAAAVAKV PSFMWLDTLD |
| | | | KTPLMEQTLA DIRTANKNGG NYAGQFVVYD LPDRDCAALA SNGEYSIADG GVAKYKNYID TIRQIVVEYS |
| | | | DIRTLLVIEP DSLANLVTNL STPKCANAQS AYLECINYAV TQLNLPNVAM YLDAGHAGWL GWPANQDPAA |
| | | | QLFANVYKNA SSPRALRGLA TNVASYNGWN ITSPPSYTQG NAVYNEKLYI HAIGPLLANH GWSNAFFITD |
| | | | QGRSGKQPTG QQQWGDWCNV IGTGFGIRPS ANTGDSLLDS FVWVKPGGEC DGTSDSSAPQ FDPHCALPDA |
| | | | LQPAPQAGAW FQAYFVQLLT NANPSFL |
| SEQ ID NO: 60 | US8110389-0037 | Trichoderma | QACSSVWGQC GGQNWSGPTC CASGSTCVYS NDYYSQCLPG AASSSSSTRA ASTTSRVSPT TSRSSSATPP |
| | | reesei | PGSTTTRVPP VGSGTATYSG NPFVGVTPWA NAYYASEVSS LAIPSLTGAM ATAAAAVAEV PSFMWLDTLD |
| | | | KTPLMEQTLA DIRTANKNGG NYAGQFVVYD LPDRDCAALA SNGEYSIADG GVAKYKNYID TIRQIVVEYS |
| | | | DIRTLLVIEP DSLANLVTNL GTPKCANAQS AYLECINYAV TQLNLPNVAM YLDAGHAGWL GWPANQDPAA |
| | | | QLFANVYKNA SSPRALRGLA TNVANYNGWN ITSPPSYTQG NAVYNEKLYI HAIGPLLANH GWSNAFFITD |
| | | | QGRSGKQPTG QQQWGDWCNV IGTGFGIRPS ANTGDSLLDS FVWVKPGGEC DGTSDSSAPR FDPHCALPDA |
| | | | LQPAPQAGAW FQAYFVQLLT NANPSFL |
| SEQ ID NO: 61 | US8110389-0038 | Trichoderma | QACSSVWGQC GGQNWSGPTC CASGSTCVYS NDYYSQCLPG AASSSSSTRA ASTTSRVSPT TSRSSSATPP |
| | | reesei | PGSTTTRVPP VGSGTATYSG NPFVGVTPWA NAYYASEVSS LAIPSLTGAM ATAAAAVAKV PSFMWLDTLD |
| | | | KTPLMEQTLA DIRTANKNGG NYAGQFVVYD LPDRDCAALA SNGEYSIADG GVAKYKNYID TIRQIVVEYS |
| | | | DIRTLLVIEP DSLANLVTNL GTPKCANAQS AYLECINYAV TQLNLPNVAM YLDAGHAGWL GWPANQDPAA |
| | | | QLFANVYKNA SSPRALRGLA TNVANYNGWN ITSPPSYTQG NAVYNEKLYI HAIGPLLANH GWSNAFFITD |
| | | | QGRSGKQPTG QQQWGDWCNV IGTGFGIRPS ANTGDSLLDS FVWVKPGGEC DGTSDSSAPR FDPHCALPDA |
| | | | LQPAPQAGAW FQAYFVQLLT NANPSFL |
| SEQ ID NO: 62 | US8110389-0039 | Trichoderma | QACSSVWGQC GGQNWSGPTC CASGSTCVYS NDYYSQCLPG AASSSSSTRA ASTTSRVSPT TSRSSSATPP |
| | | reesei | PGSTTTRVPP VGSGTATYSG NPFVGVTPWA NAYYASEVSS LAIPSLTGAM ATAAAAVAKV PSFMWLDTLD |
| | | | KTPLMEQTLA DIRTANKNGG NYAGQFVVYD LPDRDCAALA SNGEYSIADG GVAKYKNYID TIRQIVVEYS |
| | | | DIRTLLVIEP DSLANLVTNL GTPKCANAQS AYLECINYAV TQLNLPNVAM YLDAGHAGWL GWPANQDPAA |
| | | | QLFANVYKNA SSPRALRGLA TNVANYNGWN ITSPPSYTQG NDVYNEKLYI HAIGPLLANH GWSNAFFITD |
| | | | QGRSGKQPTG QQQWGDWCNV IGTGFGIRPS ANTGDSLLDS FVWVKPGGEC DGTSDSSAPR FDPHCALPDA |
| | | | LQPAPQAGAW FQAYFVQLLT NANPSFL |
| SEQ ID NO: 63 | US8110389-0040 | Trichoderma | QACSSVWGQC GGQNWSGPTC CASGSTCVYS NDYYSQCLPG AASSSSSTRA ASTTSRVSPT TSRSSSATPP |
| | | reesei | PGSTTTRVPP VGSGTATYSG NPFVGVTPWA NAYYASEVSS LAIPSLTGAM ATAAAAVAKV PSFMWLDTLD |
| | | | KTPLMEQTLA DIRTANKNGG NYAGQFVVYD LPDRDCAALA SNGEYSIADG GVAKYKNYID TIRQIVVEYS |
| | | | DIRTLLVIEP DSLANLVTNL GTPKCANAQS AYLECINYAV TQLNLPNVAM YLDAGHAGWL GWPANQDPAA |
| | | | QLFANVYKNA SSPRALRGLA TNVANYNGWN ITSPPSYTQG NAVYNEKLYI HAIGPLLANH GWSNAFFITD |
| | | | QGRSGKQPTG QQEWGDWCNV IGTGFGIRPS ANTGDSLLDS FVWVKPGGEC DGTSDSSAPR FDPHCALPDA |
| | | | LQPAPQAGAW FQAYFVQLLT NANPSFL |
| SEQ ID NO: 64 | US8110389-0041 | Trichoderma | QACSSVWGQC GGQNWSGPTC CASGSTCVYS NDYYSQCLPG AASSSSSTRA ASTTSRVSPT TSRSSSATPP |
| | | reesei | PGSTTTRVPP VGSGTATYSG NPFVGVTPWA NAYYASEVSS LAIPSLTGAM ATAAAAVAKV PSFMWLDTLD |
| | | | KTPLMEQTLA DIRTANKNGG NYAGQFVVYD LPDRDCAALA SNGEYSIADG GVAKYKNYID TIRQIVVEYS |
| | | | DIRTLLVIEP DSLANLVTNL GTPKCANAQS AYLECINYAV TQLNLPNVAM YLDAGHAGWL GWPANQDPAA |
| | | | QLFANVYKNA SSPRALRGLA TNVANYNGWN ITSPPSYTQG NAVYNEKLYI HAIGPLLANH GWSNAFFITD |
| | | | QGRSGKQPTG QQQWGDWCNV IGTGFGIRPS ANTGDSLLDS FVWVKPGGEC DGTSDSSAPG FDPHCALPDA |
| | | | LQPAPQAGAW FQAYFVQLLT NANPSFL |
| SEQ ID NO: 65 | US20100016570-0083 | Trichoderma | ASCSSVWGQC GGQNWSGPTC CASGSTCVYS NDYYSQCLPG AASSSSSTRA ASTTSRVSPT TSRSSSATPP |
| | | reesei | PGSTTTRVPP VGSGTATYSG NPFVGVTPWA NAYYASEVSS LAIPSLTGAM ATAAAAVAKV PSFMWLDTLD |
| | | | KTPLMEQTLA DIRTANKNGG NYAGQFVVYD LPDRDCAALA SSGEYSIADG GVAKYKNYID TIRQIVVEYS |
| | | | DIRTLLVIEP DSLANLVTNL GTPKCANAQS AYLECINYAV TQLNLPNVAM YLDAGHAGWL GWPANQDPAA |
| | | | QLFANVYKNA SSPRALRGLA TNVANYNGWN ITSPPSYTQG NAVYNEKLYI HAIGPLLANH GWSNAFFITD |
| | | | QGRSGKQPTG QQQWGDWCNV IGTGFGIRPS ANTGDSLLDS FVWVKPGGEC DGTSDSSAPR FDPHCALPDA |
| | | | LQPAPQAGAW FQAYFVQLLT NANPSFL |
| SEQ ID NO: 66 | US20100016570-0085 | Trichoderma | ASCSSVWGQC GGQNWSGPTC CASGSTCVYS NDYYSQCLPG AASSSSSTRA ASTTSRVSPT TSRSSSATPP |
| | | reesei | PGSTTTRVPP VGSGTATYSG NPFVGVTPWA NAYYASEVSS LAIPSLTGAM ATAAAAVAKV PSFMWLDTLD |
| | | | KTPLMEQTLA DIRTANKNGG NYAGQFVVYD LPDRDCAALA SNGEYSIADG GVAKYKNYID TIRQIVVEYS |
| | | | DIRTLLVIEP DSLANLVTNL GTPKCANAQS AYLECINYAV TQLNLPNVAM YLDAGHAGWL GWPANQDPAA |
| | | | QLFANVYKNA SSPRALRGLA TNVANYNGWN ITSPPSYTQG NAVYNEKLYI HAIGPLLANH GWSNAFFITD |
| | | | QGRSGKQPTG QQQWGDWCNV IGTGFGIRPS ANTGDSLLDS FVWVKPGGEC DGTSDSSAPR FDPHCALPDA |
| | | | LQPAPQAGAW FQAYFVQLLT NANPSFL |
| SEQ ID NO: 67 | US20100016570-0088 | Trichoderma | ASCSSVWGQC GGQNWSGPTC CASGSTCVYS NDYYSQCLPG AASSSSSTRA ASTTSRVSPT TSRSSSATPP |
| | | reesei | PGSTTTRVPP VGSGTATYSG NPFVGVTPWA NAYYASEVSS LAIPSLTGAM ATAAAAVAKV PSFMWLDTLD |
| | | | KTPLMEQTLA DIRTANKNGG NYAGQFVVYD LPDRDCAALA SNGEYSIADG GVAKYKNYID TIRQIVVEYS |
| | | | DIRTLLVIEP DSLANLVTNL GTPKCANAQS AYLECINYAV TQLNLPNVAM YLDAGHAGWL GWPANQDPAA |
| | | | QLFANVYKNA SSPRALRGLA TNVANYNGWN ITSPPSYTQG NAVYNEKLYI HAIGPLLANH GWSNAFFITD |
| | | | QGRSGKQPTG QQQWGDWCNV IGTGFGIRPS ANTGDSLLDS FVWVKPGGTC DGTSDSSAPR FDPHCALPDA |
| | | | LQPAPQAGAW FQAYFVQLLT NANPSFL |
| SEQ ID NO: 68 | US20100016570-0089 | Trichoderma | ASCSSVWGQC GGQNWSGPTC CASGSTCVYS NDYYSQCLPG AASSSSSTRA ASTTSRVSPT TSRSSSATPP |
| | | reesei | PGSTTTRVPP VGSGTATYSG NPFVGVTPWA NAYYASEVSS LAIPSLTGAM ATAAAAVAKV PSFMWLDTLD |
| | | | KTPLMEQTLA DIRTANKNGG NYAGQFVVYD LPDRDCAALA SNGEYSIADG GVAKYKNYID TIRQIVVEYS |
| | | | DIRTLLVIEP DSLANLVTNL GTPKCANAQS AYLECINYAV TQLNLPNVAM YLDAGHAGWL GWPANQDPAA |
| | | | QLFANVYKNA SSPRALRGLA TNVANYNGWN ITSPPSYTQG NAVYNEKLYI HAIGPLLANH GWSNAFFITD |
| | | | QGRSGKQPTG QQQWGDWCNV IGTGFGIRPS ANTGDSLLDS FVWVKPGGSC DGTSDSSAPR FDPHCALPDA |
| | | | LQPAPQAGAW FQAYFVQLLT NANPSFL |
| SEQ ID NO: 69 | US20100016570-0094 | Trichoderma | ASCSSVWGQC GGQNWSGPTC CASGSTCVYS NDYYSQCLPG AASSSSSTRA ASTTSRVSPT TSRSSSATPP |
| | | reesei | PGSTTTRVPP VGSGTATYSG NPFVGVTPWA NAYYASEVSS LAIPSLTGAM ATAAAAVAKV PSFMWLDTLD |
| | | | KTPLMEQTLA DIRTANKNGG NYAGQFVVYD LPDRDCAALA SNGEYSIADG GVAKYKNYID TIRQIVVEYS |
| | | | DIRTLLVIEP DSLANLVTNL GTPKCANAQS AYLECINYAV TQLNLPNVAM YLDAGHAGWL GWPANQDPAA |
| | | | QLFANVYKNA SSPRALRGLA TNVANYNGWN ITSPPSYTQG NAVYNEKLYI HAIGPLLANH GWSNAFFITD |
| | | | QGRSGKQPTG QQQWGDWCNV IGTGFGIRPS ANTGDSLLDS FVWVKPGGEC DGTSDSSAPR FDPHCALPDA |
| | | | LQPAPQVGAW FQAYFVQLLT NANPSFL |
| SEQ ID NO: 70 | US20100016570-0095 | Trichoderma | ASCSSVWGQC GGQNWSGPTC CASGSTCVYS NDYYSQCLPG AASSSSSTRA ASTTSRVSPT TSRSSSATPP |
| | | reesei | PGSTTTRVPP VGSGTATYSG NPFVGVTPWA NAYYASEVSS LAIPSLTGAM ATAAAAVAKV PSFMWLDTLD |
| | | | KTPLMEQTLA DIRTANKNGG NYAGQFVVYD LPDRDCAALA SNGEYSIADG GVAKYKNYID TIRQIVVEYS |
| | | | DIRTLLVIEP DSLANLVTNL GTPKCANAQS AYLECINYAV TQLNLPNVAM YLDAGHAGWL GWPANQDPAA |
| | | | QLFANVYKNA SSPRALRGLA TNVANYNGWN ITSPPSYTQG NAVYNEKLYI HAIGPLLANH GWSNAFFITD |
| | | | QGRSGKQPTG QQQWGDWCNV IGTGFGIRPS ANTGDSLLDS FVWVKPGGEC DGTSDSSAPR FDPHCALPDA |
| | | | LQPAPQLGAW FQAYFVQLLT NANPSFL |
| SEQ ID NO: 71 | US20100016570-0096 | Trichoderma | ASCSSVWGQC GGQNWSGPTC CASGSTCVYS NDYYSQCLPG AASSSSSTRA ASTTSRVSPT TSRSSSATPP |
| | | reesei | PGSTTTRVPP VGSGTATYSG NPFVGVTPWA NAYYASEVSS LAIPSLTGAM ATAAAAVAKV PSFMWLDTLD |
| | | | KTPLMEQTLA DIRTANKNGG NYAGQFVVYD LPDRDCAALA SNGEYSIADG GVAKYKNYID TIRQIVVEYS |
| | | | DIRTLLVIEP DSLANLVTNL GTPKCANAQS AYLECINYAV TQLNLPNVAM YLDAGHAGWL GWPANQDPAA |
| | | | QLFANVYKNA SSPRALRGLA TNVANYNGWN ITSPPSYTQG NAVYNEKLYI HAIGPLLANH GWSNAFFITD |
| | | | QGRSGKQPTG QQQWGDWCNV IGTGFGIRPS ANTGDSLLDS FVWVKPGGEC DGTSDSSAPR FDPHCALPDA |
| | | | LQPAPQSGAW FQAYFVQLLT NANPSFL |
| SEQ ID NO: 72 | US20100221778-0010 | Trichoderma | QACSSVWGQC GGQNWSGPTC CASGSTCVYS NDYYFQCLPG AASSSSSTRA ASTTSRVSPT TSRSSSATPP |
| | | reesei | PGSTTTRVPP VGSGTATYSG NPFVGVTPWA NAYYASEVSS LAIPSLTGAM ATAAAAVAKV PSFMWLDTLD |
| | | | KTPLMEQTLA DIRTANKNGG NYAGQFVVYD LPDRDCAALA SNGEYSIADG GVAKYKNYID TIRQIVVEYS |
| | | | DIRTLLVIEP DSLANLVTNL GTPKCANAQS AYLECINYAV TQLNLPNVAM YLDAGHAGWL GWPANQDPAA |
| | | | QLFANVYKNA SSPRALRGLA TNVANYNGWN ITSPPSYTQG NAVYNEKLYI HAIGPLLANH GWSNAFFITD |
| | | | QGRSGKQPTG QQQWGDWCNV IGTGFGIRPS ANTGDSLLDS FVWVKPGGEC DGTSDSSAPR FDPHCALPDA |
| | | | LQPAPQAGAW FQAYFVQLLT NANPSFL |
| SEQ ID NO: 73 | US20100221778-0011 | Trichoderma | QACSSVWGQC GGQNWSGPTC CASGSTCVYS NDYYFQCLPG AASSSSSTRA ASTTSRVSPT TSRSSSATPP |
| | | reesei | PGSTTTRVPP VGSGTATYSG NPFVGVTPWA NAYYASEVSS LAIPSLTGAM ATAAAAVAKV PSFMWLDTLD |
| | | | KTPLMEQTLA DIRTANKNGG NYAGQFVVYD LPDRDCAALA SNGEYSIADG GVAKYKNYID TIRQIVVEYS |
| | | | DIRTLLVIEP DSLANLVTNL GTPKCANAQS AYLECINYAV TQLNLPNVAM YLDAGHAGWL GWPANQDPAA |
| | | | QLFANVYKNA SSPRALRGLA TNVANYNGWN ITSPPSYTQG NAVYNEKLYI HAIGPLLANH GWSNAFFITD |
| | | | QGRSGKQPTG QQQWGDWCNV IGTGFGIRPS ANTGDSLLDS FVWVKPGGEC DGTSDSSAPR FDPHCALPDA |
| | | | LQPAPQAGAW FQAYFVQLLT NANPSFL |
| SEQ ID NO: 74 | US20100221778-0012 | Trichoderma | QACSSVWGQC GGQNWSGPTC CASGSTCVYS NDYYFQCLPG AASSSSSTRA ASTTSRVSPT TSRSSSATPP |
| | | reesei | PGSTTTRVPP VGSGTATYSG NPFVGVTPWA NAYYASEVSS LAIPSLTGAM ATAAAAVAKV PSFMWLDTLD |
| | | | KTPLMEQTLA DIRTANKNGG NYAGQFVVYD LPDRDCAALA SNGEYSIADG GVAKYKNYID TIRQIVVEYS |
| | | | DIRTLLVIEP DSLANLVTNL GTPKCANAQS AYLECINYAV TQLNLPNVAM YLDAGHAGWL GWPANQDPAA |
| | | | QLFANVYKNA SSPRALRGLA TNVANYNGWN ITSPPSYTQG NAVYNEKLYI HAIGPLLANH GWSNAFFITD |
| | | | QGRSGKQPTG QQQWGDWCNV IGTGFGIRPS ANTGDSLLDS FVWVKPGGEC DGTSDSSAPR FDPHCALPDA |
| | | | LQPAPQAGAW FQAYFVQLLT NANPSFL |
| SEQ ID NO: 75 | US20100221778-0013 | Trichoderma | QACSSVWGQC GGQNWSGPTC CASGSTCVYS NDYYFQCLPG AASSSSSTRA ASTTSRVSPT TSRSSSATPP |
| | | reesei | PGSTTTRVPP VGSGTATYSG NPFVGVTPWA NAYYASEVSS LAIPSLTGAM ATAAAAVAKV PSFMWLDTLD |
| | | | KTPLMEQTLA DIRTANKNGG NYAGQFVVYD LPDRDCAALA SNGEYSIADG GVAKYKNYID TIRQIVVEYS |
| | | | DIRTLLVIEP DSLANLVTNL GTPKCANAQS AYLECINYAV TQLNLPNVAM YLDAGHAGWL GWPANQDPAA |
| | | | QLFANVYKNA SSPRALRGLA TNVANYNGWN ITSPPSYTQG NAVYNEKLYI HAIGPLLANH GWSNAFFITD |
| | | | QGRSGKQPTG QQQWGDWCNV IGTGFGIRPS ANTGDSLLDS FVWVKPGGEC DGTSDSSAPR FDPHCALPDA |
| | | | LQPAPQAGAW FQAYFVQLLT NANPSFL |
| SEQ ID NO: 76 | US20100221778-0014 | Trichoderma | QACSSVWGQC GGQNWSGPTC CASGSTCVYS NDYYFQCLPG AASSSSSTRA ASTTSRVSPT TSRSSSATPP |
| | | reesei | PGSTTTRVPP VGSGTATYSG NPFVGVTPWA NAYYASEVSS LAIPSLTGAM ATAAAAVAKV PSFMWLDTLD |
| | | | KTPLMEQTLA DIRTANKNGG NYAGQFVVYD LPDRDCAALA SNGEYSIADG GVAKYKNYID TIRQIVVEYS |
| | | | DIRTLLVIEP DSLANLVTNL GTPKCANAQS AYLECINYAV TQLNLPNVAM YLDAGHAGWL GWPANQDPAA |
| | | | QLFANVYKNA SSPRALRGLA TNVANYNGWN ITSPPSYTQG NAVYNEKLYI HAIGPLLANH GWSNAFFITD |
| | | | QGRSGKQPTG QQQWGDWCNV IGTGFGIRPS ANTGDSLLDS FVWVKPGGEC DGTSDSSAPR FDPHCALPDA |
| | | | LQPAPQAGAW FQAYFVQLLT NANPSFL |
| SEQ ID NO: 77 | US20100221778-0015 | Trichoderma | QACSSVWGQC GGQNWSGPTC CASGSTCVYS NDYYFQCLPG AASSSSSTRA ASTTSRVSPT TSRSSSATPP |
| | | reesei | PGSTTTRVPP VGSGTATYSG NPFVGVTPWA NAYYASEVSS LAIPSLTGAM ATAAAAVAKV PSFMWLDTLD |
| | | | KTPLMEQTLA DIRTANKNGG NYAGQFVVYD LPDRDCAALA SNGEYSIADG GVAKYKNYID TIRQIVVEYS |
| | | | DIRTLLVIEP DSLANLVTNL GTPKCANAQS AYLECINYAV TQLNLPNVAM YLDAGHAGWL GWPANQDPAA |
| | | | QLFANVYKNA SSPRALRGLA TNVANYNGWN ITSPPSYTQG NAVYNEKLYI HAIGPLLANH GWSNAFFITD |
| | | | QGRSGKQPTG QQQWGDWCNV IGTGFGIRPS ANTGDSLLDS FVWVKPGGEC DGTSDSSAPR FDPHCALPDA |
| | | | LQPAPQAGAW FQAYFVQLLT NANPSFL |
| SEQ ID NO: 78 | US20100221778-0017 | Trichoderma | QACSSVWGQC GGQNWSGPTC CASGSTCVYS NDYYFQCLPG AASSSSSTRA ASTTSRVSPT TSRSSSATPP |
| | | reesei | PGSTTTRVPP VGSGTATYSG NPFVGVTPWA NAYYASEVSS LAIPSLTGAM ATAAAAVAKV PSFMWLDTLD |
| | | | KTPLMEQTLA DIRTANKNGG NYAGQFVVYD LPDRDCAALA SNGEYSIADG GVAKYKNYID TIRQIVVEYS |
| | | | DIRTLLVIEP DSLANLVTNL GTPKCANAQS AYLECINYAV TQLNLPNVAM YLDAGHAGWL GWPANQDPAA |
| | | | QLFANVYKNA SSPRALRGLA TNVANYNGWN ITSPPSYTQG NAVYNEKLYI HAIGPLLANH GWSNAFFITD |
| | | | QGRSGKQPTG QQQWGDWCNV IGTGFGIRPS ANTGDSLLDS FVWVKPGGEC DGTSDSSAPR FDPHCALPDA |
| | | | LQPAPQAGAW FQAYFVQLLT NANPSFL |
| SEQ ID NO: 79 | EP2401370-0016 | Hypocreajeunina | QACSSVWGQC GGQNWSGPTC CASGSTCVYS NDYYFQCLPG AASSSSSTRA ASTTSRVSPT TSRSSSATPP |
| | | | PGSTTTRVPP VGSGTATYSG NPFVGVTPWA NAYYASEVSS LAIPSLTGAM ATAAAAVAKV PSFMWLDTLD |
| | | | KTPLMEQTLA DIRTANKNGG NYAGQFVVYD LPDRDCAALA SNGEYSIADG GVAKYKNYID TIRQIVVEYS |
| | | | DIRTLLVIEP DSLANLVTNL GTPKCANAQS AYLECINYAV TQLNLPNVAM YLDAGHAGWL GWPANQDPAA |
| | | | QLFANVYKNA SSPRALRGLA TNVANYNGWN ITSPPSYTQG NAVYNEKLYI HAIGPLLANH GWSNAFFITD |
| | | | QGRSGKQPTG QQQWGDWCNV IGTGFGIRPS ANTGDSLLDS FVWVKPGGEC DGTSDSSAPR FDPHCALPDA |
| | | | LQPAPQAGAW FQAYFVQLLT NANPSFL |
| SEQ ID NO: 80 | JP2011523854-0016 | synthetic | QACSSVWGQC GGQNWSGPTC CASGSTCVYS NDYYFQCLPG AASSSSSTRA ASTTSRVSPT TSRSSSATPP |
| | | construct | PGSTTTRVPP VGSGTATYSG NPFVGVTPWA NAYYASEVSS LAIPSLTGAM ATAAAAVAKV PSFMWLDTLD |
| | | | KTPLMEQTLA DIRTANKNGG NYAGQFVVYD LPDRDCAALA SNGEYSIADG GVAKYKNYID TIRQIVVEYS |
| | | | DIRTLLVIEP DSLANLVTNL GTPKCANAQS AYLECINYAV TQLNLPNVAM YLDAGHAGWL GWPANQDPAA |
| | | | QLFANVYKNA SSPRALRGLA TNVANYNGWN ITSPPSYTQG NAVYNEKLYI HAIGPLLANH GWSNAFFITD |
| | | | QGRSGKQPTG QQQWGDWCNV IGTGFGIRPS ANTGDSLLDS FVWVKPGGEC DGTSDSSAPR FDPHCALPDA |
| | | | LQPAPQAGAW FQAYFVQLLT NANPSFL |
| SEQ ID NO: 81 | US20100317087-0018 | Trichoderma | QACSSVWGQC GGQNWSGPTC CASGSTCVYS NDYYFQCLPG AASSSSSTRA ASTTSRVSPT TSRSSSATPP |
| | | reesei | PGSTTTRVPP VGSGTATYSG NPFVGVTPWA NAYYASEVSS LAIPSLTGAM ATAAAAVAKV PSFMWLDTLD |
| | | | KTPLMEQTLA DIRTANKNGG NYAGQFVVYD LPDRDCAALA SNGEYSIADG GVAKYKNYID TIRQIVVEYS |
| | | | DIRTLLVIEP DSLANLVTNL GTPKCANAQS AYLECINYAV TQLNLPNVAM YLDAGHAGWL GWPANQDPAA |
| | | | QLFANVYKNA SSPRALRGLA TNVANYNGWN ITSPPSYTQG NAVYNEKLYI HAIGPLLANH GWSNAFFITD |
| | | | QGRSGKQPTG QQQWGDWCNV IGTGFGIRPS ANTGDSLLDS FVWVKPGGEC DGTSDSSAPR FDPHCALPDA |
| | | | LQPAPQAGAW FQAYFVQLLT NANPSFL |
| SEQ ID NO: 82 | US20110189744-0041 | Artificial | VPLEERQACS SVWGQCGGQN WSGPTCCASG STCVYSNDYY SQCLPGAASS SSSTRAASTT SRVSPTTSRS |
| | | Sequence | SSATPPPGST TTRVPPVGSG TATYSGNPFV GVTPWANAYY ASEVSSLAIP SLTGAMATAA AAVAKVPSFM |
| | | | WLDTLDKTPL MEQTLADIRT ANKNGGNYAG QFVVYDLPDR DCAALASNGE YSIADGGVAK YKNYIDTIRQ |
| | | | IVVEYSDIRT LLVIEPDSLA NLVTNLGTPK CANAQSAYLE CINYAVTQLN LPNVAMYLDA GHAGWLGWPA |
| | | | NQDPAAQLFA NVYKNASSPR ALRGLATNVA NYNGWNITSP PSYTQGNAVY NEKLYIHAIG PLLANHGWSN |
| | | | AFFITDQGRS GKQPTGQQQW GDWCNVIGTG FGIRPSANTG DSLLDSFVWV KPGGECDGTS DSSAPRFDSH |
| | | | CALPDALQPA PQAGAWFQAY FVQLLTNANP SFLGSGGGGS GGGGSHHHHH HGGENLYFQG GGGGSGGGGS |
| | | | GSA |
| SEQ ID NO: 83 | AAQ72468 | Gibberella | MTAYKLFLAA AFAATALAAP VEERQSCSNG VWSQCGGQNW SGTPCCTSGN KCVKVNDFYS QCQPGSADPS |
| | | zeae | PTSTIVSATT TKATTTGSGG SVTSPPPVAT NNPFSGVDLW ANNYYRSEVS TLAIPKLSGA MATAAAKVAD |
| | | | VPSFQWMDTY DHISFMEDSL ADIRKANKAG GNYAGQFVVY DLPDRDCAAA ASNGEYSLDK DGKNKYKAYI |
| | | | ADQGILQDYS DTRIILVIEP DSLANMVTNM NVPKCANAAS AYKELTIHAL KELNLPNVSM YIDAGHGGWL |
| | | | GWPANLPPAA QLYGQLYKDA GKPSRLRGLV TNVSNYNAWK LSSKPDYTES NPNYDEQKYI HALSPLLEQE |
| | | | GWPGAKFIVD QGRSGKQPTG QKAWGDWCNA PGTGFGLRPS ANTGDALVDA FVWVKPGGES DGTSDTSAAR |
| | | | YDYHCGIDGA VKPAPEAGTW FQAYFEQLLK NANPSFL |
| SEQ ID NO: 84 | AAA65585 | Fusarium | MAYKLILAAF AATALAAPVE ERQSCSNGVW AQCGGQNWSG TPCCTSGNKC VKLNDFYSQC QPGSAEPSST |
| | | oxysporum | AAGPSSTTAT KTTATGGSST TAGGSVTSAP PAASDNPYAG VDLWANNYYR SEVMNLAVPK LSGAKATAAA |
| | | | KVADVPSFQW MDTYDHISLM EDTLADIRKA NKAGGKYAGQ FVVYDLPNRD CAAAASNGEY SLDKDGANKY |
| | | | KAYIAKIKGI LQNYSDTKVI LVIEPDSLAN LVTNLNVDKC AKAESAYKEL TVYAIKELNL PNVSMYLDAG |
| | | | HGGWLGWPAN IGPAAKLYAQ IYKDAGKPSR VRGLVTNVSN YNGWKLSTKP DYTESNPNYD EQRYINAFAP |
| | | | LLAQEGWSNV KFIVDQGRSG KQPTGQKAQG DWCNAKGTGF GLRPSTNTGD ALADAFVWVK PGGESDGTSD |
| | | | TSAARYDYHC GLDDALKPAP EAGTWFQAYF EQLLDNANPS FL |
| SEQ ID NO: 85 | ADC83999 | Trichoderma | MIVGILTTLA TLATLAASVP LEERQACSSV WGQCGGQNWS GPTCCASGST CVYSNDYYSQ CLPGAASSSS |
| | | reesei | STRAASTTSR VSPTTSRSSS ATPPPGSTTT RVPPVGSGTA TYSGNPFVGV TPWANAYYAS EVSSLAIPSL |
| | | | TGAMATAAAA VAKVPSFMWL DTLDKTPLME QTLADIRTAN KNGGNYAGQF VVYDLPDRDC AALASNGEYS |
| | | | IADGGVAKYK NYIDTIRQIV VEYSDIRTLL VIEPDSLANL VTNLGTPKCA NAQSAYLECI NYAVTQLNLP |
| | | | NVAMYLDAGH AGWLGWPANQ DPAAQLFANV YKNASSPRAL RGLATNVANY NGWNITSPPS YTQGNAVYNE |
| | | | KLYIHAIGPL LANHGWSNAF FITDQGRSGK QPTGQQQWGD WCNVTGTGFG IRPSANTGDS LLDSFVWVKP |
| | | | GGECDGTSDS SAPRFDSHCA LPDALQPAPQ AGAWFQAYFV QLLTNANPSF L |
| SEQ ID NO: 86 | AAA34210 | Trichoderma | MIVGILTTLA TLATLAASVP LEERQACSSV WGQCGGQNWS GPTCCASGST CVYSNDYYSQ CLPGAASSSS |
| | | reesei | STRAASTTSR VSPTTSRSSS ATPPPGSTTT RVPPVGSGTA TYSGNPFVGV TPWANAYYAS EVSSLAIPSL |
| | | | TGAMATAAAA VAKVPSFMWL DTLDKTPLME QTLADIRTAN KNGGNYAGQF VVYDLPDRDC AALASNGEYS |
| | | | IADGGVAKYK NYIDTIRQIV VEYSDIRTLL VIEPDSLANL VTNLGTPKCA NAQSAYLECI NYAVTQLNLP |
| | | | NVAMYLDAGH AGWLGWPANQ DPAAQLFANV YKNASSPRAL RGLATNVANY NGWNITSPPS YTQGNAVYNE |
| | | | KLYIHAIGPL LANHGWSNAF FITDQGRSGK QPTGQQQWGD WCNVIGTGFG IRPSANTGDS LLDSFVWVKP |
| | | | GGECDGTSDS SAPRFDSHCA LPDALQPAPQ AGAWFQAYFV QLLTNANPSF L |
| SEQ ID NO: 87 | AAQ76094 | Hypocrearufa | MIVGILTTLA TLATLAASVP LEERQACSSV WGQCGGQNWS GPTCCASGST CVYSNDYYSQ CLPGAASSSS |
| | | | STRAASTTSR VSPTTSRSSS ATPPPGSTTT RVPPVGSGTA TYSGNPFVGV TPWANAYYAS EVSSLAIPSL |
| | | | TGAMATAAAA VAKVPSFMWL DTLDKTPLME QTLADIRTAN KNGGNYAGQF VVYDLPDRDC AALASNGEYS |
| | | | IADGGVAKYK NYIDTIRQIV VEYSDIRTLL VIEPDSLANL VTNLGTPKCA NAPSAYLECI NYAVTQLNLP |
| | | | NVAMYLDAGH AGWLGWPANQ DPAAQLFANV YKNASSPRAL RGLATNVANY NGWNITSPPS YTQGNAVYNE |
| | | | KLYIHAIGPL LANHGWSNAF FITDQGRSGK QPTGQQQWGD WCNVIGTGFG IRPSANTGDS LLDSFVWVKP |
| | | | GGECDGTSDS SAPRFDSHCA LPDALQPAPQ AGAWFQAYFV QLLTNANPSF L |
| SEQ ID NO: 88 | AAG39980 | Trichoderma | MIVGILTTLA TLATLAASVP LEERQACSSV WGQCGGQNWS GPTCCASGST CVYSNDYYSQ CLPGAASSSS |
| | | reesei | STRAASTTSR VSPTTSRSSS ATPPPGSTTT RVPPVGSGTA TYSGNPFVGV TPWANAYYAS EVSSLAIPSL |
| | | | TGAMATAAAA VAKVPSFMWL DTLDKTPLME QTLADIRTAN KNGGNYAGQF VVYDLPDRDC AALASNGEYS |
| | | | IADGGVAKYK NYIDTIRQIV VEYSDIRTLL VIEPDSLANL VTNLGTPKCA NAQSAYLECI NYAVTQLNLP |
| | | | NVAMYLDAGH AGWLGWPANQ DPAAQLFANV YKNASSPRAL RGLATNVANY NGWNITSPPS YTQGNAVYNE |
| | | | KLYIHAIGRL LANHGWSNAF FITDQGRSGK QPTGQQQWGD WCNVIGTGFG IRPSANTGDS LLDSFVWVKP |
| | | | GGECDGTSDS SAPRFDSHCA LPDALQPAPQ AGAWFQAYFV QLLTNANPSF L |
| SEQ ID NO: 89 | AAA72922 | Trichoderma | MIVGILTTLA TLATLAASVP LEERQACSSV WGQCGGQNWS GPTCCASGST CVYSNDYYSQ CLPGAASSSS |
| | | reesei | STRAASTTSR VSPTTSRSSS ATPPPGSTTT RVPPVGSGTA TYSGNPFVGV TPWANAYYAS EVSSLAIPSL |
| | | | TGAMATAAAA VAKVPSFMWL DTLDKTPLME QTLADIRTAN KNGGNYAGQF VVYDLPDRDC AALASNGEYS |
| | | | IADGGVAKYK NYIDTIRQIV VEYSDIRTLL VIEPDSLANL VTNLGTPKCA NAQSAYLECI NYAVTQLNLP |
| | | | NVAMYLDAGH AGWLGWPANQ DPAAQLFANV YKNASSPRAL RGLATNVANY NGWNITSPPS YTQGNAVYNE |
| | | | KLYIHAIGRL LANHGWSNAF FITDQGRSGK QPTGQQQWGD WCNVIGTGFG IRPSANTGDS LLDSFVWVKP |
| | | | GGECDGTSDS SAPRFDSHCA LPDALQPAAQ AGAWFQAYFV QLLTNANPSF L |
| SEQ ID NO: 90 | ABF56208 | Hypocrea | MIVGILTTLA TLATLAASVP LEERQACSSA WGQCGGQNWS GPTCCASGST CVYSNDYYSQ CLPGAASSSS |
| | | koningii | STRASSTTAR ASSTTSRSSA TPPPGSSTTR VPPVGSGTAT YSGNPFVGVT PWANAYYASE VSSLAIPSLT |
| | | | GAMATAAAAV AKVPSFMWLD TFDKTPLMEQ TLADIRTANK NGGNYAGQFV VYDLPDRDCA ALASNGEYSI |
| | | | ADGGVDKYKN YIDTIRQIVV EYSDIRTLLV IEPDSLANLV TNLGTPKCAN AQSAYLECIN YAVTQLNLPN |
| | | | VAMYLDAGHA GWLGWPANQD PAAQLFANVY KNASSPRALR GLATNVANYN GWNITSPPSY TQGNAVYNEQ |
| | | | LYIHAIGPLL ANHGWSNAFF ITDQGRSGKQ PTGQQQWGDW CNVIGTGFGI RPSANTGDSL LDSFVWIKPG |
| | | | GECDGTSDSS APRFDSHCAL PDALQPAPQA GAWFQAYFVQ LLTNANPSFL |
| SEQ ID NO: 91 | ACZ34301 | Trichoderma | MIVGILTTLA TLATLAASVP LEERQACSSV WGQCGGQNWS GPTCCASGST CVYSNDYYSQ CLPGAASSSS |
| | | longibrachiatum | STRASSTTAR ASSTTSRSSA TPPPGSSTTR VPPVGSGTAT YSGNPFVGVT PWANAYYASE VSSLAIPSLT |
| | | | GAMATAAAAV AKVPSFMWLD TFDKTPLMEQ TLADIRTANK NGGNYAGQFV VYDLPDRDCA ALASNGEYSI |
| | | | ADGGVDKYKN YIDTIRQIVV EYSDIRTLLV IEPDSLANLV TNLGTPKCAN AQSAYLECIN YAVTQLNLPN |
| | | | VAMYLDAGHA GWLGWPANQD PAAQLFANVY KNASSPRALR GLATNVANYN GWNITSPPSY TQGNAVYNEQ |
| | | | LYIHAIGPLL ANHGWSNAFF ITDQGRSGKQ PTGQQQWGDW CNVIGTGFGI RPSANTGDSL LDSFVWIKPG |
| | | | GECDGTSDSS APRFDSHCAL PDALQPAPQA GAWFQAYFVQ LLTNANPSFL |
| SEQ ID NO: 92 | ABG48766 | Hypocrea | MIVGILTTLA TLATLAASVP LEERQACSSV WGQCGGQNWS GPTCCASGST CVYSNDYYSQ CLPGAASSSS |
| | | koningii | STRASSTTAR ASSTTSRSSA TPPPGSSTTR VPPVGSGTAT YSGNPFVGVT PWANAYYASE VSSLAIPSLT |
| | | | GAMATAAAAV AKVPSFMWLD TFDKTPLMEQ TLADIRTANK NGGNYAGQFV VYDLPDRDCA ALASNGEYSI |
| | | | ADGGVDKYKN YIDTIRQIVV EYSDIRTLLV IEPDSLANLV TNLGTPKCAN AQSAYLECIN YAVTQLNLPN |
| | | | VAMYLDAGHA GWLGWPANQD PAAQLFANVY KNASSPRALR GLATNVANYN GWNITSPPSY TQGNAVYNEQ |
| | | | LYIHAIGPLL ANHGWSNAFF ITDQGRSGKQ PTGQQQWGDW CNVIGTGFGI RPSANTGDSL LDSFVWIKPG |
| | | | GECDGTSDSS APRFDSHCAL PDALQPAPQA GAWFQAYFVQ LLTNANPSFL |
| SEQ ID NO: 93 | ACH96126 | Trichoderma | MIVGILTTLA TLATLAASVP LEERQACSSV WGQCGGQNWS GPTCCASGST CVYSNDYYSQ CLPGAASSSS |
| | | sp XSTI | STRASSTTAR ASSTTSRSSA TPPPGSSTTR VPPVGSGTAT YSGNPFVGVT PWANAYYASE VSSLAIPSLT |
| | | | GAMATAAAAV AKVPSFMWLD TFDKTPLMEQ TLADIRTANK NGGNYAGQFV VYDLPDRDCA ALASNGEYSI |
| | | | ADGGVDKYKN YIDTIRQIVV EYSDIRTLLV IEPDSLANLV TNLGTPKCAN AQSAYLECIN YAVTQLNLPN |
| | | | VAMYLDAGHA GWLGWPANQD PAAQLFANVY KNASSPRALR GLATNVANYN GWNITSPPSY TQGNAVYNEQ |
| | | | LYIHAIGPLL ANHGWSNAFF ITDQGRSGKQ PTGHQQWGDW CNVIGTGFGI RPSANTGDSL LDSFVWIKPG |
| | | | GECDGTSDSS APRFDSHCAL PDALQPAPQA GAWFQAYFVQ LLTNANPSFL |
| SEQ ID NO: 94 | ADJ10628 | Hypocrearufa | MIVGILTTLA TLATLAASVP LEERQACSSV WGQCGGQNWS GPTCCASGST CVYSNDYYSQ CLPGAASSSS |
| | | | STRASSTTAR ASSTTSRSSA TPPPGSSTTR VPPVGSGTAT YSGNPFVGVT PWANAYYASE VSSLAIPSLT |
| | | | GAMATAAAAV AKVPSFMWLD TFDKTPLMEQ TLADIRTANK NGGNYAGQFV VYDLPDRDCA ALASNGEYSI |
| | | | ADGGVDKYKN YIDTIRQIVV EYSDIRTLLV IEPDSLANLV TNLGTPKCAN AQFAYLECIN YAVTQLNLPN |
| | | | VAMYLDAGHA GWLGWPANQD PAAQLFANVY KNASSPRALR GLATNVANYN GWNITSPPSY TQGNAVYNEQ |
| | | | LYIHAIGPLL ANHGWSNAFF ITDQGRSGKQ PTGQQQWGDW CNVIGTGFGI RPSANTGDSL LDSFVWIKPG |
| | | | GECDGTSDSS APRFDSHCAL PDALQPAPQA GAWFQAYFVQ FLTNANPSFL |
| SEQ ID NO: 95 | AAU05379 | Trichoderma | MIVGILTTLA TLATLAASVP LEERQACSSV WGQCGGQNWS GPTCCAAGST CVYSNDYYSQ CPPGAASSSS |
| | | parceramosum | STRASSTTNR VSSTTSTSSA TPPPGSSTTR VPPVGSGTAT YSGNPFVGVT PWANAYYASE VSSLAIPSLT |
| | | | GAMATAAAAV AKVPSFMWLD TLDKTPLMEQ TLADIRTANK NGGNYAGQFV VYDLPDRDCA ALASNGEYSI |
| | | | ADGGVAKYKN YIDTIRQIVV EYSDIRTLLV IEPDSLANLV TNLGTPKCAN AQSAYLECIN YAITQLNLPN |
| | | | IAMYLDAGHA GWLGWPANQD PAAQLFANVY KNASSPSALR GLATNVANYN GWNITSPPSY TQGNAVYNEK |
| | | | LYIHAIGPLL ANHGWSNAFF ITDQGRSGKQ PTGQQQWGDW CNVIGTGFGI RPSSNTGDSL LDSFVWVKPG |
| | | | GECDGTSDSS APRFDSHCAL PDALQPAPQA GAWFQAYFVQ LLTNANPSFL |
| SEQ ID NO: 96 | AE062210 | Thielavia | MAQKLLLAAA LAASALAAPV VEERQNCGSV WSQCGGIGWS GATCCASGNT CVELNPYYSQ CLPNSQVTTS |
| | | terrestris | TSKTTSTTTR SSTTSHSSGP TSTSTTTTSS PVVTTPPSTS IPGGASSTAS WSGNPFSGVQ MWANDYYASE |
| | | NRRL 8126 | VSSLAIPSMT GAMATKAAEV AKVPSFQWLD RNVTIDTLFA HTLSQIRAAN QKGANPPYAG IFVVYDLPDR |
| | | | DCAAAASNGE FSIANNGAAN YKTYIDAIRS LVIQYSDIRI IFVIEPDSLA NMVTNLNVAK CANAESTYKE |
| | | | LTVYALQQLN LPNVAMYLDA GHAGWLGWPA NIQPAANLFA EIYTSAGKPA AVRGLATNVA NYNGWSLATP |
| | | | PSYTQGDPNY DESHYVQALA PLLTANGFPA HFITDTGRNG KQPTGQRQWG DWCNVIGTGF GVRPTTNTGL |
| | | | DIEDAFVWVK PGGECDGTSN TTSPRYDYHC GLSDALQPAP EAGTWFQAYF EQLLTNANPP F |
| SEQ ID NO: 97 | CBX74420 | Synthetic | MRVLLVALAL LALAASATSV PLEERQSCSS VWGQCGGQNW AGPFCCASGS TCVYSNDYYS QCLPGTASSS |
| | | construct | SSTRASSTTS RVSSATSTRS SSSTPPPASS TTPAPPVGSG TATYSGNPFA GVTPWANSFY ASEVSTLAIP |
| | | | SLTGPMATKA AAVAKVPSFM WLDTLDKTPL MSSTLSDIRA ANKAGGNYAG QFVVYDLPDR DCAAAASNGE |
| | | | YSIADGGVAK YKNYIDTIRG IVTTFSDVRI LLVIEPDSLA NLVTNLATPK CSNAQSAYLE CINYAITQLN |
| | | | LPNVAMYLDA GHAGWLGWPA NQDPAAQLFA NVYKNASSPR AVRGLATNVA NYNAWNITTP PSYTQGNAVY |
| | | | NEKLYIHALG PLLANHGWSN AFFITDQGRS GKQPTGQLEW GNWCNAVGTG FGIRPSANTG DSLLDSFVWI |
| | | | KPGGECDGTS NSSAPRFDYH CASADALQPA PQAGSWFQAY FEQLLTNANP SFL |
| SEQ ID NO: 98 | AE055787 | Myceliophthora | MAKKLFITAA LAAAVLAAPV IEERQNCGAV WTQCGGNGWQ GPTCCASGST CVAQNEWYSQ CLPNSQVTSS |
| | | thennophila | TTPSSTSTSQ RSTSTSSSTT RSGSSSSSST TPPPVSSPVT SIPGGATSTA SYSGNPFSGV RLFANDYYRS |
| | | ATCC 42464 | EVHNLAIPSM TGTLAAKASA VAEVPSFQWL DRNVTIDTLM VQTLSQVRAL NKAGANPPYA AQLVVYDLPD |
| | | | RDCAAAASNG EFSIANGGAA NYRSYIDAIR KHIIEYSDIR IILVIEPDSM ANMVTNMNVA KCSNAASTYH |
| | | | ELTVYALKQL NLPNVAMYLD AGHAGWLGWP ANIQPAAELF AGIYNDAGKP AAVRGLATNV ANYNAWSIAS |
| | | | APSYTSPNPN YDEKHYIEAF SPLLNSAGFP ARFIVDTGRN GKQPTGQQQW GDWCNVKGTG FGIRPSANTG |
| | | | HELVDAFVWV KPGGESDGTS DTSAARYDYH CGLSDALQPA PEAGQWFQAY FEQLLTNANP PF |
| SEQ ID NO: 99 | AAY88915 | Chaetomium | MAKQLLLTAA LAATSLAAPL LEERQSCSSV WGQCGGINYN GPTCCQSGSV CTYLNDWYSQ CIPGQAQPGT |
| | | thermophilum | TSTTARTTST STTSTSSVRP TTSNTPVTTA PPTTTIPGGA SSTASYNGNP FSGVQLWANT YYSSEVHTLA |
| | | | IPSLSPELAA KAAKVAEVPS FQWLDRNVTV DTLFSGTLAE IRAANQRGAN PPYAGIFVVY DLPDRDCAAA |
| | | | ASNGEWSIAN NGANNYKRYI DRIRELLIQY SDIRTILVIE PDSLANMVTN MNVQKCSNAA STYKELTVYA |
| | | | LKQLNLPHVA MYMDAGHAGW LGWPANIQPA AELFAQIYRD AGRPAAVRGL ATNVANYNAW SIASPPSYTS |
| | | | PNPNYDEKHY IEAFAPLLRN QGFDAKFIVD TGRNGKQPTG QLEWGHWCNV KGTGFGVRPT ANTGHELVDA |
| | | | FVWVKPGGES DGTSDTSAAR YDYHCGLSDA LTPAPEAGQW FQAYFEQLLI NANPPF |
| SEQ ID NO: 100 | CAP60942 | Podospora | MAKRLLLTAA LAATTLAAPV IEERQNCGSV WSQCGGQGWT GATCCASGST CVAQNQWYSQ CLPGSQVTTT |
| | | anserina | AQAPSSTRTT TSSSSRPTSS SISTSAVNVP TTTTSAGASV TVPPGGGASS TASYSGNPFL GVQQWANSYY |
| | | S mat+ | SSEVHTLAIP SLTGPMATKA AAVAKVPSFQ WMDRNVTVDT LFSGTLADIR AANRAGANPP YAGIFVVYDL |
| | | | PDRDCAAAAS NGEWAIADGG AAKYKAYIDR IRHHLVQYSD IRTILVIEPD SLANMVTNMN VPKCQGAANT |
| | | | YKELTVYALK QLNLPNVAMY LDAGHAGWLG WPANIGPAAE LFAGIYKDAG RPTSLRGLAT NVANYNGWSL |
| | | | SSAPSYTTPN PNFDEKRFVQ AFSPLLTAAG FPAHFITDTG RSGKQPTGQL EWGHWCNAIG TGFGPRPTTD |
| | | | TGLDIEDAFV WIKPGGECDG TSDTTAARYD HHCGFADALK PAPEAGQWFQ AYFEQLLTNA NPPF |
| SEQ ID NO: 101 | AAW64927 | Chaetomium | MAKQLLLTAA LAAISLAAPL LEERQSCSSV WGQCGGINYN GPTCCQSGSV CAYLNDWYSQ CIPGQAQPGT |
| | | thermophilum | TSTTARTTST STTSTSSVRP TTSNTPVTTA PPTTTIPGGA SSTASYNGNP FSGVQLWANT YYSSEVHTLA |
| | | | IPSLSPELAA KAAKVAEVPS FQWLDRNVTV DTLFSGTLAE IRAANQRGAN PPYAGIFVVY DLPDRDCAAA |
| | | | ASNGEWSIAN NGANNLQRYI DRIRELLIQY SDIRTILVIE PDSLANMVTN MNVQKCSNAA STYKELTVYA |
| | | | LKQLNLPHVA MYMDAGHAGW LGWPANIQPA AELFAQIYRD AGRPAAVRGL ATNVANYNAW SIASPPSYTS |
| | | | PNPNYDEKHY IEAFAPLLRN QGFDAKFIVD TGRNGKQPTG QLEWGHWCNV KGTGFGVRPT ANTGHELVDA |
| | | | FVWVKPGGES DGTSDTSAAR YDYHCGLSDA LTPAPEAGQW FQAYFEQLLI NANPPF |
| SEQ ID NO: 102 | ADZ99361 | Phialophora | MTAKHVFLAA ALAATALAAP VSESQNCASE WGQCGGTGFT GASCCASGST CTQQNEYYSQ CVPGSQVTTG |
| | | sp CGMCC 3328 | QIASTPAATV VGSATMGSSP SQMTAPAASA SGTTSYSGNP FEGVQMWANA YYASEVLNLA VPSLSGDMVA |
| | | | KASAVAKVPS FQWLDTAAKV PTVMADTLAD IAKANQAGAS PAYAGLFVVY DLPDRDCAAA ASNGEYSIAD |
| | | | NGVANYKAYI DAIKAQLVAN SDTRILLVVE PDSLANLVTN MNVAKCANAH DAYLECINYA VTQLNLPNVA |
| | | | MYLDAGHAGW LGWSANLQPA ATLFANVYSN AGKPASLRGL ATNVANYNAW TIASAPSYTQ GDSNYDEKLY |
| | | | VQALSPLLSS AGWDAHFITD QSRSGKQPTG QNAWGDWCNV IGTGFGTRPT TDTGLDIEDA VWVKPGGECD |
| | | | GTSNTTAARY DYHCGLSDAL QPAPEAGTWF QAYFVQLLQN ANPAF |
| SEQ ID NO: 103 | CAD70733 | Neurosporacrassa | MAAKKLLLAA ALTASALAAP VLEDRQNCGS AWSQCGGIGWSGATCCSSGNS CVEINSYYSQ CLPGAQVTTT |
| | | | AGASSTSPTS TSKVSSTTSK VTSSSAAQPI TTTTAPSVPT TTIAGGASST ASFTGNPFLG VQGWANSYYS |
| | | | SEIYNHAIPS MTGSLAAQAS AVAKVPTFQW LDRNVTVDTL MKSTLEEIRA ANKAGANPPY AAHFVVYDLP |
| | | | DRDCAAAASN GEFSIANGGV ANYKTYINAI RKLLIEYSDI RTILVIEPDS LANLVTNTNV AKCANAASAY |
| | | | RECTNYAITQ LDLPHVAQYL DAGHGGWLGW PANIQPAATL FADIYKAAGK PKSVRGLVTN VSNYNGWSLS |
| | | | SAPSYTTPNP NYDEKKYIEA FSPLLNAAGF PAQFIVDTGR SGKQPTGQIE QGDWCNAIGT GFGVRPTTNT |
| | | | GSSLADAFVW VKPGGESDGT SDTSATRYDY HCGLSDALKP APEAGQWFQA YFEQLLKNAN PAF |
| SEQ ID NO: 104 | BAB39154 | Humicola | MAKFFLTAAF AAAALAAPVV EERQNCAPTW GQCGGIGFNG PTCCQSGSTC VKQNDWYSQC LPGSQVTTTS |
| | | insolens | TTSTSSSSTT SRATSTTRTG GVTSITTAPT RTVTIPGGAT TTASYNGNPF EGVQLWANNY YRSEVHTLAI |
| | | | PQITDPALRA AASAVAEVPS FQWLDRNVTV DTLLVETLSE IRAANQAGAN PPYAAQIVVY DLPDRDCAAA |
| | | | ASNGEWAIAN NGANNYKGYI NRIREILISF SDVRTILVIE PDSLANMVTN MNVAKCSGAA STYRELTIYA |
| | | | LKQLDLPHVA MYMDAGHAGW LGWPANIQPA AELFAKIYED AGKPRAVRGL ATNVANYNAW SISSPPPYTS |
| | | | PNPNYDEKHY IEAFRPLLEA RGFPAQFIVD QGRSGKQPTG QKEWGHWCNA IGTGFGMRPT ANTGHQYVDA |
| | | | FVWVKPGGEC DGTSDTTAAR YDYHCGLEDA LKPAPEAGQW FQAYFEQLLR NANPPF |
| SEQ ID NO: 105 | ABF50873 | Emericella | MHYSASGLAL AFLLPAIQAQ QTLYGQCGGS GWTGATSCVA GAACSTLNQW YAQCLPAATT TSTTLTTTTS |
| | | nidulans | SVTTTSNPGS TTTTSSVTVT ATASGNPFSG YQLYVNPYYS SEVQSIAIPS LTGTLSSLAP AATAAAKVPS |
| | | | FVWLDVAAKV PTMATYLADI RSQNAAGANP PIAGQFVVYD LPDRDCAALA SNGEFAISDG GVQHYKDYID |
| | | | SIREILVEYS DVHVILVIEP DSLANLVTNL NVAKCANAQS AYLECTNYAV TQLNLPNVAM YLDAGHAGWL |
| | | | GWPANLQPAA NLYAGVYSDA GSPAALRGLA TNVANYNAWA IDTCPSYTQG NSVCDEKDYI NALAPLLRAQ |
| | | | GFDAHFITDT GRNGKQPTGQ QAWGDWCNVI GTGFGARPST NTGDSLLDAF VWVKPGGESD GTSDTSAARY |
| | | | DAHCGYSDAL QPAPEAGTWF QAYFVQLLQN ANPSF |
| SEQ ID NO: 106 | CAK41068 | Aspergillus | MHYPLSLALA FLPFGIQAQQ TLWGQCGGQG YSGATSCVAG ATCATVNEYY AQCTPAAGTS SATTLKTTTS |
| | | niger | STTAAVTTTT TTQSPTGSAS PTTTASASGN PFSGYQLYVN PYYSSEVASL AIPSLTGSLS SLQAAATAAA |
| | | | KVPSFVWLDT AAKVPTMGDY LADIQSQNAA GANPPIAGQF VVYDLPDRDC AALASNGEYS IADNGVEHYK |
| | | | SYIDSIREIL VQYSDVHTLL VIEPDSLANL VTNLNVAKCA NAESAYLECT NYALTQLNLP NVAMYLDAGH |
| | | | AGWLGWPANQ QPAADLFASV YKNASSPAAV RGLATNVANY NAWTISSCPS YTQGNSVCDE QQYINAIAPL |
| | | | LQAQGFDAHF IVDTGRNGKQ PTGQQAWGDW CNVINTGFGE RPTTDTGDAL VDAFVWVKPG GESDGTSDSS |
| | | | ATRYDAHCGY SDALQPAPEA GTWFQAYFVQ LLTNANPAF |
| SEQ ID NO: 107 | CAP93233 | Penicillium | MRSFIPFVSL LATSAAAAAI SSAASPTVTA AAAGNPFSGY QLYANSYYAS EVSSLALPSM TGAAKAAASV |
| | | chrysogenum | AAKVPSFYWL DTAAKVPTMG EFLADIRAKN KAGASPPIAG QFVVYDLPDR DCAALASNGE YSIADGGVAK |
| | | Wisconsin | YKAYIDAIRE ILVEYSDIQT ILVVEPDSLA NLVTNMAVSK CANAHDAYLE CTNYAVTQLN LDNVAMYLDA |
| | | 54-1255 | GHAGWLGWPA NLGPAAELYA NVYKTANKPA SMRGLATNVA NYNGWSLSTC PSYTSGNSNC DEKKYINALG |
| | | | PLLKTAGWDA HFITDTGRNG VQPTSQSAWG DWCNVKGTGF GVRPTTETGD ALADAFVWVK PGGESDGTSD |
| | | | SSAARYDAHC GYSDALQPAP EAGTWFQAYF AQLVENANPS L |
| SEQ ID NO: 108 | CAK39856 | Aspergillus | MRAIWPLVSL FSAVKALPAA SATASASVAA SSSPAPTASA TGNPFEGYQL YANPYYKSQV ESSAIPSLSA |
| | | niger | SSLVAQASAA ADVPSFYWLD TADKVPTMGE YLEDIQTQNA AGASPPIAGI FVVYDLPDRD CSALASNGEY |
| | | | SISDGGVEKY KAYIDSIREQ VETYSDVQTI LIIEPDSLAN LVTNLDVAKC ANAESAYLEC TNYALEQLNL |
| | | | PNVAMYLDAG HAGWLGWPAN IGPAAQLYAS VYKNASSPAA VRGLATNVAN FNAWSIDSCP SYTSGNDVCD |
| | | | EKSYINAIAP ELSSAGFDAH FITDTGRNGK QPTGQSAWGD WCNVKDTGFG AQPTTDTGDE LADAFVWVKP |
| | | | GGESDGTSDT SSSRYDAHCG YSDALQPAPE AGTWFQAYFE QLLTNANPSL |
| SEQ ID NO: 109 | BAI65845 | Aspergillus | MHTLNMQALV ALSPLLFSAA TALPQASVTP SPSSSVPASS GPAPTATAGG NPFEGYDLYV NPYYKSEVES |
| | | oryzae | LAIPSMTGSL AEKASAAANV PSFHWLDTTD KVPQMGEFLE DIKTKNAAGA NPPTAGIFVV YDLPDRDCAA |
| | | | LASNGEFLIS DGGVEKYKAY IDSIREQVEK YSDTQIILVI EPDSLANLVT NLNVQKCANA QDAYLECTNY |
| | | | ALTQLNLPNV AMYLDAGHAG WLGWPANIGP AAELYASVYK NASSPAAVRG LATNVANYNA FSIDSCPSYT |
| | | | QGSTVCDEKT YINNFAPQLK SAGFDAHFIV DTGRNGNQPT GQSQWGDWCN VKNTGFGVRP TTDTGDELVD |
| | | | AFVWVKPGGE SDGTSDTSAE RYDAHCGYAD ALTPAPEAGT WFQAYFEQLV ENANPSL |
| SEQ ID NO: 110 | CCD44345 | Botryotinia | MGFKNALLAA AAVAPTVYAQ GAAYAQCGGQ GWSGATTCVS GYTCVVNNAY YSQCLPGSAV TTTATTAPTA |
| | | fuckeliana | TTPTTIITST TKATTTTGGS SATTTAAVAG NPFSGKALYA NPYYASEISA SAIPSLTGAM ATKAAAVAKV |
| | | | PTFYWLLSDT AAKVPLMGTY LANIRALNKA GANPPVAGTF VVYDLPDRDC AAAASNGEYS IADGGLVKYK |
| | | | AYIDSIVALL KTYSDVSVIL VIEPDSLANL VTNLSVAKCS NAQAAYLEGT EYAIAQLNLP NVAMYLDAGH |
| | | | AGWLGWPANI GPAAQLFGQI YKAAGSPAAV RGLATNVANY NAWTSTTCPS YTSGDSNCNE KLYINALAPL |
| | | | LTAQGFPAHF IMDTSRNGVQ PTAQQAWGDW CNLIGTGFGV RPTTNTGDAL EDAFVWIKPG GEGDGTSDTT |
| | | | AARYDFHCGL ADALKPAPEA GTWFQAYFEQ LLTNANPLF |
| SEQ ID NO: 111 | AAL78165 | Rasamsonia | MRNLLALAPA ALLVGAAEAQ QSLWGQCGGS SWTGATSCAA GATCSTINPY YAQCVPATAT PTTLTTTTKP |
| | | emersonii | TSTGGAAPTT PPPTTTGTTT SPVVTRPASA SGNPFEGYQL YANPYYASEV ISLAIPSLSS ELVPKASEVA |
| | | | KVPSFVWLDQ AAKVPSMGDY LKDIQSQNAA GADPPIAGIF VVYDLPDRDC AAAASNGEFS IANNGVALYK |
| | | | QYIDSIREQL TTYSDVHTIL VIEPDSLANV VTNLNVPKCA NAQDAYLECI NYAITQLDLP NVAMYLDAGH |
| | | | AGWLGWQANL APAAQLFASV YKNASSPASV RGLATNVANY NAWSISRCPS YTQGDANCDE EDYVNALGPL |
| | | | FQEQGFPAYF IIDTSRNGVR PTKQSQWGDW CNLIGTGFGV RPTTDTGNPL EDAFVWVKPG GESDGTSNTT |
| | | | SPRYDYHCGL SDALQPAPEA GTWFQAYFEQ LLTNANPLF |
| SEQ ID NO: 112 | ACH91035 | Penicillium | MLRYLSIVAA TAILTGVEAQ QSVWGQCGGQ GWSGATSCAA GSTCSTLNPY YAQCIPGTAT STTLVKTTSS |
| | | funiculosum | TSVGTTSPPT TTTTKASTTA TTTAAASGNP FSGYQLYANP YYSSEVHTLA IPSLTGSLAA AATKAAEIPS |
| | | | FVWLDTAAKV PTMGTYLANI EAANKAGASP PIAGIFVVYD LPDRDCAAAA SNGEYTVANN GVANYKAYID |
| | | | SIVAQLKAYP DVHTILIIEP DSLANMVTNL STAKCAEAQS AYYECVNYAL IKPHLAHVAM YIDAGHAGWL |
| | | | GWSANLSPAA QLFATVYKNA SAPASLRGLA TNVANYNAWS ISSPPSYTSG DSNYDEKLYI NALSPLLTSN |
| | | | GWPDAHFIMD TSRNGVQPTK QQAWGDWCNV IGTGFGVQPT TNTGDPLEDA FVWVKPGGES DGTSNSSATR |
| | | | YDFHCGYSGA LQPAPEAGTW FQAYFVQLLT NANPALV |
| SEQ ID NO: 113 | ADX86895 | Penicillium | MQRTSAWALL LLAQIATAQQ TVWGQCGGIG YSGPTSCVAG SSCSTQNSYY AQCLPGSGNG GGGAATTTTT |
| | | decumbens | AGQTTKTTMA TTTTTSTKTS AGSGGSTTTA PPASNSGNPF KGYQPYVNPY YASEVQSLAI PSLAASLAPK |
| | | | ASAVAKVPSF VWLDTAAKVP TMGTYLADIK AKNAAGANPP IAGIFVVYDL PDRDCAALAS NGEYSIANGG |
| | | | VANYKKYIDS IRAQLLKYPD VHTILVIEPD SLANLVTNMN VAKCSGAHDA YLECTDYALK QLNLPNVAMY |
| | | | LDAGHAGWLG WPANIGPAAD LFASVYKNAG SPAAVRGLAT NVANYNAWSI STCPSYTQGD QNCDEKRYIN |
| | | | ALAPLLRANG FDAHFIMDTS RNGVQPTKQQ AWGDWCNVIG TGFGTPFTTD TGDALQDAFI WVKPGGECDG |
| | | | TSDTSSPRYD AHCGYSDALK PAPEAGTWFQ AYFEQLLVNA NPSF |
| SEQ ID NO: 114 | BAA74458 | Acremonium | MLRYLSIVAA TAILTGVEAQ QSVWGQCGGQ GWSGATSCAA GSTCSTLNPY YAQCIPGTAT STTLVKTTSS |
| | | cellulolyticus | TSVGTTSPPT TTTTKASTTA TTTAAASGNP FSGYQLYANP YYSSEVHTLA IPSLTGSLAA AATKAAEIPS |
| | | Y-94 | FVWLDTAAKV PTMGTYLANI EAANKAGASP PIAGIFVVYD LPDRDCAAAA SNGEYTVANN GVANYKAYID |
| | | | SIVAQLKAYP DVHTILIIEP DSLANMVTNL STAKCAEAQS AYYECVNYAL INLNLANVAM YIDAGHAGWL |
| | | | GWSANLSPAA QLFATVYKNA SAPASLRGLA TNVANYNAWS ISSPPSYTSG DSNYDEKLYI NALSPLLTSN |
| | | | GWPNAHFIMD TSRNGVQPTK QQAWGDWCNV IGTGFGVQPT TNTGDPLEDA FVWVKPGGES DGTSNSSATR |
| | | | YDFHCGYSDA LQPAPEAGTW FQAYFVQLLT NANPALV |
| SEQ ID NO: 115 | CBX97039 | Leptosphaeria | MLNIFLTAAF AAGLSQALPQ ATSAPASSSQ SSMTTMAPAA TGNPFADKNF YANPYYSSEV HTLAMPSLPA |
| | | maculans JN3 | SLKPAATAVA NVGSFVWMDT RAKVPTMDTY LADIKAKNAA GANLMGTFVV YNLPDRDCAA LASNGELKIA |
| | | | EDGANIYKTD YIDKIAAIIQ KYPDVKINLA IEPDSLANMV TNMGVAKCSN AAPYYRNLTS YALEKLNFDN |
| | | | VDMYLDGGHA GWLGWDANIG PAAKLYAEVY KAAGSPRGVR GLVTNVSNYN AFRAATCPAI TSGNKNCDEE |
| | | | RYINAFAPLL SAEGFPAHFI VDTGRSGKQP TDQAAWGEWC NVRGAGFGIR PTTTTDNALV DAFVWVKPGG |
| | | | ESDGTSNTTS ARYDGFCGRD SAFKPAPEAG TWFQAYFEML LQNANPKLA |
| SEQ ID NO: 116 | AAM76664 | Cochliobolus | MLSNVFLTAA LAAGLAQALP QATPTPTAAP SGNPFAGKNF YANPYYSSEV HTLAMPSLPA SLKPAATAVA |
| | | heterostrophus | KVGSFVWMDT MAKVPLMDTY LADIKAKNAA GANLMGTFVV YDLPDRDCAA LASNGELKID EGGVEKYKTQ |
| | | | YIDKIAAIIK KYPDVKINLA IEPDSLANMV TNMGVQKCSR AAPYYKELTA YALKTLNFNN VDMYMDGGHA |
| | | | GWLGWDANIG PAAKLYAEVY KAAGSPRGVR GIVTNVSNYN ALRVSSCPSI TQGNKNCDEE RYINAFAPLL |
| | | | KNEGFPAHFI VDQGRSGKVP TNQQEWGDWC NVSGAGFGTR PTTNTGNALI DAIVWVKPGG ESDGTSDTSA |
| | | | ARYDAHCGRN SAFKPAPEAG TWFQAYFEML LKNANPALA |
| SEQ ID NO: 117 | AAA50607.1 | Agaricus | MFKFAALLAL ASLVPGFVQA QSPVWGQCGG NGWTGPTTCA SGSTCVKQND FYSQCLPNNQ APPSTTTQPG |
| | | bisporus | TTPPATTTSG GTGPTSGAGN PYTGKTVWLS PFYADEVAQA AADISNPSLA TKAASVAKIP TFVWFDTVAK |
| | | | VPDLGGYLAD ARSKNQLVQI VVYDLPDRDC AALASNGEFS LANDGLNKYK NYVDQIAAQI KQFPDVSVVA |
| | | | VIEPDSLANL VTNLNVQKCA NAQSAYKEGV IYAVQKLNAV GVTMYIDAGH AGWLGWPANL SPAAQLFAQI |
| | | | YRDAGSPRNL RGIATNVANF NALRASSPDP ITQGNSNYDE IHYIEALAPM LSNAGFPAHF IVDQGRSGVQ |
| | | | NIRDQWGDWC NVKGAGFGQR PTTNTGSSLI DAIVWVKPGG ECDGTSDNSS PRFDSHCSLS DAHQPAPEAG |
| | | | TWFQAYFETL VANANPAL |
| SEQ ID NO: 118 | AAQ38151.1 | US6573086-015 | MKFVQSATLA FAATALAAPS RTTPQKPRQA SAGCASAVTL DASTNVFQQY TLHPNNFYRA EVEAAAEAIS |
| | | | DSALAEKARK VADVGTFLWL DTIENIGRLE PALEDVPCEN IVGLVIYDLP GRDCAAKASN GELKVGELDR |
| | | | YKTEYIDKIA EILKAHSNTA FALVIEPDSL PNLVTNSDLQ TCQQSASGYR EGVAYALKQL NLPNVVMYID |
| | | | AGHGGWLGWD ANLKPGAQEL ASVYKSAGSP SQVRGISTNV AGWNAWDQEP GEFSDASDAQ YNKCQNEKIY |
| | | | INTFGAELKS AGMPNHAIID TGRNGVTGLR DEWGDWCNVN GAGFGVRPTA NTGDELADAF VWVKPGGESD |
| | | | GTSDSSAARY DSFCGKPDAF KPSPEAGTWN QAYFEMLLKN ANPSF |
| SEQ ID NO: 119 | BAH08702.1 | Coprinopsis | MLKGSKFFAL SLALLPALVQ AQRPLYAQCG GTGWTGETTC VSGAVCEVIN QWYHQCLPGS NQPQPPVTTQ |
| | | cinerea | PPVVVPTTSQ PPVVVPTNPP GGTPVPSTGN PFEGYDIYLS PYYAEEVEAA AAMIDDPVLK AKALKVKEIP |
| | | | TFIWFDVVRK TPDLGRYLAD ATAIQQRTGR KQLVQIVVYD LPDRDCAAAA SNGEFSLADG GMEKYKDYVD |
| | | | RLASEIRKYP DVRIVAVIEP DSLANMVTNM NVAKCRGAEA AYKEGVIYAL RQLSALGVYS YVDAGHAGWL |
| | | | GWNANLAPSA RLFAQIYKDA GRSAFIRGLA TNVSNYNALS ATTRDPVTQG NDNYDELRFI NALAPLLRNE |
| | | | GWDAKFIVDQ GRSGVQNIRQ EWGNWCNVYG AGFGMRPTLN TPSSAIDAIV WIKPGGEADG TSDTSAPRYD |
| | | | THCGKSDSHK PAPEAGTWFQ EYFVNLVKNA NPPL |
| SEQ ID NO: 120 | BAH08703.1 | Coprinopsis | MKYLNLLAAL LAVAPLSLAA PSIEARQSNV NPYIGKSPLV IRSYAQKLEE TVRTFQQRGD QLNAARTRTV |
| | | cinerea | QNVATFAWIS DTNGIGAIRP LIQDALAQQA RTGQKVIVQI VVYNLPDRDC SANASTGEFT VGNDGLNRYK |
| | | | NFVNTIAREL STADADKLHF ALLLEPDALA NLVTNANAPR CRIAAPAYKE GIAYTLATLS KPNVDVYIDA |
| | | | ANGGWLGWND NLRPSAELFK EVYDLARRIN PNAKVRGLAV NVSNYNQYRA EVREPFTEWN DAWDESRYVN |
| | | | VLTPHLNAVG FPAHFIVDQG RGGKGGIRTE WGQWCNVRNA GFGIRPTADQ GVLQNPNVDA IVWVKPGGES |
| | | | DGTSDLNSNR YDPTCRSPVA HVPAPEAGQW FNEYVVNLVL NANPPLEPTW |
| SEQ ID NO: 121 | BAH08704.1 | Coprinopsis | MKFLNLLAAL VAVAPLSLAA PSASFERRQG SVNPYIGRSP LVIKSYAEKL EETIAYFEAQ GDELNAARTR |
| | | cinerea | TVQGIPTFAW ISDSATIDTI QPLIADAVAH QEASGEQVLV QLVIYNLPDR DCAAKASDGE FHLDDDGANK |
| | | | YRAYVDRIVA ELSTADADKL HFSIVLEPDS LGNMVTNMHV PKCQGAATAY KEGIAYTIAS LQKPNIDLYI |
| | | | DAAHGGWLGW NDNLRPSAEI FKETLDLARQ ITPNATVRGL AINVSNYNPY KTRAREDYTE WNNAYDEWNY |
| | | | VKTLTPHLQA VGFPAQFIVD QGRSGREGIR TEWGQWCNIR NAGFGIRPTT DQAIVDSANV DAIVWVKPGG |
| | | | ESDGTSDVNA VRFDENCRSP ASHVPAPEAG EWFNEFVVNL VINANPPLEP TYA |
| SEQ ID NO: 122 | Q7SIG5 | Humicola | QSGNPFSGRT LLVNSDYSSK LDQTRQAFLS RGDQTNAAKV KYVQEKVGTF YWISNIFLLR DIDVAIQNAR |
| | | insolens | AAKARGENPI VGLVLYNLPD RDCSAGESSG ELKLSQNGLN RYKNEYVNPF AQKLKAASDV QFAVILEPDA |
| | | | IGNMVTGTSA FCRNARGPQQ EAIGYAISQL QASHIHLYLD VANGGWLGWA DKLEPTAQEV ATILQKAGNN |
| | | | AKIRGFSSNV SNYNPYSTSN PPPYTSGSPS PDESRYATNI ANAMRQRGLP TQFIIDQSRV ALSGARSEWG |
| | | | QWCNVNPAGF GQPFTTNTNN PNVDAIVWVK PGGESDGQCG MGGAPAAGMW FDAYAQMLTQ NAHDEIAR |
| SEQ ID NO: 123 | BAG48183.1 | Irpexlacteus | MKSAAFLAAL AAILPAYVAG QAQTWAQCGG IGFTGPTTCV AGSVCTKQND YYSQCIPGSA TTPTSAPTSA |
| | | | PTSQPSQPSS TSSAPSGPSS TPTPSANNPW TGYQIYLSPY YANEVAAAAK AITDPTLAAK AASVANIPNF |
| | | | TWLDSVSKIA DLKTYLADAS ALGKSSGQKQ LLQIVVYDLP DRDCAAKASN GEFSIADNGL ANYQNYIDQI |
| | | | VAAVKQFPDV RVVAVIEPDS LANLVTNLNV QKCANAKSTY LTAVNYALKQ LSSVGVYQYM DAGHAGWLGW |
| | | | PANLTPAAQL FAQVYSDAGK SPFIKGLATN VANYNALSAA SPDPITQGDP NYDEIHYINA LAPALQSAGF |
| | | | PATFIVDQGR SGQQNHRQQW GDWCNIKGAG FGTRPTTNTG SSLIDSIVWV KPGGESDGTS NSSSPRFDST |
| | | | CSLSDATQPA PEAGTWFQAY FETLVSKANP PL |
| SEQ ID NO: 124 | AAK28357.1 | Lentinula | MKITSTGLLA LSSLLPFALG QSQLYAQCGG IGWSGATTCV SGATCTVVNA YYSQCIPGSA SAPPTSTSSI |
| | | edodes | GTGTTTSSAP GSTGTTTPAA GNPFTEQIYL SPYYANEIAA AVTQISDPTT AAAAAKVANI PTFIWLDQVA |
| | | | KVPDLGTYLA DASAKQKSEG KNYLVQIVVY DLPDRDCAAL ASNGEFTIAD NGEANYHDYI DQIVAQIKQY |
| | | | PDVHVVAVIE PDSLANLVTN LSVAKCANAQ TTYLECVTYA MQQLSAVGVT MYLDAGHAGW LGWPANLSPA |
| | | | AQLFTSLYSN AGSPSGVRGL ATNVANYNAL VATTPDPITQ GDPNYDEMLY IEALAPLLGS FPAHFIVDQG |
| | | | RSGVQDIRQQ WGDWCNVLGA GFGTQPTTNT GSSLIDSIVW VKPGGECDGT SNTSSPRYDA HCGLPDATPN |
| | | | APEAGTWFQA YFETLVEKAN PPL |
| SEQ ID NO: 125 | CAH05679.1 | Malbranchea | MRDSLFTLLS LALGSASASP FLLPRQANSS NPFAGHTIYP NPYYSNEIDE FAIPALQETD PALVEKAALV |
| | | cinnamomea | KEVGTFFWID VVAKVPDIGP YLQGIQEANA AGQNPPYIGA IVVYDLPNRD CAAAASNGEF SLEDGGEEKY |
| | | | RGYIDGIREQ IEKYPDVRVA LVIEPDSLAN MVTNLNVPKC AESEQAYRDG VAYALKQLDL PNVWTYIDAG |
| | | | HSGWLGWPAN IEPAAEIFVE VWNAAGRPKS TRGFATNVSN YNGYSLSTAP PYTEPNPNFD EVRYINAFRP |
| | | | LLEARGFPAY FIVDQGRSGV QPTAQIEQGH WCNVIDTGFG TRPTTDTGNE YVDSIVWVKP GGESDGTSDT |
| | | | SAERYDYHCG LEDALKPAPE AGQWFQAYFE QLLRNANPPF |
| SEQ ID NO: 126 | AAC09066.1 | Orpinomyces | MKFLTIASLF IAGTLASQCH PNYPCCQNCG EVFYTDSDGQ WGIENNDWCL IQPSKCNSNQ SCKFNALGYS |
| | | sp. PC-2 | CCSHCNSVYS DNDGQWGIEN GNWCGLKDSG FGNVTPTTTR NSNPTTSVNT NDPDNFFNNR IYCNDDRKKR |
| | | | VQSSINQLSG ELRAKAEKIK DVPTALWLSW DRAPESVSGH LSQAGDQTAV FILYWIPTRD CNSYASQGGA |
| | | | QDMNRYQQYV QRIYNAFRSY PNSKIVVVIE PDTLGNMVTS QSNQHCRDVH DLHKQAIAYA LNTLGSLNNV |
| | | | RAYIDAAHGR WLGPHTDEVA KIIKDIVSMA PQGKLRGLST NVSNYQSTRD EYAYHQKLNS ALENVGIRNM |
| | | | KFIVDTARNG VDVAESLVRT GTWCNVIGTG FGERPKGTPD PVNMPLLDAY MWLKPGGDSD GSSSGPYADP |
| | | | NCAHSDSLPG AGNAGDWFHE YFVQLIKNAN PPIQA |
| SEQ ID NO: 127 | AAB92678.1 | Orpinomyces | MKFSTVLATL FATGALASEC HWQYPCCKDC TVYYTDTEGK WGVLNNDWCM IDNRRCSSNN NNCSSSITSQ |
| | | sp. PC-2 | GYPCCSNNNC KVEYTDNDGK WGVENNNWCG ISNSCGGGQQ QQPTQPTQPT QPQQPTQPSS DNFFENEIYS |
| | | | NYKFQGEVDI SIKKLNGDLK AKAEKVKYVP TAVWLAWDGA PQEVPRYLQE AGNKTVVFVL YMIPTRDCGA |
| | | | NASAGGSATI DKYKGYINNI YNTSNQYKNS KIVMILEPDT IGNLVTNNND NCRNVRNMHK QALSYAISKF |
| | | | GTQSHVKVYL DAAHGAWLNQ YADQTANVIK EILNNAGSGK LRGISTNVSN YQSIESEYKY HQNLNRALES |
| | | | KGVRGLKFIV DTSRNGANVE GAFNASGTWC NFKGAGLGQR PKGNPNPGSM PLLDAYMWIK TPGEADGSSQ |
| | | | GSRADPVCAR GDSLQGAPDA GSWFHEYFTM LIQNANPPF |
| SEQ ID NO: 128 | AAB92679.1 | Orpinomyces | MKFSALISTL FAAGAMASRC HPSYPCCNGC NVEYTDTEGN WGVENFDWCF IDESRCNPGY CKFEALGYSC |
| | | sp. PC-2 | CKGCEVVYSD EDGNWGVENQ QWCGIRDNCT PNVPATSART TTRTTTTTRT TTVNSLPTSD NFFENELYSN |
| | | | YKFQGEVDQS IQRLSGSLQE KAKKVKYVPT AAWLAWSGAT NEVARYLNEA GSKTVVFVLY MIPTRDCNAG |
| | | | GSNGGADNLS TYQGYVNSIY NTINQYPNSR IVMIIEPDTI GNLVTANNAN CRNVHDMHKQ ALSYAISKFG |
| | | | TQKNVRVYLD AAHGGWLNSS ADRTAEVIAE ILRNAGNGKI RGISTNVSNY QPVYSEYQYH QNLNRALESR |
| | | | GVRGMKFIVD TSRNGRNPSS ATWCNLKGAG LGARPQANPD PNMPLLDAYV WIKTPGESDS ASSADPVCRN |
| | | | SDSLQGAPAA GSWFHDYFVM LLENANPPF |
| SEQ ID NO: 129 | AAB32942.1 | Phanerochaete | MKSTAFFAAL VTLLPAYVAG QASEWGQCGG IGWTGPTTCV SGTTCTVLNP YYSQCLPGSA VTTTSVITSH |
| | | chrysosporium | SSSVSSVSSH SGSSTSTSSP TGPTGTNPPP PPSANNPWTG FQIFLSPYYA NEVAAAAKQI TDPTLSSKAA |
| | | | SVANIPTFTW LDSVAKIPDL GTYLASASAL GKSTGTKQLV QIVIYDLPDR DCAAKASNGE FSIANNGQAN |
| | | | YENYIDQIVA QIQQFPDVRV VAVIEPDSLA NLVTNLNVQK CANAKTTYLA CVNYALTNLA KVGVYMYMDA |
| | | | GHAGWLGWPA NLSPAAQLFT QVWQNAGKSP FIKGLATNVA NYNALQAASP DPITQGNPNY DEIHYINALA |
| | | | PLLQQAGWDA TFIVDQGRSG VQNIRQQWGD WCNIKGAGFG TRPTTNTGSQ FIDSIVWVKP GGECDGTSNS |
| | | | SSPRYDSTCS LPDAAQPAPE AGTWFQAYFQ TLVSAANPPL |
| SEQ ID NO: 130 | AAM94167.1 | Piromyces | MKTSIALTAV AALAAKASAA CWSEKLGYKC CSSANAPVVY QDADGDWSVE NNDWCGIPAA TPIQSCWSEK |
| | | equi | LGYPCCKSTS AVVYQDADGD WGVENNDWCG ISGDIKPIPT DDPXPGEQYT HVGNPFKGHK FFINPXYTDE |
| | | | VDKAIAQMSD SSLIKKAEKM KEFSNAIWLD NMENMNNWLE RNLKTALAEQ QSGSQTVLTV FVVYDLPGRD |
| | | | CHALASNGEL LANDADFERY KTDYIDVIAE KLAYYKSQPV VAVIEPDSLA NMVTNIESTP ACAKSEKYYM |
| | | | DGHAYLIKKL GQFPHVAMYL DIGHAFXLGW DDNREKGGKV YSKVIKSGSP GKVRGFASNV ANYTPWEDPE |
| | | | LSRGPETEWN SCPDEKRYIQ AMYKDFKAAG IESVYFIDDS SRNGVKNDRF HPGEWCNQTG SGIGARPEAN |
| | | | PVSGMDYLDA FYWVKPYGES DGTSDESAKR YDGYCGHRTA MKPAPEAGQW FQAFFEEGLK NANPPL |
| SEQ ID NO: 131 | AAD51055.1 | Piromyces | MKFSTLIGTL FATGALASSC HRDYPCCNDC NVVYQDWERD WGVLNGQEWC FIDKNRCNGG GYCKFESLGY |
| | | rhizinflatus | PCCNGCDVYY TDNDGRWGVE NGNWCGIRDD KCNGYQQPRT TTTTRTTTRT TTTQRPVQTN VSDNFFENTL |
| | | | YSNFKFQGEV QSSIQKLSGD MAKKAEKVKY VPTAVWLAWE GAPREVPQYL DDAGSKTVVF VLYMIPTRDC |
| | | | NANASVGGSA TLEKYKGYID NIYNTFNQYP NSKIVMILEP DTIGNLVTAN NANCMNVQNL HKQGLAYAIS |
| | | | KFGTQKNVRV YLDAAHGAWL SSHADKTAQV IKEILNNAGS GKLRGITTNV SNYQTVNDEY SYQMRLNSAL |
| | | | QNLGVRDLHY IIDTSRNGAN IAQQFNQSGT WCNFKGAGLG ARPQANPDSS KPLLDAYMWI KTPGEADGSS |
| | | | SGSRADPVCG RWDSLQGAPD AGSWFHDYFV MLLQNANPPF |
| SEQ ID NO: 132 | AAL92497.1 | Piromyces | MKASIALTAI AALAANASAA CFSERLGYPC CRGNEVFYTD NDGDWGVENG NWCGIGGASA TTCWSQALGY |
| | | sp. E2 | PCCTSTSDVA YVDGDGNWGV ENGNWCGIIA GGNSSNNNSG STINVGDVTI GNQYTHTGNP FAGHKFFINP |
| | | | YYTAEVDGAI AQISNASLRA KAEKMKEFSN AIWLDTIKNM NEWLEKNLKY ALAEQNETGK TVLTVFVVYD |
| | | | LPGRDCHALA SNGELLANDS DWARYQSEYI DVIEEKLKTY KSQPVVLVVE PDSLANMVTN LDSTPACRDS |
| | | | EKYYMDGHAY LIKKLGVLPH VAMYLDIGHA FWLGWDDNRL KAGKVYSKVI QSGAPGNVRG FASNVANYTP |
| | | | WEDPTLSRGP DTEWNPCPDE KRYIEAMYKD FKSAGIKSVY FIDDTSRNGH KTDRTHPGEW CNQTGVGIGA |
| | | | RPQANPISGM DYLDAFYWVK PLGESDGYSD TTAVRYDGYC GHATAMKPAP EAGQWFQKHF EQGLENANPP |
| | | | L |
| SEQ ID NO: 133 | CAH05678.1 | Stilbella | MAGRFFLSAA FLASAALAVP LEERQNCSPQ WAQCGGNGWS GPTCCASGSN CQVTNEWYSQ CVPGAAPPPP |
| | | annulata | PVTTTRSTTT PPTTTTRTTA DAPPPTGGAT YTGNPFLGVN QWANNFYRSE IMNIAVPSLS GAMATAAAKV |
| | | | ADVPTFQWID KMDKLPLIDE ALADVRAANA RGGNYASILV VYNLPDRDCA AAASNGEFAI ADGGVAKYKN |
| | | | YIDEIRKLVI KYNDLRIILV IEPDSLANMV TNMNVAKCQN AASAYRECTN YALTNLDLPN VAQYMDAGHA |
| | | | GWLGWPANIT PAAQLFAEVY KQAGSPKSVR GLAINVSNYN AWSVSSPPPY TSPNPNYDER HFVEAFAPLL |
| | | | RQNGWDAKFI VDQGRSGRQP TGQQEWGHWC NAIGTGFGQR PTSNTGHADV DAFVWIKPGG ECDGTSDTSA |
| | | | ARYDHFCGNP DALKPAPEAG EWFQAYFEQL LRNANPAF |

TABLE 2A

| | Percent | | Codon | | Amino Acid | | |
|---|---|---|---|---|---|---|---|
| | Improvement | | in | Wild- | Location in | Amino Acid | |
| | Compared | Wild- | Poly- | type | BD23134 | in | |
| | to Wild- | type | peptide | Amino | sequence | Polypeptide | |
| Rank | type BD23134 | Codon | Variant | Acid | (SEQ ID NO: 2) | variant | Type of Amino Acid Change |
| 1 | 32.1 | ATC | GTG | I | 235 | V | Neutral to neutral |
| 2 | 17.3 | CCT | TGG | P | 64 | W | Neutral to neutral |
| 3 | 16.0 | CCT | GAG | P | 64 | E | Neutral to negative |
| 4 | 14.4 | CTT | AGG | L | 21 | R | Neutral to positive |
| 5 | 12.5 | AGC | GTT | S | 104 | V | Neutral to neutral |
| 6 | 9.8 | | | | (see Table 2B) | | Loop reassembly mutant |
| 7 | 7.9 | GGC | TCG | G | 37 | S | Neutral to neutral |
| 8 | 5.9 | GGA | CTT | G | 65 | L | Neutral to neutral |
| 9 | 5.6 | AAG | CAT | K | 309 | H | Positive to positive |
| 10 | 5.4 | GAG | CGG | E | 66 | R | Negative to positive |
| 11 | 4.4 | TCT | GCT | S | 115 | A | Neutral to neutral |
| 12 | 4.4 | GGT | AAG | G | 67 | K | Neutral to positive |
| 13 | 4.3 | GAG | AAG | E | 23 | K | Negative to positive |
| 14 | 3.6 | TCT | ATG | S | 115 | M | Neutral to neutral |
| 15 | 2.7 | GCC | AAG | A | 33 | K | Neutral to positive |
| 16 | 2.4 | GAG | AAT | E | 23 | N | Negative to neutral |

TABLE 2B

| | Percent | | Codon | | Amino Acid | | |
|---|---|---|---|---|---|---|---|
| | Improvement | | in | Wild- | Location in | Amino Acid | |
| | Compared | Wild- | Poly- | type | BD23134 | in | |
| | to Wild- | type | peptide | Amino | sequence | Polypeptide | |
| Rank | type BD23134 | Codon | Variant | Acid | (SEQ ID NO: 2) | variant | Type of Amino Acid Change |
| 6 | 9.8 | GAT | AAC | D | 194 | N | Negative to neutral |
| | | GCC | CTG | A | 200 | L | Neutral to neutral |
| | | TCT | TGC | S | 421 | C | Neutral to neutral |
| | | GAC | AAC | D | 426 | N | Negative to neutral |
| | | GCT | TCC | A | 429 | S | Neutral to neutral |
| | | ACC | CCT | T | 430 | P | Neutral to neutral |
| | | TAC | GCC | Y | 434 | A | Neutral to neutral |
| | | GCT | CTG | A | 438 | L | Neutral to neutral |
| | | TCT | CCC | S | 439 | P | Neutral to neutral |
| | | GCC | GAT | A | 440 | D | Neutral to negative |
| | | CTT | ACC | L | 442 | T | Neutral to neutral |
| | | CAG | CCT | Q | 443 | P | Neutral to neutral |
| | | CCG | AAC | P | 444 | N | Neutral to neutral |

TABLE 3

| Sequence Identifier | | |
|---|---|---|
| (SEQ ID NO: ) | BD23134 variant | Nucleotide Sequence |
| SEQ ID NO: 100 | Wild-type | ATGACTGTCT ATCAACTCTT GTTTACGGCC GCTTTGGCTG GTACAGCACT TGCTGCCCCT CTTGTCGAGG AACGCCAGGC TTGCGCCAGC |
| | | CAGTGGGCCC AGTGTGGTGG CTTCAGCTGG AATGGTGCTA CTTGCTGCCA GTCTGGTAGT TACTGTAGCA AGATCAATGA CTATTACTCT |
| | | CAGTGTATTC CTGGAGAGGG TCCCGCCACT TCCAAGACAA GCACGCTTCC TGCTTCTACC ACCACCAGCA AGCCGACTTC CACTTCCACT |
| | | GCTGGTACTT CTTCCACTAC GAAGCCTCCA CCTGCTGGAA GCGGCACTGC CACTTATAGC GGAAACCCCT ACTCTGGTGT TAACCTTTGG |
| | | GCCAACAGCT ACTATCGCTC AGAGGTTACC AACTTGGCCA TCCCCAAGTT GAGCGGTGCC ATGGCCACGG CTGCTGCCAA GGTCGCTGAT |
| | | GTTCCCTCTT ATCAGTGGAT GGACTCTTTC GAGCACATCT CCCTGATGGA GGATACTCTT GTTGACATTC GAAAGGCCAA CCAGGCTGGT |
| | | GGTAACTACG CCGGCCAGTT TGTCGTCTAT GATCTCCCTG ATCGTGACTG CGCTGCTGCC GCTTCCAACG GAGAGTATTC CCTTGACAAG |
| | | GATGGTGCCA ACAAGTACAA GAACTACATC AACACTATCA AGAAGATCAT CCAGAGCTAC TCTGATATCC GAATCCTCCT TGTTATTGAG |
| | | CCTGACTCCC TGGCTAACCT GGTCACCAAC ATGGATGTTG CCAAGTGCGC CAAGGCCCAT GATGCGTACA TCAGCCTGAC GAACTACGCT |
| | | GTCACGGAAC TGAACCTACC CAACGTCGCC ATGTATCTTG ATGCAGGCCA CGCTGGCTGG CTCGGCTGGC CCAACAACCA AGGCCCTGCT |
| | | GCGAAGCTCT TTGCTAGCAT CTACAAGGAT GCCGGCAAGC CAGCTGCGCT CCGTGGACTC GCCACCAACG TTGCTAACTA CAACGCCTGG |
| | | AGCCTCAGCA GTGCTCCCCC TTATACCCAA GGCGCCTCCA TCTACGACGA GAAGAGTTTC ATTCACGCAA TGGGTCCTCT CCTGGAGCAG |
| | | AATGGCTGGC CTGGCGCTCA CTTCATTACC GACCAGGGCC GTTCTGGCAA GCAGCCCACC GGCCAGATCC AGTGGGGTGA CTGGTGCAAC |
| | | TCCAAAGGCA CTGGCTTTGG TATCCGTCCC TCTGCCAACA CTGGTGACAG CCTCCTCGAT GCTTTTGTCT GGGTCAAGCC TGGTGGTGAG |
| | | TCTGATGGTA CCTCGGACAC GAGTGCTACC CGTTACGACT ACCACTGCGG TGCTTCTGCC GCTCTTCAGC CGGCACCTGA GGCAGGAACC |
| | | TGGTTCCAGG CCTACTTCGA GCAGCTTCTT ACCAATGCCA ACCCTTCGTT CCTGTAA |
| SEQ ID NO: 134 | 1235V | ATGACTGTCT ATCAACTCTT GTTTACGGCC GCTTTGGCTG GTACAGCACT TGCTGCCCCT CTTGTCGAGG AACGCCAGGC TTGCGCCAGC |
| | | CAGTGGGCCC AGTGTGGTGG CTTCAGCTGG AATGGTGCTA CTTGCTGCCA GTCTGGTAGT TACTGTAGCA AGATCAATGA CTATTACTCT |
| | | CAGTGTATTC CTGGAGAGGG TCCCGCCACT TCCAAGACAA GCACGCTTCC TGCTTCTACC ACCACCAGCA AGCCGACTTC CACTTCCACT |
| | | GCTGGTACTT CTTCCACTAC GAAGCCTCCA CCTGCTGGAA GCGGCACTGC CACTTATAGC GGAAACCCCT ACTCTGGTGT TAACCTTTGG |
| | | GCCAACAGCT ACTATCGCTC AGAGGTTACC AACTTGGCCA TCCCCAAGTT GAGCGGTGCC ATGGCCACGG CTGCTGCCAA GGTCGCTGAT |
| | | GTTCCCTCTT ATCAGTGGAT GGACTCTTTC GAGCACATCT CCCTGATGGA GGATACTCTT GTTGACATTC GAAAGGCCAA CCAGGCTGGT |
| | | GGTAACTACG CCGGCCAGTT TGTCGTCTAT GATCTCCCTG ATCGTGACTG CGCTGCTGCC GCTTCCAACG GAGAGTATTC CCTTGACAAG |
| | | GATGGTGCCA ACAAGTACAA GAACTACATC AACACTATCA AGAAGATCAT CCAGAGCTAC TCTGATATCC GAGTGCTCCT TGTTATTGAG |
| | | CCTGACTCCC TGGCTAACCT GGTCACCAAC ATGGATGTTG CCAAGTGCGC CAAGGCCCAT GATGCGTACA TCAGCCTGAC GAACTACGCT |
| | | GTCACGGAAC TGAACCTACC CAACGTCGCC ATGTATCTTG ATGCAGGCCA CGCTGGCTGG CTCGGCTGGC CCAACAACCA AGGCCCTGCT |
| | | GCGAAGCTCT TTGCTAGCAT CTACAAGGAT GCCGGCAAGC CAGCTGCGCT CCGTGGACTC GCCACCAACG TTGCTAACTA CAACGCCTGG |
| | | AGCCTCAGCA GTGCTCCCCC TTATACCCAA GGCGCCTCCA TCTACGACGA GAAGAGTTTC ATTCACGCAA TGGGTCCTCT CCTGGAGCAG |
| | | AATGGCTGGC CTGGCGCTCA CTTCATTACC GACCAGGGCC GTTCTGGCAA GCAGCCCACC GGCCAGATCC AGTGGGGTGA CTGGTGCAAC |
| | | TCCAAAGGCA CTGGCTTTGG TATCCGTCCC TCTGCCAACA CTGGTGACAG CCTCCTCGAT GCTTTTGTCT GGGTCAAGCC TGGTGGTGAG |
| | | TCTGATGGTA CCTCGGACAC GAGTGCTACC CGTTACGACT ACCACTGCGG TGCTTCTGCC GCTCTTCAGC CGGCACCTGA GGCAGGAACC |
| | | TGGTTCCAGG CCTACTTCGA GCAGCTTCTT ACCAATGCCA ACCCTTCGTT CCTGTAA |
| SEQ ID NO: 135 | P64W | ATGACTGTCT ATCAACTCTT GTTTACGGCC GCTTTGGCTG GTACAGCACT TGCTGCCCCT CTTGTCGAGG AACGCCAGGC TTGCGCCAGC |
| | | CAGTGGGCCC AGTGTGGTGG CTTCAGCTGG AATGGTGCTA CTTGCTGCCA GTCTGGTAGT TACTGTAGCA AGATCAATGA CTATTACTCT |
| | | CAGTGTATTT GGGGAGAGGG TCCCGCCACT TCCAAGACAA GCACGCTTCC TGCTTCTACC ACCACCAGCA AGCCGACTTC CACTTCCACT |
| | | GCTGGTACTT CTTCCACTAC GAAGCCTCCA CCTGCTGGAA GCGGCACTGC CACTTATAGC GGAAACCCCT ACTCTGGTGT TAACCTTTGG |
| | | GCCAACAGCT ACTATCGCTC AGAGGTTACC AACTTGGCCA TCCCCAAGTT GAGCGGTGCC ATGGCCACGG CTGCTGCCAA GGTCGCTGAT |
| | | GTTCCCTCTT ATCAGTGGAT GGACTCTTTC GAGCACATCT CCCTGATGGA GGATACTCTT GTTGACATTC GAAAGGCCAA CCAGGCTGGT |
| | | GGTAACTACG CCGGCCAGTT TGTCGTCTAT GATCTCCCTG ATCGTGACTG CGCTGCTGCC GCTTCCAACG GAGAGTATTC CCTTGACAAG |
| | | GATGGTGCCA ACAAGTACAA GAACTACATC AACACTATCA AGAAGATCAT CCAGAGCTAC TCTGATATCC GAATCCTCCT TGTTATTGAG |
| | | CCTGACTCCC TGGCTAACCT GGTCACCAAC ATGGATGTTG CCAAGTGCGC CAAGGCCCAT GATGCGTACA TCAGCCTGAC GAACTACGCT |
| | | GTCACGGAAC TGAACCTACC CAACGTCGCC ATGTATCTTG ATGCAGGCCA CGCTGGCTGG CTCGGCTGGC CCAACAACCA AGGCCCTGCT |
| | | GCGAAGCTCT TTGCTAGCAT CTACAAGGAT GCCGGCAAGC CAGCTGCGCT CCGTGGACTC GCCACCAACG TTGCTAACTA CAACGCCTGG |
| | | AGCCTCAGCA GTGCTCCCCC TTATACCCAA GGCGCCTCCA TCTACGACGA GAAGAGTTTC ATTCACGCAA TGGGTCCTCT CCTGGAGCAG |
| | | AATGGCTGGC CTGGCGCTCA CTTCATTACC GACCAGGGCC GTTCTGGCAA GCAGCCCACC GGCCAGATCC AGTGGGGTGA CTGGTGCAAC |
| | | TCCAAAGGCA CTGGCTTTGG TATCCGTCCC TCTGCCAACA CTGGTGACAG CCTCCTCGAT GCTTTTGTCT GGGTCAAGCC TGGTGGTGAG |
| | | TCTGATGGTA CCTCGGACAC GAGTGCTACC CGTTACGACT ACCACTGCGG TGCTTCTGCC GCTCTTCAGC CGGCACCTGA GGCAGGAACC |
| | | TGGTTCCAGG CCTACTTCGA GCAGCTTCTT ACCAATGCCA ACCCTTCGTT CCTGTAA |
| SEQ ID NO: 136 | P64E | ATGACTGTCT ATCAACTCTT GTTTACGGCC GCTTTGGCTG GTACAGCACT TGCTGCCCCT CTTGTCGAGG AACGCCAGGC TTGCGCCAGC |
| | | CAGTGGGCCC AGTGTGGTGG CTTCAGCTGG AATGGTGCTA CTTGCTGCCA GTCTGGTAGT TACTGTAGCA AGATCAATGA CTATTACTCT |
| | | CAGTGTATTG AGGGAGAGGG TCCCGCCACT TCCAAGACAA GCACGCTTCC TGCTTCTACC ACCACCAGCA AGCCGACTTC CACTTCCACT |
| | | GCTGGTACTT CTTCCACTAC GAAGCCTCCA CCTGCTGGAA GCGGCACTGC CACTTATAGC GGAAACCCCT ACTCTGGTGT TAACCTTTGG |
| | | GCCAACAGCT ACTATCGCTC AGAGGTTACC AACTTGGCCA TCCCCAAGTT GAGCGGTGCC ATGGCCACGG CTGCTGCCAA GGTCGCTGAT |
| | | GTTCCCTCTT ATCAGTGGAT GGACTCTTTC GAGCACATCT CCCTGATGGA GGATACTCTT GTTGACATTC GAAAGGCCAA CCAGGCTGGT |
| | | GGTAACTACG CCGGCCAGTT TGTCGTCTAT GATCTCCCTG ATCGTGACTG CGCTGCTGCC GCTTCCAACG GAGAGTATTC CCTTGACAAG |
| | | GATGGTGCCA ACAAGTACAA GAACTACATC AACACTATCA AGAAGATCAT CCAGAGCTAC TCTGATATCC GAATCCTCCT TGTTATTGAG |
| | | CCTGACTCCC TGGCTAACCT GGTCACCAAC ATGGATGTTG CCAAGTGCGC CAAGGCCCAT GATGCGTACA TCAGCCTGAC GAACTACGCT |
| | | GTCACGGAAC TGAACCTACC CAACGTCGCC ATGTATCTTG ATGCAGGCCA CGCTGGCTGG CTCGGCTGGC CCAACAACCA AGGCCCTGCT |
| | | GCGAAGCTCT TTGCTAGCAT CTACAAGGAT GCCGGCAAGC CAGCTGCGCT CCGTGGACTC GCCACCAACG TTGCTAACTA CAACGCCTGG |
| | | AGCCTCAGCA GTGCTCCCCC TTATACCCAA GGCGCCTCCA TCTACGACGA GAAGAGTTTC ATTCACGCAA TGGGTCCTCT CCTGGAGCAG |
| | | AATGGCTGGC CTGGCGCTCA CTTCATTACC GACCAGGGCC GTTCTGGCAA GCAGCCCACC GGCCAGATCC AGTGGGGTGA CTGGTGCAAC |
| | | TCCAAAGGCA CTGGCTTTGG TATCCGTCCC TCTGCCAACA CTGGTGACAG CCTCCTCGAT GCTTTTGTCT GGGTCAAGCC TGGTGGTGAG |
| | | TCTGATGGTA CCTCGGACAC GAGTGCTACC CGTTACGACT ACCACTGCGG TGCTTCTGCC GCTCTTCAGC CGGCACCTGA GGCAGGAACC |
| | | TGGTTCCAGG CCTACTTCGA GCAGCTTCTT ACCAATGCCA ACCCTTCGTT CCTGTAA |
| SEQ ID NO: 137 | L21R | ATGACTGTCT ATCAACTCTT GTTTACGGCC GCTTTGGCTG GTACAGCACT TGCTGCCCCT AGGGTCGAGG AACGCCAGGC TTGCGCCAGC |
| | | CAGTGGGCCC AGTGTGGTGG CTTCAGCTGG AATGGTGCTA CTTGCTGCCA GTCTGGTAGT TACTGTAGCA AGATCAATGA CTATTACTCT |
| | | CAGTGTATTC CTGGAGAGGG TCCCGCCACT TCCAAGACAA GCACGCTTCC TGCTTCTACC ACCACCAGCA AGCCGACTTC CACTTCCACT |
| | | GCTGGTACTT CTTCCACTAC GGCGCCTCCA CCTGCTGGAA GCGGCACTGC CACTTATAGC GGAAACCCCT ACTCTGGTGT TAACCTTTGG |
| | | GCCAACAGCT ACTATCGCTC AGAGGTTACC AACTTGGCCA TCCCCAAGTT GAGCGGTGCC ATGGCCACGG CTGCTGCCAA GGTCGCTGAT |
| | | GTTCCCTCTT ATCAGTGGAT GGACTCTTTC GAGCACATCT CCCTGATGGA GGATACTCTT GTTGACATTC GAAAGGCCAA CCAGGCTGGT |
| | | GGTAACTACG CCGGCCAGTT TGTCGTCTAT GATCTCCCTG ATCGTGACTG CGCTGCTGCC GCTTCCAACG GAGAGTATTC CCTTGACAAG |
| | | GATGGTGCCA ACAAGTACAA GAACTACATC AACACTATCA AGAAGATCAT CCAGAGCTAC TCTGATATCC GAATCCTCCT TGTTATTGAG |
| | | CCTGACTCCC TGGCTAACCT GGTCACCAAC ATGGATGTTG CCAAGTGCGC CAAGGCCCAT GATGCGTACA TCAGCCTGAC GAACTACGCT |
| | | GTCACGGAAC TGAACCTACC CAACGTCGCC ATGTATCTTG ATGCAGGCCA CGCTGGCTGG CTCGGCTGGC CCAACAACCA AGGCCCTGCT |
| | | GCGAAGCTCT TTGCTAGCAT CTACAAGGAT GCCGGCAAGC CAGCTGCGCT CCGTGGACTC GCCACCAACG TTGCTAACTA CAACGCCTGG |
| | | AGCCTCAGCA GTGCTCCCCC TTATACCCAA GGCGCCTCCA TCTACGACGA GAAGAGTTTC ATTCACGCAA TGGGTCCTCT CCTGGAGCAG |
| | | AATGGCTGGC CTGGCGCTCA CTTCATTACC GACCAGGGCC GTTCTGGCAA GCAGCCCACC GGCCAGATCC AGTGGGGTGA CTGGTGCAAC |
| | | TCCAAAGGCA CTGGCTTTGG TATCCGTCCC TCTGCCAACA CTGGTGACAG CCTCCTCGAT GCTTTTGTCT GGGTCAAGCC TGGTGGTGAG |
| | | TCTGATGGTA CCTCGGACAC GAGTGCTACC CGTTACGACT ACCACTGCGG TGCTTCTACC GCTCTTCAGC CGGCACCTGA GGCAGGAACC |
| | | TGGTTCCAGG CCTACTTCGA GCAGCTTCTT ACCAATGCCA ACCCTTCGTT CCTGTAA |
| SEQ ID NO: 38 | S104V | ATGACTGTCT ATCAACTCTT GTTTACGGCC GCTTTGGCTG GTACAGCACT TGCTGCCCCT CTTGTCGAGG AACGCCAGGC TTGCGCCAGC |
| | | CAGTGGGCCC AGTGTGGTGG CTTCAGCTGG AATGGTGCTA CTTGCTGCCA GTCTGGTAGT TACTGTAGCA AGATCAATGA CTATTACTCT |
| | | CAGTGTATTC CTGGAGAGGG TCCCGCCACT TCCAAGACAA GCACGCTTCC TGCTTCTACC ACCACCAGCA AGCCGACTTC CACTTCCACT |
| | | GCTGGTACTT CTTCCACTAC GAAGCCTCCA CCTGCTGGAG TTGGCACTGC CACTTATAGC GGAAACCCCT ACTCTGGTGT TAACCTTTGG |
| | | GCCAACAGCT ACTATCGCTC AGAGGTTACC AACTTGGCCA TCCCCAAGTT GAGCGGTGCC ATGGCCACGG CTGCTGCCAA GGTCGCTGAT |
| | | GTTCCCTCTT ATCAGTGGAT GGACTCTTTC GAGCACATCT CCCTGATGGA GGATACTCTT GTTGACATTC GAAAGGCCAA CCAGGCTGGT |
| | | GGTAACTACG CCGGCCAGTT TGTCGTCTAT GATCTCCCTG ATCGTGACTG CGCTGCTGCC GCTTCCAACG GAGAGTATTC CCTTGACAAG |
| | | GATGGTGCCA ACAAGTACAA GAACTACATC AACACTATCA AGAAGATCAT CCAGAGCTAC TCTGATATCC GAATCCTCCT TGTTATTGAG |
| | | CCTGACTCCC TGGCTAACCT GGTCACCAAC ATGGATGTTG CCAAGTGCGC CAAGGCCCAT GATGCGTACA TCAGCCTGAC GAACTACGCT |
| | | GTCACGGAAC TGAACCTACC CAACGTCGCC ATGTATCTTG ATGCAGGCCA CGCTGGCTGG CTCGGCTGGC CCAACAACCA AGGCCCTGCT |
| | | GCGAAGCTCT TTGCTAGCAT CTACAAGGAT GCCGGCAAGC CAGCTGCGCT CCGTGGACTC GCCACCAACG TTGCTAACTA CAACGCCTGG |
| | | AGCCTCAGCA GTGCTCCCCC TTATACCCAA GGCGCCTCCA TCTACGACGA GAAGAGTTTC ATTCACGCAA TGGGTCCTCT CCTGGAGCAG |
| | | AATGGCTGGC CTGGCGCTCA CTTCATTACC GACCAGGGCC GTTCTGGCAA GCAGCCCACC GGCCAGATCC AGTGGGGTGA CTGGTGCAAC |
| | | TCCAAAGGCA CTGGCTTTGG TATCCGTCCC TCTGCCAACA CTGGTGACAG CCTCCTCGAT GCTTTTGTCT GGGTCAAGCC TGGTGGTGAG |
| | | TCTGATGGTA CCTCGGACAC GAGTGCTACC CGTTACGACT ACCACTGCGG TGCTTCTGCC GCTCTTCAGC CGGCACCTGA GGCAGGAACC |
| | | TGGTTCCAGG CCTACTTCGA GCAGCTTCTT ACCAATGCCA ACCCTTCGTT CCTGTAA |
| SEQ ID NO: 139 | G37S | ATGACTGTCT ATCAACTCTT GTTTACGGCC GCTTTGGCTG GTACAGCACT TGCTGCCCCT CTTGTCGAGG AACGCCAGGC TTGCGCCAGC |
| | | CAGTGGGCCC AGTGTGGTTC GTTCAGCTGG AATGGTGCTA CTTGCTGCCA GTCTGGTAGT TACTGTAGCA AGATCAATGA CTATTACTCT |
| | | CAGTGTATTC CTGGAGAGGG TCCCGCCACT TCCAAGACAA GCACGCTTCC TGCTTCTACC ACCACCAGCA AGCCGACTTC CACTTCCACT |
| | | GCTGGTACTT CTTCCACTAC GAAGCCTCCA CCTGCTGGAA GCGGCACTGC CACTTATAGC GGAAACCCCT ACTCTGGTGT TAACCTTTGG |
| | | GCCAACAGCT ACTATCGCTC AGAGGTTACC AACTTGGCCA TCCCCAAGTT GAGCGGTGCC ATGGCCACGG CTGCTGCCAA GGTCGCTGAT |
| | | GTTCCCTCTT ATCAGTGGAT GGACTCTTTC GAGCACATCT CCCTGATGGA GGATACTCTT GTTGACATTC GAAAGGCCAA CCAGGCTGGT |
| | | GGTAACTACG CCGGCCAGTT TGTCGTCTAT GATCTCCCTG ATCGTGACTG CGCTGCTGCC GCTTCCAACG GAGAGTATTC CCTTGACAAG |
| | | GATGGTGCCA ACAAGTACAA GAACTACATC AACACTATCA AGAAGATCAT CCAGAGCTAC TCTGATATCC GAATCCTCCT TGTTATTGAG |
| | | CCTGACTCCC TGGCTAACCT GGTCACCAAC ATGGATGTTG CCAAGTGCGC CAAGGCCCAT GATGCGTACA TCAGCCTGAC GAACTACGCT |
| | | GTCACGGAAC TGAACCTACC CAACGTCGCC ATGTATCTTG ATGCAGGCCA CGCTGGCTGG CTCGGCTGGC CCAACAACCA AGGCCCTGCT |
| | | GCGAAGCTCT TTGCTAGCAT CTACAAGGAT GCCGGCAAGC CAGCTGCGCT CCGTGGACTC GCCACCAACG TTGCTAACTA CAACGCCTGG |
| | | AGCCTCAGCA GTGCTCCCCC TTATACCCAA GGCGCCTCCA TCTACGACGA GAAGAGTTTC ATTCACGCAA TGGGTCCTCT CCTGGAGCAG |
| | | AATGGCTGGC CTGGCGCTCA CTTCATTACC GACCAGGGCC GTTCTGGCAA GCAGCCCACC GGCCAGATCC AGTGGGGTGA CTGGTGCAAC |
| | | TCCAAAGGCA CTGGCTTTGG TATCCGTCCC TCTGCCAACA CTGGTGACAG CCTCCTCGAT GCTTTTGTCT GGGTCAAGCC TGGTGGTGAG |
| | | TCTGATGGTA CCTCGGACAC GAGTGCTACC CGTTACGACT ACCACTGCGG TGCTTCTGCC GCTCTTCAGC CGGCACCTGA GGCAGGAACC |
| | | TGGTTCCAGG CCTACTTCGA GCAGCTTCTT ACCAATGCCA ACCCTTCGTT CCTGTAA |
| SEQ ID NO: 140 | G65L | ATGACTGTCT ATCAACTCTT GTTTACGGCC GCTTTGGCTG GTACAGCACT TGCTGCCCCT CTTGTCGAGG AACGCCAGGC TTGCGCCAGC |
| | | CAGTGGGCCC AGTGTGGTGG CTTCAGCTGG AATGGTGCTA CTTGCTGCCA GTCTGGTAGT TACTGTAGCA AGATCAATGA CTATTACTCT |
| | | CAGTGTATTC CTCTTGAGGG TCCCGCCACT TCCAAGACAA GCACGCTTCC TGCTTCTACC ACCACCAGCA AGCCGACTTC CACTTCCACT |
| | | GCTGGTACTT CTTCCACTAC GAAGCCTCCA CCTGCTGGAA GCGGCACTGC CACTTATAGC GGAAACCCCT ACTCTGGTGT TAACCTTTGG |
| | | GCCAACAGCT ACTATCGCTC AGAGGTTACC AACTTGGCCA TCCCCAAGTT GAGCGGTGCC ATGGCCACGG CTGCTGCCAA GGTCGCTGAT |
| | | GTTCCCTCTT ATCAGTGGAT GGACTCTTTC GAGCACATCT CCCTGATGGA GGATACTCTT GTTGACATTC GAAAGGCCAA CCAGGCTGGT |
| | | GGTAACTACG CCGGCCAGTT TGTCGTCTAT GATCTCCCTG ATCGTGACTG CGCTGCTGCC GCTTCCAACG GAGAGTATTC CCTTGACAAG |
| | | GATGGTGCCA ACAAGTACAA GAACTACATC AACACTATCA AGAAGATCAT CCAGAGCTAC TCTGATATCC GAATCCTCCT TGTTATTGAG |
| | | CCTGACTCCC TGGCTAACCT GGTCACCAAC ATGGATGTTG CCAAGTGCGC CAAGGCCCAT GATGCGTACA TCAGCCTGAC GAACTACGCT |
| | | GTCACGGAAC TGAACCTACC CAACGTCGCC ATGTATCTTG ATGCAGGCCA CGCTGGCTGG CTCGGCTGGC CCAACAACCA AGGCCCTGCT |
| | | GCGAAGCTCT TTGCTAGCAT CTACCATGAT GCCGGCAAGC CAGCTGCGCT CCGTGGACTC GCCACCAACG TTGCTAACTA CAACGCCTGG |
| | | AGCCTCAGCA GTGCTCCCCC TTATACCCAA GGCGCCTCCA TCTACGACGA GAAGAGTTTC ATTCACGCAA TGGGTCCTCT CCTGGAGCAG |
| | | AATGGCTGGC CTGGCGCTCA CTTCATTACC GACCAGGGCC GTTCTGGCAA GCAGCCCACC GGCCAGATCC AGTGGGGTGA CTGGTGCAAC |
| | | TCCAAAGGCA CTGGCTTTGG TATCCGTCCC TCTGCCAACA CTGGTGACAG CCTCCTCGAT GCTTTTGTCT GGGTCAAGCC TGGTGGTGAG |
| | | TCTGATGGTA CCTCGGACAC GAGTGCTACC CGTTACGACT ACCACTGCGG TGCTTCTGCC GCTCTTCAGC CGGCACCTGA GGCAGGAACC |
| | | TGGTTCCAGG CCTACTTCGA GCAGCTTCTT ACCAATGCCA ACCCTTCGTT CCTGTAA |
| SEQ ID NO: 141 | K309H | ATGACTGTCT ATCAACTCTT GTTTACGGCC GCTTTGGCTG GTACAGCACT TGCTGCCCCT CTTGTCGAGG AACGCCAGGC TTGCGCCAGC |
| | | CAGTGGGCCC AGTGTGGTGG CTTCAGCTGG AATGGTGCTA CTTGCTGCCA GTCTGGTAGT TACTGTAGCA AGATCAATGA CTATTACTCT |
| | | CAGTGTATTC CTGGAGAGGG TCCCGCCACT TCCAAGACAA GCACGCTTCC TGCTTCTACC ACCACCAGCA AGCCGACTTC CACTTCCACT |
| | | GCTGGTACTT CTTCCACTAC GAAGCCTCCA CCTGCTGGAA GCGGCACTGC CACTTATAGC GGAAACCCCT ACTCTGGTGT TAACCTTTGG |
| | | GCCAACAGCT ACTATCGCTC AGAGGTTACC AACTTGGCCA TCCCCAAGTT GAGCGGTGCC ATGGCCACGG CTGCTGCCAA GGTCGCTGAT |
| | | GTTCCCTCTT ATCAGTGGAT GGACTCTTTC GAGCACATCT CCCTGATGGA GGATACTCTT GTTGACATTC GAAAGGCCAA CCAGGCTGGT |
| | | GGTAACTACG CCGGCCAGTT TGTCGTCTAT GATCTCCCTG ATCGTGACTG CGCTGCTGCC GCTTCCAACG GAGAGTATTC CCTTGACAAG |
| | | GATGGTGCCA ACAAGTACAA GAACTACATC AACACTATCA AGAAGATCAT CCAGAGCTAC TCTGATATCC GAATCCTCCT TGTTATTGAG |
| | | CCTGACTCCC TGGCTAACCT GGTCACCAAC ATGGATGTTG CCAAGTGCGC CAAGGCCCAT GATGCGTACA TCAGCCTGAC GAACTACGCT |
| | | GTCACGGAAC TGAACCTACC CAACGTCGCC ATGTATCTTG ATGCAGGCCA CGCTGGCTGG CTCGGCTGGC CCAACAACCA AGGCCCTGCT |
| | | GCGAAGCTCT TTGCTAGCAT CTACCATGAT GCCGGCAAGC CAGCTGCGCT CCGTGGACTC GCCACCAACG TTGCTAACTA CAACGCCTGG |
| | | AGCCTCAGCA GTGCTCCCCC TTATACCCAA GGCGCCTCCA TCTACGACGA GAAGAGTTTC ATTCACGCAA TGGGTCCTCT CCTGGAGCAG |
| | | AATGGCTGGC CTGGCGCTCA CTTCATTACC GACCAGGGCC GTTCTGGCAA GCAGCCCACC GGCCAGATCC AGTGGGGTGA CTGGTGCAAC |
| | | TCCAAAGGCA CTGGCTTTGG TATCCGTCCC TCTGCCAACA CTGGTGACAG CCTCCTCGAT GCTTTTGTCT GGGTCAAGCC TGGTGGTGAG |
| | | TCTGATGGTA CCTCGGACAC GAGTGCTACC CGTTACGACT ACCACTGCGG TGCTTCTGCC GCTCTTCAGC CGGCACCTGA GGCAGGAACC |
| | | TGGTTCCAGG CCTACTTCGA GCAGCTTCTT ACCAATGCCA ACCCTTCGTT CCTGTAA |
| SEQ ID NO: 142 | E66R | ATGACTGTCT ATCAACTCTT GTTTACGGCC GCTTTGGCTG GTACAGCACT TGCTGCCCCT CTTGTCGAGG AACGCCAGGC TTGCGCCAGC |
| | | CAGTGGGCCC AGTGTGGTGG CTTCAGCTGG AATGGTGCTA CTTGCTGCCA GTCTGGTAGT TACTGTAGCA AGATCAATGA CTATTACTCT |
| | | CAGTGTATTC CTGGACGGGG TCCCGCCACT TCCAAGACAA GCACGCTTCC TGCTTCTACC ACCACCAGCA AGCCGACTTC CACTTCCACT |
| | | GCTGGTACTT CTTCCACTAC GAAGCCTCCA CCTGCTGGAA GCGGCACTGC CACTTATAGC GGAAACCCCT ACTCTGGTGT TAACCTTTGG |
| | | GCCAACAGCT ACTATCGCTC AGAGGTTACC AACTTGGCCA TCCCCAAGTT GAGCGGTGCC ATGGCCACGG CTGCTGCCAA GGTCGCTGAT |
| | | GTTCCCTCTT ATCAGTGGAT GGACTCTTTC GAGCACATCT CCCTGATGGA GGATACTCTT GTTGACATTC GAAAGGCCAA CCAGGCTGGT |
| | | GGTAACTACG CCGGCCAGTT TGTCGTCTAT GATCTCCCTG ATCGTGACTG CGCTGCTGCC GCTTCCAACG GAGAGTATTC CCTTGACAAG |
| | | GATGGTGCCA ACAAGTACAA GAACTACATC AACACTATCA AGAAGATCAT CCAGAGCTAC TCTGATATCC GAATCCTCCT TGTTATTGAG |
| | | CCTGACTCCC TGGCTAACCT GGTCACCAAC ATGGATGTTG CCAAGTGCGC CAAGGCCCAT GATGCGTACA TCAGCCTGAC GAACTACGCT |
| | | GTCACGGAAC TGAACCTACC CAACGTCGCC ATGTATCTTG ATGCAGGCCA CGCTGGCTGG CTCGGCTGGC CCAACAACCA AGGCCCTGCT |
| | | GCGAAGCTCT TTGCTAGCAT CTACAAGGAT GCCGGCAAGC CAGCTGCGCT CCGTGGACTC GCCACCAACG TTGCTAACTA CAACGCCTGG |
| | | AGCCTCAGCA GTGCTCCCCC TTATACCCAA GGCGCCTCCA TCTACGACGA GAAGAGTTTC ATTCACGCAA TGGGTCCTCT CCTGGAGCAG |
| | | AATGGCTGGC CTGGCGCTCA CTTCATTACC GACCAGGGCC GTTCTGGCAA GCAGCCCACC GGCCAGATCC AGTGGGGTGA CTGGTGCAAC |
| | | TCCAAAGGCA CTGGCTTTGG TATCCGTCCC TCTGCCAACA CTGGTGACAG CCTCCTCGAT GCTTTTGTCT GGGTCAAGCC TGGTGGTGAG |
| | | TCTGATGGTA CCTCGGACAC GAGTGCTACC CGTTACGACT ACCACTGCGG TGCTTCTGCC GCTCTTCAGC CGGCACCTGA GGCAGGAACC |
| | | TGGTTCCAGG CCTACTTCGA GCAGCTTCTT ACCAATGCCA ACCCTTCGTT CCTGTAA |
| SEQ ID NO: 143 | S115A | ATGACTGTCT ATCAACTCTT GTTTACGGCC GCTTTGGCTG GTACAGCACT TGCTGCCCCT CTTGTCGAGG AACGCCAGGC TTGCGCCAGC |
| | | CAGTGGGCCC AGTGTGGTGG CTTCAGCTGG AATGGTGCTA CTTGCTGCCA GTCTGGTAGT TACTGTAGCA AGATCAATGA CTATTACTCT |
| | | CAGTGTATTC CTGGAGAGGG TCCCGCCACT TCCAAGACAA GCACGCTTCC TGCTTCTACC ACCACCAGCA AGCCGACTTC CACTTCCACT |
| | | GCTGGTACTT CTTCCACTAC GAAGCCTCCA CCTGCTGGAA GCGGCACTGC CACTTATAGC GGAAACCCCT ACGCTGGTGT TAACCTTTGG |
| | | GCCAACAGCT ACTATCGCTC AGAGGTTACC AACTTGGCCA TCCCCAAGTT GAGCGGTGCC ATGGCCACGG CTGCTGCCAA GGTCGCTGAT |
| | | GTTCCCTCTT ATCAGTGGAT GGACTCTTTC GAGCACATCT CCCTGATGGA GGATACTCTT GTTGACATTC GAAAGGCCAA CCAGGCTGGT |
| | | GGTAACTACG CCGGCCAGTT TGTCGTCTAT GATCTCCCTG ATCGTGACTG CGCTGCTGCC GCTTCCAACG GAGAGTATTC CCTTGACAAG |
| | | GATGGTGCCA ACAAGTACAA GAACTACATC AACACTATCA AGAAGATCAT CCAGAGCTAC TCTGATATCC GAATCCTCCT TGTTATTGAG |
| | | CCTGACTCCC TGGCTAACCT GGTCACCAAC ATGGATGTTG CCAAGTGCGC CAAGGCCCAT GATGCGTACA TCAGCCTGAC GAACTACGCT |
| | | GTCACGGAAC TGAACCTACC CAACGTCGCC ATGTATCTTG ATGCAGGCCA CGCTGGCTGG CTCGGCTGGC CCAACAACCA AGGCCCTGCT |
| | | GCGAAGCTCT TTGCTAGCAT CTACAAGGAT GCCGGCAAGC CAGCTGCGCT CCGTGGACTC GCCACCAACG TTGCTAACTA CAACGCCTGG |
| | | AGCCTCAGCA GTGCTCCCCC TTATACCCAA GGCGCCTCCA TCTACGACGA GAAGAGTTTC ATTCACGCAA TGGGTCCTCT CCTGGAGCAG |
| | | AATGGCTGGC CTGGCGCTCA CTTCATTACC GACCAGGGCC GTTCTGGCAA GCAGCCCACC GGCCAGATCC AGTGGGGTGA CTGGTGCAAC |
| | | TCCAAAGGCA CTGGCTTTGG TATCCGTCCC TCTGCCAACA CTGGTGACAG CCTCCTCGAT GCTTTTGTCT GGGTCAAGCC TGGTGGTGAG |
| | | TCTGATGGTA CCTCGGACAC GAGTGCTACC CGTTACGACT ACCACTGCGG TGCTTCTGCC GCTCTTCAGC CGGCACCTGA GGCAGGAACC |
| | | TGGTTCCAGG CCTACTTCGA GCAGCTTCTT ACCAATGCCA ACCCTTCGTT CCTGTAA |
| SEQ ID NO: 144 | G67K | ATGACTGTCT ATCAACTCTT GTTTACGGCC GCTTTGGCTG GTACAGCACT TGCTGCCCCT CTTGTCGAGG AACGCCAGGC TTGCGCCAGC |
| | | CAGTGGGCCC AGTGTGGTGG CTTCAGCTGG AATGGTGCTA CTTGCTGCCA GTCTGGTAGT TACTGTAGCA AGATCAATGA CTATTACTCT |
| | | CAGTGTATTC CTGGAGAGAA GCCCGCCACT TCCAAGACAA GCACGCTTCC TGCTTCTACC ACCACCAGCA AGCCGACTTC CACTTCCACT |
| | | GCTGGTACTT CTTCCACTAC GAAGCCTCCA CCTGCTGGAA GCGGCACTGC CACTTATAGC GGAAACCCCT ACTCTGGTGT TAACCTTTGG |
| | | GCCAACAGCT ACTATCGCTC AGAGGTTACC AACTTGGCCA TCCCCAAGTT GAGCGGTGCC ATGGCCACGG CTGCTGCCAA GGTCGCTGAT |
| | | GTTCCCTCTT ATCAGTGGAT GGACTCTTTC GAGCACATCT CCCTGATGGA GGATACTCTT GTTGACATTC GAAAGGCCAA CCAGGCTGGT |
| | | GGTAACTACG CCGGCCAGTT TGTCGTCTAT GATCTCCCTG ATCGTGACTG CGCTGCTGCC GCTTCCAACG GAGAGTATTC CCTTGACAAG |
| | | GATGGTGCCA ACAAGTACAA GAACTACATC AACACTATCA AGAAGATCAT CCAGAGCTAC TCTGATATCC GAATCCTCCT TGTTATTGAG |
| | | CCTGACTCCC TGGCTAACCT GGTCACCAAC ATGGATGTTG CCAAGTGCGC CAAGGCCCAT GATGCGTACA TCAGCCTGAC GAACTACGCT |
| | | GTCACGGAAC TGAACCTACC CAACGTCGCC ATGTATCTTG ATGCAGGCCA CGCTGGCTGG CTCGGCTGGC CCAACAACCA AGGCCCTGCT |
| | | GCGAAGCTCT TTGCTAGCAT CTACAAGGAT GCCGGCAAGC CAGCTGCGCT CCGTGGACTC GCCACCAACG TTGCTAACTA CAACGCCTGG |
| | | AGCCTCAGCA GTGCTCCCCC TTATACCCAA GGCGCCTCCA TCTACGACGA GAAGAGTTTC ATTCACGCAA TGGGTCCTCT CCTGGAGCAG |
| | | AATGGCTGGC CTGGCGCTCA CTTCATTACC GACCAGGGCC GTTCTGGCAA GCAGCCCACC GGCCAGATCC AGTGGGGTGA CTGGTGCAAC |
| | | TCCAAAGGCA CTGGCTTTGG TATCCGTCCC TCTGCCAACA CTGGTGACAG CCTCCTCGAT GCTTTTGTCT GGGTCAAGCC TGGTGGTGAG |
| | | TCTGATGGTA CCTCGGACAC GAGTGCTACC CGTTACGACT ACCACTGCGG TGCTTCTGCC GCTCTTCAGC CGGCACCTGA GGCCAGATCC |
| | | TGGTTCCAGG CCTACTTCGA GCAGCTTCTT ACCAATGCCA ACCCTTCGTT CCTGTAA |
| SEQ ID NO: 145 | E23K | ATGACTGTCT ATCAACTCTT GTTTACGGCC GCTTTGGCTG GTACAGCACT TGCTGCCCCT CTTGTCAAGG AACGCCAGGC TTGCGCCAGC |
| | | CAGTGGGCCC AGTGTGGTGG CTTCAGCTGG AATGGTGCTA CTTGCTGCCA GTCTGGTAGT TACTGTAGCA AGATCAATGA CTATTACTCT |
| | | CAGTGTATTC CTGGAGAGGG TCCCGCCACT TCCAAGACAA GCACGCTTCC TGCTTCTACC ACCACCAGCA AGCCGACTTC CACTTCCACT |
| | | GCTGGTACTT CTTCCACTAC GAAGCCTCCA CCTGCTGGAA GCGGCACTGC CACTTATAGC GGAAACCCCT ACTCTGGTGT TAACCTTTGG |
| | | GCCAACAGCT ACTATCGCTC AGAGGTTACC AACTTGGCCA TCCCCAAGTT GAGCGGTGCC ATGGCCACGG CTGCTGCCAA GGTCGCTGAT |
| | | GTTCCCTCTT ATCAGTGGAT GGACTCTTTC GAGCACATCT CCCTGATGGA GGATACTCTT GTTGACATTC GAAAGGCCAA CCAGGCTGGT |
| | | GGTAACTACG CCGGCCAGTT TGTCGTCTAT GATCTCCCTG ATCGTGACTG CGCTGCTGCC GCTTCCAACG GAGAGTATTC CCTTGACAAG |
| | | GATGGTGCCA ACAAGTACAA GAACTACATC AACACTATCA AGAAGATCAT CCAGAGCTAC TCTGATATCC GAATCCTCCT TGTTATTGAG |
| | | CCTGACTCCC TGGCTAACCT GGTCACCAAC ATGGATGTTG CCAAGTGCGC CAAGGCCCAT GATGCGTACA TCAGCCTGAC GAACTACGCT |
| | | GTCACGGAAC TGAACCTACC CAACGTCGCC ATGTATCTTG ATGCAGGCCA CGCTGGCTGG CTCGGCTGGC CCAACAACCA AGGCCCTGCT |
| | | GCGAAGCTCT TTGCTAGCAT CTACAAGGAT GCCGGCAAGC CAGCTGCGCT CCGTGGACTC GCCACCAACG TTGCTAACTA CAACGCCTGG |
| | | AGCCTCAGCA GTGCTCCCCC TTATACCCAA GGCGCCTCCA TCTACGACGA GAAGAGTTTC ATTCACGCAA TGGGTCCTCT CCTGGAGCAG |
| | | AATGGCTGGC CTGGCGCTCA CTTCATTACC GACCAGGGCC GTTCTGGCAA GCAGCCCACC GGCCAGATCC AGTGGGGTGA CTGGTGCAAC |
| | | TCCAAAGGCA CTGGCTTTGG TATCCGTCCC TCTGCCAACA CTGGTGACAG CCTCCTCGAT GCTTTTGTCT GGGTCAAGCC TGGTGGTGAG |
| | | TCTGATGGTA CCTCGGACAC GAGTGCTACC CGTTACGACT ACCACTGCGG TGCTTCTGCC GCTCTTCAGC CGGCACCTGA GGCCAGATCC |
| | | TGGTTCCAGG CCTACTTCGA GCAGCTTCTT ACCAATGCCA ACCCTTCGTT CCTGTAA |
| SEQ ID NO: 146 | S115M | ATGACTGTCT ATCAACTCTT GTTTACGGCC GCTTTGGCTG GTACAGCACT TGCTGCCCCT CTTGTCGAGG AACGCCAGGC TTGCGCCAGC |
| | | CAGTGGGCCC AGTGTGGTGG CTTCAGCTGG AATGGTGCTA CTTGCTGCCA GTCTGGTAGT TACTGTAGCA AGATCAATGA CTATTACTCT |
| | | CAGTGTATTC CTGGAGAGGG TCCCGCCACT TCCAAGACAA GCACGCTTCC TGCTTCTACC ACCACCAGCA AGCCGACTTC CACTTCCACT |
| | | GCTGGTACTT CTTCCACTAC GAAGCCTCCA CCTGCTGGAA GCGGCACTGC CACTTATAGC GGAAACCCCT ACATGGGTGT TAACCTTTGG |
| | | GCCAACAGCT ACTATCGCTC AGAGGTTACC AACTTGGCCA TCCCCAAGTT GAGCGGTGCC ATGGCCACGG CTGCTGCCAA GGTCGCTGAT |
| | | GTTCCCTCTT ATCAGTGGAT GGACTCTTTC GAGCACATCT CCCTGATGGA GGATACTCTT GTTGACATTC GAAAGGCCAA CCAGGCTGGT |
| | | GGTAACTACG CCGGCCAGTT TGTCGTCTAT GATCTCCCTG ATCGTGACTG CGCTGCTGCC GCTTCCAACG GAGAGTATTC CCTTGACAAG |
| | | GATGGTGCCA ACAAGTACAA GAACTACATC AACACTATCA AGAAGATCAT CCAGAGCTAC TCTGATATCC GAATCCTCCT TGTTATTGAG |
| | | CCTGACTCCC TGGCTAACCT GGTCACCAAC ATGGATGTTG CCAAGTGCGC CAAGGCCCAT GATGCGTACA TCAGCCTGAC GAACTACGCT |
| | | GTCACGGAAC TGAACCTACC CAACGTCGCC ATGTATCTTG ATGCAGGCCA CGCTGGCTGG CTCGGCTGGC CCAACAACCA AGGCCCTGCT |
| | | GCGAAGCTCT TTGCTAGCAT CTACAAGGAT GCCGGCAAGC CAGCTGCGCT CCGTGGACTC GCCACCAACG TTGCTAACTA CAACGCCTGG |
| | | AGCCTCAGCA GTGCTCCCCC TTATACCCAA GGCGCCTCCA TCTACGACGA GAAGAGTTTC ATTCACGCAA TGGGTCCTCT CCTGGAGCAG |
| | | AATGGCTGGC CTGGCGCTCA CTTCATTACC GACCAGGGCC GTTCTGGCAA GCAGCCCACC GGCCAGATCC AGTGGGGTGA CTGGTGCAAC |
| | | TCCAAAGGCA CTGGCTTTGG TATCCGTCCC TCTGCCAACA CTGGTGACAG CCTCCTCGAT GCTTTTGTCT GGGTCAAGCC TGGTGGTGAG |
| | | TCTGATGGTA CCTCGGACAC GAGTGCTACC CGTTACGACT ACCACTGCGG TGCTTCTGCC GCTCTTCAGC CGGCACCTGA GGCAGGAACC |
| | | TGGTTCCAGG CCTACTTCGA GCAGCTTCTT ACCAATGCCA ACCCTTCGTT CCTGTAA |
| SEQ ID NO: 147 | A33K | ATGACTGTCT ATCAACTCTT GTTTACGGCC GCTTTGGCTG GTACAGCACT TGCTGCCCCT CTTGTCGAGG AACGCCAGGC TTGCGCCAGC |
| | | CAGTGGAAGC AGTGTGGTGG CTTCAGCTGG AATGGTGCTA CTTGCTGCCA GTCTGGTAGT TACTGTAGCA AGATCAATGA CTATTACTCT |
| | | CAGTGTATTC CTGGAGAGGG TCCCGCCACT TCCAAGACAA GCACGCTTCC TGCTTCTACC ACCACCAGCA AGCCGACTTC CACTTCCACT |
| | | GCTGGTACTT CTTCCACTAC GAAGCCTCCA CCTGCTGGAA GCGGCACTGC CACTTATAGC GGAAACCCCT ACTCTGGTGT TAACCTTTGG |
| | | GCCAACAGCT ACTATCGCTC AGAGGTTACC AACTTGGCCA TCCCCAAGTT GAGCGGTGCC ATGGCCACGG CTGCTGCCAA GGTCGCTGAT |
| | | GTTCCCTCTT ATCAGTGGAT GGACTCTTTC GAGCACATCT CCCTGATGGA GGATACTCTT GTTGACATTC GAAAGGCCAA CCAGGCTGGT |
| | | GGTAACTACG CCGGCCAGTT TGTCGTCTAT GATCTCCCTG ATCGTGACTG CGCTGCTGCC GCTTCCAACG GAGAGTATTC CCTTGACAAG |
| | | GATGGTGCCA ACAAGTACAA GAACTACATC AACACTATCA AGAAGATCAT CCAGAGCTAC TCTGATATCC GAATCCTCCT TGTTATTGAG |
| | | CCTGACTCCC TGGCTAACCT GGTCACCAAC ATGGATGTTG CCAAGTGCGC CAAGGCCCAT GATGCGTACA TCAGCCTGAC GAACTACGCT |
| | | GTCACGGAAC TGAACCTACC CAACGTCGCC ATGTATCTTG ATGCAGGCCA CGCTGGCTGG CTCGGCTGGC CCAACAACCA AGGCCCTGCT |
| | | GCGAAGCTCT TTGCTAGCAT CTACAAGGAT GCCGGCAAGC CAGCTGCGCT CCGTGGACTC GCCACCAACG TTGCTAACTA CAACGCCTGG |
| | | AGCCTCAGCA GTGCTCCCCC TTATACCCAA GGCGCCTCCA TCTACGACGA GAAGAGTTTC ATTCACGCAA TGGGTCCTCT CCTGGAGCAG |
| | | AATGGCTGGC CTGGCGCTCA CTTCATTACC GACCAGGGCC GTTCTGGCAA GCAGCCCACC GGCCAGATCC AGTGGGGTGA CTGGTGCAAC |
| | | TCCAAAGGCA CTGGCTTTGG TATCCGTCCC TCTGCCAACA CTGGTGACAG CCTCCTCGAT GCTTTTGTCT GGGTCAAGCC TGGTGGTGAG |
| | | TCTGATGGTA CCTCGGACAC GAGTGCTACC CGTTACGACT ACCACTGCGG TGCTTCTGCC GCTCTTCAGC CGGCACCTGA GGCAGGAACC |
| | | TGGTTCCAGG CCTACTTCGA GCAGCTTCTT ACCAATGCCA ACCCTTCGTT CCTGTAA |
| SEQ ID NO: 148 | E23N | ATGACTGTCT ATCAACTCTT GTTTACGGCC GCTTTGGCTG GTACAGCACT TGCTGCCCCT CTTGTCAATG AACGCCAGGC TTGCGCCAGC |
| | | CAGTGGGCCC AGTGTGGTGG CTTCAGCTGG AATGGTGCTA CTTGCTGCCA GTCTGGTAGT TACTGTAGCA AGATCAATGA CTATTACTCT |
| | | CAGTGTATTC CTGGAGAGGG TCCCGCCACT TCCAAGACAA GCACGCTTCC TGCTTCTACC ACCACCAGCA AGCCGACTTC CACTTCCACT |
| | | GCTGGTACTT CTTCCACTAC GGCGCCTCCA CCTGCTGGAA GCGGCACTGC CACTTATAGC GGAAACCCCT ACTCTGGTGT TAACCTTTGG |
| | | GCCAACAGCT ACTATCGCTC AGAGGTTACC AACTTGGCCA TCCCCAAGTT GAGCGGTGCC ATGGCCACGG CTGCTGCCAA GGTCGCTGAT |
| | | GTTCCCTCTT ATCAGTGGAT GGACTCTTTC GAGCACATCT CCCTGATGGA GGATACTCTT GTTGACATTC GAAAGGCCAA CCAGGCTGGT |
| | | GGTAACTACG CCGGCCAGTT TGTCGTCTAT GATCTCCCTG ATCGTGACTG CGCTGCTGCC GCTTCCAACG GAGAGTATTC CCTTGACAAG |
| | | GATGGTGCCA ACAAGTACAA GAACTACATC AACACTATCA AGAAGATCAT CCAGAGCTAC TCTGATATCC GAATCCTCCT TGTTATTGAG |
| | | CCTGACTCCC TGGCTAACCT GGTCACCAAC ATGGATGTTG CCAAGTGCGC CAAGGCCCAT GATGCGTACA TCAGCCTGAC GAACTACGCT |
| | | GTCACGGAAC TGAACCTACC CAACGTCGCC ATGTATCTTG ATGCAGGCCA CGCTGGCTGG CTCGGCTGGC CCAACAACCA AGGCCCTGCT |
| | | GCGAAGCTCT TTGCTAGCAT CTACAAGGAT GCCGGCAAGC CAGCTGCGCT CCGTGGACTC GCCACCAACG TTGCTAACTA CAACGCCTGG |
| | | AGCCTCAGCA GTGCTCCCCC TTATACCCAA GGCGCCTCCA TCTACGACGA GAAGAGTTTC ATTCACGCAA TGGGTCCTCT CCTGGAGCAG |
| | | AATGGCTGGC CTGGCGCTCA CTTCATTACC GACCAGGGCC GTTCTGGCAA GCAGCCCACC GGCCAGATCC AGTGGGGTGA CTGGTGCAAC |
| | | TCCAAAGGCA CTGGCTTTGG TATCCGTCCC TCTGCCAACA CTGGTGACAG CCTCCTCGAT GCTTTTGTCT GGGTCAAGCC TGGTGGTGAG |
| | | TCTGATGGTA CCTCGGACAC GAGTGCTACC CGTTACGACT ACCACTGCGG TGCTTCTACC GCTCTTCAGC CGGCACCTGA GGCAGGAACC |
| | | TGGTTCCAGG CCTACTTCGA GCAGCTTCTT ACCAATGCCA ACCCTTCGTT CCTGTAA |

TABLE 4A

| Sequence | Database Accession | | | | | | | | | | | | |
|---|---|---|---|---|---|---|---|---|---|---|---|---|---|
| Identifier | Number or Patent | Position Corresposnding to Amino Acid Position of BD23134 | | | | | | | | | | | |
| (SEQ ID NO:) | Document Number | 21 | 23 | 33 | 37 | 64 | 65 | 66 | 67 | 104 | 115 | 235 | 309 |
| SEQ ID NO: 3 | BD21660* | L-12 | C-14 | G-24 | G-28 | P-55 | A-56 | A-57 | G-58 | T-103 | S-114 | T-239 | K-313 |
| SEQ ID NO: 4 | BD22435* | P-20 | E-22 | G-32 | G-36 | P-63 | G-64 | T-65 | A-66 | S-108 | A-119 | I-239 | K-313 |
| SEQ ID NO: 5 | US8101393-0098 | L-12 | C-14 | G-24 | G-28 | P-55 | A-56 | A-57 | G-58 | T-103 | S-114 | T-239 | K-313 |
| SEQ ID NO: 6 | WO2011059740-0002 | L-12 | C-14 | G-24 | G-28 | P-55 | A-56 | A-57 | G-58 | T-102 | S-113 | T-238 | K-312 |
| SEQ ID NO: 7 | US8168863-0013 | P-3 | E-5 | G-15 | G-19 | P-46 | G-47 | T-48 | A-49 | S-91 | A-102 | I-222 | K-296 |
| SEQ ID NO: 8 | US20090320831-0082 | P-3 | E-5 | G-15 | G-19 | P-46 | G-47 | A-48 | A-49 | S-90 | V-101 | T-221 | K-295 |
| SEQ ID NO: 9 | US20120142046-0060 | P-41 | E-43 | G-53 | G-57 | P-84 | G-85 | A-86 | A-87 | S-128 | V-139 | T-259 | K-333 |
| SEQ ID NO: 10 | US20100313307-0046 | L-17 | S-19 | G-29 | G-33 | P-60 | G-61 | A-62 | A-63 | S-104 | V-115 | T-235 | K-309 |
| SEQ ID NO: 11 | US20100189706-0282 | L-21 | E-23 | A-33 | G-37 | P-64 | G-65 | E-66 | G-67 | S-104 | S-115 | I-235 | K-309 |
| SEQ ID NO: 12 | US20100189706-0358 | P-20 | E-22 | G-32 | G-36 | P-63 | G-64 | T-65 | A-66 | S-108 | A-119 | I-239 | K-313 |
| SEQ ID NO: 13 | US20100189706-0401 | I-21 | E-23 | — | G-36 | S-63 | G-64 | S-65 | N-66 | A-110 | S-121 | I-241 | K-315 |
| SEQ ID NO: 14 | US4894338-0002 | P-20 | E-22 | G-32 | G-36 | P-63 | G-64 | A-65 | A-66 | S-107 | V-118 | T-238 | K-312 |
| SEQ ID NO: 15 | US6114158-0008 | P-20 | E-22 | G-32 | G-36 | P-63 | G-64 | A-65 | A-66 | S-106 | V-117 | T-237 | K-311 |
| SEQ ID NO: 16 | US8008056-0089 | P-20 | E-22 | G-32 | G-36 | P-63 | G-64 | A-65 | A-66 | S-107 | V-118 | T-238 | K-312 |
| SEQ ID NO: 17 | US20090325240-0923 | P-20 | E-22 | G-32 | G-36 | P-63 | G-64 | A-65 | A-66 | S-106 | V-117 | T-237 | K-311 |
| SEQ ID NO: 18 | US20090325240-0946 | P-20 | E-22 | G-32 | G-36 | P-63 | G-64 | A-65 | A-66 | S-106 | V-117 | T-237 | K-311 |
| SEQ ID NO: 19 | US20090325240-0970 | P-20 | E-22 | G-32 | G-36 | P-63 | G-64 | A-65 | A-66 | S-107 | V-118 | T-238 | K-312 |
| SEQ ID NO: 20 | US20120129229-0028 | P-20 | E-22 | G-32 | G-36 | P-63 | G-64 | A-65 | A-66 | S-107 | V-118 | T-238 | K-312 |
| SEQ ID NO: 21 | US8012734-0037 | — | — | G-8 | G-12 | P-39 | G-40 | A-41 | A-42 | S-83 | V-94 | T-214 | K-288 |
| SEQ ID NO: 22 | US8012734-0038 | — | — | G-8 | G-12 | P-39 | G-40 | A-41 | A-42 | S-83 | V-94 | T-214 | K-288 |
| SEQ ID NO: 23 | US8012734-0039 | — | — | G-8 | G-12 | P-39 | G-40 | A-41 | A-42 | S-83 | V-94 | T-214 | K-288 |
| SEQ ID NO: 24 | US8012734-0040 | — | — | G-8 | G-12 | P-39 | G-40 | A-41 | A-42 | S-83 | V-94 | T-214 | K-288 |
| SEQ ID NO: 25 | US8012734-0041 | — | — | G-8 | G-12 | P-39 | G-40 | A-41 | A-42 | S-83 | V-94 | T-214 | K-288 |
| SEQ ID NO: 26 | US8012734-0042 | — | — | G-8 | G-12 | P-39 | G-40 | A-41 | A-42 | S-83 | V-94 | T-214 | K-288 |
| SEQ ID NO: 27 | US8012734-0043 | — | — | G-8 | G-12 | P-39 | G-40 | A-41 | A-42 | S-83 | V-94 | T-214 | K-288 |
| SEQ ID NO: 28 | US8012734-0044 | — | — | G-8 | G-12 | P-39 | G-40 | A-41 | A-42 | S-83 | V-94 | T-214 | K-288 |
| SEQ ID NO: 29 | US8012734-0045 | — | — | G-8 | G-12 | P-39 | G-40 | A-41 | A-42 | S-83 | V-94 | T-214 | K-288 |
| SEQ ID NO: 30 | US8012734-0046 | — | — | G-8 | G-12 | P-39 | G-40 | A-41 | A-42 | S-83 | V-94 | T-214 | K-288 |
| SEQ ID NO: 31 | US8012734-0047 | — | — | G-8 | G-12 | P-39 | G-40 | A-41 | A-42 | S-83 | V-94 | T-214 | K-288 |
| SEQ ID NO: 32 | US8012734-0048 | — | — | G-8 | G-12 | P-39 | G-40 | A-41 | A-42 | S-83 | V-94 | T-214 | K-288 |
| SEQ ID NO: 33 | US8012734-0049 | — | — | G-8 | G-12 | P-39 | G-40 | A-41 | A-42 | S-83 | V-94 | T-214 | K-288 |
| SEQ ID NO: 34 | US8012734-0050 | — | — | G-8 | G-12 | P-39 | G-40 | A-41 | A-42 | S-83 | V-94 | T-214 | K-288 |
| SEQ ID NO: 35 | US8012734-0051 | — | — | G-8 | G-12 | P-39 | G-40 | A-41 | A-42 | S-83 | V-94 | T-214 | K-288 |
| SEQ ID NO: 36 | US8012734-0052 | — | — | G-8 | G-12 | P-39 | G-40 | A-41 | A-42 | S-83 | V-94 | T-214 | K-288 |
| SEQ ID NO: 37 | US8012734-0053 | — | — | G-8 | G-12 | P-39 | G-40 | A-41 | A-42 | S-83 | V-94 | T-214 | K-288 |
| SEQ ID NO: 38 | US8012734-0054 | — | — | G-8 | G-12 | P-39 | G-40 | A-41 | A-42 | S-83 | V-94 | T-214 | K-288 |
| SEQ ID NO: 39 | US8012734-0055 | — | — | G-8 | G-12 | P-39 | G-40 | A-41 | A-42 | S-83 | V-94 | T-214 | K-288 |
| SEQ ID NO: 40 | US8012734-0057 | — | — | G-8 | G-12 | P-39 | G-40 | A-41 | A-42 | S-83 | V-94 | T-214 | K-288 |
| SEQ ID NO: 41 | US8012734-0058 | — | — | G-8 | G-12 | P-39 | G-40 | A-41 | A-42 | S-83 | V-94 | T-214 | K-288 |
| SEQ ID NO: 42 | US8012734-0061 | — | — | G-8 | G-12 | P-39 | G-40 | A-41 | A-42 | S-83 | V-94 | T-214 | K-288 |
| SEQ ID NO: 43 | US8012734-0062 | — | — | G-8 | G-12 | P-39 | G-40 | A-41 | A-42 | S-83 | V-94 | T-214 | K-288 |
| SEQ ID NO: 44 | US8012734-0063 | — | — | G-8 | G-12 | P-39 | G-40 | A-41 | A-42 | S-83 | V-94 | T-214 | K-288 |
| SEQ ID NO: 45 | US8012734-0064 | — | — | G-8 | G-12 | P-39 | G-40 | A-41 | A-42 | S-83 | V-94 | T-214 | K-288 |
| SEQ ID NO: 46 | US8012734-0066 | — | — | G-8 | G-12 | P-39 | G-40 | A-41 | A-42 | S-83 | V-94 | T-214 | K-288 |
| SEQ ID NO: 47 | US8012734-0071 | — | — | G-8 | G-12 | P-39 | G-40 | A-41 | A-42 | S-83 | V-94 | T-214 | K-288 |
| SEQ ID NO: 48 | US8012734-0157 | — | — | G-8 | G-12 | P-39 | G-40 | A-41 | A-42 | S-83 | V-94 | T-214 | K-288 |
| SEQ ID NO: 49 | US8012734-0158 | — | — | G-8 | G-12 | P-39 | G-40 | A-41 | A-42 | S-83 | V-94 | T-214 | K-288 |
| SEQ ID NO: 50 | US8012734-0159 | — | — | G-8 | G-12 | P-39 | G-40 | A-41 | A-42 | S-83 | V-94 | T-214 | K-288 |
| SEQ ID NO: 51 | US8012734-0160 | — | — | G-8 | G-12 | P-39 | G-40 | A-41 | A-42 | S-83 | V-94 | T-214 | K-288 |
| SEQ ID NO: 52 | US8012734-0161 | — | — | G-8 | G-12 | P-39 | G-40 | A-41 | A-42 | S-83 | V-94 | T-214 | K-288 |
| SEQ ID NO: 53 | US7785854-0023 | K-8 | F-10 | G-20 | G-24 | P-51 | G-52 | A-53 | A-54 | S-95 | V-106 | T-226 | K-300 |
| SEQ ID NO: 54 | US8101398-0012 | — | — | G-8 | G-12 | P-39 | G-40 | A-41 | A-42 | S-83 | V-94 | T-214 | K-288 |
| SEQ ID NO: 55 | US8101398-0014 | — | — | G-8 | G-12 | P-39 | G-40 | A-41 | A-42 | S-83 | V-94 | T-214 | K-288 |
| SEQ ID NO: 56 | US8101398-0015 | — | — | G-8 | G-12 | P-39 | G-40 | A-41 | A-42 | S-83 | V-94 | T-214 | K-288 |
| SEQ ID NO: 57 | US8101398-0016 | — | — | G-8 | G-12 | P-39 | G-40 | A-41 | A-42 | S-83 | V-94 | T-214 | K-288 |
| SEQ ID NO: 58 | US8101398-0017 | — | — | G-8 | G-12 | P-39 | G-40 | A-41 | A-42 | S-83 | V-94 | T-214 | K-288 |
| SEQ ID NO: 59 | US8101398-0020 | — | — | G-8 | G-12 | P-39 | G-40 | A-41 | A-42 | S-83 | V-94 | T-214 | K-288 |
| SEQ ID NO: 60 | US8110389-0037 | — | — | G-8 | G-12 | P-39 | G-40 | A-41 | A-42 | S-83 | V-94 | T-214 | K-288 |
| SEQ ID NO: 61 | US8110389-0038 | — | — | G-8 | G-12 | P-39 | G-40 | A-41 | A-42 | S-83 | V-94 | T-214 | K-288 |
| SEQ ID NO: 62 | US8110389-0039 | — | — | G-8 | G-12 | P-39 | G-40 | A-41 | A-42 | S-83 | V-94 | T-214 | K-288 |
| SEQ ID NO: 63 | US8110389-0040 | — | — | G-8 | G-12 | P-39 | G-40 | A-41 | A-42 | S-83 | V-94 | T-214 | K-288 |
| SEQ ID NO: 64 | US8110389-0041 | — | — | G-8 | G-12 | P-39 | G-40 | A-41 | A-42 | S-83 | V-94 | T-214 | K-288 |
| SEQ ID NO: 65 | US20100016570-0083 | — | — | G-8 | G-12 | P-39 | G-40 | A-41 | A-42 | S-83 | V-94 | T-214 | K-288 |
| SEQ ID NO: 66 | US20100016570-0085 | — | — | G-8 | G-12 | P-39 | G-40 | A-41 | A-42 | S-83 | V-94 | T-214 | K-288 |
| SEQ ID NO: 67 | US20100016570-0088 | — | — | G-8 | G-12 | P-39 | G-40 | A-41 | A-42 | S-83 | V-94 | T-214 | K-288 |
| SEQ ID NO: 68 | US20100016570-0089 | — | — | G-8 | G-12 | P-39 | G-40 | A-41 | A-42 | S-83 | V-94 | T-214 | K-288 |
| SEQ ID NO: 69 | US20100016570-0094 | — | — | G-8 | G-12 | P-39 | G-40 | A-41 | A-42 | S-83 | V-94 | T-214 | K-288 |
| SEQ ID NO: 70 | US20100016570-0095 | — | — | G-8 | G-12 | P-39 | G-40 | A-41 | A-42 | S-83 | V-94 | T-214 | K-288 |
| SEQ ID NO: 71 | US20100016570-0096 | — | — | G-8 | G-12 | P-39 | G-40 | A-41 | A-42 | S-83 | V-94 | T-214 | K-288 |
| SEQ ID NO: 72 | US20100221778-0010 | — | — | G-8 | G-12 | P-39 | G-40 | A-41 | A-42 | S-83 | V-94 | T-214 | K-288 |
| SEQ ID NO: 73 | US20100221778-0011 | — | — | G-8 | G-12 | P-39 | D-40 | A-41 | A-42 | S-83 | V-94 | T-214 | K-288 |
| SEQ ID NO: 74 | US20100221778-0012 | — | — | G-8 | G-12 | P-39 | G-40 | A-41 | A-42 | S-83 | V-94 | T-214 | K-288 |
| SEQ ID NO: 75 | US20100221778-0013 | — | — | G-8 | G-12 | P-39 | G-40 | A-41 | A-42 | S-83 | V-94 | T-214 | K-288 |
| SEQ ID NO: 76 | US20100221778-0014 | — | — | G-8 | G-12 | P-39 | G-40 | A-41 | A-42 | S-83 | V-94 | T-214 | K-288 |
| SEQ ID NO: 77 | US20100221778-0015 | — | — | G-8 | G-12 | P-39 | G-40 | A-41 | A-42 | S-83 | V-94 | T-214 | K-288 |
| SEQ ID NO: 78 | US20100221778-0017 | — | — | G-8 | G-12 | P-39 | G-40 | A-41 | A-42 | S-83 | V-94 | T-214 | K-288 |
| SEQ ID NO: 79 | EP2401370-0016 | — | — | G-8 | G-12 | P-39 | G-40 | A-41 | A-42 | S-83 | V-94 | T-214 | K-288 |
| SEQ ID NO: 80 | JP2011523854-0016 | — | — | G-8 | G-12 | P-39 | G-40 | A-41 | A-42 | S-83 | V-94 | T-214 | K-288 |
| SEQ ID NO: 81 | US20100317087-0018 | — | — | G-8 | G-12 | P-39 | G-40 | A-41 | A-42 | S-83 | V-94 | T-214 | K-288 |
| SEQ ID NO: 82 | US20110189744-0041 | P-2 | E-4 | G-14 | G-18 | P-45 | G-46 | A-47 | A-48 | S-89 | V-100 | T-220 | K-294 |
| SEQ ID NO: 83 | AAQ72468 | — | E-22 | S-33 | G-37 | P-64 | G-65 | S-66 | — | S-94 | S-105 | I-224 | K-298 |
| SEQ ID NO: 84 | AAA65585 | — | E-20 | A-31 | G-35 | P-62 | G-63 | S-64 | — | S-98 | A-109 | V-229 | K-303 |
| SEQ ID NO: 85 | ADC83999 | P-20 | E-22 | G-32 | G-36 | P-63 | G-64 | A-65 | A-66 | S-107 | V-118 | T-238 | K-312 |
| SEQ ID NO: 86 | AAA34210 | P-20 | E-22 | G-32 | G-36 | P-63 | G-64 | A-65 | A-66 | S-107 | V-118 | T-238 | K-312 |
| SEQ ID NO: 87 | AAQ76094 | P-20 | E-22 | G-32 | G-36 | P-63 | G-64 | A-65 | A-66 | S-107 | V-118 | T-238 | K-312 |
| SEQ ID NO: 88 | AAG39980 | P-20 | E-22 | G-32 | G-36 | P-63 | G-64 | A-65 | A-66 | S-107 | V-118 | T-238 | K-312 |
| SEQ ID NO: 89 | AAA72922 | P-20 | E-22 | G-32 | G-36 | P-63 | G-64 | A-65 | A-66 | S-107 | V-118 | T-238 | K-312 |
| SEQ ID NO: 90 | ABF56208 | P-20 | E-22 | G-32 | G-36 | P-63 | G-64 | A-65 | A-66 | S-106 | V-117 | T-237 | K-311 |
| SEQ ID NO: 91 | ACZ34301 | P-20 | E-22 | G-32 | G-36 | P-63 | G-64 | A-65 | A-66 | S-106 | V-117 | T-237 | K-311 |
| SEQ ID NO: 92 | ABG48766 | P-20 | E-22 | G-32 | G-36 | P-63 | G-64 | A-65 | A-66 | S-106 | V-117 | T-237 | K-311 |
| SEQ ID NO: 93 | ACH96126 | P-20 | E-22 | G-32 | G-36 | P-63 | G-64 | A-65 | A-66 | S-106 | V-117 | T-237 | K-311 |
| SEQ ID NO: 94 | ADJ10628 | P-20 | E-22 | G-32 | G-36 | P-63 | G-64 | A-65 | A-66 | S-106 | V-117 | T-237 | K-311 |
| SEQ ID NO: 95 | AAU05379 | P-20 | E-22 | G-32 | G-36 | P-63 | G-64 | A-65 | A-66 | S-106 | V-117 | T-237 | K-311 |
| SEQ ID NO: 96 | AEO62210 | V-20 | E-22 | S-32 | G-36 | P-63 | N-64 | S-65 | Q-66 | S-116 | S-127 | I-250 | T-324 |
| SEQ ID NO: 97 | CBX74420 | P-21 | E-23 | G-33 | G-37 | P-64 | G-65 | T-66 | A-67 | S-109 | A-120 | I-240 | K-314 |
| SEQ ID NO: 98 | AEO55787 | V-20 | E-22 | T-32 | G-36 | P-63 | N-64 | S-65 | Q-66 | T-117 | S-128 | I-251 | N-325 |
| SEQ ID NO: 99 | AAY88915 | L-20 | E-22 | G-32 | G-36 | P-63 | G-64 | Q-65 | A-66 | S-111 | S-122 | T-245 | R-319 |
| SEQ ID NO: 100 | CAP60942 | V-20 | E-22 | S-32 | G-36 | P-63 | G-64 | S-65 | Q-66 | S-119 | L-130 | T-253 | K-327 |
| SEQ ID NO: 101 | AAW64927 | L-20 | E-22 | G-32 | G-36 | P-63 | G-64 | Q-65 | A-66 | S-111 | S-122 | T-245 | R-319 |
| SEQ ID NO: 102 | ADZ99361 | A-13 | T-15 | G-32 | G-36 | P-63 | G-64 | S-65 | — | S-101 | E-112 | I-235 | S-309 |
| SEQ ID NO: 103 | CAD70733 | V-21 | E-23 | S-33 | G-37 | P-64 | G-65 | A-66 | Q-67 | S-119 | L-130 | T-253 | K-327 |
| SEQ ID NO: 104 | BAB39154 | V-19 | E-21 | G-31 | G-35 | P-62 | G-63 | — | S-64 | T-110 | E-121 | T-245 | E-319 |
| SEQ ID NO: 105 | ABF50873 | L-13 | P-15 | G-25 | G-29 | P-56 | A-57 | A-58 | T-59 | T-88 | S-99 | V-224 | S-298 |
| SEQ ID NO: 106 | CAK41068 | L-12 | F-14 | G-24 | G-28 | P-55 | A-56 | A-57 | G-58 | T-92 | S-103 | T-228 | K-302 |
| SEQ ID NO: 107 | CAP93233 | — | — | — | — | — | — | — | — | T-27 | S-38 | T-160 | K-234 |
| SEQ ID NO: 108 | CAK39856 | — | — | — | — | — | — | — | — | A-35 | E-46 | T-169 | K-243 |
| SEQ ID NO: 109 | BAI65845 | — | — | — | — | — | — | — | — | A-43 | E-54 | I-176 | K-250 |
| SEQ ID NO: 110 | CCD44345 | V-13 | P-15 | A-25 | G-29 | P-56 | G-57 | — | — | T-93 | S-104 | V-228 | K-302 |
| SEQ ID NO: 111 | AAL78165 | L-13 | G-15 | G-25 | G-29 | P-56 | A-57 | T-58 | — | T-95 | E-106 | T-228 | K-302 |
| SEQ ID NO: 112 | ACH91035 | I-13 | T-15 | G-25 | G-29 | P-56 | G-57 | — | — | T-91 | S-102 | T-224 | K-298 |
| SEQ ID NO: 113 | ADX86895 | L-12 | Q-14 | G-24 | G-28 | P-55 | G-56 | S-57 | G-58 | A-100 | K-111 | T-233 | K-307 |
| SEQ ID NO: 114 | BAA74458 | I-13 | T-15 | G-25 | G-29 | P-56 | G-57 | — | — | T-91 | S-102 | T-224 | K-298 |
| SEQ ID NO: 115 | CBX97039 | — | — | — | — | — | — | — | — | — | A-46 | I-167 | K-241 |
| SEQ ID NO: 116 | AAM76664 | — | — | — | — | — | — | — | — | — | A-36 | I-157 | K-231 |
| SEQ ID NO: 117 | AAA50607.1 | V-14 | G-16 | G-26 | G-30 | P-57 | — | N-58 | N-59 | — | T-93 | V-208 | R-282 |
| SEQ ID NO: 118 | AAQ38151.1 | — | — | — | — | — | — | — | — | A-37 | Q-48 | F-161 | K-235 |
| SEQ ID NO: 119 | BAH08702.1 | L-15 | A-17 | A-27 | G-31 | P-58 | G-59 | S-60 | N-61 | — | E-103 | I-224 | K-298 |
| SEQ ID NO: 120 | BAH08703.1 | — | — | — | — | — | — | — | — | — | I-34 | F-160 | R-237 |
| SEQ ID NO: 121 | BAH08704.1 | — | — | — | — | — | — | — | — | — | I-36 | F-162 | R-239 |
| SEQ ID NO: 122 | Q7SIG5 | — | — | — | — | — | — | — | — | — | S-7 | F-132 | Q-205 |
| SEQ ID NO: 123 | BAG48183.1 | L-14 | A-16 | A-26 | G-30 | P-57 | G-58 | S-59 | A-60 | — | T-101 | V-222 | S-296 |
| SEQ ID NO: 124 | AAK28357.1 | L-15 | — | A-26 | G-30 | P-57 | G-58 | S-59 | A-60 | — | T-95 | V-215 | S-289 |
| SEQ ID NO: 125 | CAH05679.1 | — | — | — | — | — | — | — | — | — | A-34 | V-159 | N-233 |
| SEQ ID NO: 126 | AAC09066.1 | — | — | — | — | — | — | — | — | — | F-127 | I-235 | V-307 |
| SEQ ID NO: 127 | AAB92678.1 | — | — | — | — | — | — | — | — | Q-124 | F-134 | I-242 | L-313 |
| SEQ ID NO: 128 | AAB92679.1 | — | — | — | — | — | — | — | — | V-123 | F-133 | I-241 | L-312 |
| SEQ ID NO: 129 | AAB32942.1 | L-14 | A-16 | G-26 | G-30 | P-57 | G-58 | S-59 | A-60 | — | T-109 | V-230 | Q-304 |
| SEQ ID NO: 130 | AAM94167.1 | — | — | — | — | — | — | — | — | G-116 | K-127 | V-250 | I-325 |
| SEQ ID NO: 131 | AAD51055.1 | — | — | — | — | — | — | — | — | R-125 | F-136 | I-244 | L-315 |
| SEQ ID NO: 132 | AAL92497.1 | — | — | — | — | — | — | — | — | G-121 | A-132 | V-255 | I-330 |
| SEQ ID NO: 133 | CAH05678.1 | P-20 | E-22 | A-32 | G-36 | P-63 | G-64 | A-65 | A-66 | T-100 | L-107 | I-227 | K-301 |

TABLE 4B

| Sequence | Database Accession | | | | | | | | | | | | | |
|---|---|---|---|---|---|---|---|---|---|---|---|---|---|---|
| Identifier | Number or Patent | Position Corresponding to Amino Acid Position of BD23134 | | | | | | | | | | | | |
| (SEQ ID NO:) | Document Number | 194 | 200 | 421 | 426 | 429 | 430 | 434 | 438 | 439 | 440 | 442 | 443 | 444 |
| SEQ ID NO: 3 | BD21660* | D-198 | L-204 | S-424 | D-429 | A-432 | T-433 | A-437 | Y-441 | S-442 | D-443 | L-445 | Q-446 | P-447 |
| SEQ ID NO: 4 | BD22435* | D-198 | A-204 | C-425 | N-430 | A-433 | P-434 | Y-438 | S-442 | A-443 | D-444 | L-446 | Q-447 | P-448 |
| SEQ ID NO: 5 | US8101393-0098 | D-198 | L-204 | S-424 | D-429 | A-432 | T-433 | A-437 | Y-441 | S-442 | D-443 | L-445 | Q-446 | P-447 |
| SEQ ID NO: 6 | WO2011059740-0002 | D-197 | L-203 | S-423 | D-428 | A-431 | T-432 | A-436 | Y-440 | S-441 | D-442 | L-444 | Q-445 | P-446 |
| SEQ ID NO: 7 | US8168863-0013 | D-181 | A-187 | C-408 | N-413 | A-416 | P-417 | Y-421 | S-425 | A-426 | D-427 | L-429 | Q-430 | P-431 |
| SEQ ID NO: 8 | US20090320831-0082 | D-180 | L-186 | C-407 | D-412 | A-415 | P-416 | S-420 | L-424 | P-425 | D-426 | L-428 | Q-429 | P-430 |
| SEQ ID NO: 9 | US20120142046-0060 | D-218 | L-224 | C-445 | D-450 | A-453 | P-454 | S-458 | L-462 | P-463 | D-464 | L-466 | Q-467 | P-468 |
| SEQ ID NO: 10 | US20100313307-0046 | D-194 | L-200 | C-421 | D-426 | A-429 | P-430 | S-434 | L-438 | P-439 | D-440 | L-442 | Q-443 | P-444 |
| SEQ ID NO: 11 | US20100189706-0282 | D-194 | A-200 | S-421 | D-426 | A-429 | T-430 | Y-434 | A-438 | S-439 | A-440 | L-442 | Q-443 | P-444 |
| SEQ ID NO: 12 | US20100189706-0358 | D-198 | A-204 | C-425 | N-430 | A-433 | P-434 | Y-438 | S-442 | A-443 | D-444 | L-446 | Q-447 | P-448 |
| SEQ ID NO: 13 | US20100189706-0401 | E-200 | A-206 | S-427 | D-432 | A-435 | A-436 | Y-440 | Y-444 | S-445 | D-446 | L-448 | Q-449 | P-450 |
| SEQ ID NO: 14 | US4894338-0002 | D-197 | L-203 | C-424 | D-429 | A-432 | P-433 | S-437 | L-441 | P-442 | D-443 | L-445 | Q-446 | P-447 |
| SEQ ID NO: 15 | US6114158-0008 | D-196 | L-202 | C-423 | D-428 | A-431 | P-432 | S-436 | L-440 | P-441 | D-442 | L-444 | Q-445 | P-446 |
| SEQ ID NO: 16 | US8008056-0089 | D-197 | L-203 | C-424 | D-429 | A-432 | P-433 | S-437 | L-441 | P-442 | D-443 | L-445 | Q-446 | P-447 |
| SEQ ID NO: 17 | US20090325240-0923 | D-196 | L-202 | C-423 | D-428 | A-431 | P-432 | S-436 | L-440 | P-441 | D-442 | L-444 | Q-445 | P-446 |
| SEQ ID NO: 18 | US20090325240-0946 | D-196 | L-202 | C-423 | D-428 | A-431 | P-432 | S-436 | L-440 | P-441 | D-442 | L-444 | Q-445 | P-446 |
| SEQ ID NO: 19 | US20090325240-0970 | D-197 | L-203 | C-424 | D-429 | A-432 | P-433 | S-437 | L-441 | P-442 | D-443 | L-445 | Q-446 | P-447 |
| SEQ ID NO: 20 | US20120129229-0028 | D-197 | L-203 | C-424 | D-429 | A-432 | P-433 | S-437 | L-441 | P-442 | D-443 | L-445 | Q-446 | P-447 |
| SEQ ID NO: 21 | US8012734-0037 | D-173 | L-179 | C-400 | D-405 | A-408 | P-409 | P-413 | L-417 | P-418 | D-419 | L-421 | Q-422 | P-423 |
| SEQ ID NO: 22 | US8012734-0038 | D-173 | L-179 | C-400 | D-405 | A-408 | P-409 | P-413 | L-417 | P-418 | D-419 | L-421 | Q-422 | P-423 |
| SEQ ID NO: 23 | US8012734-0039 | D-173 | L-179 | C-400 | D-405 | A-408 | P-409 | P-413 | L-417 | P-418 | D-419 | L-421 | Q-422 | P-423 |
| SEQ ID NO: 24 | US8012734-0040 | D-173 | L-179 | C-400 | D-405 | A-408 | P-409 | P-413 | L-417 | P-418 | D-419 | L-421 | Q-422 | P-423 |
| SEQ ID NO: 25 | US8012734-0041 | D-173 | L-179 | C-400 | D-405 | A-408 | P-409 | P-413 | L-417 | P-418 | D-419 | L-421 | Q-422 | P-423 |
| SEQ ID NO: 26 | US8012734-0042 | D-173 | L-179 | C-400 | D-405 | A-408 | P-409 | P-413 | L-417 | P-418 | D-419 | L-421 | Q-422 | P-423 |
| SEQ ID NO: 27 | US8012734-0043 | D-173 | L-179 | C-400 | D-405 | A-408 | P-409 | P-413 | L-417 | P-418 | D-419 | L-421 | Q-422 | P-423 |
| SEQ ID NO: 28 | US8012734-0044 | D-173 | L-179 | C-400 | D-405 | A-408 | P-409 | P-413 | L-417 | P-418 | D-419 | L-421 | Q-422 | P-423 |
| SEQ ID NO: 29 | US8012734-0045 | D-173 | L-179 | C-400 | D-405 | A-408 | P-409 | P-413 | L-417 | P-418 | D-419 | L-421 | Q-422 | P-423 |
| SEQ ID NO: 30 | US8012734-0046 | D-173 | L-179 | C-400 | D-405 | A-408 | P-409 | P-413 | L-417 | P-418 | D-419 | L-421 | Q-422 | P-423 |
| SEQ ID NO: 31 | US8012734-0047 | D-173 | L-179 | C-400 | D-405 | A-408 | P-409 | P-413 | L-417 | P-418 | D-419 | L-421 | Q-422 | P-423 |
| SEQ ID NO: 32 | US8012734-0048 | D-173 | L-179 | C-400 | D-405 | A-408 | P-409 | P-413 | L-417 | P-418 | D-419 | L-421 | Q-422 | P-423 |
| SEQ ID NO: 33 | US8012734-0049 | D-173 | L-179 | C-400 | D-405 | A-408 | P-409 | P-413 | L-417 | P-418 | D-419 | L-421 | Q-422 | P-423 |
| SEQ ID NO: 34 | US8012734-0050 | D-173 | L-179 | C-400 | D-405 | A-408 | P-409 | P-413 | L-417 | P-418 | D-419 | L-421 | Q-422 | P-423 |
| SEQ ID NO: 35 | US8012734-0051 | D-173 | L-179 | C-400 | D-405 | A-408 | P-409 | P-413 | L-417 | P-418 | D-419 | L-421 | Q-422 | P-423 |
| SEQ ID NO: 36 | US8012734-0052 | D-173 | L-179 | C-400 | D-405 | A-408 | P-409 | P-413 | L-417 | P-418 | D-419 | L-421 | Q-422 | P-423 |
| SEQ ID NO: 37 | US8012734-0053 | D-173 | L-179 | C-400 | D-405 | A-408 | P-409 | P-413 | L-417 | P-418 | D-419 | L-421 | Q-422 | P-423 |
| SEQ ID NO: 38 | US8012734-0054 | D-173 | L-179 | C-400 | D-405 | A-408 | P-409 | P-413 | L-417 | P-418 | D-419 | L-421 | Q-422 | P-423 |
| SEQ ID NO: 39 | US8012734-0055 | D-173 | L-179 | C-400 | D-405 | A-408 | P-409 | P-413 | L-417 | P-418 | D-419 | L-421 | Q-422 | P-423 |
| SEQ ID NO: 40 | US8012734-0057 | D-173 | L-179 | C-400 | D-405 | A-408 | P-409 | P-413 | L-417 | P-418 | D-419 | L-421 | Q-422 | P-423 |
| SEQ ID NO: 41 | US8012734-0058 | D-173 | L-179 | C-400 | D-405 | A-408 | P-409 | P-413 | L-417 | P-418 | D-419 | L-421 | Q-422 | P-423 |
| SEQ ID NO: 42 | US8012734-0061 | D-173 | L-179 | C-400 | D-405 | A-408 | P-409 | P-413 | L-417 | P-418 | D-419 | L-421 | Q-422 | P-423 |
| SEQ ID NO: 43 | US8012734-0062 | D-173 | L-179 | C-400 | D-405 | A-408 | P-409 | P-413 | L-417 | P-418 | D-419 | L-421 | Q-422 | P-423 |
| SEQ ID NO: 44 | US8012734-0063 | D-173 | L-179 | C-400 | D-405 | A-408 | P-409 | P-413 | L-417 | P-418 | D-419 | L-421 | Q-422 | P-423 |
| SEQ ID NO: 45 | US8012734-0064 | D-173 | L-179 | C-400 | D-405 | A-408 | P-409 | P-413 | L-417 | P-418 | D-419 | L-421 | Q-422 | P-423 |
| SEQ ID NO: 46 | US8012734-0066 | D-173 | L-179 | C-400 | D-405 | A-408 | P-409 | P-413 | L-417 | P-418 | D-419 | L-421 | Q-422 | P-423 |
| SEQ ID NO: 47 | US8012734-0071 | D-173 | L-179 | C-400 | D-405 | A-408 | P-409 | P-413 | L-417 | P-418 | D-419 | L-421 | Q-422 | P-423 |
| SEQ ID NO: 48 | US8012734-0157 | D-173 | L-179 | C-400 | D-405 | A-408 | P-409 | P-413 | L-417 | P-418 | D-419 | L-421 | Q-422 | P-423 |
| SEQ ID NO: 49 | US8012734-0158 | D-173 | L-179 | C-400 | D-405 | A-408 | P-409 | P-413 | L-417 | P-418 | D-419 | L-421 | Q-422 | P-423 |
| SEQ ID NO: 50 | US8012734-0159 | D-173 | L-179 | C-400 | D-405 | A-408 | P-409 | P-413 | L-417 | P-418 | D-419 | L-421 | Q-422 | P-423 |
| SEQ ID NO: 51 | US8012734-0160 | D-173 | L-179 | C-400 | D-405 | A-408 | P-409 | P-413 | L-417 | P-418 | D-419 | L-421 | Q-422 | P-423 |
| SEQ ID NO: 52 | US8012734-0161 | D-173 | L-179 | C-400 | D-405 | A-408 | P-409 | P-413 | L-417 | P-418 | D-419 | L-421 | Q-422 | P-423 |
| SEQ ID NO: 53 | US7785854-0023 | D-185 | L-191 | C-412 | D-417 | A-420 | P-421 | S-425 | L-429 | P-430 | D-431 | L-433 | Q-434 | P-435 |
| SEQ ID NO: 54 | US8101398-0012 | D-173 | L-179 | C-400 | D-405 | A-408 | P-409 | P-413 | L-417 | P-418 | D-419 | L-421 | Q-422 | P-423 |
| SEQ ID NO: 55 | US8101398-0014 | D-173 | L-179 | C-400 | D-405 | A-408 | P-409 | P-413 | L-417 | P-418 | D-419 | L-421 | Q-422 | P-423 |
| SEQ ID NO: 56 | US8101398-0015 | D-173 | L-179 | C-400 | D-405 | A-408 | P-409 | P-413 | L-417 | P-418 | D-419 | L-421 | Q-422 | P-423 |
| SEQ ID NO: 57 | US8101398-0016 | D-173 | L-179 | C-400 | D-405 | A-408 | P-409 | P-413 | L-417 | P-418 | D-419 | L-421 | Q-422 | P-423 |
| SEQ ID NO: 58 | US8101398-0017 | D-173 | L-179 | C-400 | D-405 | A-408 | P-409 | P-413 | L-417 | P-418 | D-419 | L-421 | Q-422 | P-423 |
| SEQ ID NO: 59 | US8101398-0020 | D-173 | L-179 | C-400 | D-405 | A-408 | P-409 | P-413 | L-417 | P-418 | D-419 | L-421 | Q-422 | P-423 |
| SEQ ID NO: 60 | US8110389-0037 | D-173 | L-179 | C-400 | D-405 | A-408 | P-409 | P-413 | L-417 | P-418 | D-419 | L-421 | Q-422 | P-423 |
| SEQ ID NO: 61 | US8110389-0038 | D-173 | L-179 | C-400 | D-405 | A-408 | P-409 | P-413 | L-417 | P-418 | D-419 | L-421 | Q-422 | P-423 |
| SEQ ID NO: 62 | US8110389-0039 | D-173 | L-179 | C-400 | D-405 | A-408 | P-409 | P-413 | L-417 | P-418 | D-419 | L-421 | Q-422 | P-423 |
| SEQ ID NO: 63 | US8110389-0040 | D-173 | L-179 | C-400 | D-405 | A-408 | P-409 | P-413 | L-417 | P-418 | D-419 | L-421 | Q-422 | P-423 |
| SEQ ID NO: 64 | US8110389-0041 | D-173 | L-179 | C-400 | D-405 | A-408 | P-409 | P-413 | L-417 | P-418 | D-419 | L-421 | Q-422 | P-423 |
| SEQ ID NO: 65 | US20100016570-0083 | D-173 | L-179 | C-400 | D-405 | A-408 | P-409 | P-413 | L-417 | P-418 | D-419 | L-421 | Q-422 | P-423 |
| SEQ ID NO: 66 | US20100016570-0085 | D-173 | L-179 | C-400 | D-405 | A-408 | P-409 | P-413 | L-417 | P-418 | D-419 | L-421 | Q-422 | P-423 |
| SEQ ID NO: 67 | US20100016570-0088 | D-173 | L-179 | C-400 | D-405 | A-408 | P-409 | P-413 | L-417 | P-418 | D-419 | L-421 | Q-422 | P-423 |
| SEQ ID NO: 68 | US20100016570-0089 | D-173 | L-179 | C-400 | D-405 | A-408 | P-409 | P-413 | L-417 | P-418 | D-419 | L-421 | Q-422 | P-423 |
| SEQ ID NO: 69 | US20100016570-0094 | D-173 | L-179 | C-400 | D-405 | A-408 | P-409 | P-413 | L-417 | P-418 | D-419 | L-421 | Q-422 | P-423 |
| SEQ ID NO: 70 | US20100016570-0095 | D-173 | L-179 | C-400 | D-405 | A-408 | P-409 | P-413 | L-417 | P-418 | D-419 | L-421 | Q-422 | P-423 |
| SEQ ID NO: 71 | US20100016570-0096 | D-173 | L-179 | C-400 | D-405 | A-408 | P-409 | P-413 | L-417 | P-418 | D-419 | L-421 | Q-422 | P-423 |
| SEQ ID NO: 72 | US20100221778-0010 | D-173 | L-179 | C-400 | D-405 | A-408 | P-409 | P-413 | L-417 | P-418 | D-419 | L-421 | Q-422 | P-423 |
| SEQ ID NO: 73 | US20100221778-0011 | D-173 | L-179 | C-400 | D-405 | A-408 | P-409 | P-413 | L-417 | P-418 | D-419 | L-421 | Q-422 | P-423 |
| SEQ ID NO: 74 | US20100221778-0012 | D-173 | L-179 | C-400 | D-405 | A-408 | P-409 | P-413 | L-417 | P-418 | D-419 | L-421 | Q-422 | P-423 |
| SEQ ID NO: 75 | US20100221778-0013 | D-173 | L-179 | C-400 | D-405 | A-408 | P-409 | P-413 | L-417 | P-418 | D-419 | L-421 | Q-422 | P-423 |
| SEQ ID NO: 76 | US20100221778-0014 | D-173 | L-179 | C-400 | D-405 | A-408 | P-409 | P-413 | L-417 | P-418 | D-419 | L-421 | Q-422 | P-423 |
| SEQ ID NO: 77 | US20100221778-0015 | D-173 | L-179 | C-400 | D-405 | A-408 | P-409 | P-413 | L-417 | P-418 | D-419 | L-421 | Q-422 | P-423 |
| SEQ ID NO: 78 | US20100221778-0017 | D-173 | L-179 | C-400 | D-405 | A-408 | P-409 | P-413 | L-417 | P-418 | D-419 | L-421 | Q-422 | P-423 |
| SEQ ID NO: 79 | EP2401370-0016 | D-173 | L-179 | C-400 | D-405 | A-408 | P-409 | P-413 | L-417 | P-418 | D-419 | L-421 | Q-422 | P-423 |
| SEQ ID NO: 80 | JP2011523854-0016 | D-173 | L-179 | C-400 | D-405 | A-408 | P-409 | Y-413 | L-417 | P-418 | D-419 | L-421 | Q-422 | P-423 |
| SEQ ID NO: 81 | US20100317087-0018 | D-173 | L-179 | C-400 | D-405 | A-408 | P-409 | P-413 | L-417 | P-418 | D-419 | L-421 | Q-422 | P-423 |
| SEQ ID NO: 82 | US20110189744-0041 | D-179 | L-185 | C-406 | D-411 | A-414 | P-415 | S-419 | L-423 | P-424 | D-425 | L-427 | Q-428 | P-429 |
| SEQ ID NO: 83 | AAQ72468 | D-184 | A-190 | S-410 | D-415 | A-418 | A-419 | Y-423 | I-427 | D-428 | G-429 | V-431 | K-432 | P-433 |
| SEQ ID NO: 84 | AAA65585 | N-188 | A-194 | S-415 | D-420 | A-423 | A-424 | Y-428 | L-432 | D-433 | D-434 | L-436 | K-437 | P-438 |
| SEQ ID NO: 85 | ADC83999 | D-197 | L-203 | C-424 | D-429 | A-432 | P-433 | S-437 | L-441 | P-442 | D-443 | L-445 | Q-446 | P-447 |
| SEQ ID NO: 86 | AAA34210 | D-197 | L-203 | C-424 | D-429 | A-432 | P-433 | S-437 | L-441 | P-442 | D-443 | L-445 | Q-446 | P-447 |
| SEQ ID NO: 87 | AAQ76094 | D-197 | L-203 | C-424 | D-429 | A-432 | P-433 | S-437 | L-441 | P-442 | D-443 | L-445 | Q-446 | P-447 |
| SEQ ID NO: 88 | AAG39980 | D-197 | L-203 | C-424 | D-429 | A-432 | P-433 | S-437 | L-441 | P-442 | D-443 | L-445 | Q-446 | P-447 |
| SEQ ID NO: 89 | AAA72922 | D-197 | L-203 | C-424 | D-429 | A-432 | P-433 | S-437 | L-441 | P-442 | D-443 | L-445 | Q-446 | P-447 |
| SEQ ID NO: 90 | ABF56208 | D-196 | L-202 | C-423 | D-428 | A-431 | P-432 | S-436 | L-440 | P-441 | D-442 | L-444 | Q-445 | P-446 |
| SEQ ID NO: 91 | ACZ34301 | D-196 | L-202 | C-423 | D-428 | A-431 | P-432 | S-436 | L-440 | P-441 | D-442 | L-444 | Q-445 | P-446 |
| SEQ ID NO: 92 | ABG48766 | D-196 | L-202 | C-423 | D-428 | A-431 | P-432 | S-436 | L-440 | P-441 | D-442 | L-444 | Q-445 | P-446 |
| SEQ ID NO: 93 | ACH96126 | D-196 | L-202 | C-423 | D-428 | A-431 | P-432 | S-436 | L-440 | P-441 | D-442 | L-444 | Q-445 | P-446 |
| SEQ ID NO: 94 | ADJ10628 | D-196 | L-202 | C-423 | D-428 | A-431 | P-432 | S-436 | L-440 | P-441 | D-442 | L-444 | Q-445 | P-446 |
| SEQ ID NO: 95 | AAU05379 | D-196 | L-202 | C-423 | D-428 | A-431 | P-432 | S-436 | L-440 | P-441 | D-442 | L-444 | Q-445 | P-446 |
| SEQ ID NO: 96 | AEO62210 | D-209 | A-215 | C-435 | N-440 | S-443 | P-444 | Y-448 | L-452 | S-453 | D-454 | L-456 | Q-457 | P-458 |
| SEQ ID NO: 97 | CBX74420 | D-199 | A-205 | C-426 | N-431 | A-434 | P-435 | Y-439 | S-443 | A-444 | D-445 | L-447 | Q-448 | P-449 |
| SEQ ID NO: 98 | AEO55787 | D-210 | A-216 | S-436 | D-441 | A-444 | A-445 | Y-449 | L-453 | S-454 | D-455 | L-457 | Q-458 | P-459 |
| SEQ ID NO: 99 | AAY88915 | D-204 | A-210 | S-430 | D-435 | A-438 | A-439 | Y-443 | L-447 | S-448 | D-449 | L-451 | T-452 | P-453 |
| SEQ ID NO: 100 | CAP60942 | D-212 | A-218 | C-438 | D-443 | A-446 | A-447 | H-451 | F-455 | A-456 | D-457 | L-459 | K-460 | P-461 |
| SEQ ID NO: 101 | AAW64927 | D-204 | A-210 | S-430 | D-435 | A-438 | A-439 | Y-443 | L-447 | S-448 | D-449 | L-451 | T-452 | P-453 |
| SEQ ID NO: 102 | ADZ99361 | D-194 | A-200 | C-420 | N-425 | A-428 | A-429 | Y-433 | L-437 | S-438 | D-439 | L-441 | Q-442 | P-443 |
| SEQ ID NO: 103 | CAD70733 | D-212 | A-218 | S-438 | D-443 | A-446 | T-447 | Y-451 | L-455 | S-456 | D-457 | L-459 | K-460 | P-461 |
| SEQ ID NO: 104 | BAB39154 | D-204 | A-210 | C-430 | D-435 | A-438 | A-439 | Y-443 | L-447 | E-448 | D-449 | L-451 | K-452 | P-453 |
| SEQ ID NO: 105 | ABF50873 | D-183 | L-189 | S-409 | D-414 | A-417 | A-418 | A-422 | Y-426 | S-427 | D-428 | L-430 | Q-431 | P-432 |
| SEQ ID NO: 106 | CAK41068 | D-187 | L-193 | S-413 | D-418 | A-421 | T-422 | A-426 | Y-430 | S-431 | D-432 | L-434 | Q-435 | P-436 |
| SEQ ID NO: 107 | CAP93233 | D-119 | L-125 | S-345 | D-350 | A-353 | A-354 | A-358 | Y-362 | S-363 | D-364 | L-366 | Q-367 | P-368 |
| SEQ ID NO: 108 | CAK39856 | D-128 | L-134 | S-354 | D-359 | S-362 | S-363 | A-367 | Y-371 | S-372 | D-373 | L-375 | Q-376 | P-377 |
| SEQ ID NO: 109 | BAI65845 | D-135 | L-141 | S-361 | D-366 | A-369 | E-370 | A-374 | Y-378 | A-379 | D-380 | L-382 | T-383 | P-384 |
| SEQ ID NO: 110 | CCD44345 | D-187 | A-193 | G-413 | D-418 | A-421 | A-422 | F-426 | L-430 | A-431 | D-432 | L-434 | K-435 | P-436 |
| SEQ ID NO: 111 | AAL78165 | D-187 | A-193 | S-413 | N-418 | S-421 | P-422 | Y-426 | L-430 | S-431 | D-432 | L-434 | Q-435 | P-436 |
| SEQ ID NO: 112 | ACH91035 | D-183 | A-189 | S-410 | N-415 | A-418 | T-419 | F-423 | Y-427 | S-428 | G-429 | L-431 | Q-432 | P-433 |
| SEQ ID NO: 113 | ADX86895 | D-192 | L-198 | C-418 | D-423 | S-426 | P-427 | A-431 | Y-435 | S-436 | D-437 | L-439 | K-440 | P-441 |
| SEQ ID NO: 114 | BAA74458 | D-183 | A-189 | S-410 | N-415 | A-418 | T-419 | F-423 | Y-427 | S-428 | D-429 | L-431 | Q-432 | P-433 |
| SEQ ID NO: 115 | CBX97039 | D-125 | L-131 | S-352 | N-357 | S-360 | A-361 | G-365 | R-369 | D-370 | S-371 | F-373 | K-374 | P-375 |
| SEQ ID NO: 116 | AAM76664 | D-115 | L-121 | S-342 | D-347 | A-350 | A-351 | A-355 | R-359 | N-360 | S-361 | F-363 | K-364 | P-365 |
| SEQ ID NO: 117 | AAA50607.1 | D-167 | L-173 | C-392 | D-397 | S-400 | P-401 | S-405 | L-409 | S-410 | D-411 | H-413 | Q-414 | P-415 |
| SEQ ID NO: 118 | AAQ38151.1 | G-121 | K-127 | S-349 | D-354 | A-357 | A-358 | S-362 | K-366 | P-367 | D-368 | F-370 | K-371 | P-372 |
| SEQ ID NO: 119 | BAH08702.1 | D-183 | A-189 | A-408 | D-413 | A-416 | P-417 | T-421 | K-425 | S-426 | D-427 | H-429 | K-430 | P-431 |
| SEQ ID NO: 120 | BAH08703.1 | D-117 | N-123 | S-350 | D-355 | S-358 | N-359 | P-363 | S-367 | P-368 | V-369 | H-371 | V-372 | P-373 |
| SEQ ID NO: 121 | BAH08704.1 | D-119 | K-125 | S-352 | D-357 | A-360 | V-361 | E-365 | S-369 | P-370 | A-371 | H-373 | V-374 | P-375 |
| SEQ ID NO: 122 | Q7SIG5 | D-90 | G-96 | S-315 | G-320 | G-323 | — | — | — | — | — | — | — | — |
| SEQ ID NO: 123 | BAG48183.1 | D-181 | K-187 | S-406 | N-411 | S-414 | P-415 | S-419 | L-423 | S-424 | D-425 | T-427 | Q-428 | P-429 |
| SEQ ID NO: 124 | AAK28357.1 | D-174 | L-180 | C-397 | N-402 | S-405 | P-406 | A-410 | L-414 | P-415 | D-416 | T-418 | P-419 | N-420 |
| SEQ ID NO: 125 | CAH05679.1 | N-118 | A-124 | S-344 | D-349 | A-352 | E-353 | Y-357 | L-361 | E-362 | D-363 | L-365 | K-366 | P-367 |
| SEQ ID NO: 126 | AAC09066.1 | T-198 | Y-204 | S-409 | S-412 | G-415 | P-416 | P-420 | H-424 | S-425 | D-426 | L-428 | P-429 | G-430 |
| SEQ ID NO: 127 | AAB92678.1 | T-205 | N-211 | A-415 | S-418 | G-421 | S-422 | P-426 | R-430 | G-431 | D-432 | L-434 | Q-435 | G-436 |
| SEQ ID NO: 128 | AAB92679.1 | T-204 | G-210 | S-408 | A-411 | — | — | P-416 | N-420 | S-421 | D-422 | L-424 | Q-425 | G-426 |
| SEQ ID NO: 129 | AAB32942.1 | D-189 | K-195 | C-414 | N-419 | S-422 | P-423 | S-427 | L-431 | P-432 | D-433 | A-435 | Q-436 | P-437 |
| SEQ ID NO: 130 | AAM94167.1 | G-208 | L-214 | S-440 | D-445 | A-448 | K-449 | G-453 | H-457 | R-458 | T-459 | M-461 | K-462 | P-463 |
| SEQ ID NO: 131 | AAD51055.1 | T-207 | N-213 | A-416 | S-419 | G-422 | S-423 | P-427 | R-431 | W-432 | D-433 | L-435 | Q-436 | G-437 |
| SEQ ID NO: 132 | AAL92497.1 | G-213 | L-219 | S-445 | D-450 | A-453 | V-454 | G-458 | H-462 | A-463 | T-464 | M-466 | K-467 | P-468 |
| SEQ ID NO: 133 | CAH05678.1 | D-186 | A-192 | C-412 | D-417 | A-420 | A-421 | H-425 | N-429 | P-430 | D-431 | L-433 | K-434 | P-435 |

TABLE 5

| | | | | Cellulose | Cellulose | | | | |
|---|---|---|---|---|---|---|---|---|---|
| | | | | Binding | Binding | CBD- | CBD- | Catalytic | Catalytic |
| | | Signal | Signal | Domain | Domain | CD | CD | Domain | Domain |
| Sequence | Database Accession | sequence | sequence | (CBD) | (CBD) | linker | linker | (CD) | (CD) |
| Identifier | Number or Patent | (SS) start | (SS) end | CBM_1 start | CBM_1 end | start | end | start | end |
| (SEQ ID NO:) | Document Number | position | position | position | position | position | position | position | position |
| SEQ ID NO: 2 | BD23134* | M-1 | A-18 | C-28 | I-63 | P-64 | G-103 | S-104 | L-468 |
| SEQ ID NO: 3 | BD21660* | M-1 | A-18 | Q-19 | T-54 | P-55 | A-102 | T-103 | F-470 |
| SEQ ID NO: 4 | BD22435* | M-1 | S-18 | C-27 | L-62 | P-63 | G-107 | S-108 | L-472 |
| SEQ ID NO: 5 | US8101393-0098 | M-1 | S-18 | Q-19 | T-54 | P-55 | A-102 | T-103 | F-470 |
| SEQ ID NO: 6 | WO2011059740-0002 | M-1 | S-18 | Q-19 | T-54 | P-55 | A-101 | T-102 | F-469 |
| SEQ ID NO: 7 | US8168863-0013 | — | — | C-10 | L-45 | P-46 | G-90 | S-91 | L-455 |
| SEQ ID NO: 8 | US20090320831-0082 | — | — | C-10 | L-45 | P-46 | G-89 | S-90 | L-454 |
| SEQ ID NO: 9 | US20120142046-0060 | M-1 | A-19 | C-48 | L-83 | P-84 | G-127 | S-128 | L-492 |
| SEQ ID NO: 10 | US20100313307-0046 | M-1 | A-20 | C-24 | L-59 | P-60 | G-103 | S-104 | L-468 |
| SEQ ID NO: 11 | US20100189706-0282 | M-1 | A-18 | C-28 | I-63 | P-64 | G-103 | S-104 | L-468 |
| SEQ ID NO: 12 | US20100189706-0358 | M-1 | S-18 | C-27 | L-62 | P-63 | G-107 | S-108 | L-472 |
| SEQ ID NO: 13 | US20100189706-0401 | M-1 | A-18 | C-28 | L-62 | S-63 | G-109 | A-110 | L-474 |
| SEQ ID NO: 14 | US4894338-0002 | M-1 | S-18 | C-27 | L-62 | P-63 | G-106 | S-107 | L-471 |
| SEQ ID NO: 15 | US6114158-0008 | M-1 | S-18 | C-27 | L-62 | P-63 | G-105 | S-106 | L-470 |
| SEQ ID NO: 16 | US8008056-0089 | M-1 | S-18 | C-27 | L-62 | P-63 | G-106 | S-107 | L-471 |
| SEQ ID NO: 17 | US20090325240-0923 | M-1 | S-18 | C-27 | L-62 | P-63 | G-105 | S-106 | L-470 |
| SEQ ID NO: 18 | US20090325240-0946 | M-1 | S-18 | C-27 | P-62 | P-63 | G-105 | S-106 | L-470 |
| SEQ ID NO: 19 | US20090325240-0970 | M-1 | S-18 | C-27 | L-62 | P-63 | G-106 | S-107 | L-471 |
| SEQ ID NO: 20 | US20120129229-0028 | M-1 | S-18 | C-27 | L-62 | P-63 | G-106 | S-107 | L-471 |
| SEQ ID NO: 21 | US8012734-0037 | — | — | C-3 | L-38 | P-39 | G-82 | S-83 | L-447 |
| SEQ ID NO: 22 | US8012734-0038 | — | — | C-3 | L-38 | P-39 | G-82 | S-83 | L-447 |
| SEQ ID NO: 23 | US8012734-0039 | — | — | C-3 | L-38 | P-39 | G-82 | S-83 | L-447 |
| SEQ ID NO: 24 | US8012734-0040 | — | — | C-3 | L-38 | P-39 | G-82 | S-83 | L-447 |
| SEQ ID NO: 25 | US8012734-0041 | — | — | C-3 | L-38 | P-39 | G-82 | S-83 | L-447 |
| SEQ ID NO: 26 | US8012734-0042 | — | — | C-3 | L-38 | P-39 | G-82 | S-83 | L-447 |
| SEQ ID NO: 27 | US8012734-0043 | — | — | C-3 | L-38 | P-39 | G-82 | S-83 | L-447 |
| SEQ ID NO: 28 | US8012734-0044 | — | — | C-3 | L-38 | P-39 | G-82 | S-83 | L-447 |
| SEQ ID NO: 29 | US8012734-0045 | — | — | C-3 | L-38 | P-39 | G-82 | S-83 | L-447 |
| SEQ ID NO: 30 | US8012734-0046 | — | — | C-3 | L-38 | P-39 | G-82 | S-83 | L-447 |
| SEQ ID NO: 31 | US8012734-0047 | — | — | C-3 | L-38 | P-39 | G-82 | S-83 | L-447 |
| SEQ ID NO: 32 | US8012734-0048 | — | — | C-3 | L-38 | P-39 | G-82 | S-83 | L-447 |
| SEQ ID NO: 33 | US8012734-0049 | — | — | C-3 | L-38 | P-39 | G-82 | S-83 | L-447 |
| SEQ ID NO: 34 | US8012734-0050 | — | — | C-3 | L-38 | P-39 | G-82 | S-83 | L-447 |
| SEQ ID NO: 35 | US8012734-0051 | — | — | C-3 | L-38 | P-39 | G-82 | S-83 | L-447 |
| SEQ ID NO: 36 | US8012734-0052 | — | — | C-3 | L-38 | P-39 | G-82 | S-83 | L-447 |
| SEQ ID NO: 37 | US8012734-0053 | — | — | C-3 | L-38 | P-39 | G-82 | S-83 | L-447 |
| SEQ ID NO: 38 | US8012734-0054 | — | — | C-3 | L-38 | P-39 | G-82 | S-83 | L-447 |
| SEQ ID NO: 39 | US8012734-0055 | — | — | C-3 | L-38 | P-39 | G-82 | S-83 | L-447 |
| SEQ ID NO: 40 | US8012734-0057 | — | — | C-3 | L-38 | P-39 | G-82 | S-83 | L-447 |
| SEQ ID NO: 41 | US8012734-0058 | — | — | C-3 | L-38 | P-39 | G-82 | S-83 | L-447 |
| SEQ ID NO: 42 | US8012734-0061 | — | — | C-3 | L-38 | P-39 | G-82 | S-83 | L-447 |
| SEQ ID NO: 43 | US8012734-0062 | — | — | C-3 | L-38 | P-39 | G-82 | S-83 | L-447 |
| SEQ ID NO: 44 | US8012734-0063 | — | — | C-3 | L-38 | P-39 | G-82 | S-83 | L-447 |
| SEQ ID NO: 45 | US8012734-0064 | — | — | C-3 | L-38 | P-39 | G-82 | S-83 | L-447 |
| SEQ ID NO: 46 | US8012734-0066 | — | — | C-3 | L-38 | P-39 | G-82 | S-83 | L-447 |
| SEQ ID NO: 47 | US8012734-0071 | — | — | C-3 | L-38 | P-39 | G-82 | S-83 | L-447 |
| SEQ ID NO: 48 | US8012734-0157 | — | — | C-3 | L-38 | P-39 | G-82 | S-83 | L-447 |
| SEQ ID NO: 49 | US8012734-0158 | — | — | C-3 | L-38 | P-39 | G-82 | S-83 | L-447 |
| SEQ ID NO: 50 | US8012734-0159 | — | — | C-3 | L-38 | P-39 | G-82 | S-83 | L-447 |
| SEQ ID NO: 51 | US8012734-0160 | — | — | C-3 | L-38 | P-39 | G-82 | S-83 | L-447 |
| SEQ ID NO: 52 | US8012734-0161 | — | — | C-3 | L-38 | P-39 | G-82 | S-83 | L-447 |
| SEQ ID NO: 53 | US7785854-0023 | — | — | C-15 | L-50 | P-51 | G-94 | S-95 | L-459 |
| SEQ ID NO: 54 | US8101398-0012 | — | — | C-3 | L-38 | P-39 | G-82 | S-83 | L-447 |
| SEQ ID NO: 55 | US8101398-0014 | — | — | C-3 | L-38 | P-39 | G-82 | S-83 | L-447 |
| SEQ ID NO: 56 | US8101398-0015 | — | — | C-3 | L-38 | P-39 | G-82 | S-83 | L-447 |
| SEQ ID NO: 57 | US8101398-0016 | — | — | C-3 | L-38 | P-39 | G-82 | S-83 | L-447 |
| SEQ ID NO: 58 | US8101398-0017 | — | — | C-3 | L-38 | P-39 | G-82 | S-83 | L-447 |
| SEQ ID NO: 59 | US8101398-0020 | — | — | C-3 | L-38 | P-39 | G-82 | S-83 | L-447 |
| SEQ ID NO: 60 | US8110389-0037 | — | — | C-3 | L-38 | P-39 | G-82 | S-83 | L-447 |
| SEQ ID NO: 61 | US8110389-0038 | — | — | C-3 | L-38 | P-39 | G-82 | S-83 | L-447 |
| SEQ ID NO: 62 | US8110389-0039 | — | — | C-3 | L-38 | P-39 | G-82 | S-83 | L-447 |
| SEQ ID NO: 63 | US8110389-0040 | — | — | C-3 | L-38 | P-39 | G-82 | S-83 | L-447 |
| SEQ ID NO: 64 | US8110389-0041 | — | — | C-3 | L-38 | P-39 | G-82 | S-83 | L-447 |
| SEQ ID NO: 65 | US20100016570-0083 | — | — | C-3 | L-38 | P-39 | G-82 | S-83 | L-447 |
| SEQ ID NO: 66 | US20100016570-0085 | — | — | C-3 | L-38 | P-39 | G-82 | S-83 | L-447 |
| SEQ ID NO: 67 | US20100016570-0088 | — | — | C-3 | L-38 | P-39 | G-82 | S-83 | L-447 |
| SEQ ID NO: 68 | US20100016570-0089 | — | — | C-3 | L-38 | P-39 | G-82 | S-83 | L-447 |
| SEQ ID NO: 69 | US20100016570-0094 | — | — | C-3 | L-38 | P-39 | G-82 | S-83 | L-447 |
| SEQ ID NO: 70 | US20100016570-0095 | — | — | C-3 | L-38 | P-39 | G-82 | S-83 | L-447 |
| SEQ ID NO: 71 | US20100016570-0096 | — | — | C-3 | L-38 | P-39 | G-82 | S-83 | L-447 |
| SEQ ID NO: 72 | US2010221778-0010 | — | — | C-3 | L-38 | P-39 | G-82 | S-83 | L-447 |
| SEQ ID NO: 73 | US2010221778-0011 | — | — | C-3 | L-38 | P-39 | G-82 | S-83 | L-447 |
| SEQ ID NO: 74 | US2010221778-0012 | — | — | C-3 | L-38 | P-39 | G-82 | S-83 | L-447 |
| SEQ ID NO: 75 | US2010221778-0013 | — | — | C-3 | L-38 | P-39 | G-82 | S-83 | L-447 |
| SEQ ID NO: 76 | US2010221778-0014 | — | — | C-3 | L-38 | P-39 | G-82 | S-83 | L-447 |
| SEQ ID NO: 77 | US2010221778-0015 | — | — | C-3 | L-38 | P-39 | G-82 | S-83 | L-447 |
| SEQ ID NO: 78 | US2010221778-0017 | — | — | C-3 | L-38 | P-39 | G-82 | S-83 | L-447 |
| SEQ ID NO: 79 | EP2401370-0016 | — | — | C-3 | L-38 | P-39 | G-82 | S-83 | L-447 |
| SEQ ID NO: 80 | JP2011523854-0016 | — | — | C-3 | L-38 | P-39 | G-82 | S-83 | L-447 |
| SEQ ID NO: 81 | US20100317087-0018 | — | — | C-9 | L-38 | P-39 | G-82 | S-83 | L-447 |
| SEQ ID NO: 82 | US20110189744-0041 | — | — | C-9 | L-44 | P-45 | G-88 | S-89 | L-453 |
| SEQ ID NO: 83 | AAQ72468 | M-1 | A-18 | C-27 | Q-63 | P-64 | T-93 | S-94 | L-457 |
| SEQ ID NO: 84 | AAA65585 | M-1 | A-16 | C-25 | Q-61 | P-62 | T-97 | S-98 | L-462 |
| SEQ ID NO: 85 | ADC83999 | M-1 | S-18 | C-27 | L-62 | P-63 | G-106 | S-107 | L-471 |
| SEQ ID NO: 86 | AAA34210 | M-1 | S-18 | C-27 | L-62 | P-63 | G-106 | S-107 | L-471 |
| SEQ ID NO: 87 | AAQ76094 | M-1 | S-18 | C-27 | L-62 | P-63 | G-106 | S-107 | L-471 |
| SEQ ID NO: 88 | AAG39980 | M-1 | S-18 | C-27 | L-62 | P-63 | G-106 | S-107 | L-471 |
| SEQ ID NO: 89 | AAA72922 | M-1 | S-18 | C-27 | L-62 | P-63 | G-106 | S-107 | L-471 |
| SEQ ID NO: 90 | ABF56208 | M-1 | S-18 | C-27 | L-62 | P-63 | G-105 | S-106 | L-470 |
| SEQ ID NO: 91 | ACZ34301 | M-1 | S-18 | C-27 | L-62 | P-63 | G-105 | S-106 | L-470 |
| SEQ ID NO: 92 | ABG48766 | M-1 | S-18 | C-27 | L-62 | P-63 | G-105 | S-106 | L-470 |
| SEQ ID NO: 93 | ACH96126 | M-1 | S-18 | C-27 | L-62 | P-63 | G-105 | S-106 | L-470 |
| SEQ ID NO: 94 | ADJ10628 | M-1 | S-18 | C-27 | L-62 | P-63 | G-105 | S-106 | L-470 |
| SEQ ID NO: 95 | AAU05379 | M-1 | S-18 | C-27 | P-62 | P-63 | G-105 | S-106 | L-470 |
| SEQ ID NO: 96 | AEO62210 | M-1 | A-17 | C-27 | L-62 | P-63 | A-115 | S-116 | F-481 |
| SEQ ID NO: 97 | CBX74420 | M-1 | S-19 | C-28 | L-63 | P-64 | G-108 | S-109 | L-473 |
| SEQ ID NO: 98 | AEO55787 | M-1 | A-17 | C-27 | L-62 | P-63 | A-116 | T-117 | F-482 |
| SEQ ID NO: 99 | AAY88915 | M-1 | A-17 | C-27 | I-62 | P-63 | A-110 | S-111 | F-476 |
| SEQ ID NO: 100 | CAP60942 | M-1 | A-17 | C-27 | L-62 | P-63 | A-118 | S-119 | F-484 |
| SEQ ID NO: 101 | AAW64927 | M-1 | A-17 | C-27 | I-62 | P-63 | A-110 | S-111 | F-476 |
| SEQ ID NO: 102 | ADZ99361 | M-1 | A-18 | C-27 | V-62 | P-63 | A-100 | S-101 | F-466 |
| SEQ ID NO: 103 | CAD70733 | M-1 | A-18 | C-28 | L-63 | P-64 | A-118 | S-119 | F-484 |
| SEQ ID NO: 104 | BAB39154 | M-1 | A-16 | C-26 | L-61 | P-62 | A-109 | T-110 | F-476 |
| SEQ ID NO: 105 | ABF50873 | M-1 | A-19 | Q-20 | L-55 | P-56 | V-87 | T-88 | F-455 |
| SEQ ID NO: 106 | CAK41068 | M-1 | A-18 | Q-19 | T-54 | P-55 | P-91 | T-92 | F-459 |
| SEQ ID NO: 107 | CAP93233 | M-1 | A-23 | — | — | — | — | T-27 | L-391 |
| SEQ ID NO: 108 | CAK39856 | M-1 | A-16 | — | — | — | — | A-35 | L-400 |
| SEQ ID NO: 109 | BAI65845 | M-1 | A-22 | — | — | — | — | A-43 | L-407 |
| SEQ ID NO: 110 | CCD44345 | M-1 | A-19 | Q-20 | L-55 | P-56 | A-92 | T-93 | F-459 |
| SEQ ID NO: 111 | AAL78165 | M-1 | A-19 | Q-20 | V-55 | P-56 | V-94 | T-95 | F-459 |
| SEQ ID NO: 112 | ACH91035 | M-1 | A-19 | Q-20 | I-55 | P-56 | A-90 | T-91 | V-457 |
| SEQ ID NO: 113 | ADX86895 | M-1 | A-18 | Q-19 | L-54 | P-55 | T-99 | A-100 | F-464 |
| SEQ ID NO: 114 | BAA74458 | M-1 | A-19 | Q-20 | I-55 | P-56 | A-90 | T-91 | V-457 |
| SEQ ID NO: 115 | CBX97039 | M-1 | A-17 | — | — | — | — | T-35 | A-399 |
| SEQ ID NO: 116 | AAM76664 | M-1 | A-18 | — | — | — | — | T-25 | A-389 |
| SEQ ID NO: 117 | AAA50607.1 | M-1 | A-20 | Q-21 | L-56 | P-57 | G-81 | T-82 | L-438 |
| SEQ ID NO: 118 | AAQ38151.1 | M-1 | A-17 | — | — | — | — | A-37 | F-395 |
| SEQ ID NO: 119 | BAH08702.1 | M-1 | A-21 | Q-22 | L-57 | P-58 | G-91 | G-92 | L-454 |
| SEQ ID NO: 120 | BAH08703.1 | M-1 | A-19 | — | — | — | — | I-23 | L-396 |
| SEQ ID NO: 121 | BAH08704.1 | M-1 | A-19 | — | — | — | — | F-25 | L-398 |
| SEQ ID NO: 122 | Q7SIG5 | — | — | — | — | — | — | Q-1 | L-346 |
| SEQ ID NO: 123 | BAG48183.1 | M-1 | G-20 | Q-21 | I-56 | P-57 | S-89 | S-90 | L-452 |
| SEQ ID NO: 124 | AAK28357.1 | M-1 | G-20 | Q-21 | L-56 | P-57 | T-83 | G-84 | L-443 |
| SEQ ID NO: 125 | CAH05679.1 | M-1 | A-18 | — | — | — | — | L-23 | F-390 |
| SEQ ID NO: 126 | AAC09066.1 | M-1 | A-16 | S-17¹ | N-57¹ | Q-60² | G-102² | T-116 | I-453 |
| SEQ ID NO: 127 | AAB92678.1 | M-1 | A-17 | S-18¹ | S-57¹ | C-63² | G-107² | Q-123 | F-459 |
| SEQ ID NO: 128 | AAB92679.1 | M-1 | A-17 | S-18¹ | N-57¹ | G-59² | P-101² | V-123 | F-449 |
| SEQ ID NO: 129 | AAB32942.1 | M-1 | G-20 | Q-21 | L-56 | P-57 | N-97 | P-98 | L-460 |
| SEQ ID NO: 130 | AAM94167.1 | M-1 | A-19 | A-19¹ | I-63¹ | Q-64² | P-107² | G-116 | L-486 |
| SEQ ID NO: 131 | AAD51055.1 | M-1 | A-17 | S-18¹ | N-58¹ | G-61² | N-103² | R-125 | F-460 |
| SEQ ID NO: 132 | AAL92497.1 | M-1 | A-19 | A-19¹ | A-60¹ | T-61² | S-104² | G-121 | L-491 |
| SEQ ID NO: 133 | CAH05678.1 | M-1 | A-18 | C-27 | V-62 | P-63 | P-95 | T-96 | F-458 |

SEQ ID NOS: 126-128 and 130-132 have two CBM_10 domains rather than a single CBM_1 domain
¹amino acid position corresponding to the first CBM_10 domain start or end position
²amino acid position corresponding to the second CBM_10 domain start or end position

What is claimed is:

1-77. (canceled)

78. A polypeptide comprising SEQ ID NO:2 or a variant thereof, wherein the variant comprises a cellobiohydrolase II (“CBH II”) catalytic domain, wherein the catalytic domain has one or more amino acid substitutions selected from:
an I235V substitution;
a S104V substitution;
a K309H substitution;
a S115A substitution; and
a S115M substitution and/or
a polypeptide comprising a variant cellobiohydrolase II (“CBH II”) cellulose binding domain, wherein the cellulose binding domain has one or more amino acid substitutions selected from:
a G37S substitution; and
an A33K substitution and/or.
a polypeptide comprising a variant cellobiohydrolase II (“CBH II”) SS linker sequence, wherein the SS linker sequence has one or more amino acid substitutions selected from:
a L21R substitution;
an E23K substitution; and
an E23N substitution and/or.
a polypeptide comprising a variant cellobiohydrolase II (“CBH II”) CBD-CD linker sequence, wherein the CBD-CD linker sequence has one or more amino acid substitutions selected from:
a P64W substitution;
a P64E substitution;
a G65L substitution;
a E66R substitution; and
a G67K substitution and/or
a polypeptide comprising a variant cellobiohydrolase II (“CBH II”) catalytic domain, wherein the catalytic domain has a D194N substitution, an A200L substitution, a S421C substitution, a D426N substitution, an A429S substitution, a T430P substitution, a Y434A substitution, an A438L substitution, a S439P substitution, an A440D substitution, a L442T substitution, a Q443P substitution, and/or a P444N substitution.

79. The polypeptide of claim 78, which has a CBH II specific activity that is at least 2%, at least 3%, at least 4%, at least 5%, at least 10%, at least 15%, at least 20%, at least 25% or at least 30% greater than the specific activity of a reference CBH II which does not have the same substitution(s).

80. The polypeptide of claim 78, wherein the polypeptide comprises an amino acid sequence having at least 60%, at least 70%, at least 80%, at least 90%, or at least 95% sequence identity to SEQ ID NO:2.

81. The polypeptide of claim 78, wherein the polypeptide comprises an amino acid sequence having at least 90%, at least 95% or at least 97% sequence identity of the mature portion of a polypeptide according to any one of SEQ ID NOs:2-133.

82. A composition comprising a polypeptide of claim 78.

83. The composition of claim 82, in which said polypeptide represents at least 1%-25% of all polypeptides in said composition.

84. The composition of claim 82, which is a whole cellulase.

85. A fermentation broth comprising a polypeptide of claim 78.

86. A method for saccharifying biomass, comprising: treating biomass with a composition of claim 82 or with a fermentation broth of claim 85.

87. A method for producing a fermentation product, comprising:
(a) treating biomass with a composition of claim 82 or with a fermentation broth of claim 85, thereby producing fermentable sugars; and
(b) culturing a fermenting microorganism in the presence of the fermentable sugars produced in step (a) under fermentation conditions, thereby producing a fermentation product.

88. The method of claim 87, wherein said fermentable sugars comprise monosaccharides or disaccharides or a combination thereof.

89. The method of claim 87, wherein the fermentation product is ethanol.

90. The method of claim 87, wherein said fermenting microorganism is a bacterium or a yeast.

91. The method of claim 90, wherein said fermenting microorganism is a bacterium selected from Zymomonas mobilis, Escherichia coli or Klebsiella oxytoca or a yeast selected from Saccharomyces cerevisiae, Saccharomyces uvarum, Kluyveromyces fragilis, Kluyveromyces lactis, Candida pseudotropicalis, or Pachysolen tannophilus.

92. The method of claim 87, wherein said biomass is corn stover, bagasses, sorghum, giant reed, elephant grass, miscanthus, Japanese cedar, wheat straw, switchgrass, hardwood pulp, softwood pulp, crushed sugar cane, energy cane, or Napier grass.

93. An isolated nucleic acid comprising a nucleotide sequence encoding the polypeptide of claim 78.

94. A vector comprising the nucleic acid of claim 93.

95. The vector of claim 94, which further comprises a heterologous promoter sequence operably linked to said nucleotide sequence.

96. The vector of claim 95, wherein the promoter sequence is operable in yeast or in filamentous fungi.

97. An isolated recombinant cell engineered to express the nucleic acid of claim 93.

98. The recombinant cell of claim 97, wherein the cell is a filamentous fungal cell selected from the genus Aspergillus, Penicillium, Rhizopus, Chrysosporium, Myceliophthora, Trichoderma, Humicola, Acremonium or Fusarium.

99. The recombinant cell of claim 98, wherein the filamentous fungal cell is selected from the species Aspergillus niger, Aspergillus oryzae, Trichoderma reesei, Penicillium chrysogenum, Myceliophthora thermophila, or Rhizopus oryzae.

100. The recombinant cell of claim 97, wherein the cell is a yeast cell.

101. The recombinant cell of claim 100, wherein the yeast cell is selected from the genus Saccharomyces, Kluyveromyces, Candida, Pichia, Schizosaccharomtces, Hansenula, Klockera, Schwanniomyces or Yarrowia.

102. The recombinant cell of claim 101, wherein the yeast cell is selected from the species S. cerevisiae, S. bulderi, S. barnetti, S. exiguus, S. uvarum, S diastaticus, K. lactis, K. marxianus or K. fragilis.

103. An isolated host cell transformed with the vector of claim 94.

104. A method for generating a variant CBH II polypeptide having increased specific activity as compared to a reference CBH II polypeptide, comprising
modifying the nucleotide sequence of a CBH II-encoding nucleic acid so that the nucleic acid encodes a variant CBH II polypeptide, wherein said variant CBH II polypeptide comprises one or more amino acid substitutions selected from:
an I235V substitution;
a P64W substitution;
a P64E substitution;
a L21R substitution;
a S104V substitution;
a G37S substitution;
a G65L substitution;
a K309H substitution;
an E66R substitution;
an S115A substitution;
a G67K substitution;
an E23K substitution;
a S115M substitution;
an A33K substitution; and/or
an E23N substitution; and/or
modifying the nucleotide sequence of a CBH II-encoding nucleic acid so that the nucleic acid encodes a variant CBH II polypeptide, wherein said variant CBH II polypeptide comprises one or more substitutions selected from:
a D194N substitution;
an A200L substitution;
a S42 IC substitution;
a D426N substitution;
an A429S substitution;
an T430P substitution;
a Y434A substitution;
an A438L substitution;
a S439P substitution;
an A440D substitution;
a L442T substitution;
a Q443P substitution; and/or
a P444N substitution; and/or
modifying the nucleotide sequence of a CBH II-encoding nucleic acid so that the nucleic acid encodes a variant CBH II polypeptide, wherein said variant CBH II polypeptide comprises one or more substitutions selected from:
an I235V substitution;
a P64W substitution;
a P64E substitution;
a L21R substitution;
a S104V substitution;
a G37S substitution;
a G65L substitution;
a K309H substitution;
an E66R substitution;
an S115A substitution;
a G67K substitution;
an E23K substitution;
a S115M substitution;
an A33K substitution; and/or
an E23N substitution;
modifying the nucleotide sequence of a CBH II-encoding nucleic acid so that the nucleic acid encodes a variant CBH II polypeptide, wherein said variant CBH II polypeptide comprises one or more substitutions selected from:
a D194N substitution;
an A200L substitution;
a S421C substitution;
a D426N substitution;
an A429S substitution;
a T430P substitution;
a Y434A substitution;
an A438L substitution;
a S439P substitution;
an A440D substitution;
a L442T substitution;
a Q443P substitution; and/or
a P444N substitution;
thereby generating a nucleic acid that encodes a CBH II polypeptide having increased specific activity as compared to a reference CBH II polypeptide.

105. The method of claim 78 or 104, wherein the modification or substitution is achieved by site directed mutagenesis.
